(19) United States
(12) Patent Application Publication
(10) Pub. No.: US 2023/0310582 A1
(43) Pub. Date: Oct. 5, 2023

(54) SARS-2 SPIKE PROTEIN DESIGNS, COMPOSITIONS AND METHODS FOR THEIR USE

(71) Applicant: Duke University, Durham, NC (US)

(72) Inventors: Rory Henderson, Durham, NC (US); Kevin Saunders, Durham, NC (US); Barton F. Haynes, Durham, NC (US); Priyamvada Acharya, Durham, NC (US)

(21) Appl. No.: 17/919,111

(22) PCT Filed: Apr. 14, 2021
(86) PCT No.: PCT/US2021/027242

§ 371 Date: Oct. 14, 2022

Related U.S. Application Data
(60) Provisional application No. 63/009,969, filed on Apr. 14, 2020.
(60) Provisional application No. 63/026,588, filed on May 18, 2020.
(60) Provisional application No. 63/044,629, filed on Jun. 26, 2020.

(51) Int. Cl. A61K 39/215; C07K 14/005; A61P 31/14

(52) U.S. Cl. CPC A61K 39/215; C07K 14/005; A61P 31/14

(57) ABSTRACT

The invention provides SARS-2 spike protein designs and uses thereof.

This application claims the benefit of and priority to U.S. Patent Application No. 63/009,969 filed Apr. 14, 2020, U.S. Patent Application No. 63/026,588 filed May 18, 2020 and U.S. Patent Application No. 63/044,629 filed Jun. 26, 2020, the content of each application is herein incorporated by reference in its entirety.

This invention was made with government support under administrative supplement to NIH R01 AI145687 for coronavirus research and a grant from the State of North Carolina from the Federal CARES Act. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on May 21, 2021, is named 2933311-041WO1_SL and is 1,540,060 bytes in size.

### TECHNICAL FIELD

The invention relates, in general, to modified SARS-CoV-2 proteins, nucleic acids encoding these, methods of making recombinant proteins and nucleic acids, compositions comprising these and their use in vaccination regimens, and diagnostic assays.

### BACKGROUND

The ongoing global pandemic of the new SARS-CoV-2 coronavirus presents an urgent need for the development of effective preventative and treatment therapies.

Development of an effective vaccine for prevention of coronavirus (SARS-2) infection is a global priority.

### SUMMARY

In certain aspects the invention provides SARS-CoV-2 (“SARS-2”) spike protein designs. In certain embodiments, the protein design provides a stabilized protein conformation(s) of the SARS-2 spike protein trimer. In non-limiting embodiments, the modified SARS-2 spike protein comprising S383C D985C (rS2d) amino acid changes as described in FIGS. 8 and 11. The rS2d coronavirus design can comprise additional modification, for e.g. without limitations as described in FIG. 11. Modification can also include N165A or the N234A changes in the spike protein as described in Example 4. The modifications can be incorporated in full length sequences, ectodomain or any other SARS-2 protein fragment.

In certain embodiments, the inventive designs are recombinant proteins. In certain embodiments, the inventive designs are nucleic acids. Nucleic acids include without limitation modified mRNAs.

In certain aspects, the invention provides modified SARS-2 spike proteins, for example but not limited in a stabilized conformation, nucleic acid molecules and vectors encoding these proteins, and methods of their use and production are disclosed. In several embodiments, the modified SARS-2 spike proteins and/or nucleic acid molecules can be used to generate an immune response to coronavirus in a subject. In some embodiments, the proteins and/or nucleic acid molecules can be used to generate an immune response to SARS-2 in a subject. In additional embodiments, the therapeutically effective amount of the modified SARS-2 spike proteins and/or nucleic acid molecules can be administered to a subject in a method of treating or preventing coronavirus infection. In some embodiments, the proteins and/or nucleic acid molecules can be administered to a subject in a method of treating or preventing SARS-2 infection. In certain embodiments, the proteins of the invention can be used in diagnostic assays.

In certain aspects, the invention provides coronavirus (e.g. SARS-2) S protein ectodomain trimers in a stabilized conformation, nucleic acid molecules and vectors encoding these proteins, and methods of their use and production. In several embodiments, the coronavirus (e.g. SARS-2) S protein ectodomain trimers and/or nucleic acid molecules can be used to generate an immune response to coronavirus in a subject. In some embodiments, the proteins and/or nucleic acid molecules can be used to generate an immune response to SARS-2 in a subject. In additional embodiments, the therapeutically effective amount of the coronavirus (e.g. SARS-2) S protein ectodomain trimers and/or nucleic acid molecules can be administered to a subject in a method of treating or preventing coronavirus infection. In some embodiments, the proteins and/or nucleic acid molecules can be administered to a subject in a method of treating or preventing SARS-2 infection. In certain embodiments, the proteins of the invention can be used in diagnostic assays.

In certain embodiments, the modified SARS-2 spike proteins do not include modification as described in US Patent Publication 20200061185. In certain embodiments the modified SARS-2 spike proteins do not include the two proline modification (K986P+V987P (2P)) substitutions in the S2 domain. See Edwards et al. Nature Structural & Molecular Biology volume 28, pages 128-131(2021) and references therein.

The invention provides amino acid or nucleic acids sequences encoding such spike protein designs. Provided are also nucleic acids, including modified mRNAs which are stable and can be used as immunogens. Non-limiting embodiments include recombinant proteins, trimers, multimerized proteins, e.g. but not limited to nanoparticles. Provided also are nucleic acids optionally designed as vectors, for example for recombinant expression and/or stable integration, e.g. but not limited to, a DNA encoding trimer for stable expression, or virus-like particle (VLP) incorporation. In non-limiting embodiments a DNA encodes a SARS-2 spike protein for stable expression. In non-limiting embodiments a DNA encodes a SARS-2 spike protein for stable expression as a protomer which trimerizes to form a SARS-2 spike protein trimer. In non-limiting embodiments, nucleic acids are mRNA, including but not limited to modified mRNA which are used immunogens. Modified mRNAs can be formulated in any suitable formulation, including but not limited to lipidnanoparticles (LNPs) and/or liposomes.

In some embodiments a protein design is based on SARS-2 spike protein if it is characterized as having 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% similarity or identity to the designs described herein.

In non-limiting embodiments the invention provides SARS-2 S protein trimers stabilized in a prefusion conformation, nucleic acid molecules and vectors encoding these proteins, and methods of their use and production. In several embodiments, the SARS-2 S protein trimers and/or nucleic acid molecules can be used to generate an immune response to coronavirus in a subject. In some embodiments, the protein trimers and/or nucleic acid molecules can be used to generate an immune response to SARS-2 in a subject. In additional embodiments, the therapeutically effective amount of the SARS-2 S protein trimers and/or nucleic acid molecules can be administered to a subject in a method of treating or preventing coronavirus infection. In some embodiments, the protein trimers and/or nucleic acid molecules can be administered to a subject in a method of treating or preventing SARS-2 infection.

In certain aspects, the invention provides a modified SARS-2 spike protein comprising a sequence modified with amino acid changes as described herein. In certain aspects, the invention provides a recombinant, non-naturally occurring SARS-2 spike protein comprising a sequence modified with amino acid changes as described herein. Non-limiting embodiments of sequences are shown in FIG. 8, 10 and FIG. 25.

In certain aspects, the invention provides a recombinant SARS-2 spike protein comprising all the consecutive amino acids after the signal peptide of the amino acid sequences described herein. For specific non-limiting embodiments of sequences see FIG. 8, 10 and FIG. 25.

In certain aspects, the invention provides a nucleic acid encoding the modified SARS-2 spike protein described herein. In non-limiting embodiments, the nucleic acid is a modified mRNA. In certain embodiments, the mRNA is in a composition comprising LNPs. In certain embodiments, the mRNA is in a composition comprising liposomes.

In certain embodiments, the nucleic acid is comprised in a vector and is operably linked to a promoter.

In certain embodiments the sequence is modified with modifications described as Clusters 1-11. In certain embodiments the design can comprise any combination of modifications within any one Cluster, and/or combination of modifications from any of the modifications from any one of Clusters 1-11 (FIGS. 8 and 10). In non-limiting embodiments, the combinations are: D985C+S383C; D985C+S383C, T866C+G669C, L966C+A570C; K41C+A520C; D985C+S383C, T866C+G669C, F43C+G566C; K41C+A520C, T866C+G669C, L966C+A570C.

In certain embodiments, the invention provides modified SARS-2 spike protein comprising any combination of modifications within any one Cluster and further comprising N165A variation or the N234A variation as described in Example 4. Additional stabilizing mutations can be added to these modified SARS-2 spike designs.

Any one of the modifications described herein can be engineered in a full length SARS-2 S sequence or in a fragment, e.g. but not limited to the ectodomain.

In certain aspects the invention provides a composition comprising a recombinantly produced modified SARS-2 spike protein of any one of the claims and a carrier. In certain embodiments, the compositions are immunogenic. In certain embodiments the compositions comprised an adjuvant. Any suitable adjuvant can be used.

In certain aspects the invention provides a composition comprising a nucleic acid encoding any of the modified SARS-2 spike proteins and a carrier. Non-limiting embodiments of nucleic acids are shown in FIG. 25. Embodiments herein are also modified mRNA, for example comprising suitable modifications for expression as immunogens. Non-limiting examples include modified nucleosides, capping, polyA tail, and the like. In certain embodiments the compositions comprise an adjuvant.

In certain embodiments the designs produce a soluble protein. In certain embodiments the designs are comprised in a protomer which can form a trimer. In certain embodiments the designs comprise a transmembrane (TM) domain.

In certain embodiments the compositions comprise a SARS-2 S ectodomain trimer comprising protomers comprising sequence modification as described here in.

In non-limiting embodiments, the designs comprise additional modifications to allow multimerization. In non-limiting embodiments, wherein the design comprises a soluble ectodomain, additional modifications can be included to allow multimerization. In a non-limiting embodiment, a C-terminal residue of the protomers in the ectodomain of the modified SARS-2 spike protein is linked to a trimerization domain by a peptide linker, or is directly linked to the trimerization domain. In some embodiments, the trimerization domain is a T4 fibritin trimerization domain. In one example, a T4 fibritin trimerization domain comprises the amino acid sequence set forth as GYIPEAPRDGQAYVRKDGEWVLLSTF (SEQ ID NO: 1). In some embodiments, a protease cleavage site (such as a thrombin cleavage site) can be included between the C-terminus of the recombinant SARS-2 spike protein ectodomain and the T4 fibritin trimerization domain to facilitate removal of the trimerization domain as needed, for example, following expression and purification of the recombinant SARS-2 S ectodomain.

In certain embodiments, the modified SARS-2 spike protein further comprises one or more additional amino acid substitutions that stabilize the recombinant ectodomain trimer in the prefusion conformation.

In certain embodiments, the modified SARS-2 spike designs further comprise furin protease cleavage sites and/or a cathepsin L cleavage site. In certain embodiments, the modified SARS-2 spike protein trimer is soluble.

In certain embodiments, a C-terminal residue of the protomers in the ectodomain of the modified SARS-2 spike protein is linked to a transmembrane domain by a peptide linker, or is directly linked to the transmembrane domain. In certain embodiments, the modified SARS-2 spike protein is linked to form a protein nanoparticle subunit by a peptide linker, or is directly linked to the protein nanoparticle subunit. In certain embodiments, the protein nanoparticle subunit is a ferritin nanoparticle subunit.

In certain aspects the invention provides a protein nanoparticle, comprising any one of the protein immunogens of the invention.

In certain aspects the invention provides a virus-like particle comprising any one of the immunogens of the invention.

In certain aspects the invention provides an isolated nucleic acid molecule encoding a protomer of the modified SARS-2 spike protein of the invention. In certain embodiments, the nucleic acid molecule is operably linked to a promoter. In certain embodiments, the nucleic acid molecule is an RNA molecule.

In certain aspects, the invention provides a vector comprising a nucleic acid molecule encoding any one of the inventive proteins. In certain embodiments, the vector is a viral vector.

In certain aspects, the invention provides an immunogenic composition comprising any one of the proteins and/or nucleic acids of the invention, and a pharmaceutically acceptable carrier.

In certain aspects, the invention provides a method of producing a recombinant SARS-2 spike protein of the invention, comprising: expressing the nucleic acid molecule or vector comprising a nucleic acid encoding in a host cell to produce the recombinant protein, which in certain embodiments is a trimer; and purifying the recombinant protein.

In certain aspects, the invention provides a recombinant cell comprising a nucleic acid encoding the modified SARS-2 spike protein of the invention.

In certain aspects the invention provides a method for generating an immune response to an SARS-CoV-2 in a subject, comprising administering to the subject an effective amount of any one of the immunogens, wherein the immunogen is a recombinant protein, a nucleic acid, and/or a combination thereof to induce an immune response. In certain embodiments, the recombinant protein is formulated with any suitable adjuvant. In certain embodiments, the nucleic acid is DNA which can be administered by any suitable method. In certain embodiments, the nucleic acid is an mRNA, which can be administered by any suitable methods. In certain embodiments, the mRNA is formulated in an LNP. In certain embodiments, the mRNA is formulated in a liposome LNP. A skilled artisan can readily determine the dose and number of immunizations needed to induce immune response. Various assays are known and used in the art to measure to level, breadth and durability of the induced immune response.

In certain aspects the invention provides modified coronavirus spike proteins designs including but not limited to protein designs comprising spike protein and/or various spike portions/domains from SARS-CoV-2 (SARS-2), SARS-CoV-1 (CoV1), MERS, or any other coronavirus spike protein, wherein in certain embodiments these proteins are designed to form multimeric complexes. The invention provides amino acid and nucleic acid sequences of recombinant coronavirus spike proteins or portions thereof, wherein in certain embodiments these spike proteins or portions/domains are multimerized, and can be used as an antigen to induce an immunogenic response. In some embodiments the antigen comprises any suitable portion from a spike protein. In non-limiting embodiments are portions of the spike protein which comprise epitopes conserved between different coronaviruses. In some embodiments the antigen comprises RBD domain from a spike protein. In some embodiments the antigen comprises NTD domain from a spike protein. In some embodiments the antigen comprises FP domain from a spike protein. The sequence of the spike protein is any suitable sequence coronavirus sequence including without limitation SARS-CoV1, SARS-CoV2, MERS, bat coronavirus, pangolin or other animal coronaviruses. In non-limiting embodiments the spike protein sequences comprise any variation in amino acid sequences, including without limitation Wuhan SARS-CoV2 sequence, UK SARS-CoV2 variant B.1.1.7, South African variant 1.351, US SARS-CoV-2 variants with L452R mutations and Brazilian variant P.1. Additional SARS-2 spike protein sequences from circulating viruses are found in the GISAID EpiFlu™ Database. These sequences can also be designed with any of the modifications described herein. In certain embodiments, the immune response treats, prevents or inhibits infection with the SARS-CoV-2. In certain embodiments, the immune response generated by the immunogens inhibits replication of the SARS-CoV-2 in the subject.

In certain aspects the invention provides a modified SARS-2 spike protein sequence or amino acid sequence encoding the same, wherein the protein sequence comprising amino acid changes as described in FIG. 8, 10 or 25. Non-limiting embodiments of sequences comprising specific amino acid changes are shown in FIG. 8 (Clusters 1-11), FIG. 10 (sequences with rsd2 mutations and comprising additional modifications, for example selected from Cluster mutations) or FIG. 25. A modified SARS-2 spike protein sequence or amino acid sequence encoding the same, wherein the protein sequence comprising S383C D985C (rS2d) amino acid changes as described in FIG. 8, 10 or 25. In a non-limiting embodiment, modified SARS-2 spike protein comprising S383C D985C (rS2d) is shown in Table 8 and FIG. 25P.

In certain aspects the invention provides a recombinant SARS-2 spike protein comprising all the consecutive amino acids after the signal peptide of the polypeptide sequences in FIG. 8, 10 or 25. Specific non-limiting embodiments of sequences are shown in FIG. 8, 10 or FIG. 25.

In certain aspects the invention provides a nucleic acid encoding the modified SARS-2 spike protein of any of the preceding claims.

In certain embodiments, the nucleic acid of any of the preceding claims is a modified mRNA. In certain embodiments, the mRNA is in a composition comprising LNPs.

In certain embodiments, the nucleic acid is comprised in a vector and is operably linked to a promoter.

In certain aspects, the invention provides a composition comprising a recombinantly produced modified SARS-2 spike protein of any one of the claims and a carrier. In certain embodiments, the compositions comprise a trimer comprising protomers with amino changes as described herein. In certain embodiments, the compositions are immunogenic. In certain embodiments the compositions comprised an adjuvant. Any suitable adjuvant can be used.

In certain aspects, the invention provides a composition comprising a nucleic acid encoding any of the modified SARS-2 spike proteins and a carrier.

In certain aspects, the invention provides a protein nanoparticle, comprising any one of the protein immunogens of the invention. In certain embodiments, the protein nanoparticle subunit is a ferritin nanoparticle subunit.

In certain aspects, the invention provides a virus-like particle comprising any one of the immunogens of the invention.

In certain aspects, the invention provides a host cell comprising a nucleic acid molecule encoding a modified SARS-2 spike protein of the invention.

In certain aspects, the invention provides a method of producing a recombinant SARS-2 protein of the invention, comprising: expressing the nucleic acid molecule or vector comprising a nucleic acid encoding in a host cell to produce the recombinant protein, which in certain embodiments is a trimer; and purifying the recombinant protein.

In certain aspects, the invention provides an immunogenic composition comprising any one of the proteins, nucleic acids, nanoparticle or VLP of the preceding claims and a pharmaceutically acceptable carrier. The immunogenic composition of the preceding claim, further comprising an adjuvant.

In certain aspects, the invention provides a method for inducing an immune response to an SARS-2 in a subject, comprising administering to the subject an effective amount of any one of the immunogens and/or the immunogenic composition of the preceding claims to induce an immune response.

In certain aspects, the invention provides a modified SARS-2 spike protein comprising the amino acid sequence of the N165A variant or the N234A variant.

In certain aspects, the invention provides a recombinant SARS-2 spike protein comprising all the consecutive amino acids after the signal peptide of a modified SARS-2 spike protein comprising the amino acid sequence of the N165A variant or the N234A variant.

In certain aspects, the invention provides a nucleic acid encoding a modified SARS-2 spike protein of the invention or a recombinant SARS-2 spike protein of the invention.

In certain embodiments, the nucleic acid of the invention is comprised in a vector and is operably linked to a promoter. In certain embodiments, the nucleic acid of the invention is operably linked to a promoter suitable for in vitro mRNA expression.

In certain embodiments, a nucleic acid of the invention is a modified mRNA. In certain embodiments, the mRNA is in a composition comprising LNPs.

In certain aspects, the invention provides a composition comprising a recombinantly produced modified SARS-2 spike protein, or a nucleic acid encoding a recombinant protein of the invention and a carrier. In certain embodiments, the compositions comprise a trimer comprising protomers with amino changes as described herein. In certain embodiments, the compositions are immunogenic. In certain embodiments the compositions comprised an adjuvant. Any suitable adjuvant can be used.

In certain aspects the invention provides a protein nanoparticle, comprising a modified recombinant SARS-2 spike protein of the invention. In certain embodiments, the protein nanoparticle subunit is a ferritin nanoparticle subunit.

In certain aspects the invention provides a virus-like particle, comprising a modified recombinant SARS-2 spike protein of the invention.

In certain aspects the invention provides a host cell comprising a nucleic acid molecule encoding the modified SARS-2 spike protein of the invention. In certain aspects the invention provides an in vitro transcription reaction comprising a nucleic acid encoding anyone of the modified SARS-2 spike protein of the invention and reagents suitable for carrying out the in vitro transcription reaction to produce mRNA, including without limitation modified mRNA.

In certain aspects, the invention provides methods of producing a modified SARS-2 spike protein of the invention, comprising: expressing the nucleic acid molecule or vector comprising a nucleic acid encoding in a host cell to produce the recombinant protein, which in certain embodiments is a trimer; and purifying the recombinant protein.

In certain aspects, the invention provides an immunogenic composition comprising any one of the proteins, nucleic acids, nanoparticle or VLP of the invention and a pharmaceutically acceptable carrier. In certain embodiments, the immunogenic composition further comprises an adjuvant.

In certain aspects the invention provides, a method for inducing an immune response to an SARS- 2 in a subject, comprising administering to the subject an effective amount of any one of the immunogens and/or the immunogenic composition of the invention in an amount and manner sufficient to induce an immune response.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1C: structure of the SARS-CoV S-protein. A) ‘Down’ configuration of the S-protein. Single protomer colored according to (C, upper). Other two protomers colored according to (C, lower). B) ‘Up’ configuration of the S-protein colored as in (A). C) Line diagram of the SARS-CoV S-protein. The NTD′ region is highlighted in cyan. HR2 was not resolved in this structure.

FIGS. 2A-2F: Vector based analysis of the CoV S-protein. A) A single protomer of the CoV S-protein with labeled domains. B) A simplified diagram of the CoV S-protein depicting the centroids and vectors connecting them with the determine angles (θ) and dihedrals (ϕ) labeled. C) The SARS-2 (left; red) and MERS (right; blue) structures each with a single protomer depicted in a cartoon representation and the remaining two in a surface representation. D) Principal components analysis of the SARS and MERS protomers including measures between S1 and S2 domains. E) Principal components analysis of the SARS, MERS, HKU1, and Murine CoV protomers including measures only between S1 domains. F) Cluster plots of the angles and dihedrals between S1 and S2 domains.

FIGS. 3A-3C: Purification of recombinant SARS-2 S protein and binding to ACE-2 receptor. A) SDS-PAGE of the SARS-2 S protein. Lane 1: molecular weight ladder, with relevant bands labeled in kilodaltons; Lanes 2 and 3: Elution from StrepTactin resin under reducing (Lane 2) and non-reducing (Lane 3) conditions; Lanes 4 and 5: Purified protein after SEC under reducing (Lane 4) and non-reducing (Lane 5) conditions. The SARS-2 S protein band is denoted with a black arrow. (B) SEC of the affinity-purified SARS-2 S protein. (C) SPR sensorgrams showing binding of different concentrations of the human ACE2 receptor to immobilized SARS-2 S protein.

FIGS. 4A-4C: NSEM of the recombinant SARS-2 spike. A) Representative micrograph with particle picks shown in green. B) Representative 2D class averages. C) 3D reconstructed map shown as a semi-transparent grey surface with underlying fitted model (PDB 6VSB) shown in ribbon representation. 166 micrographs were collected on a Philips EM420 microscope. A total of 85,341 particles were picked. After multiple rounds of 2D and 3D classifications, an asymmetric 3D reconstruction at an overall resolution of ˜17.5 Å was obtained from a final cleaned-up stack of 41,941 particles.

FIGS. 5A-5C: Cryo-EM of the recombinant SASR-CoV-2 spike. A) Representative micrograph. B) Representative 2D class averages. C) Cryo-EM map at 5.6 Å resolution depicting side and top views.

FIGS. 6A-6E: Molecular simulation guided mechanism for CoV S-protein closed, ‘down’ to open ‘up’ configuration. A) Counts plot for the first two time-lagged independent components analysis. B) Implied time-scale plot depicting Markov model-based timescales for different processes in the dataset at differing lag-times. C) Two representative macrostates from the Markov model. A down state, tight (DTE) configuration and a down state mobile (DUtDE) configuration. D) Nested plots of vector-based dihedrals comparing RBD motion, NTD motion, and S1 to S2 motion, from top to bottom, respectively, with the DTE (left) and DUtDE (right) values with mean and standard deviation. E) Mechanism for closed to open transitions.

FIG. 7: SARS-2 sites for differential domain stabilization. Image depicts the closed, all RBD ‘down’ state trimer colored according to FIG. 1 (C). Mutation clusters are identified with c-α atoms of mutable residues shown as spheres with mutants identified next to the cluster image. Mutations developed based upon MHV, MERS, and simulation results are noted beside their respective mutants.

FIG. 8A-L shows non-limiting embodiments amino acid sequences of SARS-2 protein designs comprising certain modifications—Cluster 1-11 designs. FIG. 8A shows Parent sequence (nCoV-1 nCoV-2P), FIG. 8B (including FIGS. 8B-1 to 8B-7) shows Cluster 1 modifications, FIG. 8C (including FIGS. 8C-1 to FIG. 8C-7) shows Cluster 2 modifications, FIG. 8D (including FIG. 8D-1 to FIG. 8D-10) shows Cluster 3, FIG. 8E (including FIG. 8E-1 to FIG. 8E-6) shows Cluster 4 modifications, FIG. 8F (including FIG. 8F-1 to FIG. 8F-6) shows Cluster 5 modifications, FIG. 8G (including FIG. 8G-1 to FIG. 8G-6) shows Cluster 6 modifications, FIG. 8H (including FIG. 8H-1 to FIG. 8H-4) shows Cluster 7 modifications, FIG. 8I (including FIG. 8I-1 to FIG. 8I-7) shows Cluster 8 modifications, FIG. 8J (including FIG. 8J-1 to FIG. 8J-12) shows Cluster 9 modifications, FIG. 8K (including FIG. 8K-1 to FIG. 8K-8) shows Cluster 10 modifications, FIG. 8L (including FIG. 8L-1 to FIG. 8L-6) shows Cluster 11 modifications. Underlined amino acids indicate positions of cluster amino acid changes. A skilled artisan can readily determine the signal peptide sequences. Signal peptide sequences can be removed during recombinant production of proteins. In non-limiting embodiments, provided are amino acid sequences of recombinant proteins which do not include amino acids of comprising a signal peptide. The sequence presented here are of the ectodomain. The modifications can be incorporated in full length sequences, or any other SARS-2 protein fragment. The modifications can be incorporated in sequences which do not comprise the 2P mutations. FIG. 8A-L discloses SEQ ID NOS 6-85, respectively in order of appearance.

FIGS. 9A-B shows non-limiting embodiments of amino acid sequence of SARS-2 protein designs. FIGS. 9A-B disclose SEQ ID NOS 86-87, respectively, in order of appearance.

FIG. 10A-M shows non-limiting embodiments of amino acid sequences of SARS-2 protein designs comprising rS2d mutations and further modifications selected from the cluster designs. FIG. 10A-10H show rS2d+S2 modification. FIG. 10I shows rS2d plus SD2 to S2. FIG. 10J-10M show S2 stabilization and SD2 to S2. Additional cluster modifications can be combined with rS2d mutations. The modifications can be incorporated in full length sequences, or any other SARS-2 protein fragment. FIG. 10A-M discloses SEQ ID NOS 88-100, respectively, in order of appearance.

FIG. 11A-C. SARS-CoV-2 mRNA-lipid nanoparticle (LNP) vaccines elicited neutralizing antibodies in rhesus macaques. (A) Schematic diagram of the mRNA-LNP vaccines in this study. The mRNA-LNP vaccines that encode monomer receptor-binding domain (RBD), K986P/V987P mutations stabilized full-length Spike protein (Spike 2P), S383C/D985C/K986PN987P mutations stabilized full-length Spike protein (Spike 2P 2C), or unstabilized Spike protein were compared. A luciferase expressing mRNA-LNP vaccine was made as a control. (B) Rhesus macaque vaccination and challenge regimen. Rhesus macaque (n=8 per group) were immunized intramuscularly by mRNA-LNP vaccine for two times in Week 0 and 4, followed by 10⁵ PFU of SARS-CoV-2 challenge via intranasal and intratracheal routes. Respiratory samples including bronchoalveolar lavage (BAL) and nasal swab were collected on Day 0, 2, 4, 7 post-challenge for subgenomic RNA (sgRNA) viral load test, and were measured at the indicated pre-challenge and post-challenge timepoints. Lungs were harvested by necropsy on Week 11 and 12 for histopathology analysis. (C) Vaccine-induced SARS-CoV-2 specific antibodies. Serum IgG binding activities to Spike 2P (S-2P), S-2P D614G, RBD, n-terminal domain (NTD), and S2 domain were tested by ELISA and shown as log AUC mean value±SEM.

FIG. 12. Vaccine-induced antibodies block ACE-2 and neutralizing antibodies binding to Spike protein. The ability of serum blocking ACE-2, RBD neutralizing antibodies DH1041 and DH1047, NTD neutralizing antibodies DH1050.1 and NTD non-neutralizing antibodies DH1052 from binding to S-2P were tested by ELISA. Percentage of blocking (mean value±SEM) were shown.

FIG. 13. SARS-CoV-2 mRNA-lipid nanoparticle (LNP) vaccines elicited neutralizing antibodies in rhesus macaques.

Vaccine-induced neutralizing antibodies against pseudotyped (top panels) or live (bottom panels) SARS-CoV-2 viruses. ID50, inhibitory dilutions at which 50% viruses were neutralized. Each dot indicates one animal, and the bars show geometric means. Pseudovirus assays were performed in 293T/ACE2 cells, and live SARS-CoV-2 microneutralization assays were performed in Vero cells.

FIG. 14. Reduced SARS-CoV-2 viral replication in respiratory tract of vaccinated macaques. (A-B) SARS-CoV-2 (A) envelope gene (E gene) sgRNA and (B) nucleocapsid gene (N gene) sgRNA in bronchoalveolar lavage (BAL) samples on Day 0 (pre-challenge), Day 2, Day 4 and Day 7 post challenge.

FIG. 15. Reduced SARS-CoV-2 viral replication in respiratory tract of vaccinated macaques. SARS-CoV-2 (top panels) E gene sgRNA and (bottom panes) N gene sgRNA in nasal swab samples on Day 0 (pre-challenge), Day 2, Day 4 and Day 7 post challenge.

FIG. 16A-D. Bronchoalveolar lavage (BAL) fluid cytokine responses before and after challenge in vaccinated macaques. 16A-C show Cytokines (IL-16, IP-10, IL-1aa) concentrations in BAL samples on Day 0 (pre-challenge), Day 2, Day 4 and Day 7 post challenge are shown for each macaque. Horizontal bars indicate group means. 16D shows the symbols used in 16A-C, and FIG. 17.

FIG. 17. Bronchoalveolar lavage (BAL) fluid cytokine responses before and after challenge in vaccinated macaques. Cytokines (FGF-2, Eotaxin, Fractalkine, MIP-3a) concentrations in BAL samples on Day 0 (pre-challenge), Day 2, Day 4 and Day 7 post challenge are shown for each macaque. Horizontal bars indicate group means.

FIG. 18A-E. Bronchoalveolar lavage (BAL) fluid cytokine responses before and after challenge in vaccinated macaques. 18A-C show Cytokines concentrations in BAL samples on Day 0 (pre-challenge), Day 2, Day 4 and Day 7 post challenge are shown for each macaque. Horizontal bars indicate group means. 18E shows the symbols for 18A-D.

FIG. 19A-D. Bronchoalveolar lavage (BAL) fluid cytokine responses before and after challenge in vaccinated macaques. Cytokines concentrations in BAL samples on Day 0 (pre-challenge), Day 2, Day 4 and Day 7 post challenge are shown for each macaque. Horizontal bars indicate group means.

FIG. 20A-D. Bronchoalveolar lavage (BAL) fluid cytokine responses before and after challenge in vaccinated macaques. Cytokines concentrations in BAL samples on Day 0 (pre-challenge), Day 2, Day 4 and Day 7 post challenge are shown for each macaque. Horizontal bars indicate group means.

FIG. 21A-E. Bronchoalveolar lavage (BAL) fluid cytokine responses before and after challenge in vaccinated macaques. Cytokines (21A-D) concentrations in BAL samples on Day 0 (pre-challenge), Day 2, Day 4 and Day 7 post challenge are shown for each macaque. Horizontal bars indicate group means. FIG. 21E shows the symbols used in 21A-D.

FIG. 22A-D. Bronchoalveolar lavage (BAL) fluid cytokine responses before and after challenge in vaccinated macaques. Cytokines concentrations in BAL samples on Day 0 (pre-challenge), Day 2, Day 4 and Day 7 post challenge are shown for each macaque. Horizontal bars indicate group means.

FIG. 23A-B. Monoclonal antibody isolation from RBD and S mRNA-LNP immunized macaques. (A) FACS plot of sort strategy for each macaque. (B) summary antibody specificities based on initial binding screen of monoclonal antibodies.

FIG. 24. Cross-reactive RBD-specific monoclonal antibody were elicited in SARS-CoV-2 mRNA-LNP immunized macaques. Heatmap of binding magnitude (log AUC) for a subset of monoclonal antibodies isolated from vaccinated macaques. RBD. RBD antibodies bound to bat and pangolin coronaviruses (BCoV RaTG13 and PC0V GXP4L).

FIG. 25A-25Q shows non-limiting embodiments of SARS-2 designs comprising various modifications. The modifications can be incorporated in full length sequences, or any other SARS-2 protein fragment. FIG. 25A includes FIGS. 25A-1 to FIG. 25A-4. FIG. 25B includes FIGS. 25B-1 to FIG. 25B-4. FIG. 25C includes FIGS. 25C-1 to FIG. 25C-4. FIG. 25D includes FIGS. 25D-1 to FIG. 25D-4. FIG. 25E includes FIGS. 25E-1 to FIG. 25E-4. FIG. 25F includes FIGS. 25F-1 to FIG. 25F-4. FIG. 25H includes FIGS. 25H-1 to FIG. 25H-8. FIG. 25I includes FIGS. 25I-1 to FIG. 25I-4. FIG. 25J includes FIGS. 25J-1 to FIG. 25J-4. FIG. 25K includes FIGS. 25K-1 to FIG. 25K-4. FIG. 25L includes FIGS. 25L-1 to FIG. 25L-4. FIG. 25M includes FIGS. 25M-1 to FIG. 25M-4. FIG. 25N includes FIGS. 25N-1 to FIG. 25N-4. FIG. 25Q includes FIGS. 25Q-1 to FIG. 25Q-8. FIG. 25A-25Q disclose SEQ ID NOS 101-167, respectively, in order of appearance.

FIG. 26A-26F show vector based analysis of the CoV S-protein demonstrates remarkable variability in S-protein conformation within ‘up’ and ‘down’ states between CoV strains. A) Cartoon representations of the ‘down’ (upper left) and ‘up’ (upper right) state structures colored according to the specified domains (lower). B) A single protomer of the CoV S-protein with labeled domains. C) A simplified diagram of the CoV S-protein depicting the centroids and vectors connecting them with the determine angles (θ) and dihedrals (ϕ) labeled. D) The SARS-2 (left; red) and MERS (right; blue) structures each with a single protomer depicted in a cartoon representation and the remaining two in a surface representation. E) Principal components analysis of the SARS and MERS protomers including measures between S1 and S2 domains. F) Principal components analysis of the SARS, MERS, HKU1, and Murine CoV protomers including measures only between S1 domains.

FIG. 27A-27J show vector based analysis of the CoV S-protein demonstrates remarkable variability in S-protein conformation within ‘up’ and ‘down’ states between CoV strains. A) Angle between the subdomain 1 to subdomain 2 vector and the subdomain 1 to RBD vector. B) Dihedral about the subdomain 1 to RBD vector. C) Angle between the RBD to subdomain 1 vector and the RBD to RBD helix vector. D) Dihedral about the subdomain 2 to subdomain 1 vector. E) Angle between the NTD′ to NTD vector and the NTD to NTD sheet motif vector. F) Dihedral about the dihedral aout the NTD to NTD′ vector. G) Angle between the NTD′ to subdomain 2 vector and the NTD′ to NTD vector. H) Angle between the subdomain 2 to NTD′ vector and the subdomain 2 to subdomain 1 vector. I) Diagram of the domains and relevant angles and dihedrals for S1 J) Cartoon representation of one protomer's S1 domains in the ‘down’ state overlaid with a ribbon representation of the ‘up’ state colored according to (I). Black (‘down’ state) and grey (‘up’ state) spheres represent domain centroids with lines connecting representing the vectors. Adjacent protomers represented as transparent surfaces.

FIGS. 28A-28F show negative stain electron microscopy analysis of S-protein constructs. A) Data tables, indicating construct names, mutations, observed classes, number and percent of particles per class and final resolution (gold-standard Fourier-shell correlation, 0.143 level). B) Raw micrographs. C) Representative 2D class averages. D) 3D reconstructions of 3-RBD-down classes, shown in top view, looking down the S-protein 3-fold axis on the left and tilted view on the right. Receptor binding domains and N-terminal domains of first structure marked with R and N, respectively. E) 3D reconstructions of 1-RBD-up classes. Up-RBD is marked with an asterisk. F) 3D reconstruction of 2-RBD-up class. Density for up-RBDs is weak, indicated by asterisks.

FIGS. 29A-29C show cryo-EM dataset reveals differential stabilization of the S-protein in the mutant ectodomain constructs. A) Two structural states of the SARS-CoV-2 S-protein ectodomain with the RBDs in the all ‘down’ state or a single RBD ‘up’ state. The resolution of the structure is provided below and to the left of each structure with the state population to the right. The S-protein spike highlighting the two regions of interest for structure and computation-based design. B) The rS2d RBD to S2 locked structure displaying only the all RBD down state. C) The u1S2q SD1 to S2 mutated structure displaying the all RBD ‘down’ state, the 1-RBD ‘up’ state, and, for the first time in the SARS-2 S ectodomain, the 2-RBD ‘up’ state.

FIGS. 30A-30D show cryo-EM structures of the “down” state in the r2S2d and u1S2q constructs reveal differential stabilization of domain positions. A) Alignment between the trimers of the designed disulfide linked rS2d (dark blue) mutant structure and the u1S2q (green). B) (left) Alignment between single protomers of the designed disulfide linked rS2d(dark blue) mutant structure and the u1S2q(green). (right) Zoomed in view of SD1 in both constructs demonstrating the shift in the subdomain with the 4 mutants. C) Structure and cryo-EM map depicting the RBD to S2 bridging density between the introduced cysteine residues. D) Structure and cryo-EM map depicting the SD1 and S2 mutations.

FIGS. 31A-31C show high-resolution structure of the u1S2q 1 RBD ‘up’ state reveals increasing relaxation of the triggered RBDs toward the unmutated structure. A) Cryo-EM map shown as grey mesh with underlying model in green cartoon representation; side (left) and top (right) views. B) Zoomed-in view showing the mutated residues. C) (top) Structure of the ‘up’ state RBD coupled ‘down’ state RBD (green) highlighting the shifted subdomain 1 to NTD′ position relative to the unmutated position (blue). (middle) Structure of the uncoupled ‘down’ state RBD (green) highlighting the moderately shifted subdomain 1 to NTD′ position relative to the unmutated position (blue). (bottom) Structure of the ‘up’ state RBD (green) highlighting the close alignment of subdomain 1 and the NTD′ regions to the unmutated position (blue).

FIGS. 32A-32C show structure of the u1S2q 2 RBD ‘up’ state indicates modest differences between the 1 RBD ‘up’ state's subdomain arrangement. A) Cryo-EM map structural alignment side view. B) Cryo-EM map structural alignment top view. C) Structure (green) and cryo-EM map depicting the mutated residue dispositions. The unmutated ‘up’ state protomer alignment is depicted in ribbons (blue).

FIG. 33 shows sites identified for differential stabilization of the SARS-CoV-2 S-protein. Single protomer colored according to FIG. 26 with remaining two protomers color according to S1 (light blue) and S2 (grey). Spheres indicate candidate mutation sites.

FIGS. 34A-34F show cryo-EM data processing details for r2S2d. (A) Representative micrograph. (B) CTF fit (C) Representative 2D class averages. (D) Ab initio reconstruction for the “down” state. (E) Refined map for the “down” state. (F) Fourier shell correlation curve for the “down” state.

FIGS. 35A-35L show cryo-EM data processing details for u1s2q. (A) Representative micrograph. (B) CTF fit (C) Representative 2D class averages. (D-F) Ab initio reconstructions for the (D) “down” state, (E) “1-up” state and (F) “2-up” state. (G-I) Refined maps for the (G) “down” state, (H) “1-up” state and (I) “2-up” state. (J-L) Fourier shell correlation curves for the (J) “down” state, (E) “1-up” state and (F) “2-up” state.

FIGS. 36A-36C show alignment of the rS2d and u1S2q designs with the unmutated construct. A) Structure of rS2d (dark blue) aligned to the unmutated construct (PDB ID 6VXX; red). B) Structure of u1S2q (green) aligned to the unmutated construct (PDB ID 6VXX; red). C) The u1S2q (green) mutation sites compared to the unmutated form (red).

FIGS. 37A-37B shows RBD proximal NTD glycans of SARS-2 MERS, SARS, and other β-CoV S-proteins. A) (left) Side view of the one RBD ‘up’ state SARS-2 structure and map (PDB ID 6VSB; EMDB 21375) depicting the reconstructed N165 and N234 NTD glycans protruding into the space occupied by the RBD in the ‘down’ state. (right) top view of the N165 and N234 glycans. B) Structures of the MERS, SARS, OC43, HKU1, and Murine S-proteins (PDB IDs 5W9H, 6CRW, 6OHV, 5108, and 3JCL, respectively) depicting the location of RBD proximal N-linked glycans. Closed (red), 1-up (green), 2-up (orange), and 3-up (blue) RBD state surfaces below cartoon representations indicate whether such states have been observed for each timer.

FIGS. 38A-38D show structure and antigenicity of the N165A and N234A SARS-CoV-2 ectodomain spikes. A) Percentage change in ACE2 binding for the N234A, and N165A mutant spikes, relative to the unmutated spike. Binding was measured by SPR with ACE-2 (with a C-terminal Fc tag) captured on an anti-Fc surface, and the spike as analyte. Error bars represent results from four independent injections. B) Representative ACE2 binding SPR response curves. C) NSEM results for the N234A spike. D) NSEM results for the N165A spike. For figures (C) and (D) shown from left to right are percentages of discrete 3D populations observed, representative micrograph, representative 2D class averages, discrete populations obtained by 3D classification.

FIGS. 39A-39H show structural comparison of the N234A mutant in the ‘up’ and ‘down’ configurations to the unmutated spike. A) Side view of the symmetric ‘down’ state N234A mutant S-protein trimer aligned to the unmutated trimer (PDB ID 6VXX, grey). B) Side view of a ‘down’ state NTD (green) depicting the shifted NTD relative to the unmutated form (grey) and the N165 glycan. Adjacent RBD is colored cyan. C) (upper) Map view of the apical β-sheet motif of the NTD for the N234A ‘down’ state (lower) Map view of the apical β-sheet motif of the NTD from the unmutated ‘down’ state (PDB ID 6VXX) D) The N234A trimer map density with the NTD (green) and RBD (cyan) coordinates aligned to the unmutated form (grey). E) Side view of the ‘up’ state N234A mutant S-protein trimer aligned to the unmutated trimer (PDB ID 6VYB, grey). F) (left) Cartoon representation of N234A ‘up’ state RBD (cyan) relative to the unmutated ‘up’ state RBD (grey). (right) as in (left) with the map density. G) Side view of the N165 glycan extending into the RBD ‘down’ state region near the ‘up’ state RBD. H) Top view of the N165 glycan extending into the RBD ‘down’ state region near the ‘up’ state RBD.

FIGS. 40A-40H show structural comparison of the N165A mutant in the ‘up’ and ‘down’ configurations to the unmutated spike. A) Side view of the symmetric ‘down’ state N165A mutant S-protein trimer aligned to the unmutated trimer (PDB ID 6VXX, grey). B) Side view of a single NTD (green) and adjacent RBD (red) with the ‘down’ state structure (grey) depicting the shift in the position of the NTD. C) Map view of the apical β-sheet motif of the NTD for the N165A ‘down’ state D) Zoomed in view of the NTD as in (C) with the map identifying the NTD shift. E) Side view of the symmetric ‘up’ state N165A mutant S-protein trimer aligned to the unmutated trimer (PDB ID 6VYB, grey). F) Side view of the ‘up’ state adjacent NTD (red) with N165 and N234 alpha carbons represented as spheres. G) Zoomed out view of the NTD as in (F) depicting the alignment of unmutated spike (grey) with the RBD in cyan. H) View of the NTD adjacent to the ‘down’ free state RBD.

FIGS. 41A-41C show SARS-2 and OC43 RBD proximal NTD glycans. A) Side view of a SARS-2 NTD (green) and RBD (purple) structure (PDB ID 6VXX) depicting the N234 glycan cleft. An RBD only structure (PDB ID 6M0J) was aligned to the trimer as a portion of the RBD is not present in the trimer structure. B) Side view of a SARS-2 NTD (green) and RBD (purple) structure (PDB ID 6VXX) depicting the N165 glycan. C) Side view of an OC43 NTD (magenta) and RBD (purple) structure (PDB ID 6OHW) depicting the N133 glycan.

FIGS. 42A-42C show SDS-PAGE and yields of purified S protein constructs. A) SDS-PAGE gels of the S protein constructs. R=reducing conditions; NR=non-reducing conditions and expression yields/L of the S protein constructs. B) Independent SPR replicate measures for the unmutated, N164A, and N234A mutants. Error bars represent results from multiple injections. C) ACE2 binding Kinetics measures of unmutated, N165A, and N234A S-protein with response curves (upper) and association/dissociation/affinity values (lower). ACE-2 was captured on an anti-Fc surface via a C-terminal Fc tag, and binding was measured by flowing over different concentrations of the spike constructs in independent injections.

FIGS. 43A-43J show thermostability of the S protein constructs. A-C) SEC profile of the S proteins. The dotted lines indicate the portion of the peak that was collected for further studies. The unmutated and u1S2q spikes were run on a Superose 6 Increase 10/300 column, and 93KJ and 94KJ spike was run on an analytical Superose 6 Increase 5/150 column. D-I) Unfolding profile curves \obtained by intrinsic fluorescence measurements using Tycho NT. 6. D-F) show ratio between fluorescence at 350 nm and 330 nm. G-I) plot the first derivative of this ratio. Asterisk mark the inflection temperatures that are tabulated in J)

FIGS. 44A-44I show high-resolution cryo-EM structure determination pipeline for the N234A mutant ‘up’ and ‘down’ states. A) Representative micrograph with selected particles circled. B) Representative CTF fit. C) Representative 2D classes D) Ab initio reconstruction of the ‘down’ state trimer depicting side (left) and top (right) views. E) Ab initio reconstruction of the ‘up’ state trimer depicting side (left) and top (right) views. F) High-resolution map of the C3 symmetric refinement of the ‘down’ state depicting side (left) and top (right) views. G) High-resolution map of the C1 asymmetric refinement of the ‘up’ state depicting side (left) and top (right) views. H) (top left) Fourier shell correlation curve for the ‘down’ state map, (bottom left) representative density, (right) local map resolutions. I) (top left) Fourier shell correlation curve for the ‘up’ state map, (bottom left) representative density, (right) local map resolutions.

FIGS. 45A-45I show high-resolution cryo-EM structure determination pipeline for the N165A mutant ‘up’ and ‘down’ states. A) Representative micrograph with selected particles circled. B) Representative CTF fit. C) Representative 2D classes D) Ab initio reconstruction of the ‘down’ state trimer depicting side (left) and top (right) views. E) Ab initio reconstruction of the ‘up’ state trimer depicting side (left) and top (right) views. F) High-resolution map of the C3 symmetric refinement of the ‘down’ state depicting side (left) and top (right) views. G) High-resolution map of the C1 asymmetric refinement of the ‘up’ state depicting side (left) and top (right) views. H) (top left) Fourier shell correlation curve for the ‘down’ state map, (bottom left) representative density, (right) local map resolutions. I) (top left) Fourier shell correlation curve for the ‘up’ state map, (bottom left) representative density, (right) local map resolutions.

FIGS. 46A-46E show structure of the ‘up’ state N165A mutant NTD shifts. A) Top view of the ‘up’ state N165A structure (green, cyan, and red) aligned to the unmutated spike (PDB ID 6VYB, grey). B) View of the ‘down’ adjacent RBD (green) and NTD (cyan) aligned to the unmutated spike (grey). C) View of the ‘down’ free RBD (red) and NTD (green) aligned to the unmutated spike (grey). D) View of the ‘up’ state RBD (cyan) and NTD (red) aligned to the unmutated spike (grey). E) Map view of the apical β-sheet motifs of the NTDs for the N165A ‘up’ state.

FIG. 47A-F. Vector based analysis of the 2P, N165A, and N234A C1 symmetry ‘down’ state 3D classification coordinates. A) The SARS-CoV-3 Spike depicting adjacent RBD, SD1, NTD, and NTD′ domains used in the domain and motif centroid based vector analysis. B) Cartoon representation of the RBD, SD1, NTD, and NTD′ domains used in the vector analysis depicting the vectors, angles, and dihedrals used in the analysis. Each Spike structure contains three RBD to NTD pairings for the analysis for each 2P, N165A, and N234A structure (4, 4, and 3 classes, respectively). C) The magnitudes of the vectors connecting adjacent RBDs and NTDs. D) The dihedral angle about the vector connecting SD1 to the NTD′. E) Principal components analysis of each vector dataset for each RBD-NTD pairing for the 2P (red), N165A (green), and N234A (blue) structures. Numbers indicate the class to which each pairing belongs. F) Alignment of each asymmetric structure with C3 symmetry score.

FIG. 48A-C. Structural comparison of the 2P and N165A C1 symmetry one ‘up’ state 3D classification results. A) The one ‘up’ state structural ensemble with a representative structure in bold. B) The dihedral angle about the SD1 to NTD′ vector. The boxed points indicate the dihedral for the bold structure in (A). C) Principal components analysis of each vector dataset for each RBD-NTD pairing for the 2P (red) and N165A (green) structures.

FIGS. 49A-B: Receptor binding domain and receptor interaction site of the SARS-CoV-2 Spike protein. A. Structure of the Spike trimer with each protomer colored as pink, cyan, and blue (PDB: 6VSB). One protomer has the receptor binding domain (RBD; blue) in the up conformation. The predominant interaction between the RBD and ACE-2 is highlighted in magenta. B. Magnified view of the superposition of the RBD in in the ACE-2 bound conformation (PDB: 6M17; yellow) onto the soluble Spike trimer (blue). The peptide within the receptor binding domain that interacts predominantly with the ACE2 receptor is highlighted in magenta.

FIGS. 50A-B Interaction of the SARS-CoV-2 Spike protein with its receptor ACE-2. A. The receptor binding domain of the Spike protein (yellow) is shown binding to its receptor, ACE-2 (cyan; PDB: 6M17). The predominant interaction between the RBD and ACE-2 is highlighted in magenta. B. This polypeptide is termed the receptor interaction site and can be the target of neutralizing antibodies aiming to prevent the interaction between the RBD and its receptor.

FIGS. 51A-C: Stabilization of soluble SARS CoV-2 Spike protein. A) Diagram of the SARS-CoV-2 full-length Spike (S) protein depicting the N-terminal domain (NTD), receptor binding domain (RBD), subdomains 1 and 2 (SD1 and SD2), heptad repeat 1 (HR1), heptad repeat 2 (HR2), and transmembrane domain (TM). Spike domains S1 and S2 are depicted below line diagram. B) Spike protein truncated to generate secreted protein. Spike protein trimers are stabilized by the introduction of K986P+V987P mutations (red PP). C) The same protein design as in B, but with additional D985C and S383C mutations (red “C” connected by lines). These two cysteines link together Spike domains to further stabilize Spike protein trimers. B) Upper: Diagram of the SARS-CoV-2 construct as in (A) with the addition of the RBD ‘down’ state stabilizing disulfide, D985C and S383C.

FIGS. 52A-E. SARS CoV-2 Spike nanoparticle immunogen designs. A) Diagram of the receptor binding domain of the Spike protein produced without the surrounding portions of the Spike protein. B) Diagram of the site within the receptor binding domain of the Spike protein that interacts with the virus receptor on host cells produced without the surrounding portions of the Spike protein. C-E) Attachment of the receptor interaction site (RIS), RBD, and truncated Spike protein to subunits of self-assembling protein nanoparticles to generate safe mimics of the virus.

FIG. 53A-D shows non-limiting embodiments of SARS-2 designs comprising various modifications. The modifications can be incorporated in full length sequences, or any other SARS-2 protein fragment. FIG. 53A-D discloses SEQ ID NOS 168-171, respectively, in order of appearance.

FIG. 54A-C show non-limiting embodiments of amino acid sequences of nCoV-1 nCoV-2P (54A), N165 mutant (54B) and N234 mutant (54C). Positions 165 and 234 are underlined. FIG. 54A-C discloses SEQ ID NOS 172-174, respectively, in order of appearance.

### DETAILED DESCRIPTION

The invention provides proteins and nucleic acids, including modified mRNAs which are stable and can be used as immunogens. Provided also are nucleic acids optionally designed as vectors, for example for recombinant expression and/or stable integration, e.g. but not limited, full-length S protein DNA encoding trimer for stable expression, or VLP incorporation.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

SARS-2 Coronavirus S Protein Designs

The ongoing global pandemic of the new SARS-CoV-2 coronavirus presents an urgent need for the development of effective preventative and treatment therapies. The viral-host cell fusion (S) protein spike is a prime target for such therapies owing to its critical role in the virus lifecycle. The S protein is divided into two regions: the N-terminal S1 domain that caps the C-terminal S2 fusion domain. Binding to host receptor via the Receptor Binding Domain (RBD) in S1 is followed by proteolytic cleavage of the spike by host proteases. Large conformational changes in the S-protein result in S1 shedding and exposure of the fusion machinery in S2. Class I fusion proteins such as the coronavirus (CoV) S protein that undergo large conformational changes during the fusion process must, by necessity, be highly flexible and dynamic. Indeed, cryo-EM structures of the SARS-CoV-2 (SARS-2) spike protein reveal considerable flexibility and dynamics in the S1 domain^{1,2}, especially around the RBD that exhibits two discrete conformational states—a “down” state that is shielded from receptor binding, and an “up” state that is receptor-accessible. We will use our robust, high-throughput computational and experimental pipeline to define the detailed trajectory of the “down” to “up” transition of the SARS-2 S protein, identify early metastable intermediates in the fusion pathway, and exploit their structures and dynamics for identifying drug and vaccine candidates that target SARS-2.

A wealth of structural information on CoV spike proteins, including recently determined cryo-EM structures of the SARS-2 spike¹⁻¹¹, provides a rich source of detailed data from which to begin precise examination of macromolecular transitions underlying triggering of this fusion machine. In certain aspects the invention provides that(a) analysis quantifying CoV S1 domain movements around which structurally conserved domains undergo rigid body motions, (b) in silico, prescreened panel of differentially domain position stabilizing mutations, and (c) integrated computational and experimental approach with unprecedented, dedicated access to >300 accelerated compute devices (GPUs), rapid and priority access to a K3 direct electron detector equipped Titan Krios electron microscope, and high-throughput structural determination pipeline. Together, this puts us in a unique position to provide atomically detailed mechanistic insight into the fusion mechanism of the SARS-2 virus. The scientific premise of this study is that understanding the structural dynamics and early transition kinetics of mobile regions of the SARS-2 spike will allow optimal control of vaccine and drug responses, and facilitate the development of new antiviral drugs and protective vaccines. The goal of this study is to define mechanistically-derived transition states of the pre-fusion SARS-2 spike that can be exploited for vaccine and drug design.

The invention is based on work to define domain motions in the pre-fusion SARS-2 spike. The idea is that while the RBD undergoes a dramatic “up” and “down” hinge motion, other subtle movements in the pre-fusion SARS-2 spike play an important role in defining antibody and ligand binding specificity. Analysis of CoV S protein structures revealed subtle shifts in S1 that make and break interactions with adjacent domains, resulting in multistate or disordered behavior of the RBD in its “down” position. Here, we identify a set of mutations that lock and stabilize the SARS-2 S protein with the RBD in discrete “down” positions, each with different but specific RBD positions rather than the usual multistate behavior observed in all CoV spike structures determined to date. Deploying rapid assays to assess protein expression, thermostability, and antigenicity, we will generate a set of stabilized SARS-2 spike variants with defined reactivity to patient-sera. We will determine high-resolution cryo-EM structures to define the metastable RBD “down” state orientations in these mutants, and use the combined experimental information from structures, biochemistry and biophysics to iterate the structure-guided computational design cycle.

The invention is based on work to define the trajectory of the transition between the “down” and “up” states of the SARS-2 S protein. The idea is that the SARS-2 S protein transitions through multiple metastable intermediate states between the known “down” and “up” states. Using an integrated approach, we will interrogate the mechanism by which the SARS-2 S protein transitions from its “down” state to the receptor-accessible “up” state. Our initial examination of the available CoV S protein structures quantifies specific rigid body domain movements within each state. Using a combination of path finding and adaptive sampling molecular dynamics (MD) simulation techniques, we will develop a theoretical model of this initial triggering event. Structural details from the putative path will be used to stabilize predicted intermediate states. Provided are experiments to study the biochemical and biophysical properties of these putative intermediates, and determine their structures using high-resolution cryo-EM. We will assess the reactivity of these structures to patient-sera and known SARS-2 spike ligands to define state antigenicity.

In certain aspects the invention provides methods to determined structures of multiple “down”, “up”, and intermediate states of the SARS-2 S protein. Given the current global health emergency we will prioritize rapid dissemination of results to the community. Importantly, we will make available coordinates from the experimentally refined transition ensemble determined via MD simulation to enable close examination of the presented transition by researchers in the fields of drug and vaccine design. Overall, these studies will provide atomically detailed structural and mechanistic information that can be exploited for vaccine and therapeutics design.

On Mar. 11, 2020, the World Health Organization (WHO) characterized the ongoing spread of COVID-19, a highly contagious respiratory disease caused by the new betacoronavirus SARS-CoV-2 (SARS-2), a pandemic. Originating in the Wuhan province of China, now spread to over 100 countries, the virus has infected >150,000 individuals and caused >8000 deaths world-wide. As the virus continues to spread, there is an urgent need to understand as much as possible, as rapidly as possible, about this new virus.

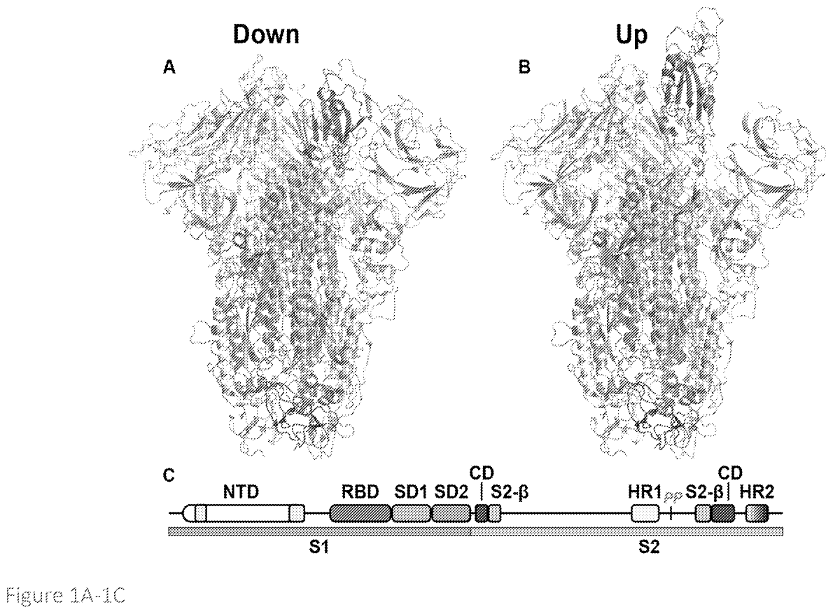

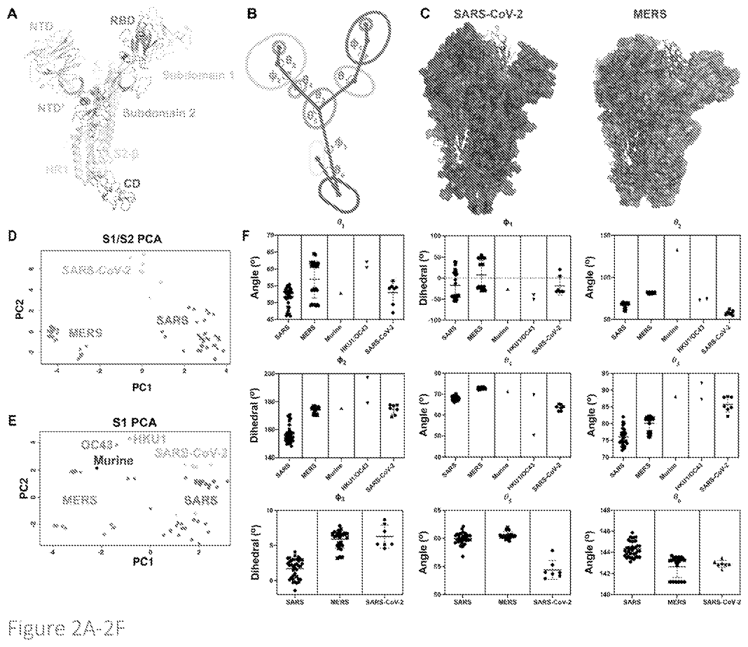

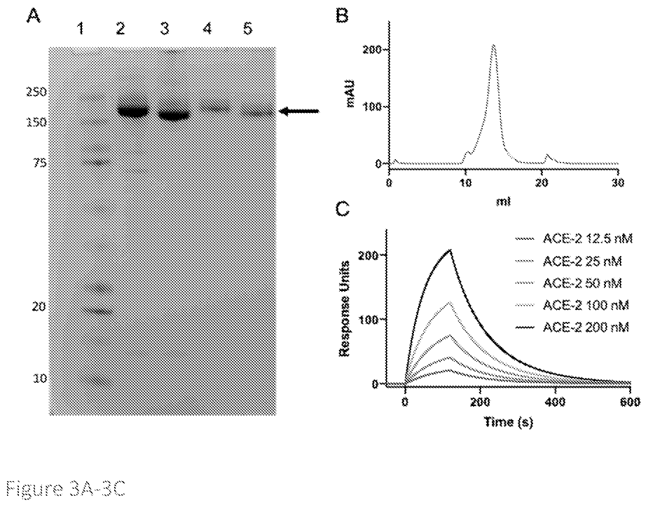

The transmembrane SARS-2 S protein spike trimer (FIG. 1) mediates attachment and fusion of the viral membrane with the host cell membrane and is therefore critical for the viral life cycle. Displayed on the surface of the virus, the S protein is a prime target for vaccine and therapeutics design.

The SARS-2 S protein displays striking structural similarities with the S proteins of the previously identified SARS-CoV, MERS-CoV, and other human and murine CoV viruses. However, most S-targeting antibodies to SARS and MERS do not cross-react with SARS-2. Conformational evasion is among the many host immune evasion tools available to viruses. Dramatic shifts in the conformational ensemble of states for CoVs have in fact been demonstrated^{1,2}. Therefore, a detailed understanding of structure and dynamics of the SARS-2 S protein in comparison to is orthologs will reveal how genetic drift can give rise to the large phenotypic differences that drive viral evolution and host immune evasion.

Thus, the urgent need to understand the SARS-2 virus that is responsible for the ongoing pandemic makes this study significant and relevant to public health.

Provided are studies that use an integrated structural biology approach to harness the latest innovations in high-throughput cryo-electron microscopy and computational methodologies to approach this urgent global healthcare problem. These studies include use of Titan Krios microscope fitted with a K3 camera for rapid determination of high-resolution structures, access to a Philips EM420 microscope, as well as to a Talos Arctica for cryo-screening and data collection at the National Institutes for Environmental Health Sciences (NIEHS), NIH.

Studies will be able to test immunogenicity in mice and rabbits of any promising SARS-2 spike variants generated in this study.

In non-limiting embodiments, aspects of the invention are based on the idea that protein dynamics impact its antigenic and immunogenic properties. Coronavirus designs are based on an integrated approach that closely couples structure and molecular dynamics-driven protein engineering with biophysical, biochemical, virological and immunological studies.

Conformationally distinct structural states of the CoV S-protein spike are well defined. The transmembrane CoV S protein spike trimer is composed of interleaved protomers that include an N-terminal receptor binding S1 domain and a C-terminal S2 domain that contains the fusion elements (FIG. 1).³ The S1 domain is subdivided into the N-terminal domain (NTD) followed by the receptor binding domain (RBD) and two structurally conserved subdomains (1 and 2). Together these domains cap the S2 domain, protecting the conserved fusion machinery. Several structures for a soluble ectodomain construct that retains the complete S1 domain and the surface-exposed S2 domain have been determined. These include SARS-2^{1,2}, SARS⁴⁻⁸, MERS^{4,9}, and other human^{3,10} and murine¹¹ beta-CoV spike proteins. These structures revealed the S-protein spike to be conformationally heterogenous, especially in the region of the RBD. Within a single protomer the RBD can adopt a closed, ‘down’ state (FIG. 1A), in which the RBD covers the apical region of the S2 protein near the C-terminus of the first histad repeat (HR1), or an open, ‘up’ state in which the RBD is dissociated from the apical central axis of S2 and the NTD (FIG. 1B). Cryo-EM structures consistently demonstrate a large degree of domain flexibility in both the ‘down’ and ‘up’ states in the NTD and RBD. While these structures have provided essential information for identifying the relative arrangement of these domains, little is understood regarding the fusogenic and antigenic consequences of instability in this region.

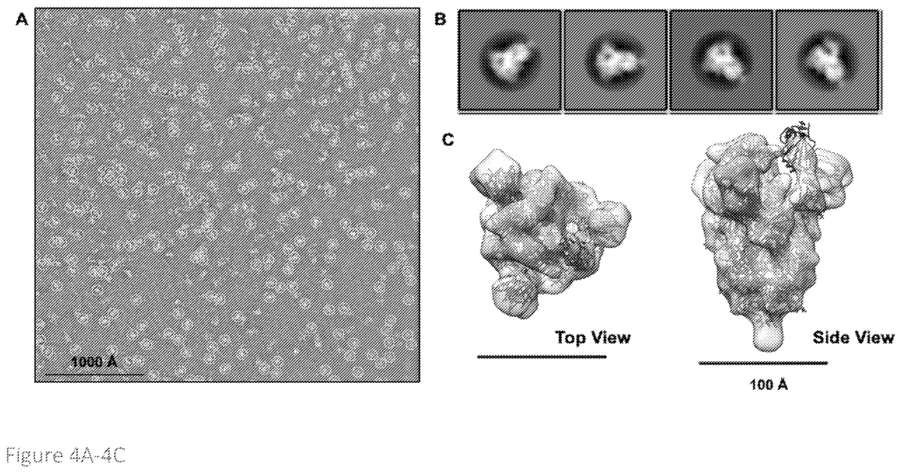

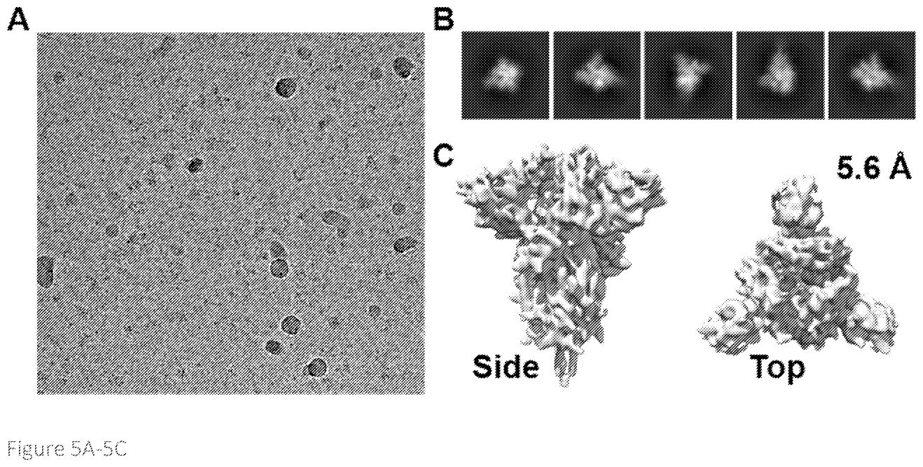

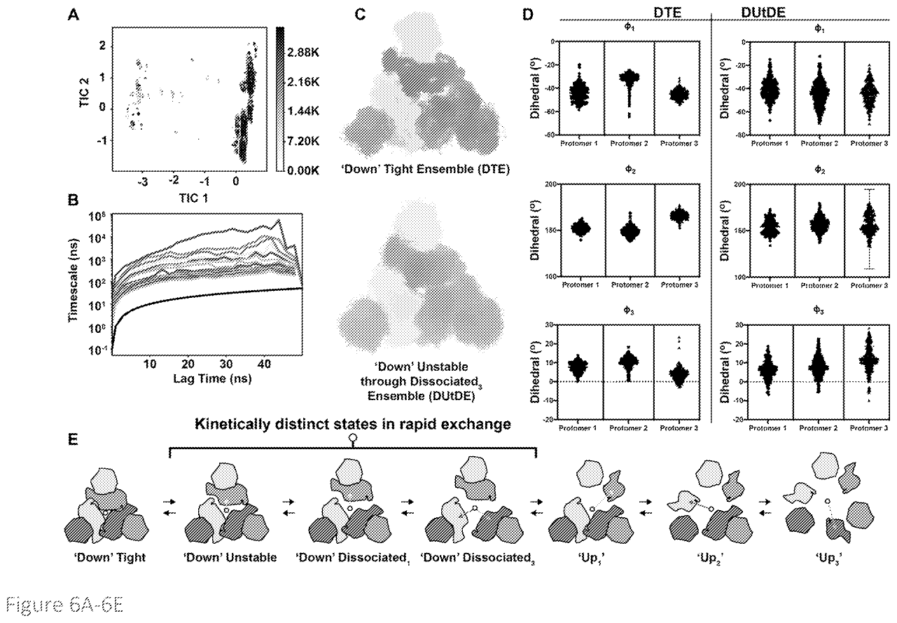

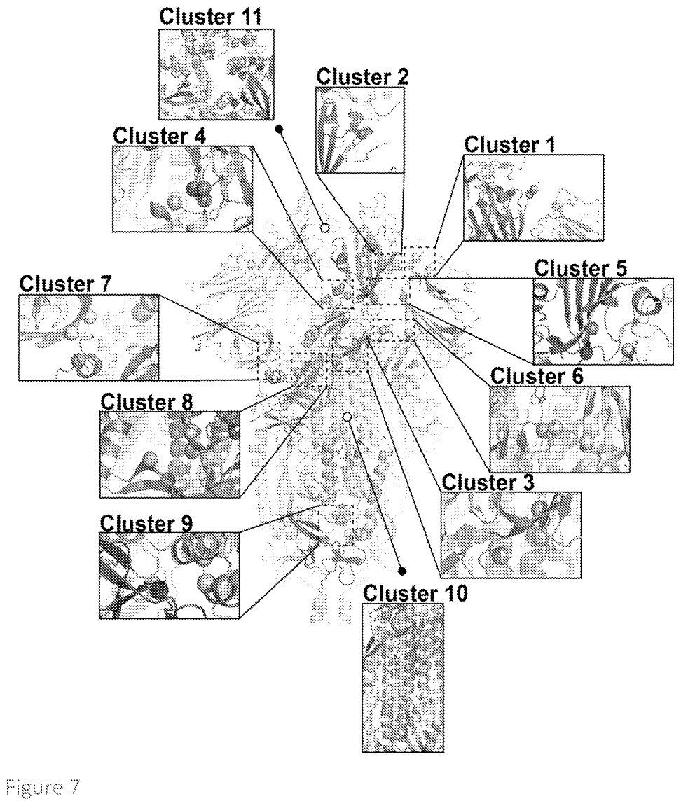

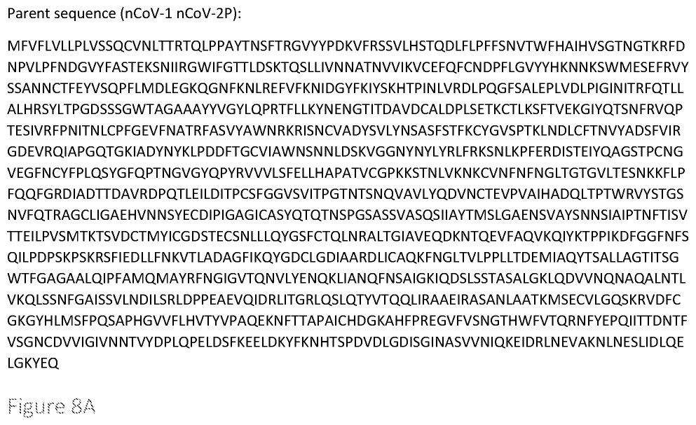

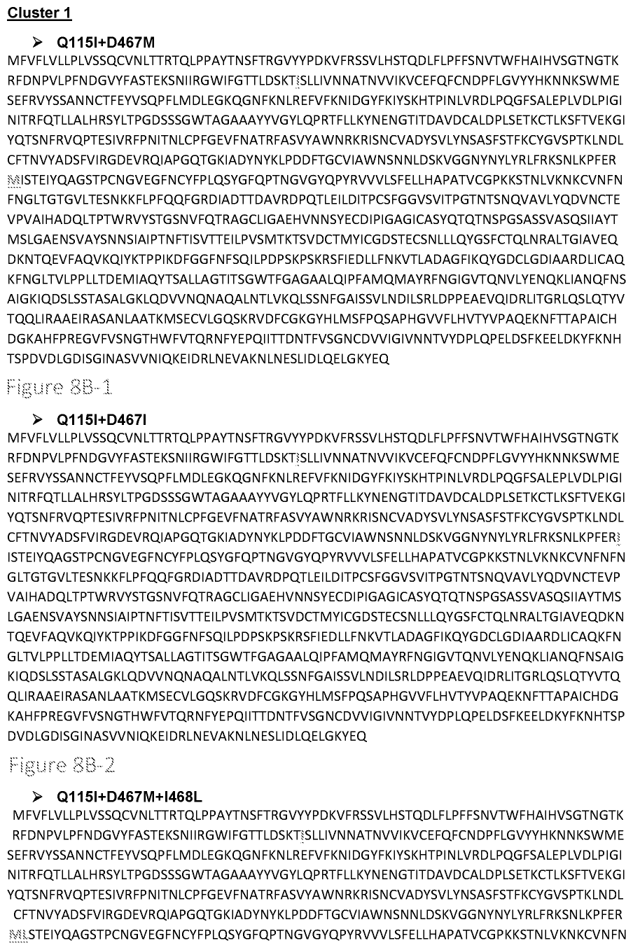

A detailed structural schematic defining the geometry and internal rearrangements of movable domains. An understanding of macromolecular structural dynamics requires a precise definition of structural states. Examination of the available SARS and MERS S-protein structures revealed: 1) the NTD, RBD, subdomains and internal S2 domains move as rigid bodies and 2) these domains display a remarkable array of relative shifts between the S1 region's domains and the S2 region's β-sheet motif and CD (FIG. 2). In order to quantify these movements, we have analyzed the relevant regions of motion and their structural disposition in all available CoV ectodomain spike structures including 15 SARS structures, 10 MERS structures, a HKU1 structure, an OC43 structure, a murine CoV structure, and the three recently released SARS-2 structures^{1,2}. Each protomer in those structures displaying asymmetric ‘up’/‘down’ RBD states was examined independently (76 structural states total). The NTD domain was split into a primary, N-terminal section and a secondary C-terminal section. The generally disordered, central segment of the RBD was not included in the analysis. A vector-based analysis similar to that used in our recent manuscript detailing motion in the HIV-1 Envelope protein¹² was applied here. Specifically, vectors connecting the region's c-α centroids were generated and used to define the relative dispositions of the domains. The vector magnitudes and select angles and dihedrals were used to identify the breadth of domain movements and compare between strains. The results indicate that CoV spike proteins in various strains differ markedly from one another and that considerable variability in the domain arrangements within strains exists, such as in the SARS ectodomains. These results revealed a large conformational space available to the CoV S-protein and indicated that subtle changes in inter-domain contacts can play a major role in shifting these distributions.

SARS-2 S Protein Production, Purification and Structural Characterization

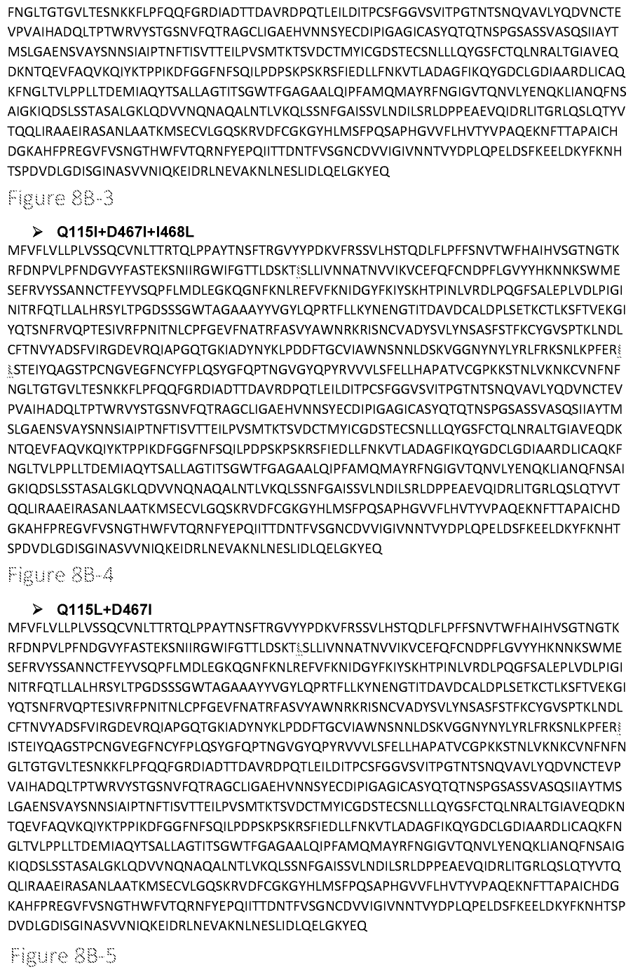

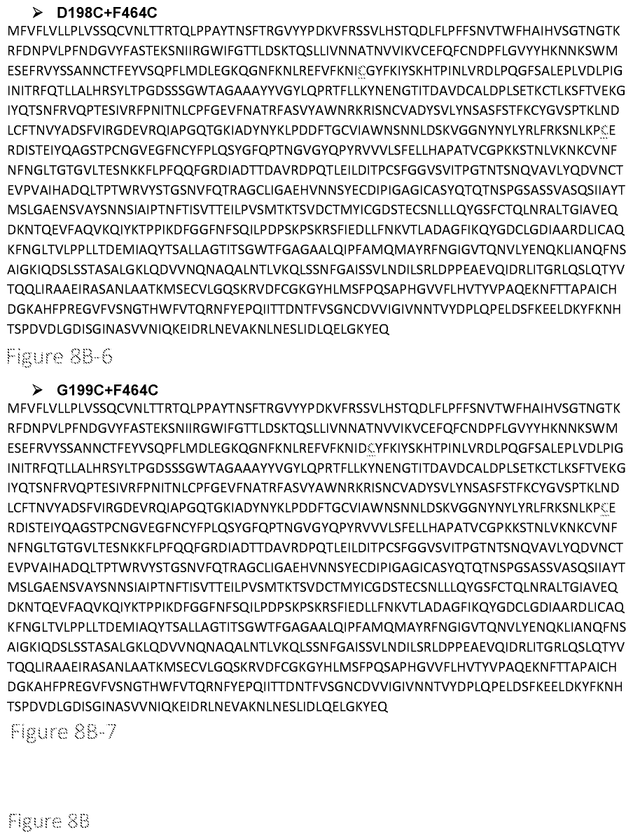

The SARS-2 S protein ectodomain² was expressed in 293F cells and purified using published methods to yield ˜4 mg/L purified spike (FIG. 3). The SARS-2 S protein ectodomain described in Wrapp, D. et al. Cryo-EM structure of the 2019-nCoV spike in the prefusion conformation. Science 367, 1260-1263, doi:10.1126/science.abb2507 (2020) is incorporated herein by reference. The purified S protein was tested for binding to ACE-2 receptor using Surface Plasmon Resonance (SPR) (FIG. 3C). Negative Stain Electron Microscopy (NSEM) (FIG. 4) and preliminary cryo-EM (FIG. 5) studies were performed. 3D reconstruction for the SARS-2 spike from NSEM recapitulated the 1-RBD-up state that was visualized in the published high resolution cryo-EM structure^{1,2}. Our NSEM pipeline enables rapid and low-cost screening of a large number of constructs, and our high-throughput cryo-EM pipeline will allow us to solve high resolution structures of the SARS-2 S protein variants in this study. These results demonstrate that, within a relatively short period of a few weeks, we were able to adapt our protein production, biochemistry and structural biology platforms optimized for HIV-1 Env to the SARS-2 S protein, and that we now have all experimental systems setup to accomplish the goals of this project.

Advanced molecular simulation results for the SARS ectodomain spike indicate rapid exchange between metastable states. In order to examine the breadth and time scales of the dynamics of CoV spike protein structure, we initiated an adaptive sampling simulation of the symmetric all ‘down’, closed state of the SARS CoV soluble S-protein (PDB ID 6ACC⁶). To overcome the sampling problem in MD, the adaptive scheme periodically monitors multiple simultaneous simulations and launches additional simulations in regions of the coordinate space along transition paths. In this way, difficult to observe slow processes become accessible. In total, we obtained 539 independent 50 ns simulations totaling ˜27 μs of simulation time. Monitoring contacts between each protomer's RBD and their adjacent RBD, NTD, and HR1 C-terminus, we further projected the data using the time-lagged independent component analysis (TICA) approach. TICA components point in the direction of the slowest processes in the simulation data which means that transitions along the so call TICs can correspond to transitions between metastable states (FIG. 6A). Analysis of the implied timescale plot (ITS) indicate simulations extended to ˜100 ns will be sufficient to produce a converged Markov model with sufficient sampling (FIG. 6B). Examination of the structures of two representative kinetics states indicates a very tightly coupled RBD to RBD interactive state (FIG. 6C; upper). The breaking of the RBD-RBD contacts results in a highly dynamic ‘down’ dissociated state in which RBD to RBD contacts rapidly form and dissociate (FIG. 6C; lower). Indeed, vector analysis of these states demonstrated a marked difference in heterogeneity in the relevant structural regions between the two states (FIG. 6D). This leads us to identify a mechanism for closed to open transitions that will involve these previously unknown kinetic intermediates (FIG. 6E). These results indicate mutations in the RBD-RBD and RBD-NTD interface can alter the state's equilibrium distribution as well as their transition kinetics meaning point mutations in the SARS-2 S-protein can readily alter the conformational distribution of the protein and therefore its antigenicity.

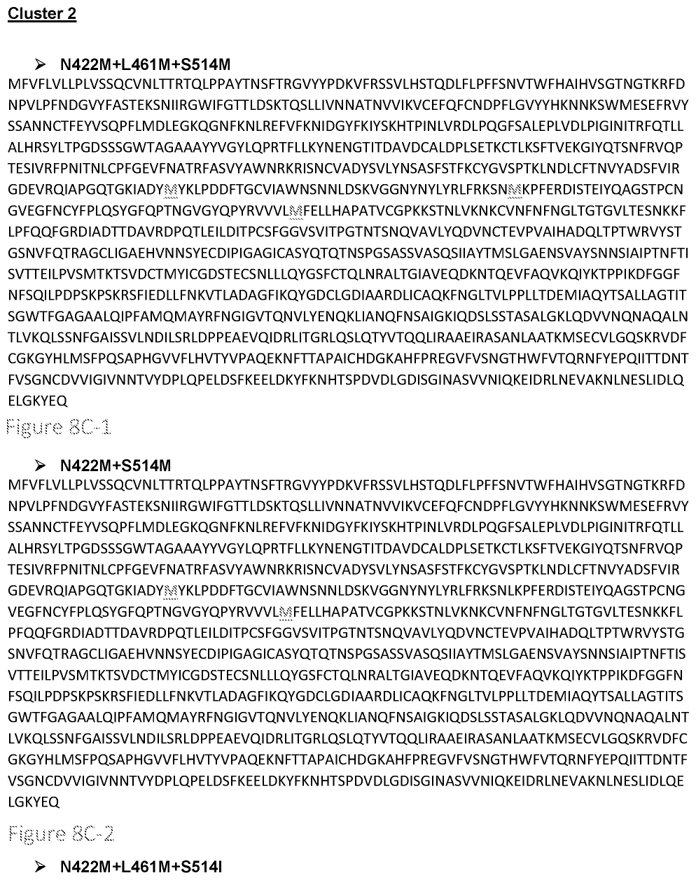

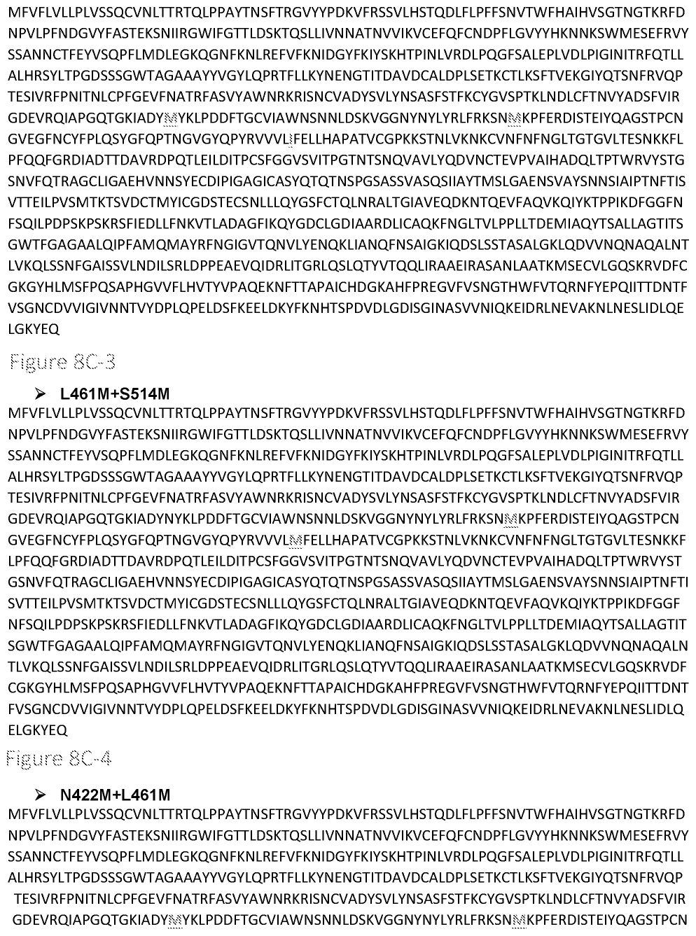

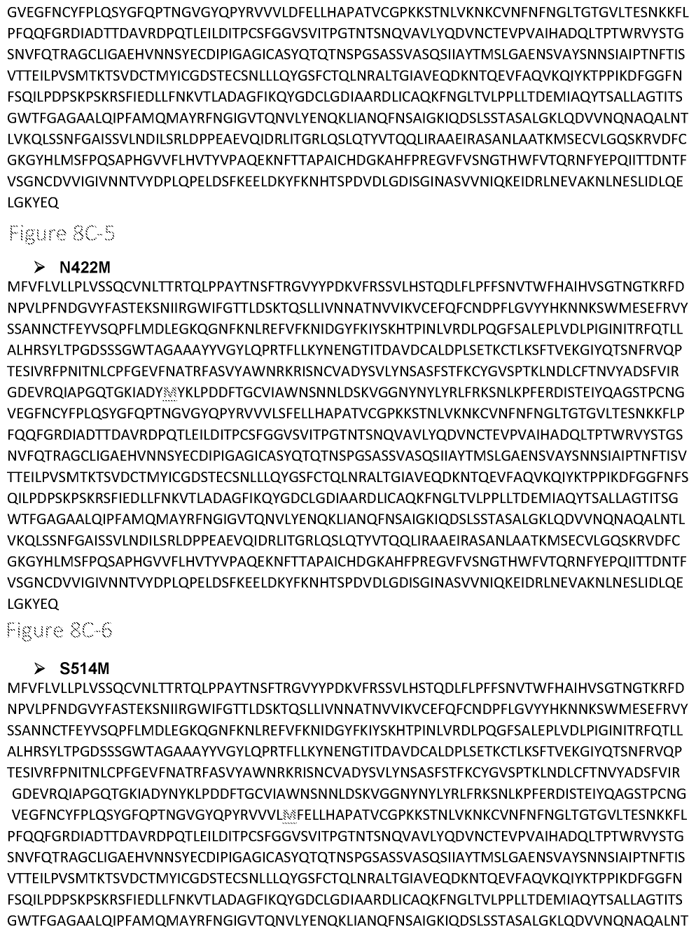

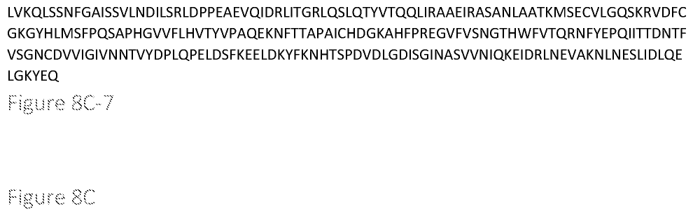

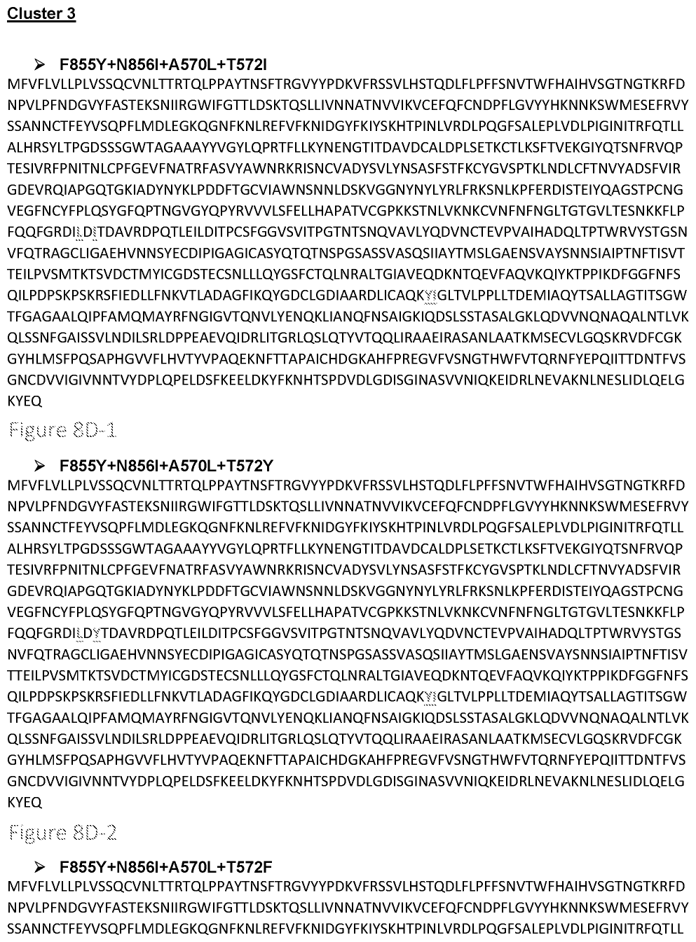

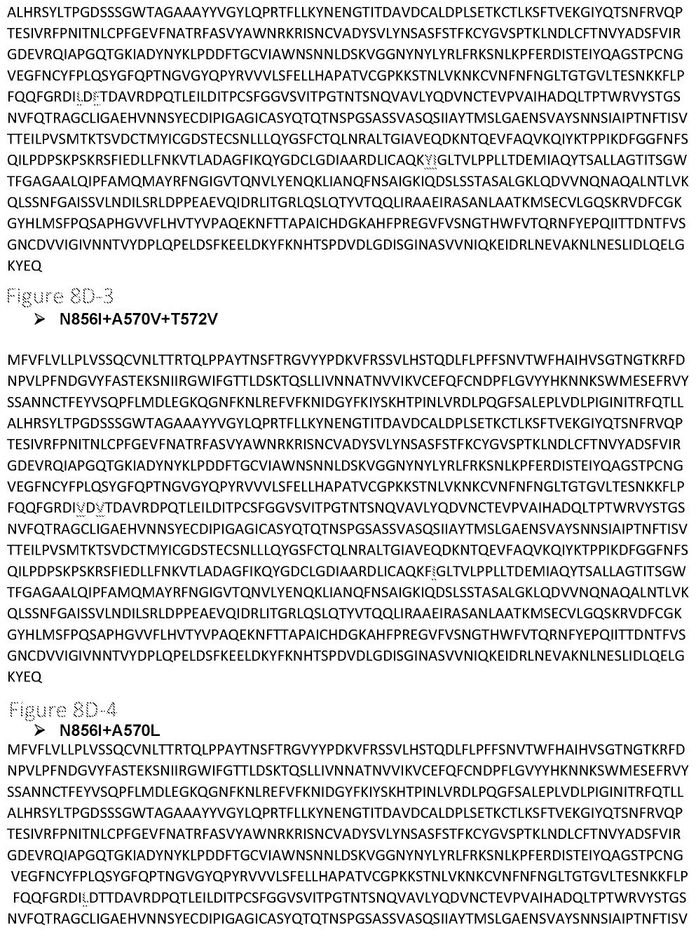

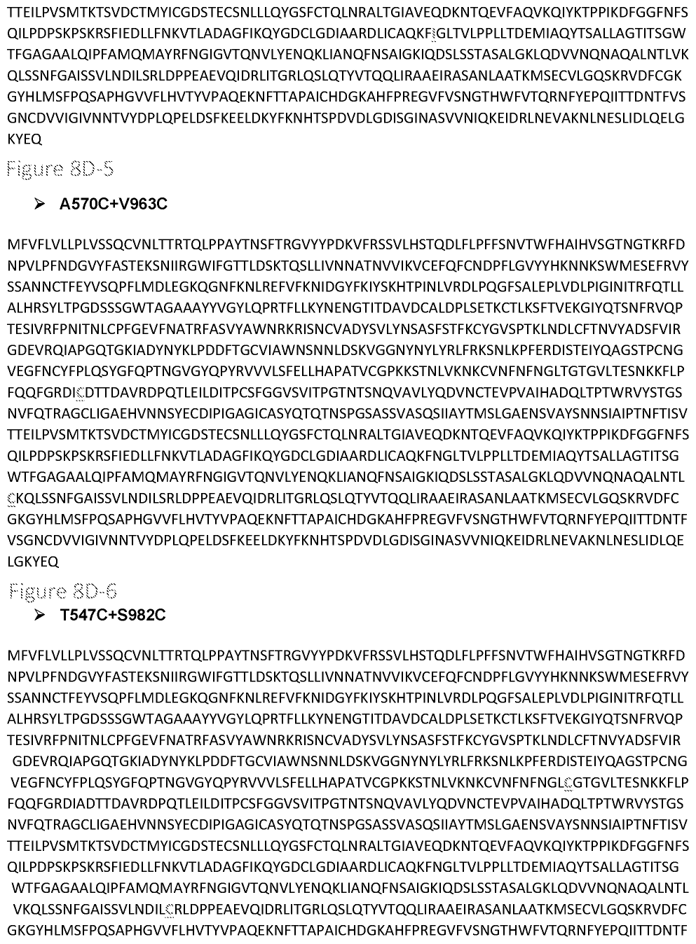

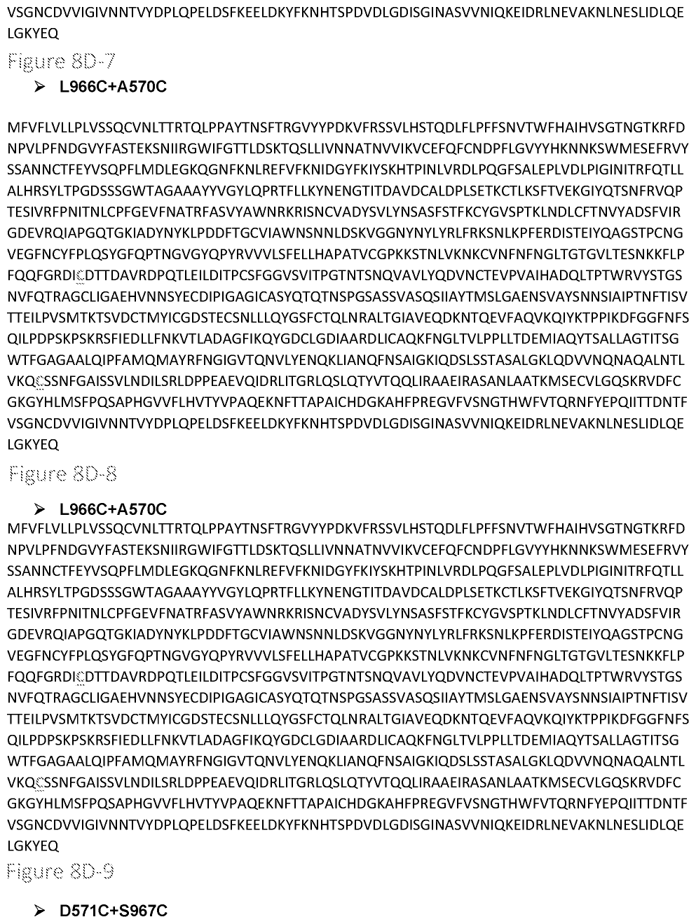

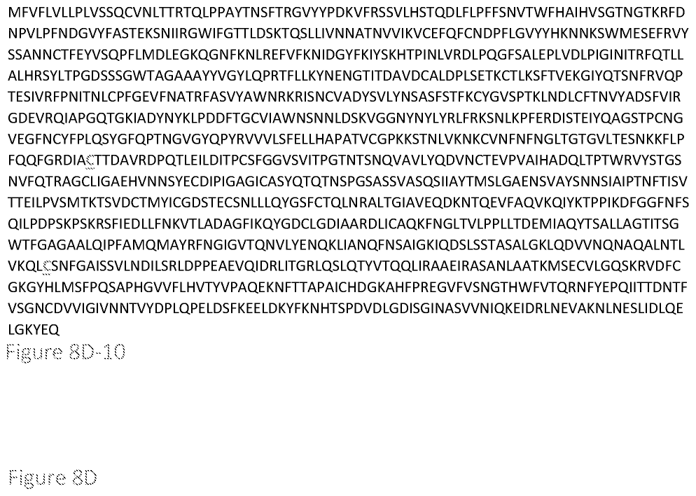

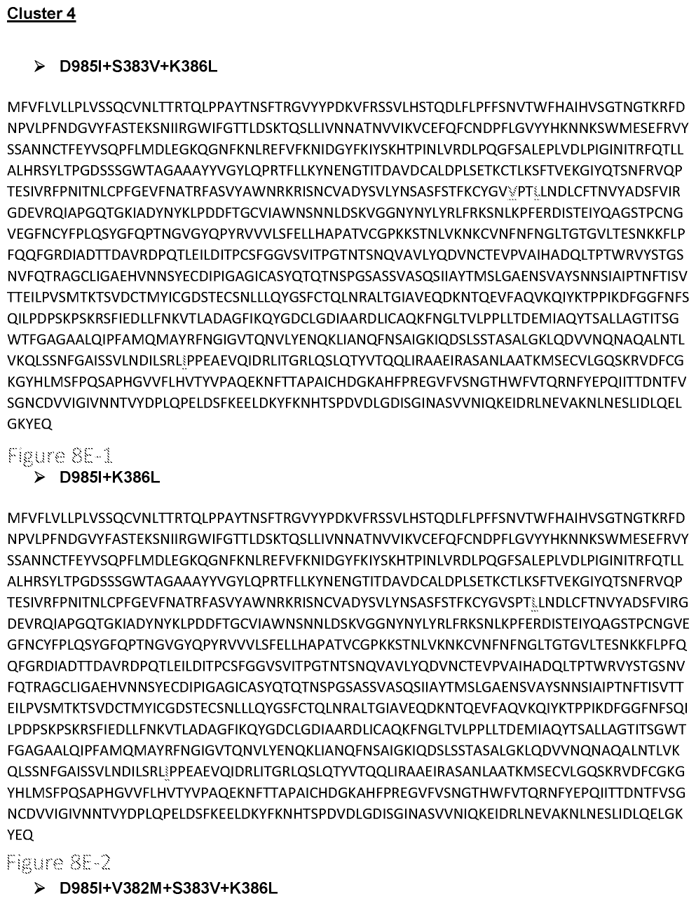

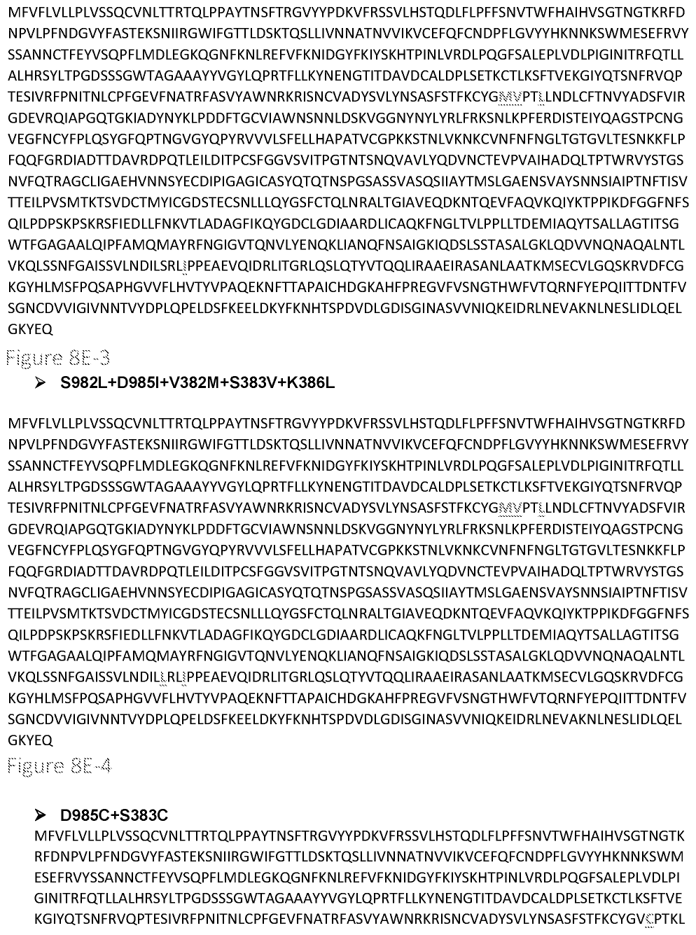

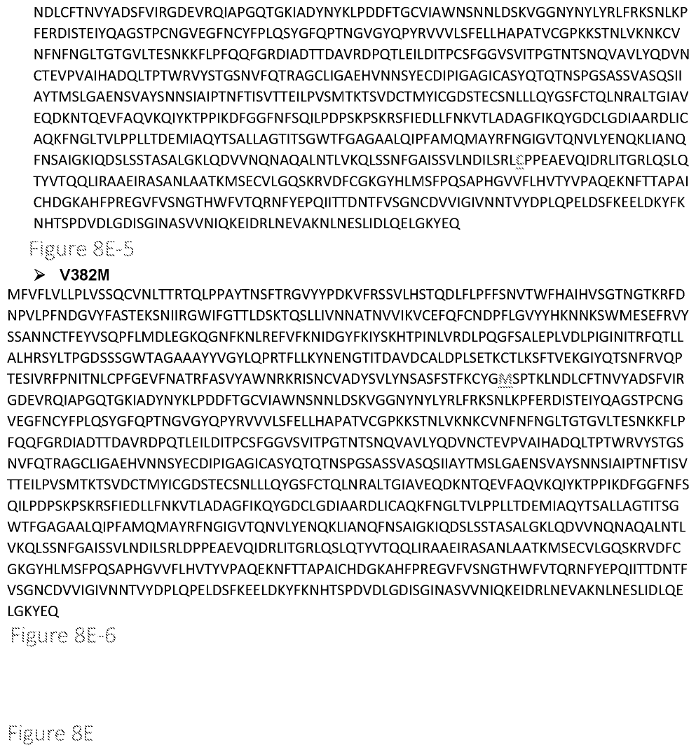

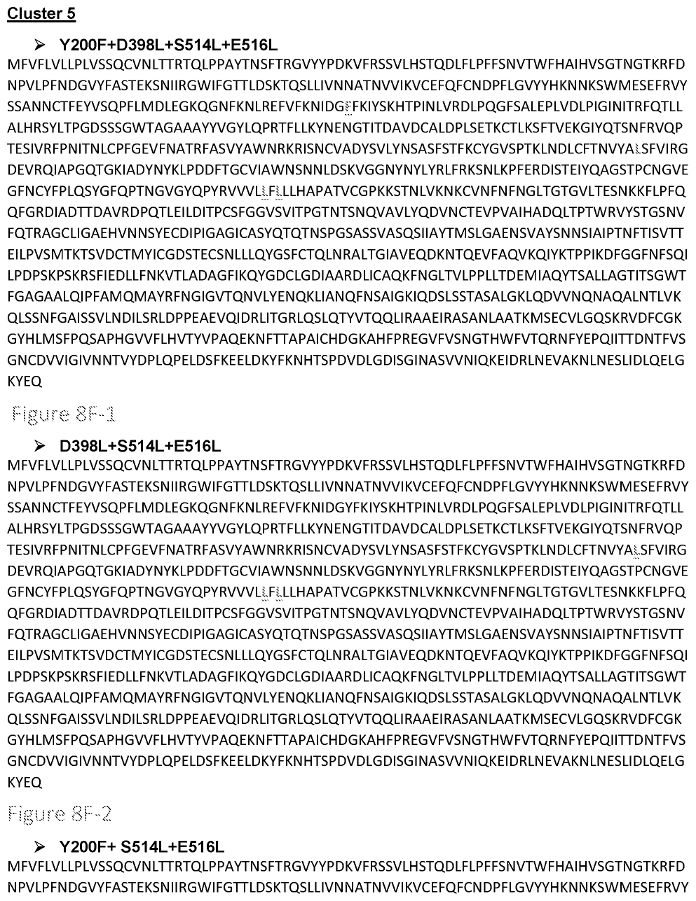

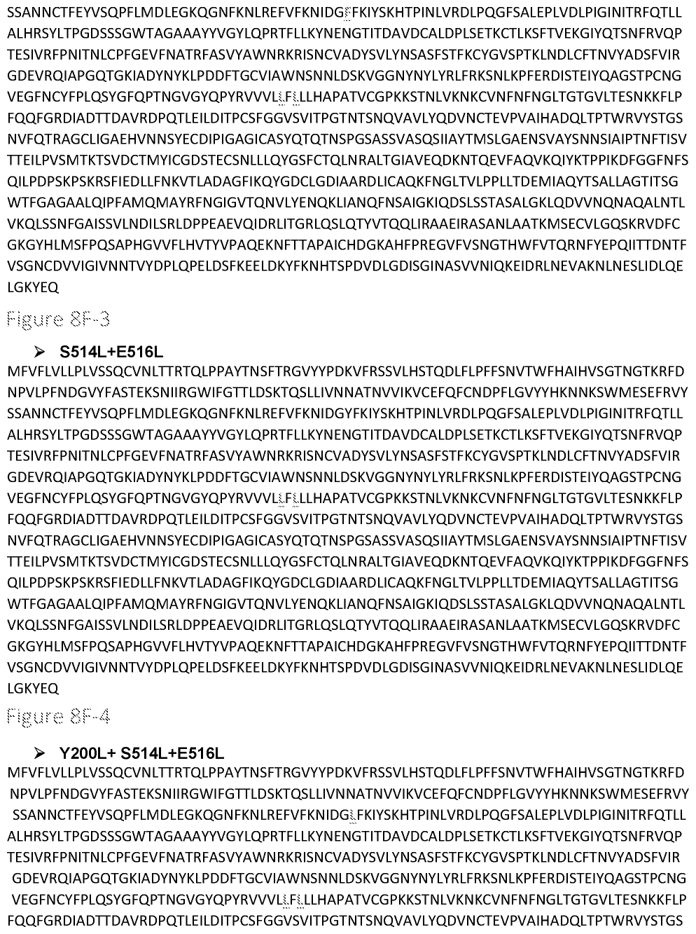

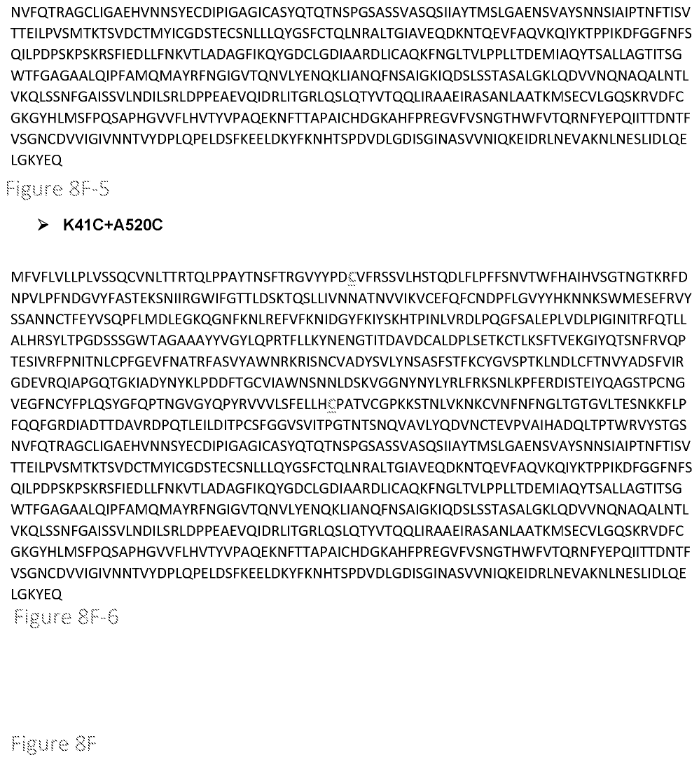

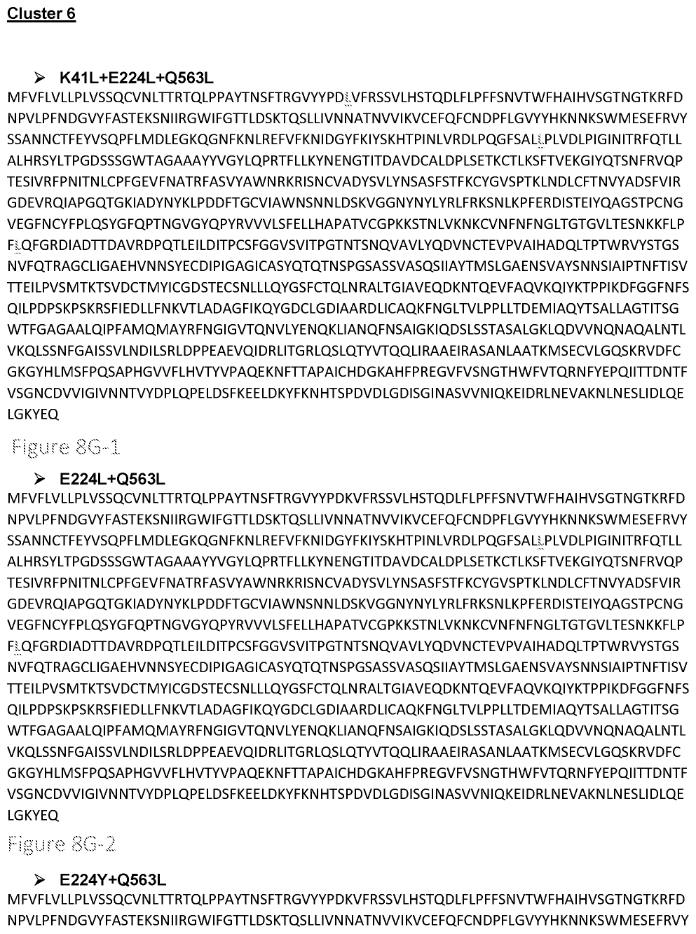

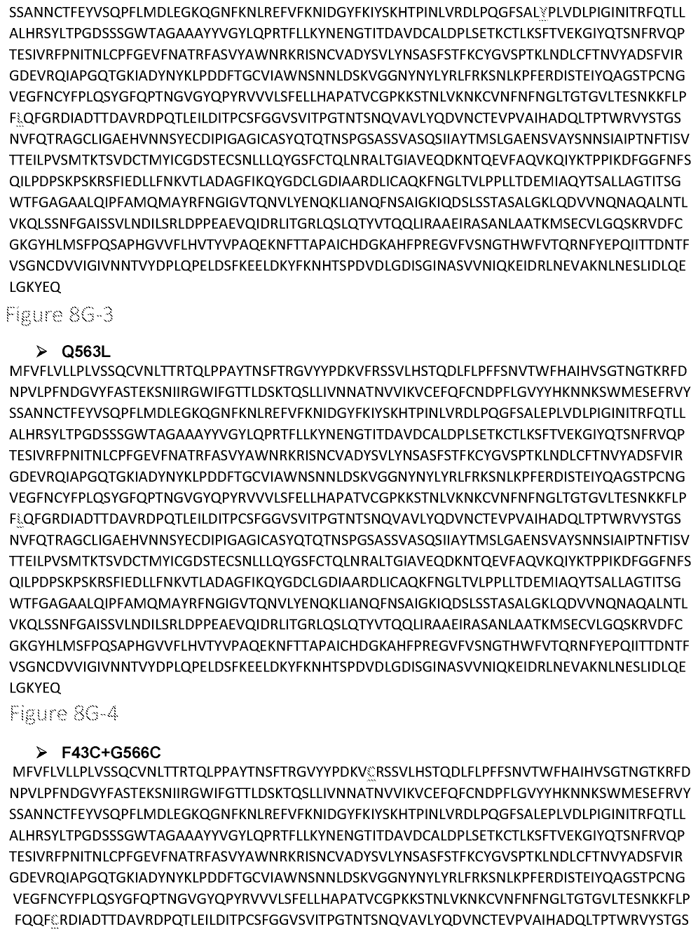

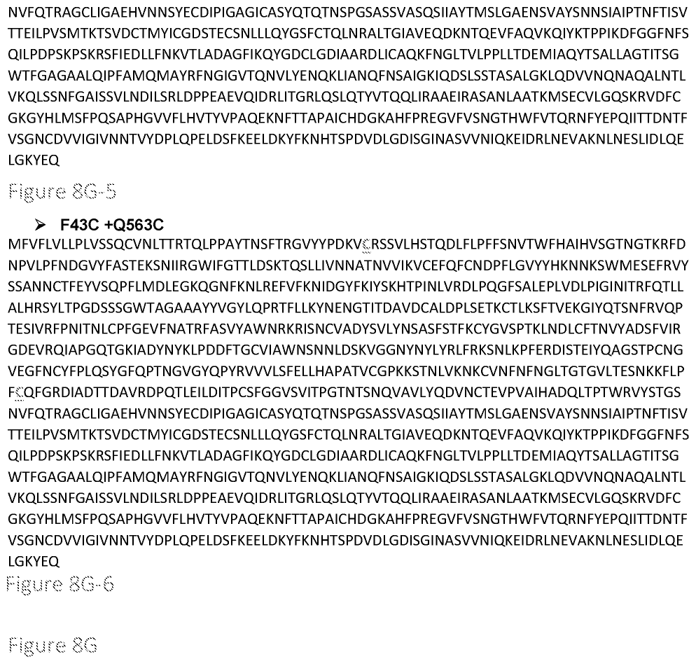

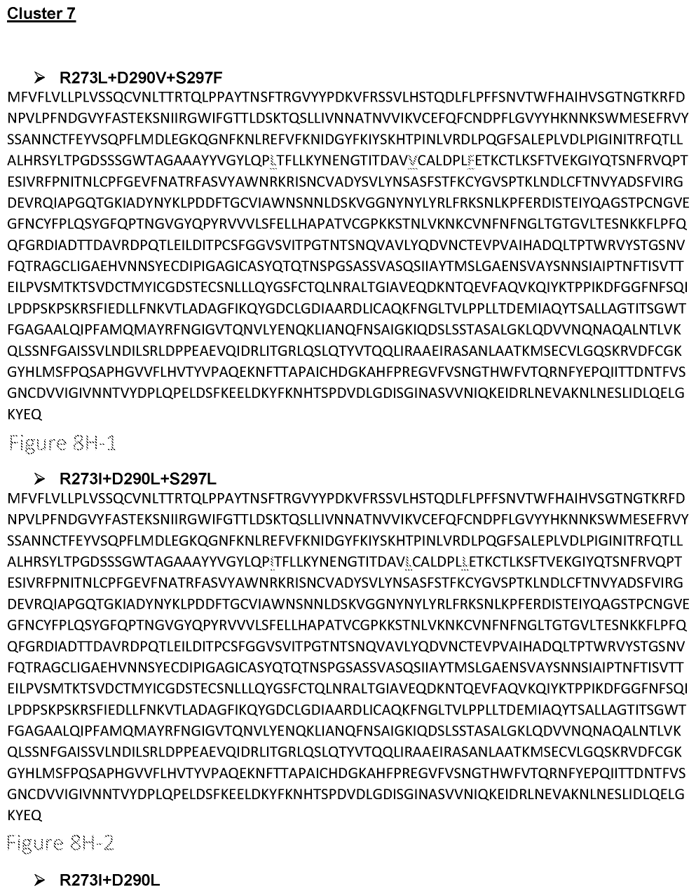

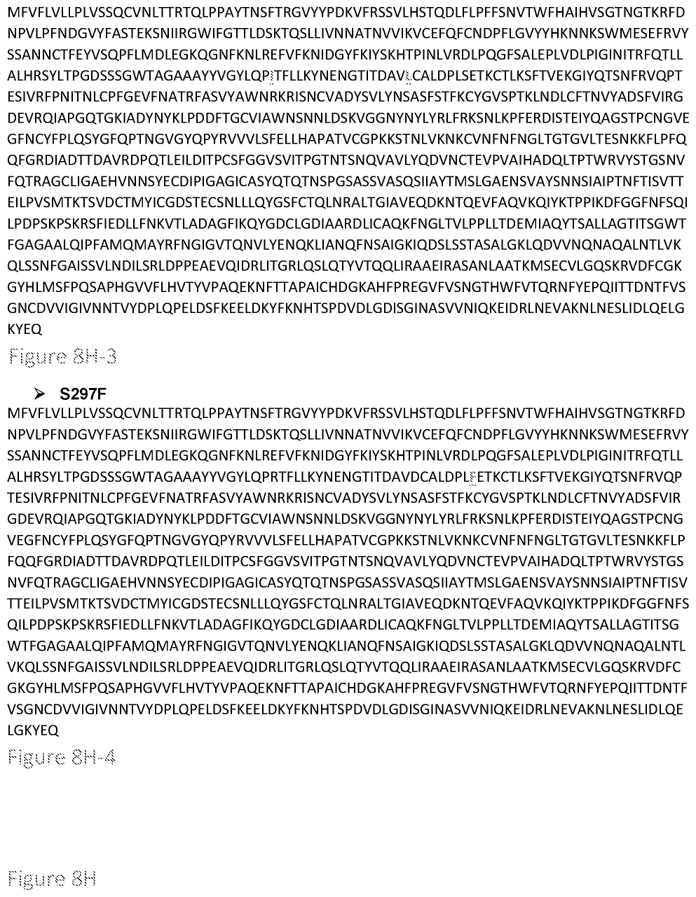

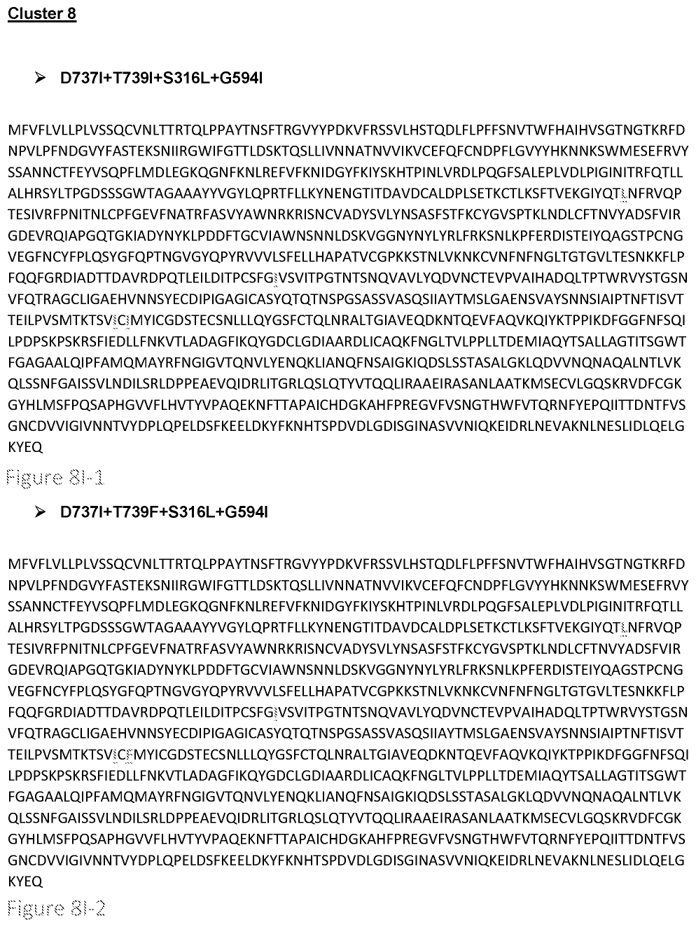

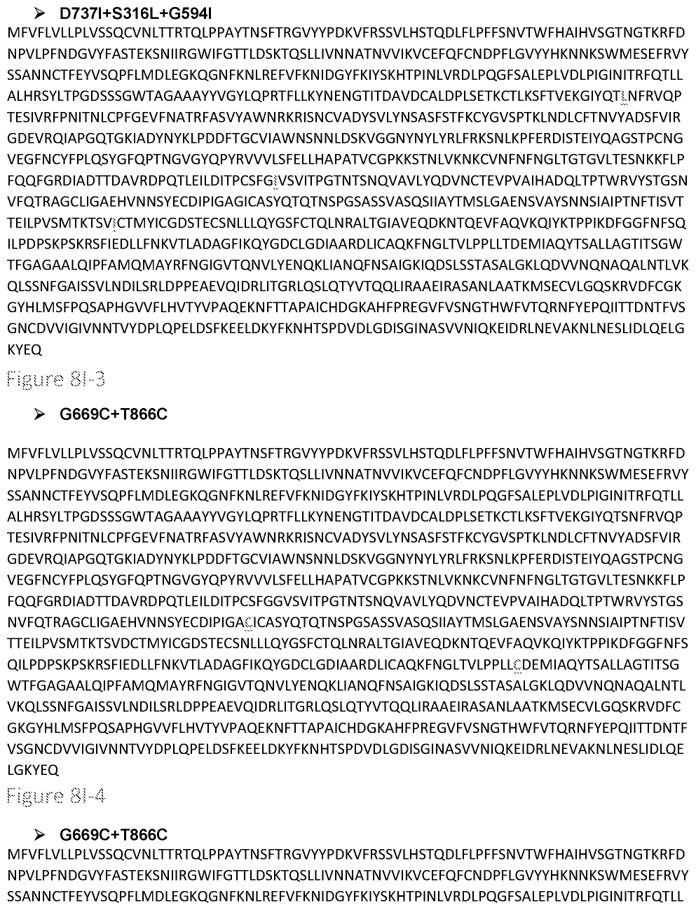

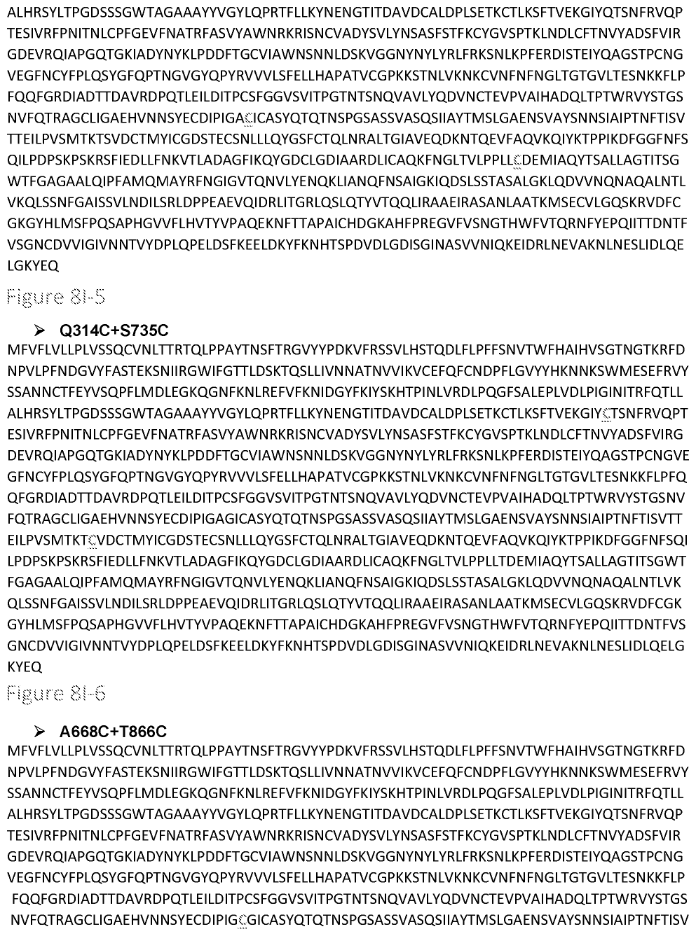

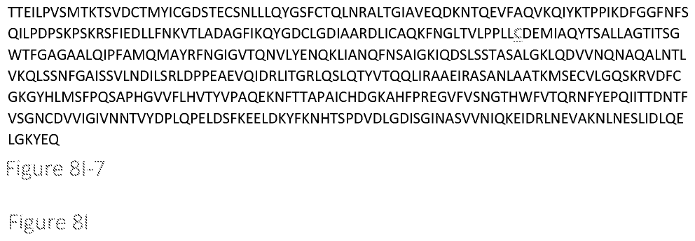

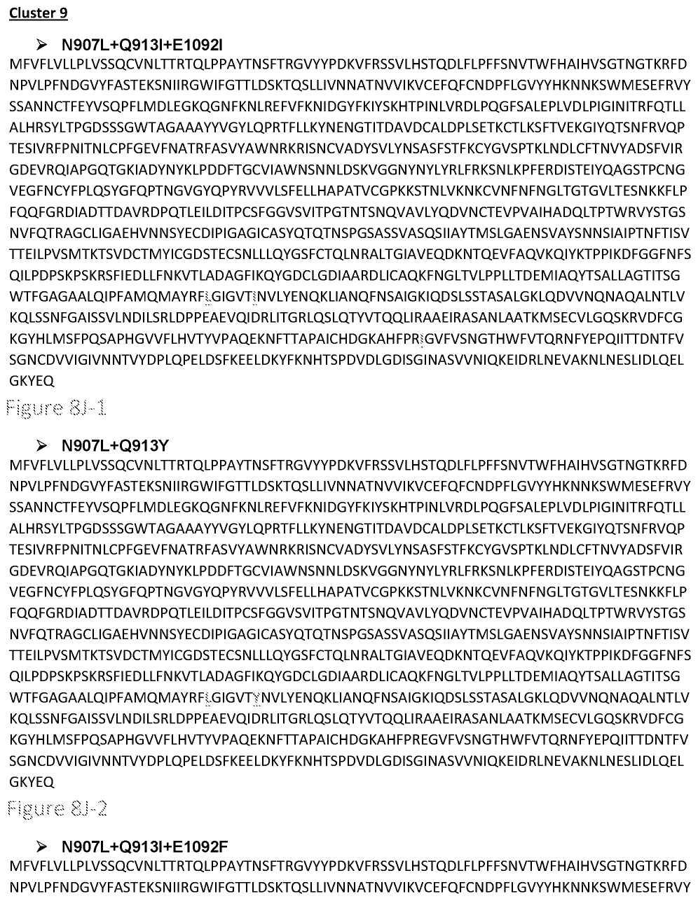

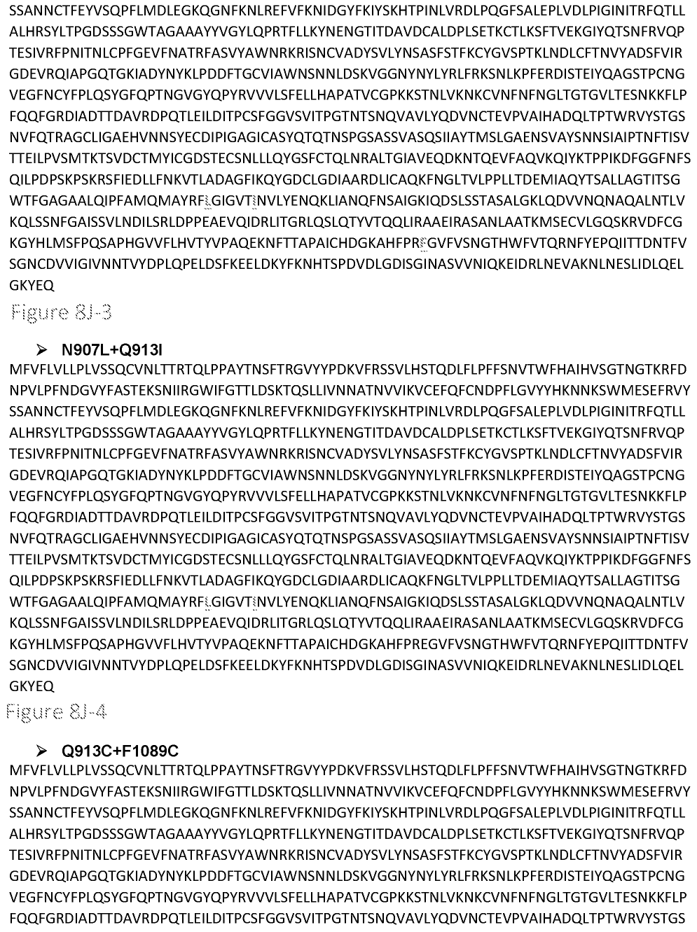

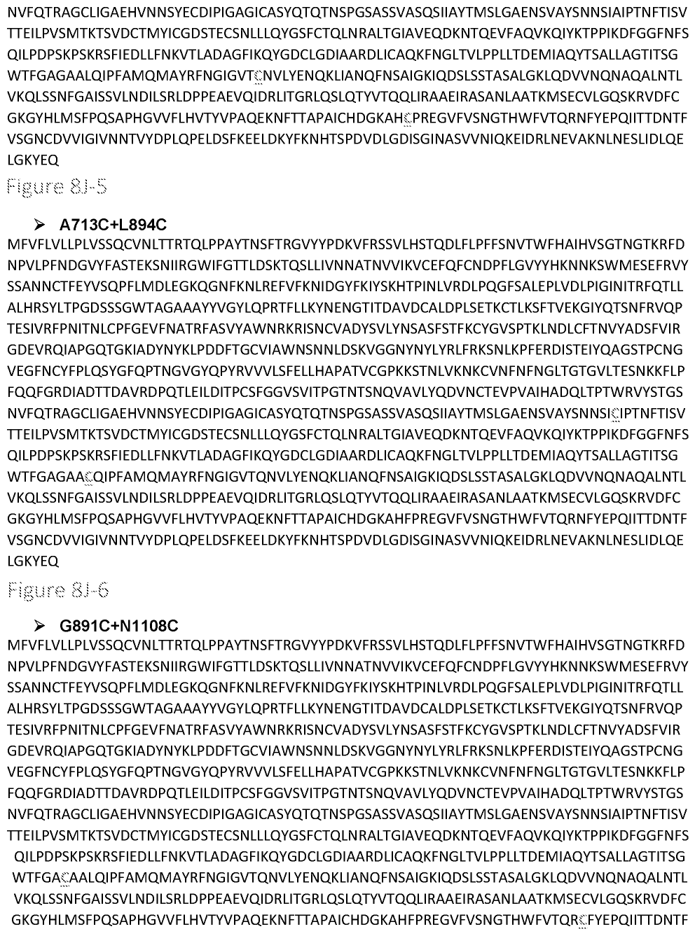

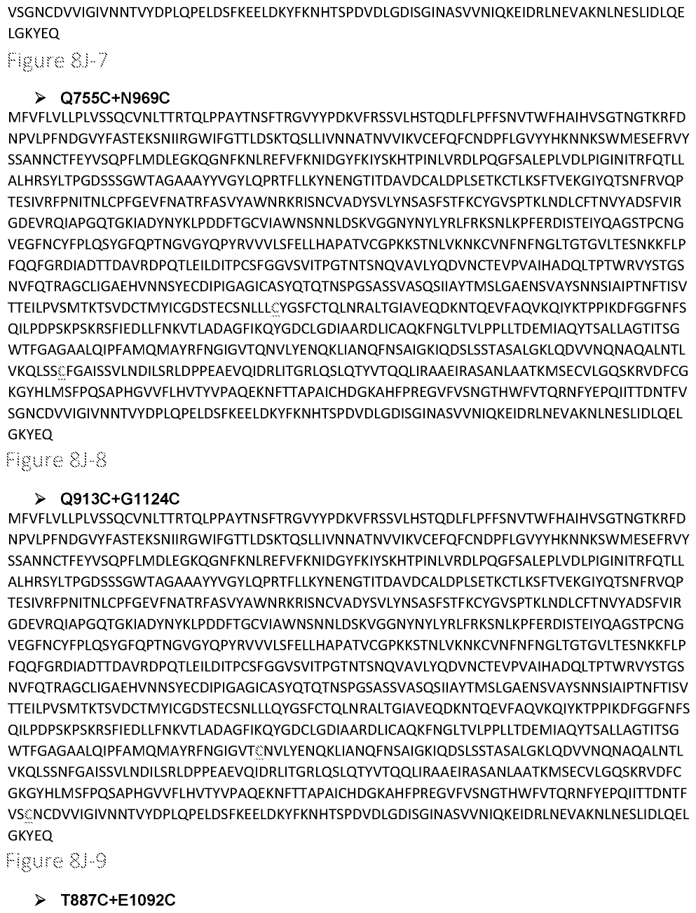

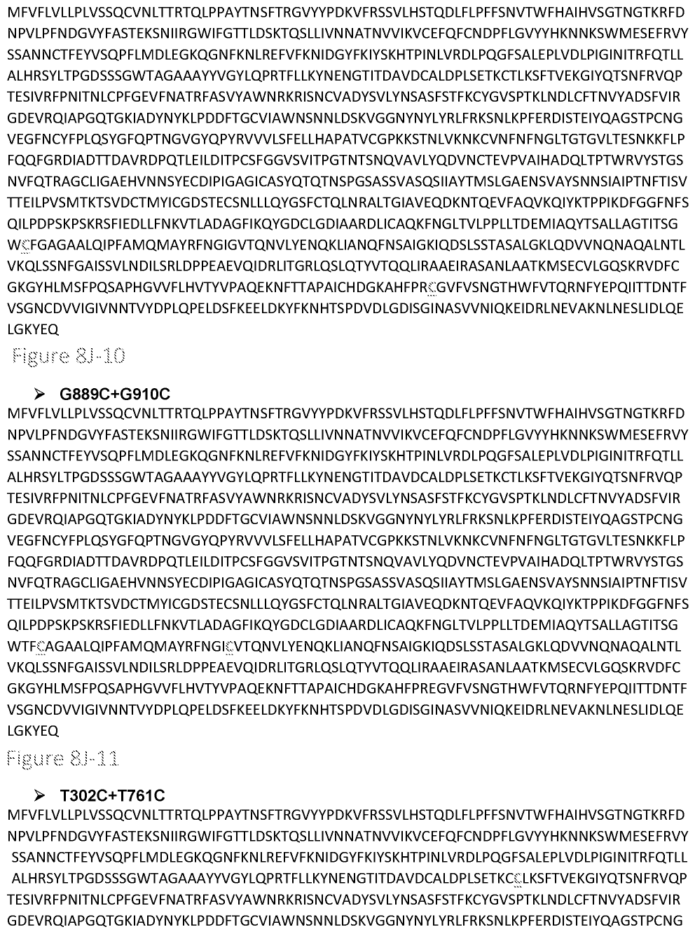

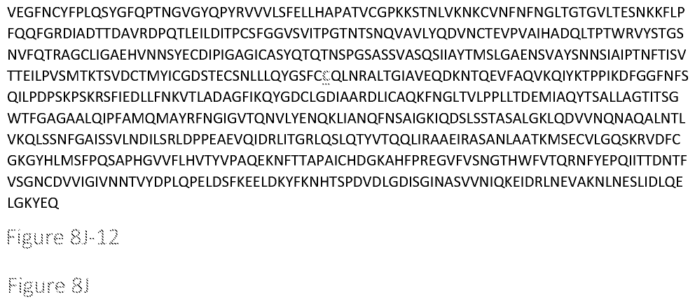

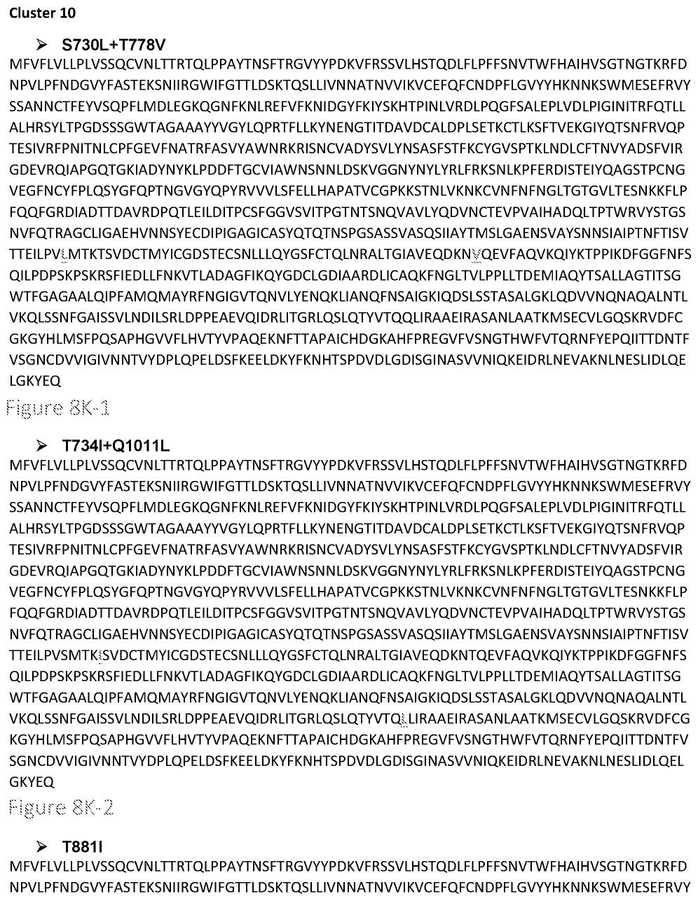

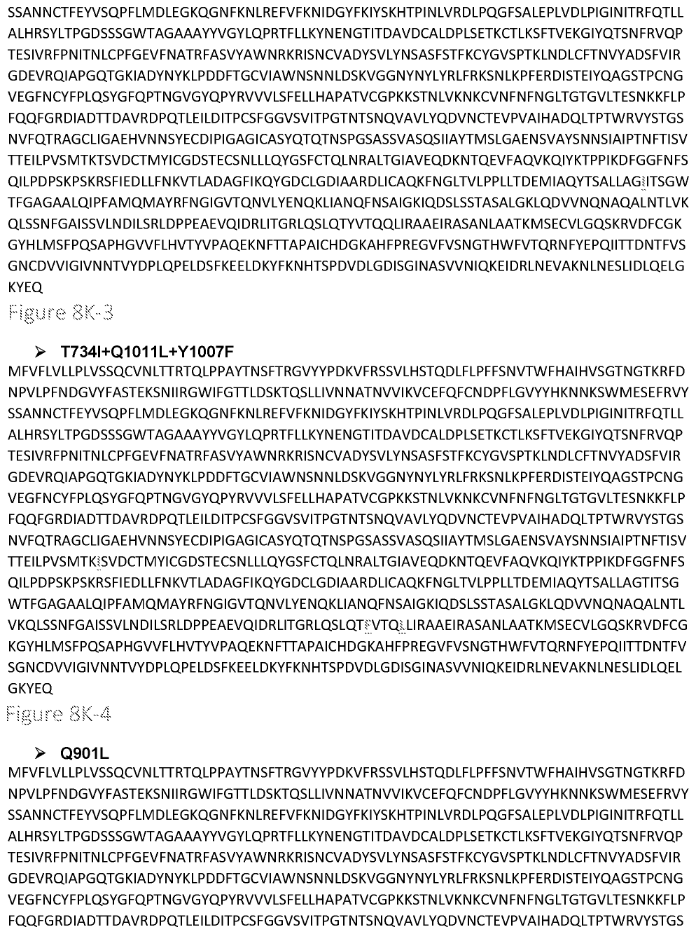

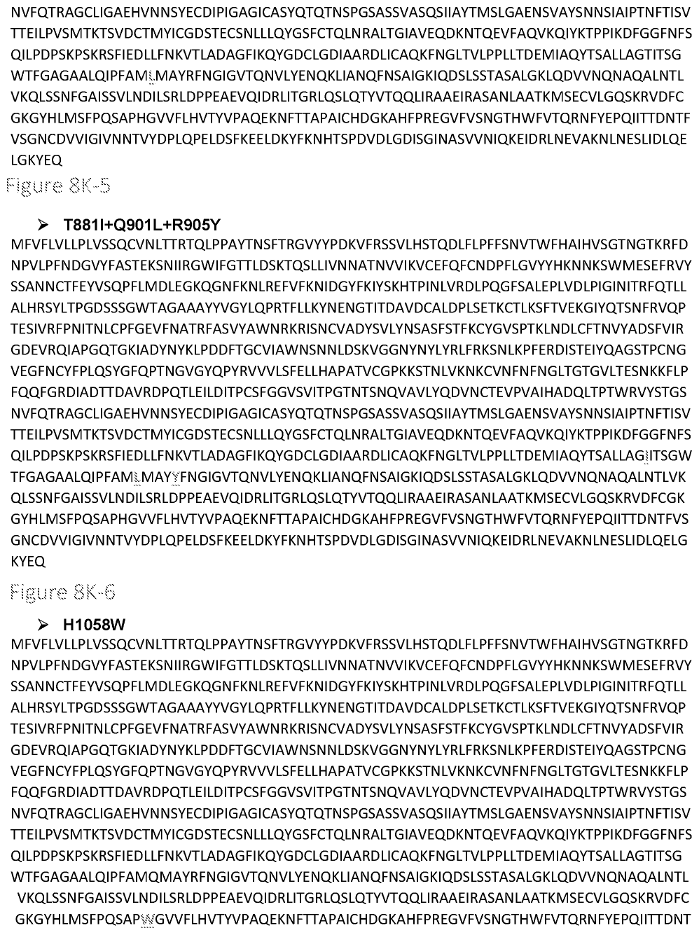

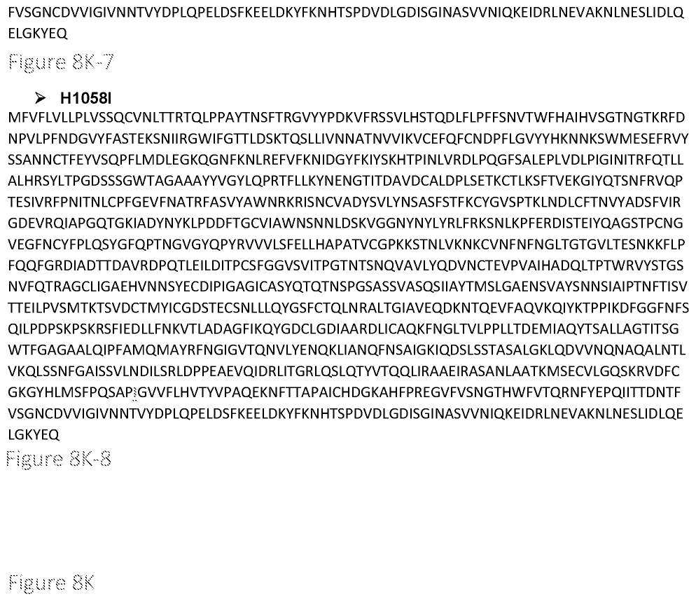

In certain aspects the invention provides methods to define symmetric and asymmetric down state domain arrangements in the SARS-2 S protein. Our analysis of the available CoV S-protein structures reveals a wide breadth of conformational states. We therefore ask the following questions: 1) Is it possible to eliminate or markedly reduce S1 flexibility? 2) How does the stabilizing strategy affect distant domain arrangements? 3) The MERS spike domain arrangement is distinct from SARS and SARS- 2; is it possible to insert MERS residue substitutions in SARS-2 to induce this arrangement? 4) Does a change in domain arrangement impact ectodomain antigenicity? In order to answer these questions, we designed differentially stabilized the S-protein domains. To this end, we have screened in silico, using the Schrödinger software suite, a large panel of mutations designed to stabilize specific regions of the S-protein (FIG. 7; see FIG. 8 and Example 3). Using a high-throughput experimental screen, we down-selected mutations that stabilize each region. We will perform biochemical and biophysical analyses, followed by structural studies using NSEM (low cost, high throughout) and cryo-EM (high resolution). High resolution structures will elucidate the structural effect of the stabilization, and will indicate the degree to which the domains have shifted position, the extent to which the ‘up’/‘down’ RBD equilibrium has shifted, and will enable identification of epitope impacts. Screening of these fully characterized constructs for changes in patient sera antigenicity will then determine the extent to which shifting the conformational ensemble has affected antigenicity and will highlight potential vaccine immunogenicity impacts.

Approach and Methods:

Small-scale transfections of plasmids encoding the mutated S-protein (FIGS. 7 and 8) in HEK293F cells.

Testing of cell-culture supernatants for binding to 1) Streptavidin, in a biolayer interferometry (BLI)-based screen similar to that performed in our recent HIV-1 stabilization manuscript¹², to determine expression levels and 2) to the ACE-2 receptor, and other RBD-reactive ligands such as antibodies CR3022 and 47D11 ¹³⁻¹⁶, that will report on the disposition of the RBD within the spike. Supernatants from untransfected cells will be used as control.

Constructs showing optimal expression and certainACE-2 binding phenotypes will be purified using the PureSpeed (Mettler Toledo) IMAC based high-throughput purification system.

Purified proteins will then be characterized using SDS-PAGE, western blotting, rapid fluorescence-based thermostability assays ¹⁷⁻¹⁹ size exclusion chromatography (SEC) and NSEM.

Constructs with confirmed expression of the trimeric spike protein (SDS-PAGE, SEC and NSEM), and improved properties, e.g. but not limited to melting temperature at least 5° C. higher than the unmutated construct, will be selected as candidates for the next round of selection. For these constructs we will determine 1) ACE2 binding and affinity, 2) thermal stability via differential scanning calorimetry, and 3) high-resolution structures via cryo-EM. Collecting large cryo-EM datasets of at least 2 million particles for each mutated construct will allow heterogenous 3D classification. We will compare the structures with that of the unmutated construct, determining changes in residue-residue contacts, epitope shape and accessibility, shifts in the probability of finding the construct in any particular state, and measures of conformational shifts using our vector-based analysis.

Finally, we will test for differential changes in antigenicity of these constructs using sera from infected patient. This will provide selection criteria for subsequent studies.

Constructs will also be tested for immunogenicity in any suitable animal model, including without limitation mouse studies, NHP studies, and so forth.

Small-scale transfections can yield relatively small quantities of protein for some constructs. NSEM and fluorescence-based thermostability measurements require very small amount of protein (less than 10 μg), increasing the chances that most of the constructs will yield sufficient protein for the small-scale screens. For those constructs that do not, we will use a larger transfection volume. The ones that fail to express we will remove from our list. Structural determination by cryo-EM also requires very small amounts of protein thus ensuring for most constructs we will be able to obtain high resolution feedback on the designs. 2. Failed designs: Some of the designs may not show expected phenotypes, a risk inherent in this type approach. The large number of in silico designs we are starting with along with the high throughput assays, can allow us to quickly select the designs that show promise and rapidly iterate the experimental and design cycles. This approach has been successful in structure-guided vaccine design^{20,21}. If a particular set fail to provide a stabilized construct, we will turn to the high-resolution cryo-EM structures determined in our heterogenous refinement of the unmutated construct to initiate additional design iterations.

At the successful conclusion of this study, without wishing to be bound by theory, we will provide a detailed, high-resolution mapping of conformational states occupied by the SAR-2 S-protein and shifts in conformational distribution with changes in domain interface interactions. Further, we will demonstrate the degree to which changes in the conformational distribution alter S-protein antigenicity. These results will provide a framework from which to consider how genetic drift in the SARS-2 can affect the spread of the disease and how containment by vaccination can be affected by the selection of conformationally varying mutants.

In certain aspects the invention provides methods to define, in atomic detail, the transition between the down and up states of the SARS-2 S protein spike. While the HIV-1 Env utilizes a complex network of allosteric machinery to signal receptor binding, the CoV S protein appears to use a kinetic strategy toward receptor recognition and triggering (FIG. 6E). The receptor binding site is buried in the closed, all RBD ‘down’ state, and initial receptor interactions can be governed by the probability of encountering an ‘up’ state RBD. In the absence of a robust allosteric network, further receptor binding can be governed by the probability of an additional RBD transitioning to the ‘up’ state. Thus, a purely equilibrium perspective of the states can miss important physical characteristics of the transitions and limit a robust, predictive framework from which to understand and exploit structural data. To this end, we will monitor the exchange dynamics of domain repositioning in the SARS-2 spike ectodomain using adaptive sampling. Accumulating simulated data on the order of hundreds of thousands of microseconds, we will stitch together the resulting dynamics using proven Markov state modelling approaches^{22,23}. We will then identify key, as yet undetermined, long-lived transition states for structural interrogation by high-resolution cryo-EM. Combining an advanced framework for simulating the SARS-2 spike ectodomain along with high-resolution cryo-EM structures, will provide a robust, validated model for the conformational transitions.

Approach and Methods:

An in-house developed projection method calculating the pairwise relative angles between the NTD, NTD′, RBD, subdomains 1 and 2, the S2-sheet motif, and the CD of each protomer will used in the adaptive scheme.

Converged Markov model transition intermediates will be used as “bait” to isolate minor populations of intermediates by heterogenous classification of cryo-EM data

These MD based particle sets will be unbiased via independent ab initio map reconstruction and subsequent high-resolution refinement for comparison against the MD state.

The equilibrium distribution of states determined by cryo-EM will be compared to the MD predicted equilibrium. Upon validation, we will analyze the MD transition kinetics, thermodynamics, and path(s).

All atom simulations will be carried out using HTMD²⁴ and Amberz18²⁵ for the adaptive sampling protocol. The Amber ﬀ14SB²⁶ and Glycam²⁷ forcefields using a truncated octahedral TIP3P²⁸ water box and a time-step of 4 fs using hydrogen mass repartitioning²⁹ in the NVT ensemble will be used throughout for production runs. Simulations will be lengthened and the number of iterations increased if model validation demonstrate a need. Markov models will be built using the PyEMMA²² software package. Markov model convergence will be monitored based upon linearity in the implied timescale plots and the Chapman-Kolmogorov test and uncertainty will be determined via bootstrapping of the simulated data.

As blind sampling of the adaptive states can lead to significant simulation time spent in irrelevant states, we will use the FAST′ algorithm to focus sampling in the direction of the known open, ‘up’ state. Due to the size and complexity of the CoV structure, a divide and conquer approach toward simulating the opening process can be necessary. We will split the approach into several distinct modelling steps via a combination of proven approaches³¹⁻³³ as needed. The coordinate projection method and the Markov model lag time must be optimized as well. We will test multiple projection methods and compare using the so-called VAMP scoring criteria³⁴. Finally, if inconsistencies between the simulated and experimental results arise our path forward will involve a sequential shift toward relying upon the experimental data to drive the description of the transition. Even if the model rates and equilibrium values disagree with the cryo-EM data we will still be able to discern possible paths and identify mutations that affect the distribution. Determination of transition kinetics via ACE2 binding, thermal melting temperatures, and cryo-EM state distributions can instead be used to define the transition while still retaining the utility of the MD approach.

These studies can provide key details important for understanding the transition from the prefusion, closed to the post fusion open structures of the SARS-2 fusion protein. This will include a detailed description of metastable intermediate states, transition states, transition kinetics, and transition free energies. This mechanism will be supported by high-resolution structures. Together, this information will provide atomic details important for both drug and vaccine design as well as in the prediction of conformational evasion mutations in the evolving SARS-2 virus.

### REFERENCES

1 Walls, A. C. et al. Structure, Function, and Antigenicity of the SARS-CoV-2 Spike Glycoprotein. Cell, doi: doi.org/10.1016/j.cell.2020.02.058 (2020).
2 Wrapp, D. et al. Cryo-EM structure of the 2019-nCoV spike in the prefusion conformation. Science 367, 1260-1263, doi:10.1126/science.abb2507 (2020).
3 Kirchdoerfer, R. N. et al. Pre-fusion structure of a human coronavirus spike protein. Nature 531, 118-121, doi:10.1038/nature17200 (2016).
4 Yuan, Y. et al. Cryo-EM structures of MERS-CoV and SARS-CoV spike glycoproteins reveal the dynamic receptor binding domains. Nature Communications 8, 15092, doi:10.1038/ncomms15092 (2017).
5 Gui, M. et al. Cryo-electron microscopy structures of the SARS-CoV spike glycoprotein reveal a prerequisite conformational state for receptor binding. Cell Research 27, 119-129, doi:10.1038/cr.2016.152 (2017).
6 Song, W., Gui, M., Wang, X. & Xiang, Y. Cryo-EM structure of the SARS coronavirus spike glycoprotein in complex with its host cell receptor ACE2. PLOS Pathogens 14, e1007236, doi:10.1371/journal.ppat.1007236 (2018).
7 Kirchdoerfer, R. N. et al. Stabilized coronavirus spikes are resistant to conformational changes induced by receptor recognition or proteolysis. Scientific Reports 8, 15701, doi:10.1038/s41598-018-34171-7 (2018).
8 Walls, A. C. et al. Unexpected Receptor Functional Mimicry Elucidates Activation of Coronavirus Fusion. Cell 176, 1026-1039.e1015, doi:https://doi.org/10.1016/j.cell.2018.12.028 (2019).
9 Pallesen, J. et al. Immunogenicity and structures of a rationally designed prefusion MERS-CoV spike antigen. Proceedings of the National Academy of Sciences 114, E7348, doi:10.1073/pnas.1707304114 (2017).
10 Tortorici, M. A. et al. Structural basis for human coronavirus attachment to sialic acid receptors. Nature Structural & Molecular Biology 26, 481-489, doi:10.1038/s41594-019-0233-y (2019).
11 Walls, A. C. et al. Cryo-electron microscopy structure of a coronavirus spike glycoprotein trimer. Nature 531, 114-117, doi:10.1038/nature16988 (2016).
12 Henderson, R. et al. Disruption of the HIV-1 Envelope allosteric network blocks CD4-induced rearrangements. Nature Communications 11, 520, doi:10.1038/s41467-019-14196-w (2020).
13 Hodgson, J. The pandemic pipeline. Nature Biotechnology (2020).
14 Wang, C. e. a. A human monoclonal antibody blocking SARS-CoV-2 infection. bioRxiv doi: doi.org/10.1101/2020.03.11.987958 (2020).
15 ter Meulen, J. et al. Human monoclonal antibody combination against SARS coronavirus: synergy and coverage of escape mutants. PLoS Med 3, e237, doi:10.1371/journal.pmed.0030237 (2006).
16 Tian, X. et al. Potent binding of 2019 novel coronavirus spike protein by a SARS coronavirus-specific human monoclonal antibody. Emerg Microbes Infect 9, 382-385, doi:10.1080/22221751.2020.1729069 (2020).
17 Nilsen, J. et al. Human and mouse albumin bind their respective neonatal Fc receptors differently. Sci Rep 8, 14648, doi:10.1038/s41598-018-32817-0 (2018).
18 Hendus-Altenburger, R. et al. Molecular basis for the binding and selective dephosphorylation of Na(+)/H(+) exchanger 1 by calcineurin. Nat Commun 10, 3489, doi:10.1038/s41467-019-11391-7 (2019).
19 Nosrati, M. et al. Functionally critical residues in the aminoglycoside resistance-associated methyltransferase RmtC play distinct roles in 30S substrate recognition. J Biol Chem 294, 17642-17653, doi:10.1074/jbc.RA119.011181 (2019).
20 McLellan, J. S. et al. Structure-based design of a fusion glycoprotein vaccine for respiratory syncytial virus. Science 342, 592-598, doi:10.1126/science.1243283 (2013).
21 Kwon, Y. D. et al. Crystal structure, conformational fixation and entry-related interactions of mature ligand-free HIV-1 Env. Nat Struct Mol Biol 22, 522-531, doi:10.1038/nsmb.3051 (2015).
22 Scherer, M. K. et al. PyEMMA 2: A Software Package for Estimation, Validation, and Analysis of Markov Models. Journal of Chemical Theory and Computation 11, 5525-5542, doi:10.1021/acs.jctc.5b00743 (2015).
23 Chodera, J. D. & Noe, F. Markov state models of biomolecular conformational dynamics. Current Opinion in Structural Biology 25, 135-144, doi:doi.org/10.1016/j.sbi.2014.04.002 (2014).
24 Doerr, S., Harvey, M. J., Noe, F. & De Fabritiis, G. HTMD: High-Throughput Molecular Dynamics for Molecular Discovery. Journal of Chemical Theory and Computation 12, 1845-1852, doi:10.1021/acs.jctc.6b00049 (2016).
25 D. A. Case, D. S. C., T. E. Cheatham, I I I, T. A. Darden, R. E. Duke, T. J. Giese, H. Gohlke, A. W. Goetz, D. Greene, N. Homeyer, S. Izadi, A. Kovalenko, T. S. Lee, S. LeGrand, P. Li, C. Lin, J. Liu, T. Luchko, R. Luo, D. Mermelstein, K. M. Merz, G. Monard, H. Nguyen, I. Omelyan, A. Onufriev, F. Pan, R. Qi, D. R. Roe, A. Roitberg, C. Sagui, C. L. Simmerling, W. M. Botello-Smith, J. Swails, R. C. Walker, J. Wang, R. M. Wolf, X. Wu, L. Xiao, D. M. York and P. A. Kollman. (2017).
26 Maier, J. A. et al. ﬀ14SB: Improving the Accuracy of Protein Side Chain and Backbone Parameters from ﬀ99SB. Journal of Chemical Theory and Computation 11, 3696-3713, doi:10.1021/acs.jctc.5b00255 (2015).
27 Kirschner, K. N. et al. GLYCAM06: a generalizable biomolecular force field. Carbohydrates. Journal of computational chemistry 29, 622-655, doi:10.1002/jcc.20820 (2008).
28 Jorgensen, W. L., Chandrasekhar, J., Madura, J. D., Impey, R. W. & Klein, M. L. Comparison of simple potential functions for simulating liquid water. The Journal of Chemical Physics 79, 926-935, doi:10.1063/1.445869 (1983).
29 Hopkins, C. W., Le Grand, S., Walker, R. C. & Roitberg, A. E. Long-Time-Step Molecular Dynamics through Hydrogen Mass Repartitioning. Journal of Chemical Theory and Computation 11, 1864-1874, doi:10.1021/ct5010406 (2015).
30 Zimmerman, M. I. & Bowman, G. R. FAST Conformational Searches by Balancing Exploration/Exploitation Trade-Offs. Journal of Chemical Theory and Computation 11, 5747-5757, doi:10.1021/acs.jctc.5b00737 (2015).
31 Moradi, M. & Tajkhorshid, E. Computational Recipe for Efficient Description of Large-Scale Conformational Changes in Biomolecular Systems. Journal of Chemical Theory and Computation 10, 2866-2880, doi:10.1021/ct5002285 (2014).
32 Wang, W., Cao, S., Zhu, L. & Huang, X. Constructing Markov State Models to elucidate the functional conformational changes of complex biomolecules. WIREs Computational Molecular Science 8, e1343, doi:10.1002/wcms.1343 (2018).
33 Singharoy, A. & Chipot, C. Methodology for the Simulation of Molecular Motors at Different Scales. The Journal of Physical Chemistry B 121, 3502-3514, doi:10.1021/acs.jpcb.6b09350 (2017).
34 Mardt, A., Pasquali, L., Wu, H. & Noe, F. VAMPnets for deep learning of molecular kinetics. Nature Communications 9, 5, doi:10.1038/s41467-017-02388-1 (2018).

The SARS-2 S protein includes the receptor binding domain and is a target for neutralizing antibodies. We have designed recombinant DNA constructs that express SARS-2 coronavirus S protein (GenBank Accession number: YP_009724390.1, which is incorporated by reference) as the full-length, transmembrane S protein or a truncated version of the S protein that lacks the C-terminal transmembrane domain and cytoplasmic tail. The truncated S protein is secreted from expressing cells, whereas the full-length version of the plasmid is expressed on the cell surface. Additional SARS-2 S protein sequences from circulating viruses are found in the GISAID EpiFlu™ Database. These sequences can also be modified with any of the modifications described herein.

The S protein designs have several modifications from the wildtype reference sequence from GenBank. First, the SARS-2 protein sequence encodes furin cleavage sites and a cathepsin L cleavage site. The recombinant protein will be made with and without these protease cleavage sites to see if they affect protein quality, yield, and immunogenicity. Second, the natural signal peptide that directs intracellular trafficking of the S protein will be exchanged for the bovine prolactin signal peptide. The bovine prolactin signal peptide is a strong signal peptide that directs proteins into the secretory pathway. This signal peptide is predicted by the SignalP 5.0 program to be cleaved off of the mature S protein more efficiently than the natural virus signal peptide sequence. Third, the secreted S protein can trimerize in order to resemble the native, membrane-bound S protein on coronavirus virions. However, the truncated, secreted S protein lacks the transmembrane domain and thus may not form a stable trimeric protein. To facilitate trimerization, we added a trimerization domain to the C-terminus of some truncated S proteins. The trimerization domain can be a 29 amino acid sequence called foldon for T4 bacteriophage fibritin protein (Strelkov S V et al. Biochemistry. 1999; Frank S et al. J Mol Biol. 2001). Fourth, we have encoded de novo cysteines to the protein sequence to create new intramolecular and intermolecular disulfide bonds. The bonds prevent conformational changes within the S protein. Non-limiting examples are represented by Cluster modifications 1-11. Fifth, we have encoded two new prolines in between HR1 and the central helix in the S protein to stabilize the polypeptide turns in the S2 protein (Pallesen et al. PNAS. 2017). Sixth, we have added an AviTag to the truncated S proteins to facilitate functionalization by streptavidin binding.

For development as a vaccine immunogen, we have also created multimeric nanoparticles that display SARS-CoV-2 S protein on their surface. The rationale for creating such immunogens is that presenting multiple copies of the immunogen allows for a more avid interaction between the immunogen and naïve B cell receptors during the immune response. Thus, weak affinity interactions between the B cell receptor and immunogen are enhanced due to the multiple interactions that work in concert. This improved interaction with B cells can underlies the improved uptake of multimeric immunogens by B cells. The internalized immunogen is then presented to T cells in the context of MHC molecules. The T cells in turn provide the required costimulatory signals to the B cells to promote B cell maturation. Additionally, the SARS-CoV-2 S protein has 22 glycosylation sites, which can interact with lectins to facilitate trafficking to secondary lymphoid organs. Multimerization of viral spike glycoproteins improves their interaction with mannose binding lectin, thereby increasing antigen trafficking to sites with abundant immune cells.

The nanoparticle immunogens are composed of various fragments of SARS-CoV-2 S protein and self-assembling ferritin protein derived from Helicobacter pylori. Each nanoparticle displays 24 copies of the S protein on its surface. The S protein is displayed as a soluble spike trimer that has the transmembrane domain and cytoplasmic tail removed and a foldon trimerization domain added. To focus antibodies to neutralizing targets, the S protein will be truncated down to only the receptor binding domain (RBD), which is a known target for neutralizing antibodies. This construct has the potential to generate neutralizing antibodies, while not eliciting binding antibodies to other sites that mediate antibody-dependent enhancement of virus infectivity (Wang et al. Biochem Biophys Res Commun. 2014 Aug. 22; 451(2):208-14; Jaume et al. J Virol. 2011 October; 85(20):10582-97).

Nucleic Acid Sequences

In certain aspects, the invention provides nucleic acids comprising sequences encoding proteins of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs) or liposomes. In certain embodiments, the mRNAs are modified. Modifications include without limitations modified ribonucleotides, poly-A tail, and/or 5′ cap.

In certain aspects the invention provides nucleic acids encoding the inventive protein designs. In non-limiting embodiments, the nucleic acids are mRNA, modified or unmodified, suitable for use any use, e.g but not limited to use as pharmaceutical compositions. In certain embodiments, the nucleic acids are formulated in lipid, such as but not limited to LNPs or liposomes.

In some embodiments the antibodies are administered as nucleic acids, including but not limited to mRNAs which can be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, US Pub 20180344838A1 at least at paragraphs [0260]-[0281] for non-limiting embodiments of chemical modifications, wherein the content of each is incorporated by reference in its entirety.

mRNAs delivered in LNP formulations have advantages over non-LNPs formulations. See US Pub 20180028645A1.

In certain embodiments the nucleic acid encoding a protein is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequences described herein, or a variant thereof or a fragment thereof. Accordingly, in one embodiment, the invention provides an RNA molecule encoding one or more of inventive antibodies. The RNA can be plus-stranded. Accordingly, in some embodiments, the RNA molecule can be translated by cells without needing any intervening replication steps such as reverse transcription.

In some embodiments, an RNA molecule of the invention can have a 5′ cap (e.g. but not limited to a 7-methylguanosine, 7mG(5′)ppp(5′)NlmpNp). This cap can enhance in vivo translation of the RNA. The 5′ nucleotide of an RNA molecule useful with the invention can have a 5′ triphosphate group. In a capped RNA this can be linked to a 7-methylguanosine via a 5′-to-5′ bridge. An RNA molecule may have a 3′ poly-A tail. It can also include a poly-A polymerase recognition sequence (e.g. AAUAAA) near its 3′ end. In some embodiments, a RNA molecule useful with the invention can be single-stranded. In some embodiments, a RNA molecule useful with the invention can comprise synthetic RNA.

The recombinant nucleic acid sequence can be an optimized nucleic acid sequence. Such optimization can increase or alter the immunogenicity of the protein. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of protein expression are known in the art.

Methods for expression and immunogenicity determination of nucleic acid encoded proteins are known in the art.

A non-limiting embodiment of a neutralization assay is described in Zhao, G., Du, L., Ma, C. et al. A safe and convenient pseudovirus-based inhibition assay to detect neutralizing antibodies and screen for viral entry inhibitors against the new human coronavirus MERS-CoV. Virol J 10, 266 (2013). doi.org/10.1186/1743-422X-10-266, which content is incorporated by reference in its entirety. This assay can be adapted for use for SARS CoV-2.

Non-limiting embodiments of determining antibody responses are described in the following publication: “SARS-CoV-2 specific antibody responses in COVID-19 patients” Okba et al. doi.org/10.1101/2020.03.18.20038059. See also US Patent Publication 20200061185 which is incorporated by reference in its entirety.

Non-limiting embodiments of various assays, reagents, and technologies for evaluating the immunogens of the invention are described in Muthumani et al. Science Translational Medicine 19 Aug. 2015: Vol. 7, Issue 301, pp. 301ra132, DOI: 10.1126/scitranslmed.aac7462. The assays, reagents, and techniques can be adapted for use for SARS CoV-2.

Recombinant protein production of coronavirus proteins is known. See e.g. in US Patent Pub 20200061185 which disclosure is incorporated by reference in its entirety.

In some embodiments the SARS-2 S proteins of the invention are in a trimeric configuration. In some embodiments the SARS-2 S proteins of the invention are expressed as protomers which form trimers. These designs can comprise any suitable trimerization domain.

Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to a recombinant coronavirus (e.g. SARS-2) S protein ectodomain described herein (e.g., by linkage to the C-terminus of S2) to promote trimerization of the recombinant coronavirus (e.g. SARS-2) S protein ectodomain.

In some examples, the C-terminus of the S2 subunit of the SARS-2 S protein ectodomain can be linked to a T4 fibritin Foldon domain. In specific examples, the T4 fibritin Foldon domain can include the amino acid sequence GYIPEAPRDGQAYVRKDGEWVLLSTF (SEQ ID NO: 1), which adopts a .beta.-propeller conformation, and can fold and trimerize in an autonomous way (Tao et al. 1997 Structure 5:789-798). Optionally, the heterologous trimerization is connected to the recombinant coronavirus (e.g. SARS-2) S protein ectodomain via a peptide linker, such as an amino acid linker. Non-limiting examples of peptide linkers that can be used include glycine, serine, and glycine-serine linkers.

In some embodiments, the SARS-2 spike protein ectodomain trimer can be membrane anchored, for example, for embodiments where the coronavirus (e.g. SARS-2) S protein ectodomain trimer is expressed on an attenuated viral vaccine, or a virus like particle. In such embodiments, the protomers in the trimer can each comprise a C-terminal linkage to a transmembrane domain, such as the transmembrane domain (and optionally the cytosolic tail) of the corresponding coronavirus. For example, the protomers of a disclosed SARS-2 S protein ectodomain trimer can be linked to a SARS-2 S protein transmembrane and cytosolic tail. In some embodiments, one or more peptide linkers (such as a gly-ser linker, for example, a 10 amino acid glycine-serine peptide linker can be used to link the recombinant SARS-2 S protein ectodomain protomer to the transmembrane domain.

The protomers linked to the transmembrane domain can include any of the modifications provided herein (or combinations thereof) as long as the recombinant coronavirus (e.g. SARS-2) S protein ectodomain trimer formed from the protomers linked to the transmembrane domain retains certain properties (e.g., the coronavirus S protein prefusion conformation).

The inventive protein or fragments thereof can be produced using recombinant techniques, or chemically or enzymatically synthesized.

In some embodiments a protein nanoparticle is provided that includes one or more of the disclosed recombinant SARS-2 S proteins, including but not limited to SARS-2 S protein trimers. Non-limiting example of nanoparticles include ferritin nanoparticles, encapsulin nanoparticles, Sulfur Oxygenase Reductase (SOR) nanoparticles, and lumazine synthase nanoparticles, which are comprised of an assembly of monomeric subunits including ferritin proteins, encapsulin proteins, SOR proteins, and lumazine synthase, respectively. Additional protein nanoparticle structures are described by Heinze et al., J Phys Chem B., 120(26):5945-52, 2016; Hsia et al., Nature, 535(7610):136-9, 2016; and King et al., Nature, 510(7503):103-8, 2014; each of which is incorporated by reference herein. To construct such protein nanoparticles a protomer of the SARS-2 S protein ectodomain trimer can be linked to a subunit of the protein nanoparticle (such as a ferritin protein, an encapsulin protein, a SOR protein, or a lumazine synthase protein) and expressed in cells under appropriate conditions. The fusion protein self-assembles into a nanoparticle and can be purified.

In some embodiments, a protomer of a disclosed recombinant SARS-2 S protein ectodomain trimer can be linked to a ferritin subunit to construct a ferritin nanoparticle. Ferritin nanoparticles and their use for immunization purposes (e.g., for immunization against influenza antigens) have been disclosed in the art (see, e.g., Kanekiyo et al., Nature, 499:102-106, 2013, incorporated by reference herein in its entirety). Ferritin is a globular protein that is found in all animals, bacteria, and plants, and which acts primarily to control the rate and location of polynuclear Fe(III)₂O₃ formation through the transportation of hydrated iron ions and protons to and from a mineralized core. The globular form of the ferritin nanoparticle is made up of monomeric subunits, which are polypeptides having a molecule weight of approximately 17-20 kDa. In certain embodiments, the modified coronavirus spike protein or the portion thereof is linked to form a protein multimerizing/nanoparticle subunit by a peptide linker in a sortase reaction, or is directly linked to the protein multimerizing/nanoparticle subunit. In certain embodiments, the protein nanoparticle subunit is a ferritin nanoparticle subunit.

In non-limiting embodiments the multimeric complexes comprising a ferritin sequence are designed and are assembled via sortase reaction. In non-limiting embodiments the multimeric complexes comprise encapsulin.

Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are known to the person of ordinary skill in the art and are further described herein (see, e.g., Zhang, Int. J. Mol. Sci., 12:5406-5421, 2011, which is incorporated herein by reference in its entirety).

In non-specific examples, the ferritin polypeptide is E. coli ferritin, Helicobacter pylori ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use to make a ferritin nanoparticle including a recombinant SARS-2 S protein can be found in GENBANK, for example at accession numbers ZP_03085328, ZP_06990637, EJB64322.1, AAA35832, NP 000137 AAA49532, AAA49525, AAA49524 and AAA49523, which are specifically incorporated by reference herein in their entirety. In some embodiments, a recombinant protein of the invention can be linked to a ferritin subunit to form a nanoparticle.

Polynucleotides encoding a protomer of any of the disclosed recombinant proteins are also provided. These polynucleotides include DNA, cDNA and RNA sequences which encode the protomer, as well as vectors including the DNA, cDNA and RNA sequences, such as a DNA or RNA vector used for immunization. The genetic code to construct a variety of functionally equivalent nucleic acids, such as nucleic acids which differ in sequence but which encode the same protein sequence, or encode a conjugate or fusion protein including the nucleic acid sequence.

Another approach to multimerize expression constructs uses staphylococcus Sortase A transpeptidase ligation to conjugate inventive spike ectodomain trimers or spike subunits, for e.g. but not limited to cholesterol or self multimerizing protein. The trimers can be embedded into liposomes via the conjugated cholesterol.

To conjugate the trimer a C-terminal LPXTG tag (SEQ ID NO: 2) or a N-terminal pentaglycine repeat tag is added to the spike trimer gene, where X signifies any amino acid, such as Ala, Ser, Glu. Cholesterol is also synthesized with these two tags. Sortase A is then used to covalently bond the tagged spike subunit to the cholesterol. The sortase A-tagged spike trimer protein or portion thereof can also be used to conjugate the trimer to other peptides, proteins, or fluorescent labels. In non-limiting embodiments, the sortase A tagged trimers or spike portions are conjugated to ferritin to form nanoparticles.

In several embodiments, the nucleic acid molecule encodes a precursor of the protomer, that, when expressed in an appropriate cell, is processed into a recombinant SARS-2 S protein protomer that can self-assemble into the corresponding recombinant trimer. For example, the nucleic acid molecule can encode a recombinant SARS-2 S protein ectodomain including a N-terminal signal sequence for entry into the cellular secretory system that is proteolytically cleaved in the during processing of the recombinant protein in the cell. Recombinant proteins with different signal peptide sequences are embodied by the invention.

In certain embodiments, amino acid sequences of the invention described herein comprise a signal peptide. A skilled artisan can readily determine the signal peptide sequences. Signal peptide sequences can be removed during recombinant production of proteins. In non-limiting embodiments, provided are amino acid sequences of recombinant proteins which do not include amino acids of comprising a signal peptide.

In some embodiments, the nucleic acid molecule encodes a precursor SARS-2 S polypeptide that, when expressed in an appropriate cell, is processed into a recombinant SARS-2 S protomer including S1 and S2 polypeptides, wherein the recombinant protein includes any of the appropriate modifications described herein, and optionally can be linked to a trimerization domain, such as a T4 Fibritin trimerization domain.

Exemplary nucleic acids can be prepared by molecular and cloning techniques. A wide variety of cloning methods, host cells, and in vitro amplification methodologies are well known to persons of skill, and can be used to make the nucleic acids and proteins of the invention.

The polynucleotides encoding a disclosed recombinant protomer can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a disclosed recombinant protomer can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the disclosed recombinant protomer can be expressed in vitro by DNA transfer into a suitable host cell. The cell can be prokaryotic or eukaryotic. The term also includes any progeny of the subject host cell. All progeny need not be identical to the parental cell since there can be mutations that occur during replication. Methods of stable transfer, meaning that the foreign DNA is continuously maintained in the host, are known in the art.

Host systems for recombinant production can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4.sup.th Ed., Humana Press). Examples of mammalian host cell lines are VERO and HeLa cells, CHO cells, and WI38, BHK, and COS cell lines, although cell lines can be used, such as cells designed to provide higher expression, desirable glycosylation patterns, or other features. In some embodiments, the host cells include HEK293 cells or derivatives thereof, such as GnTI^{-/-} cells, or HEK-293F cells.

In some embodiments, the disclosed recombinant coronavirus (e.g. SARS-2) S protein ectodomain protomer can be expressed in cells under conditions where the recombinant coronavirus (e.g. SARS-2) S protein ectodomain protomer can self-assemble into trimers which are secreted from the cells into the cell media. In such embodiments, each recombinant coronavirus (e.g. SARS-2) S protein ectodomain protomer contains a leader sequence (signal peptide) that causes the protein to enter the secretory system, where the signal peptide is cleaved and the protomers form a trimer, before being secreted in the cell media. The medium can be centrifuged and recombinant coronavirus (e.g. SARS-2) S protein ectodomain trimer can be purified from the supernatant.

A nucleic acid molecule encoding a protomer can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and can be obtained from commercial sources (such as PharMingen, San Diego, Calif.; Protein Sciences Corp., Meriden, Conn.; Stratagene, La Jolla, Calif).

In several embodiments, the viral vector can include an adenoviral vector that expresses a protomer of the invention. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/02231 1.

In some embodiments, a virus-like particle (VLP) is provided that comprises a recombinant protomer of the invention. In some embodiments, a virus-like particle (VLP) is provided that includes a recombinant trimer of the invention. Such VLPs can include a recombinant coronavirus (e.g. SARS-2) S protein ectodomain trimer that is membrane anchored by a C-terminal transmembrane domain, for example the recombinant coronavirus (e.g. SARS-2) S protein ectodomain protomers in the trimer each can be linked to a transmembrane domain and cytosolic tail from the corresponding coronavirus. VLPs lack the viral components that are required for virus replication and thus represent a highly attenuated, replication-incompetent form of a virus. However, the VLP can display a polypeptide (e.g., a recombinant coronavirus (e.g. SARS-2) S protein ectodomain trimer) that is analogous to that expressed on infectious virus particles and can eliciting an immune response to the corresponding coronavirus (e.g. SARS-2) when administered to a subject. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

The immunogens of the invention can be combined with any suitable adjuvant.

A skilled artisan can readily determine the dose and number of immunizations needed to induce immune response. Various assays are known and used in the art to measure to level, breadth and durability of the induced immune response. In non-limiting embodiments the methods comprise two immunizations. The interval between immunizations can be readily determined by a skilled artisan. In non-limiting embodiments, the first and second immunization are about 2, 3, 4, 5, 6, 7, 8, 9, 10, 11, 12, 13, 14, 15, 16 weeks apart.

In certain embodiments the protein dose is in the range of 1-1000 micrograms. In certain embodiments the protein dose is in the range of 10-1000 micrograms. In certain embodiments the protein dose is in the range of 100-1000 micrograms. In certain embodiments the protein dose is in the range of 100-200 micrograms. In certain embodiments the protein dose is in the range of 100-300 micrograms. In certain embodiments the protein dose is in the range of 100-400 micrograms. In certain embodiments the protein dose is in the range of 100-500 micrograms. In certain embodiments the protein dose is in the range of 100-600 micrograms. In certain embodiments the protein dose is in the range of 50-100 micrograms. In certain embodiments the protein dose is in the range of 50-150 micrograms. In certain embodiments the protein dose is in the range of 50-200 micrograms. In certain embodiments the protein dose is in the range of 50-250 micrograms. In certain embodiments the protein dose is in the range of 50-300 micrograms. In certain embodiments the protein dose is in the range of 50-350 micrograms. In certain embodiments the protein dose is in the range of 50-400 micrograms. In certain embodiments the protein dose is in the range of 50-450 micrograms. In certain embodiments the protein dose is in the range of 50-500 micrograms. In certain embodiments the protein dose is in the range of 50-550 micrograms. In certain embodiments the protein dose is in the range of 50-600 micrograms. In certain embodiments the protein dose is in the range of 75-100 micrograms. In certain embodiments the protein dose is in the range of 75-125 micrograms. In certain embodiments the protein dose is in the range of 75-150 micrograms. In certain embodiments the protein dose is in the range of 75-175 micrograms. In certain embodiments the protein dose is in the range of 75-200 micrograms. In certain embodiments the protein dose is in the range of 75-225 micrograms. In certain embodiments the protein dose is in the range of 75-250 micrograms. In certain embodiments the protein dose is 10, 25, 50, 75, 100, 125, 150, 175, 200, 225, 250, 275, 300, 325, 350, 375, 400, 425, 450, 475, 500, 525, 550, 575, 600, 625, 650, 700, 750, 800, 850, 900, 950 or 1000 micrograms.

In certain embodiments adjuvant dose is in the range of 1-200 micrograms. In certain embodiments adjuvant dose is in the range of 1-100 micrograms. In certain embodiments the adjuvant dose is 1-50 micrograms. In certain embodiments the adjuvant dose is 1-25 micrograms. In certain embodiments the adjuvant dose is 1-50 micrograms. In certain embodiments the adjuvant dose is 1-20 micrograms. In certain embodiments the adjuvant dose is 1-50 micrograms. In certain embodiments the adjuvant dose is 1-15 micrograms. In certain embodiments the adjuvant dose is 1-50 micrograms. In certain embodiments the adjuvant dose is 1-10 micrograms. In certain embodiments the adjuvant dose is 1-5 micrograms. In certain embodiments the adjuvant dose is 5-10 micrograms. In certain embodiments the adjuvant dose is 5-15 micrograms. In certain embodiments the adjuvant dose is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 45-50 micrograms. Non-limiting examples of evaluating the immunogenicity and effectiveness of the immunogens of the invention are shown in US Patent Pub 20200061185 which disclosure is incorporated by reference in its entirety.

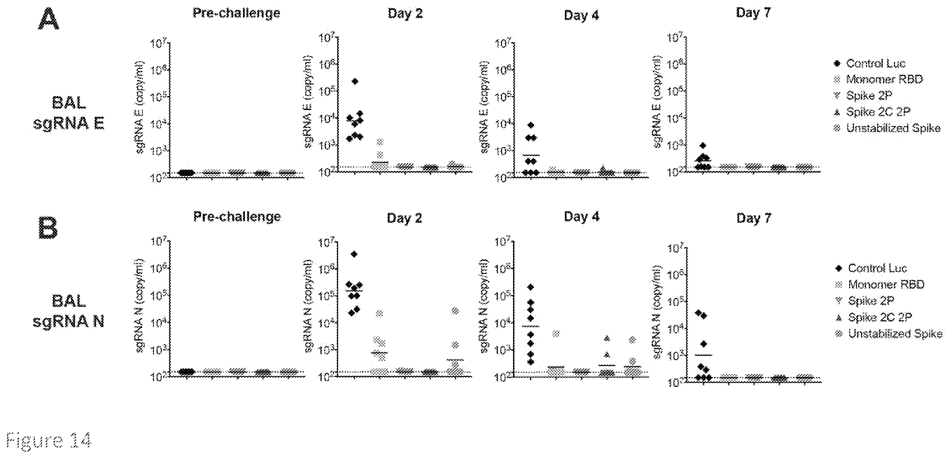

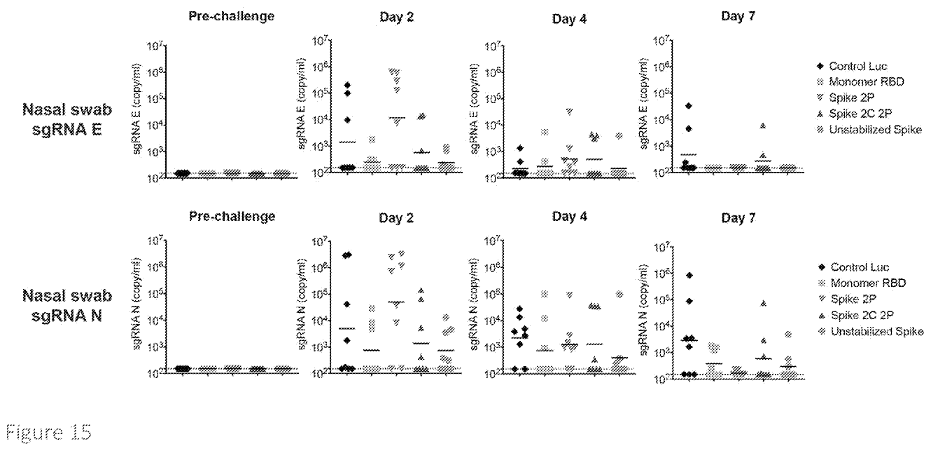

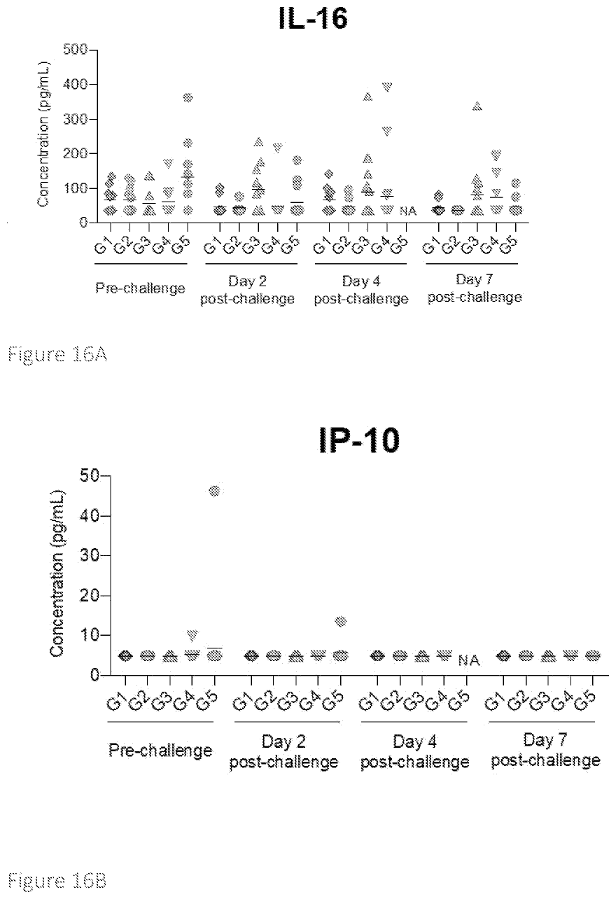

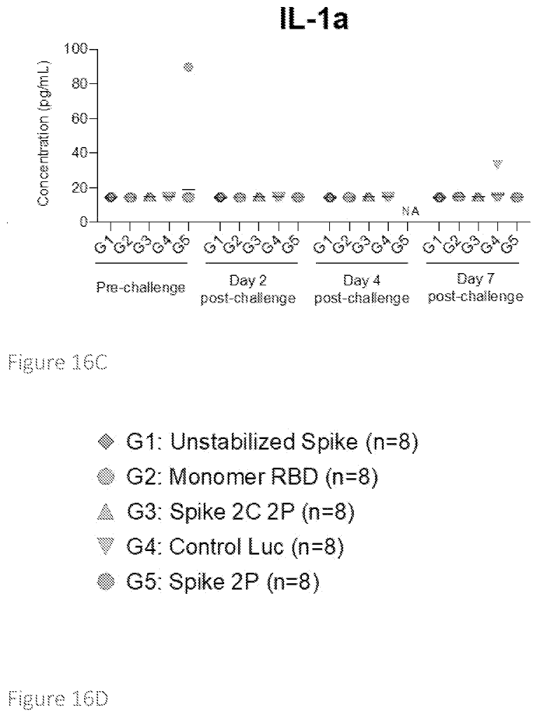

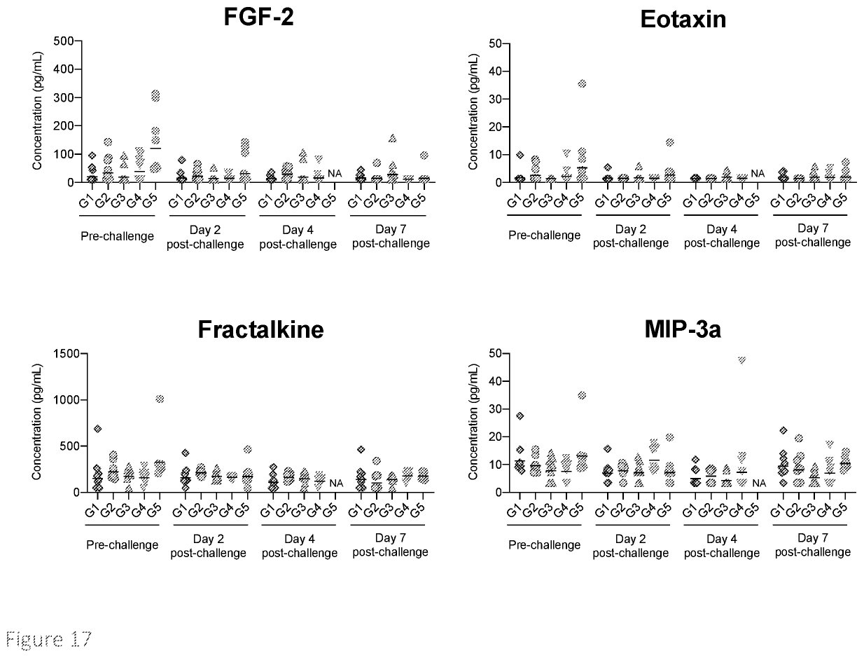

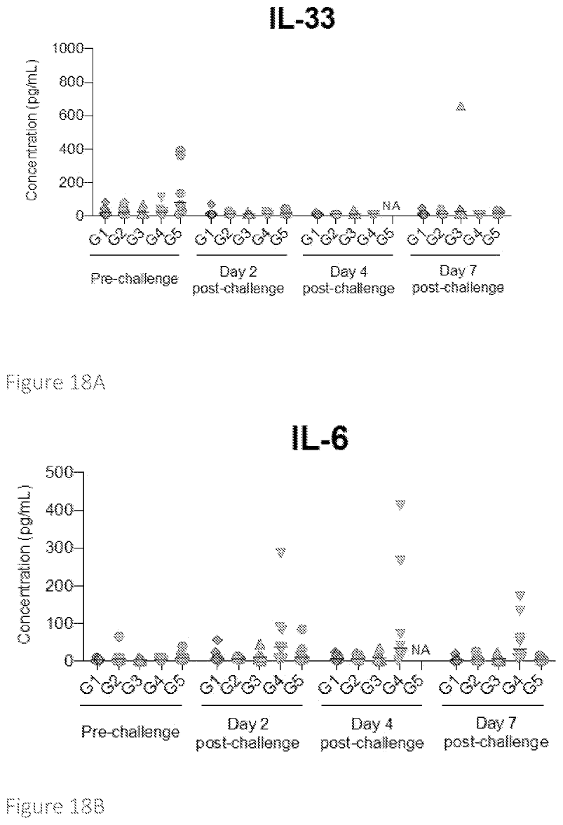

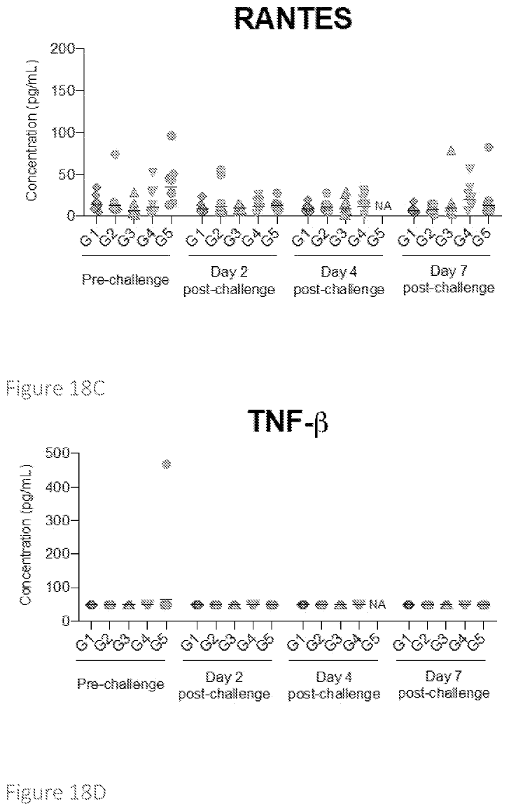

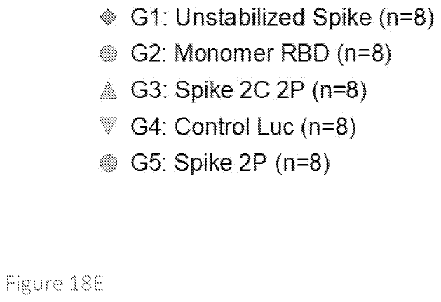

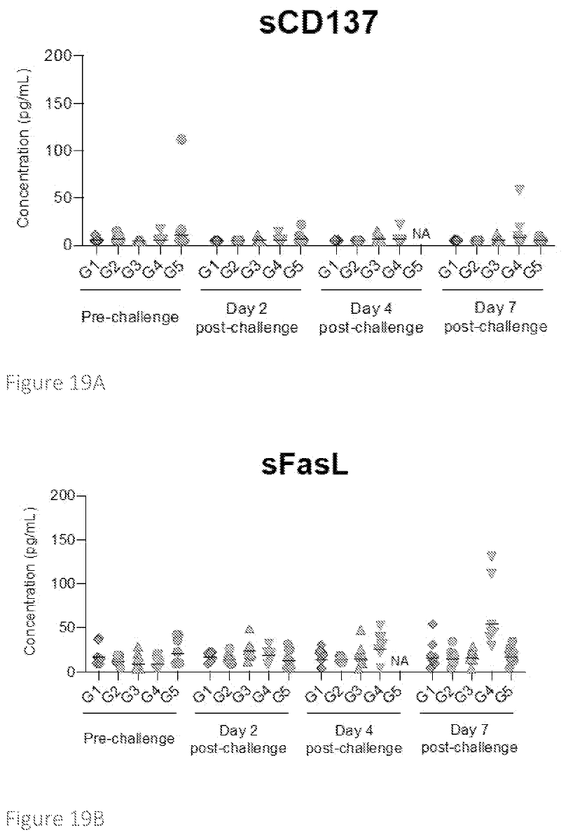

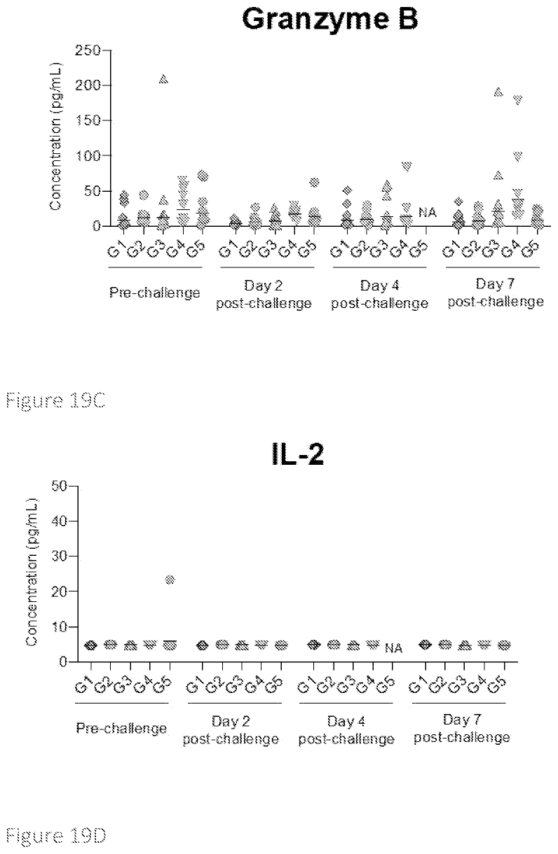

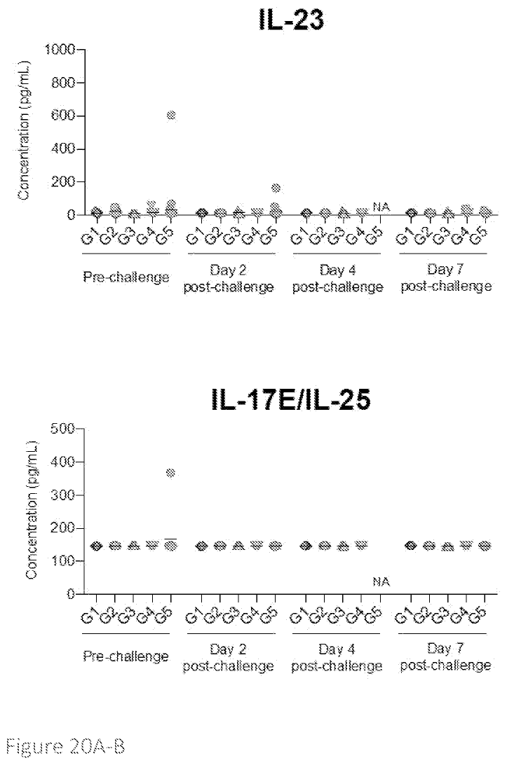

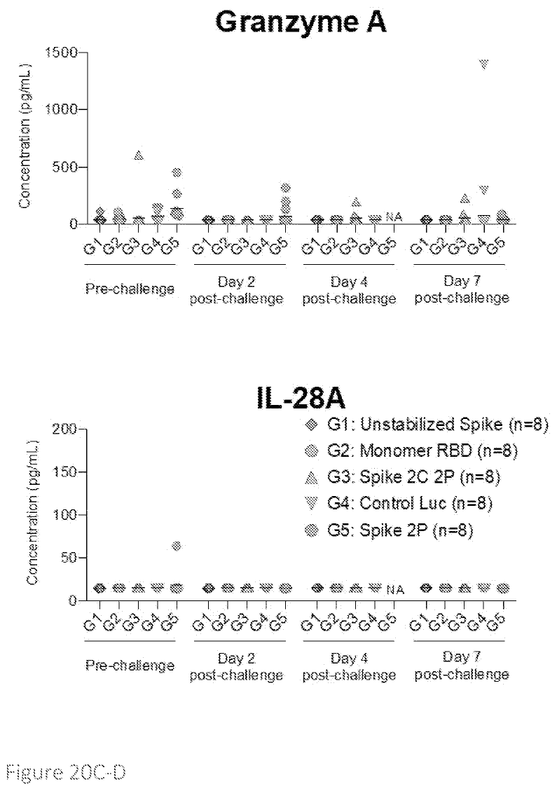

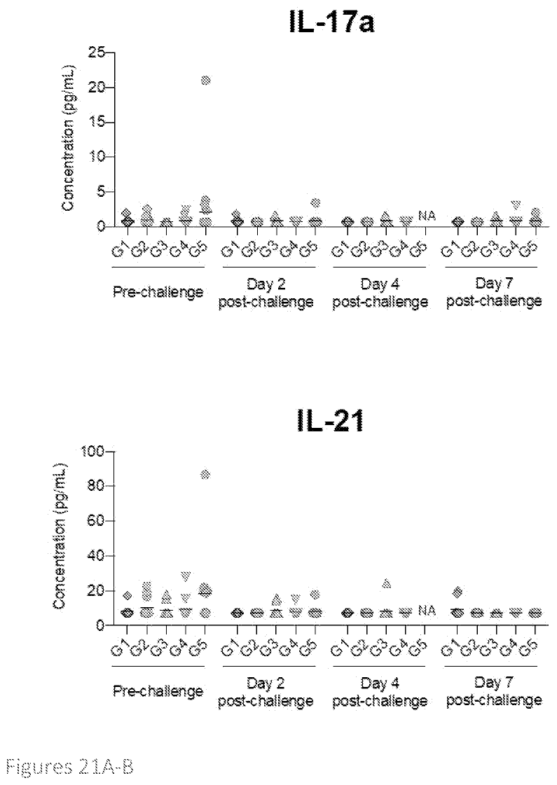

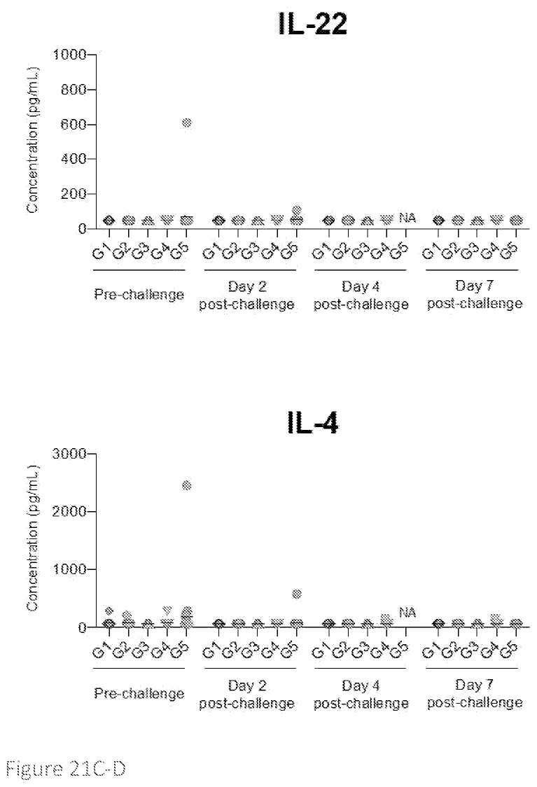

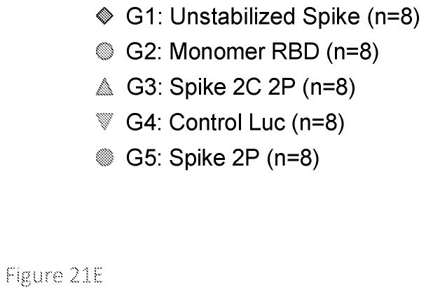

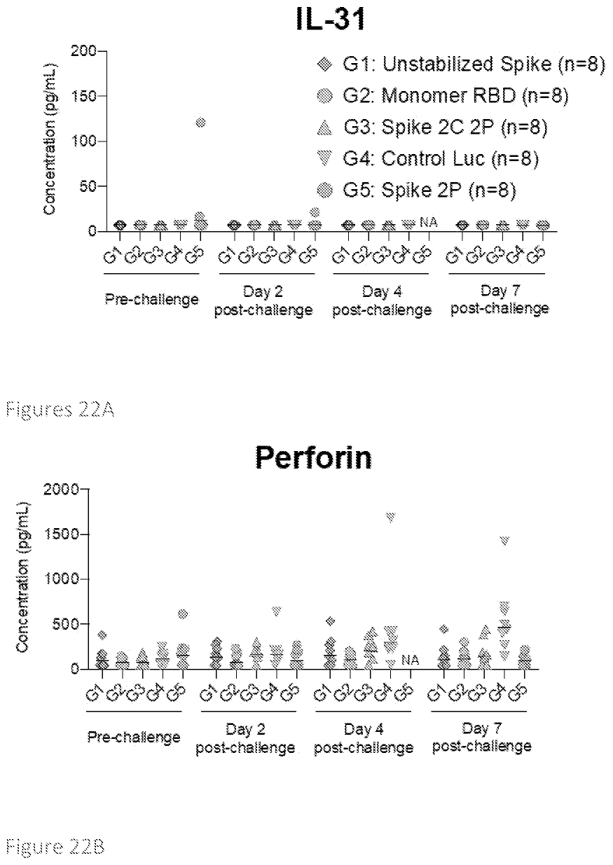

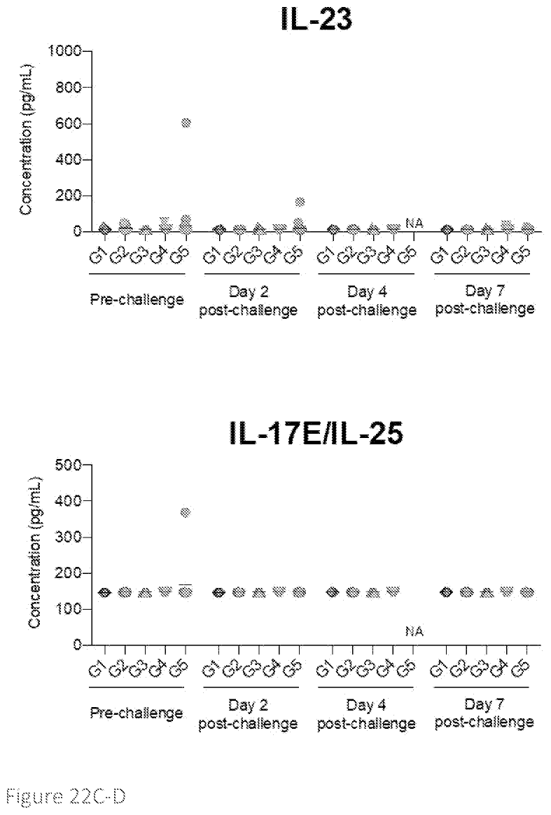

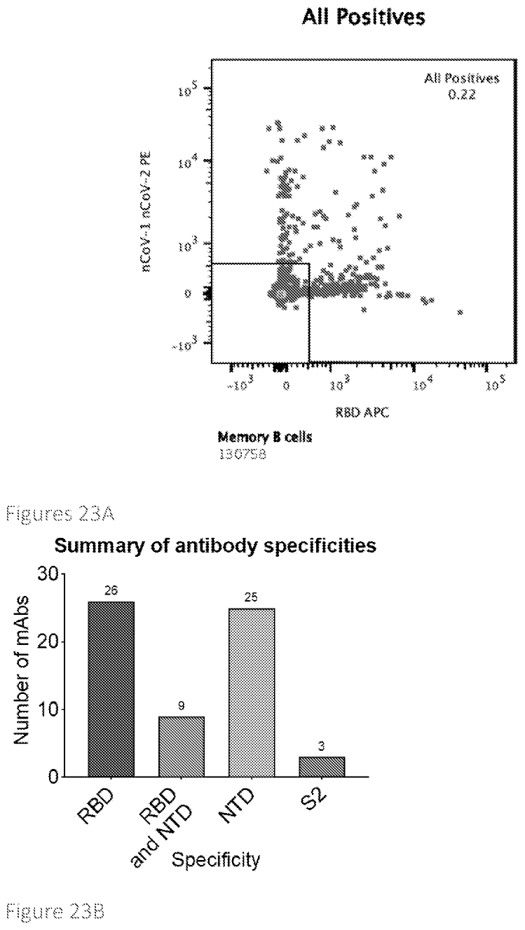

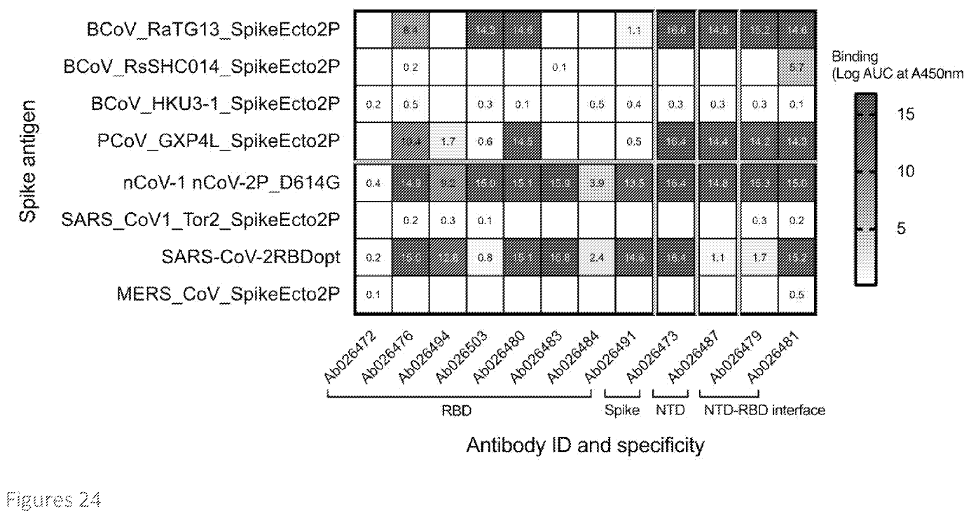

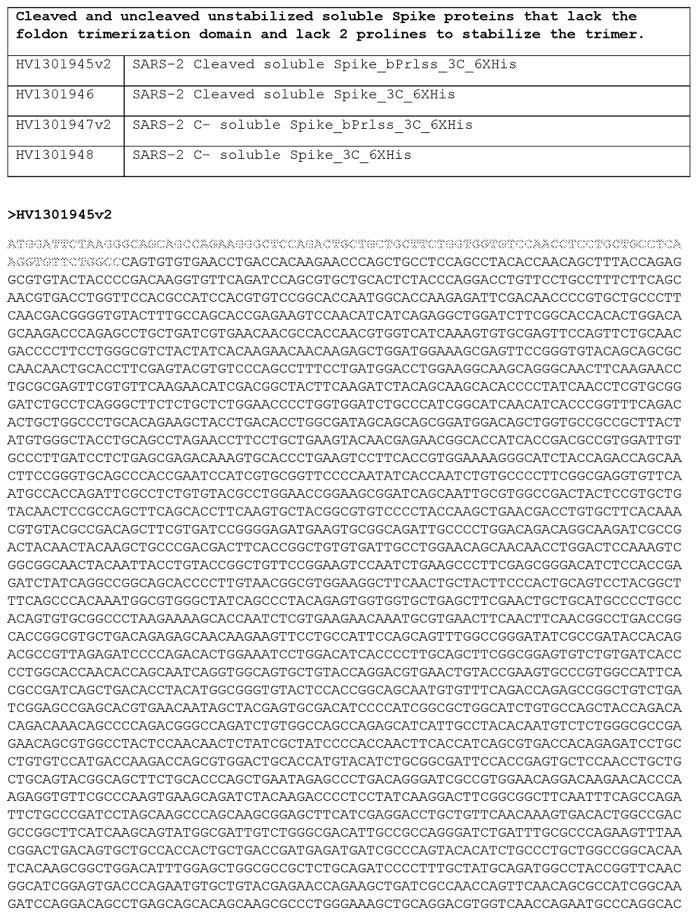

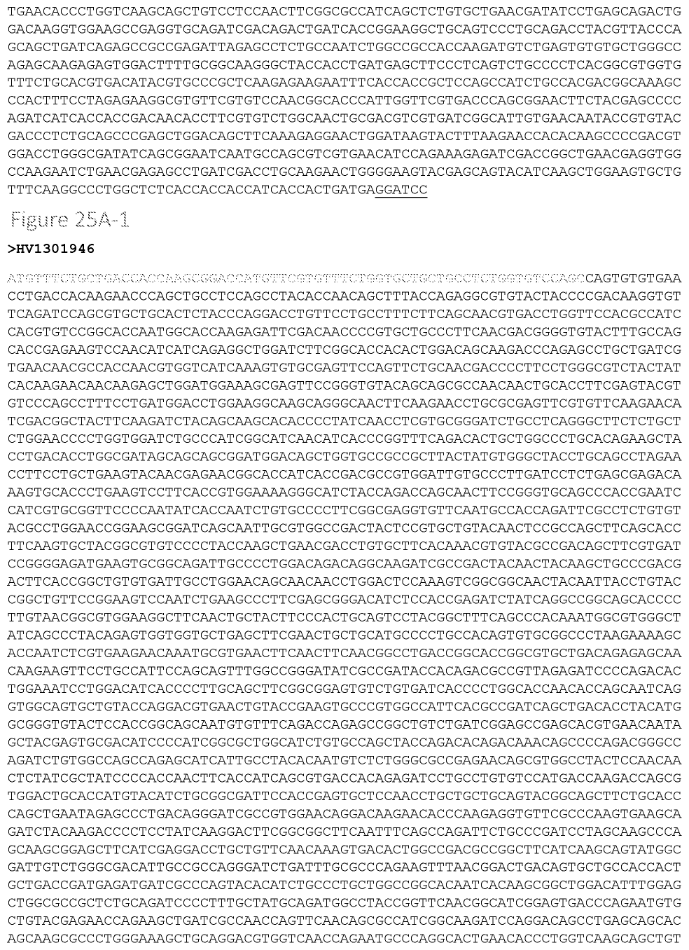

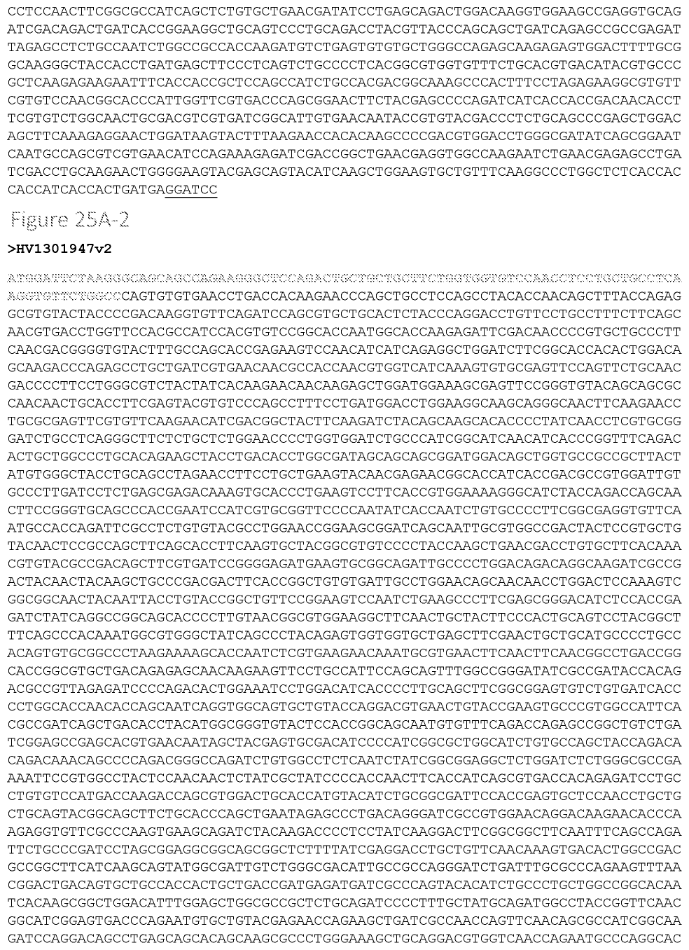

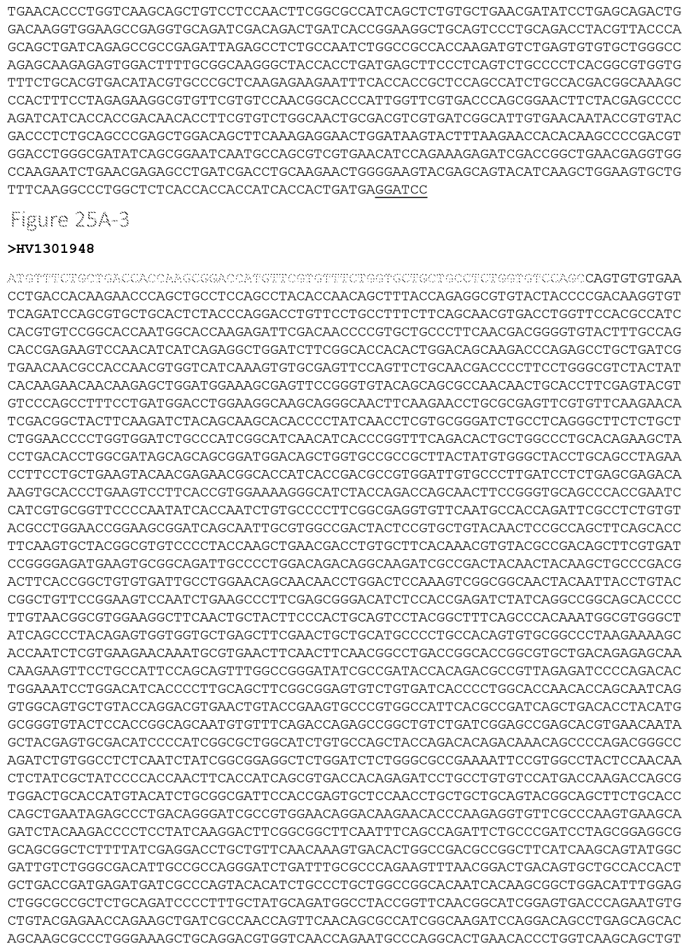

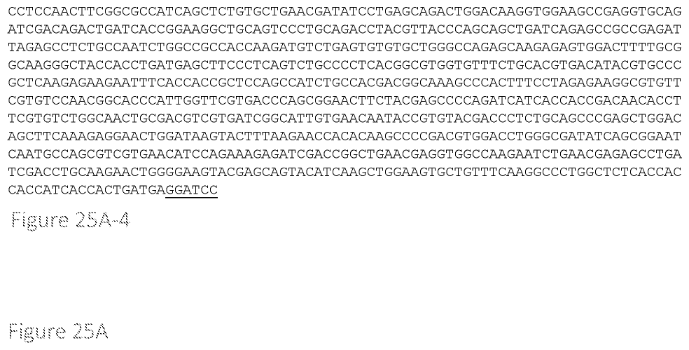

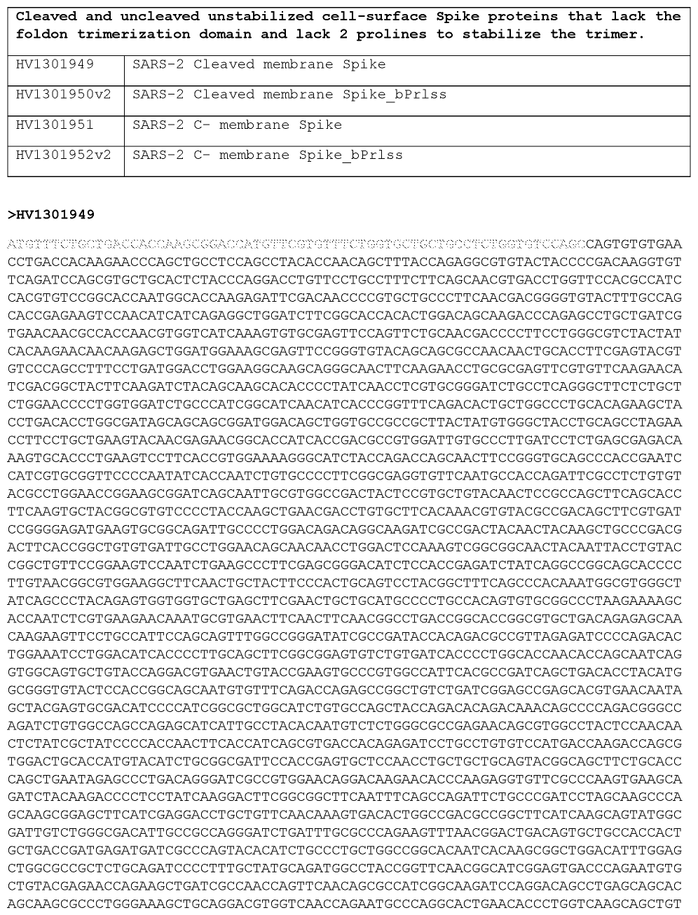

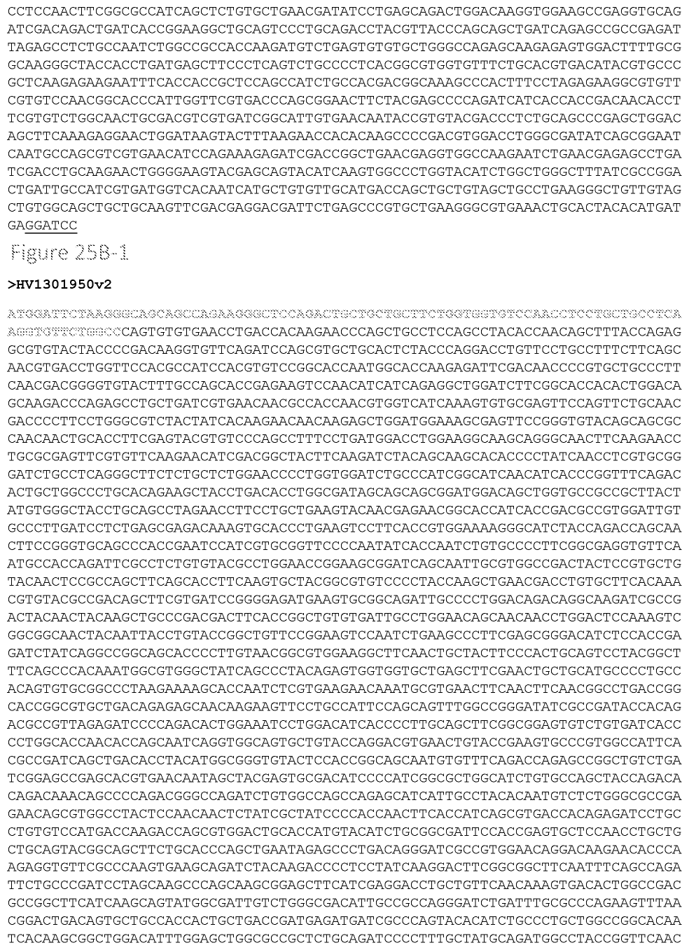

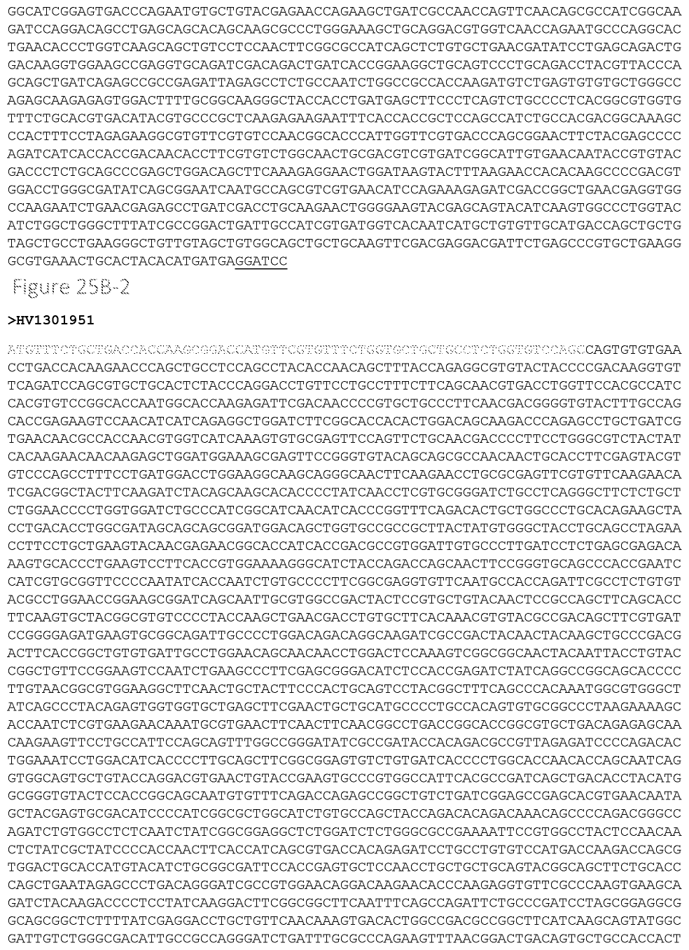

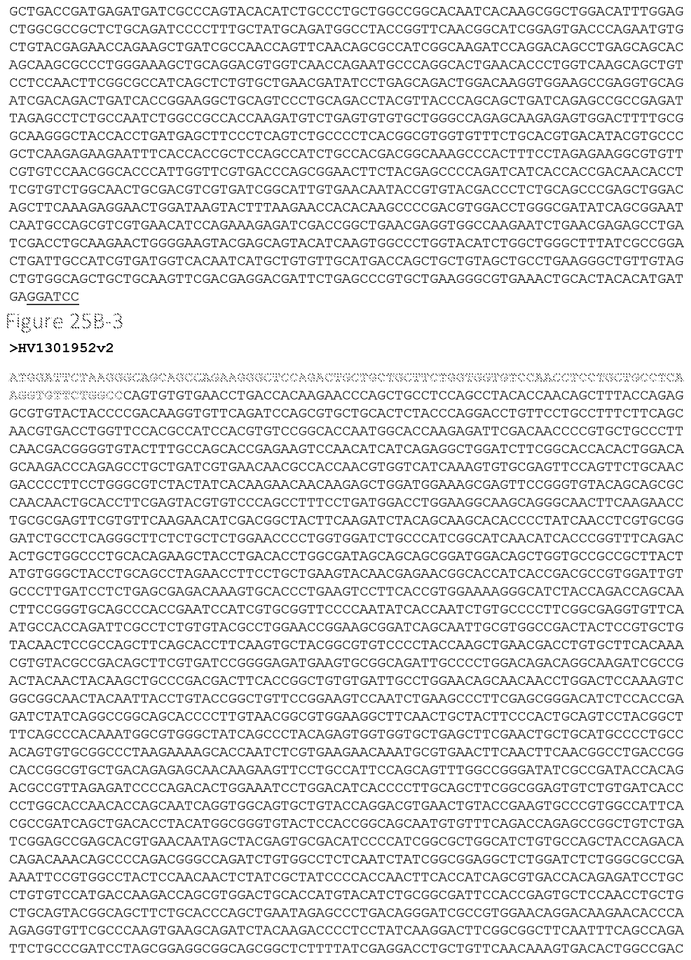

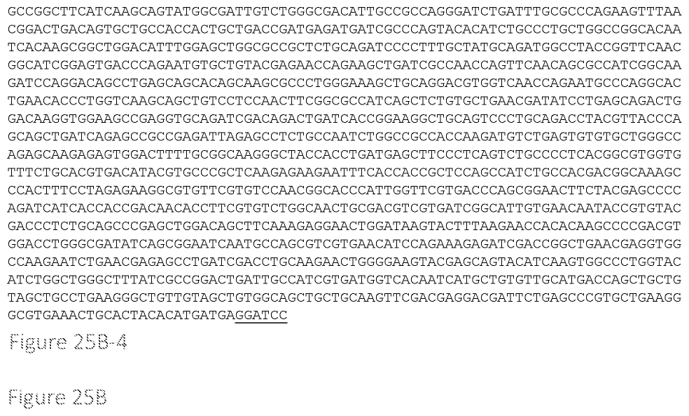

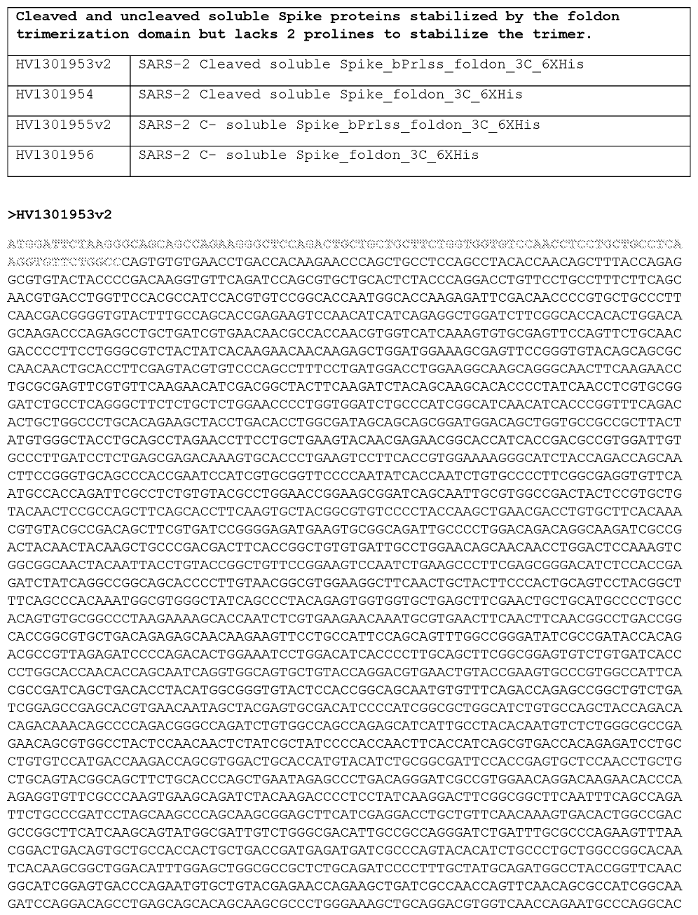

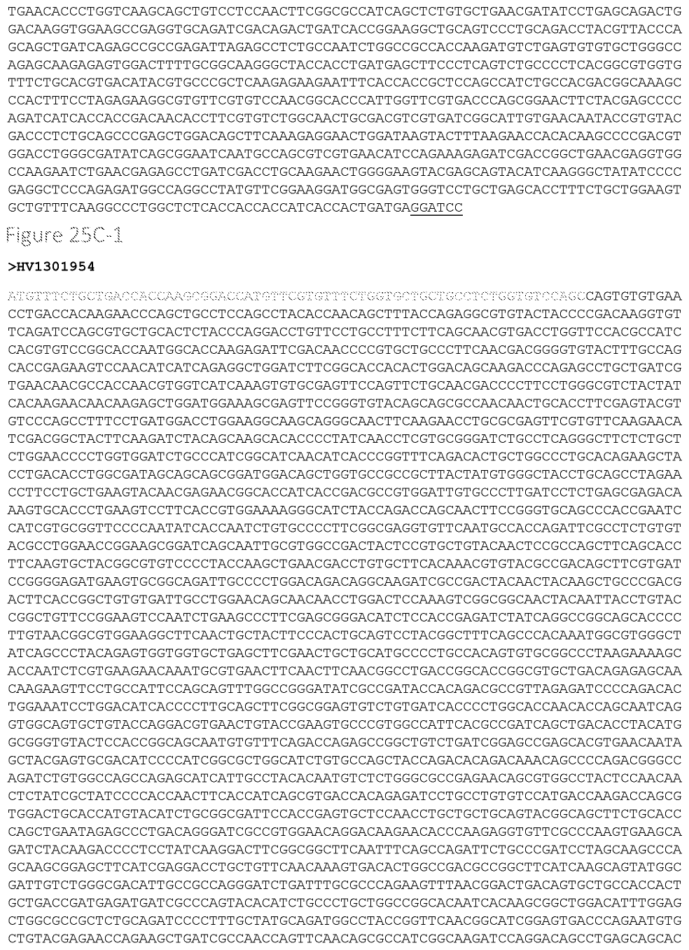

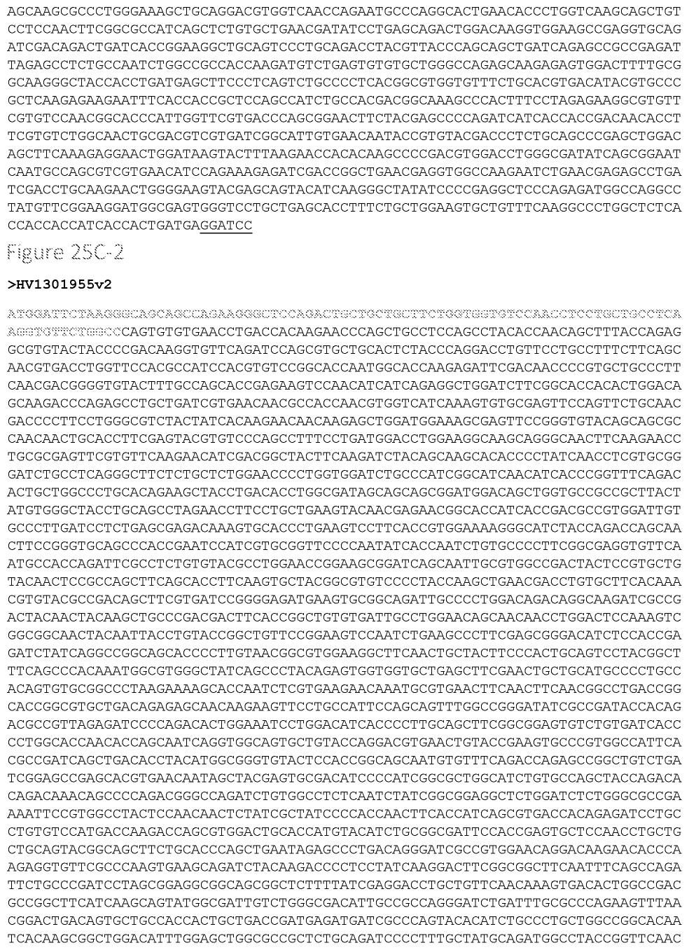

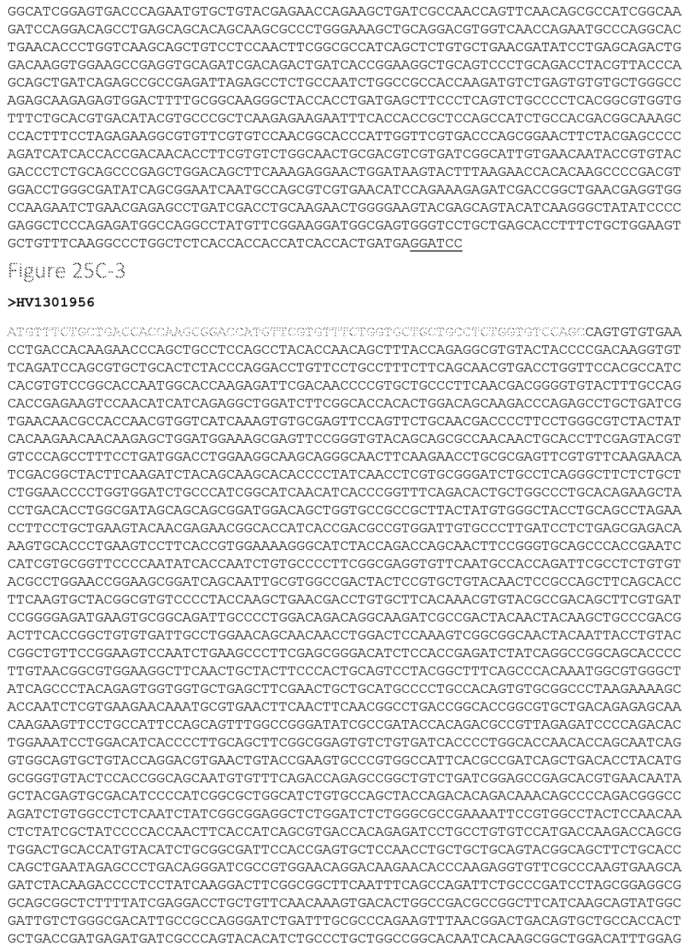

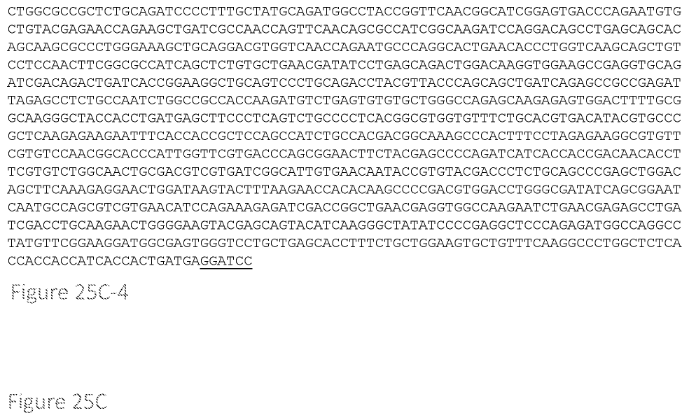

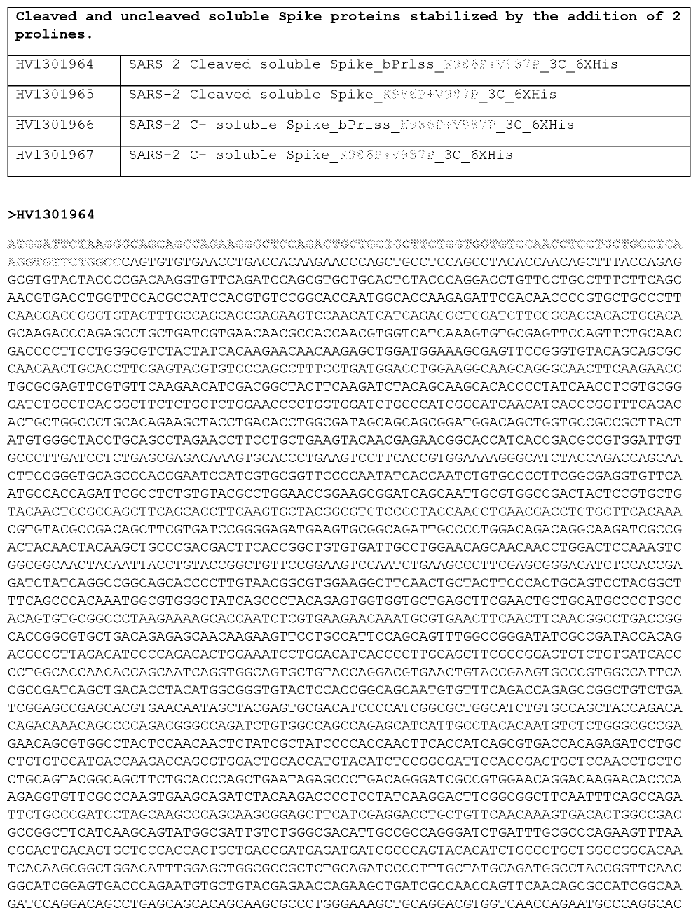

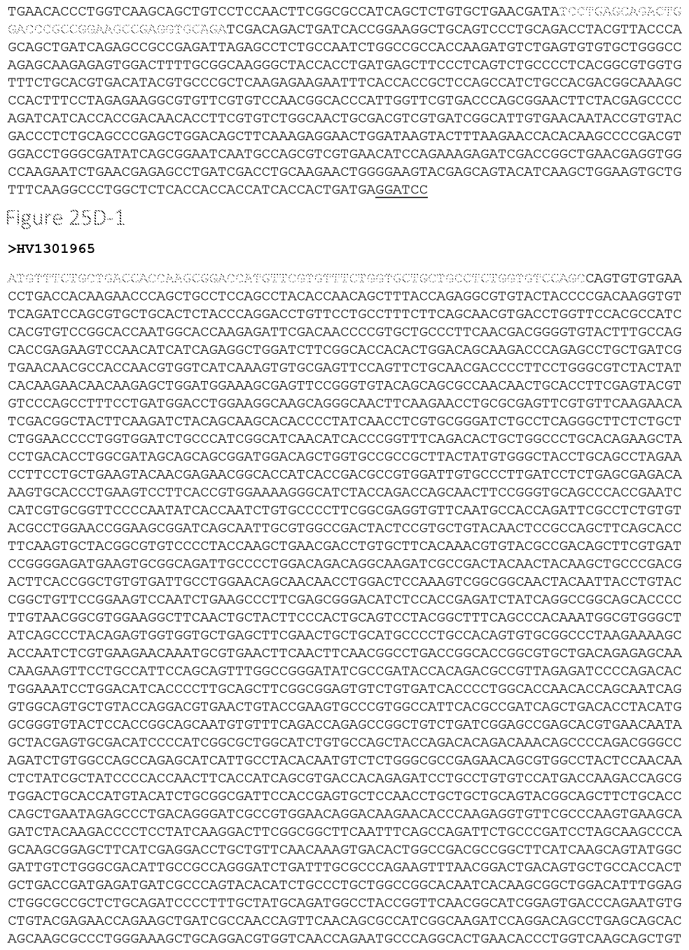

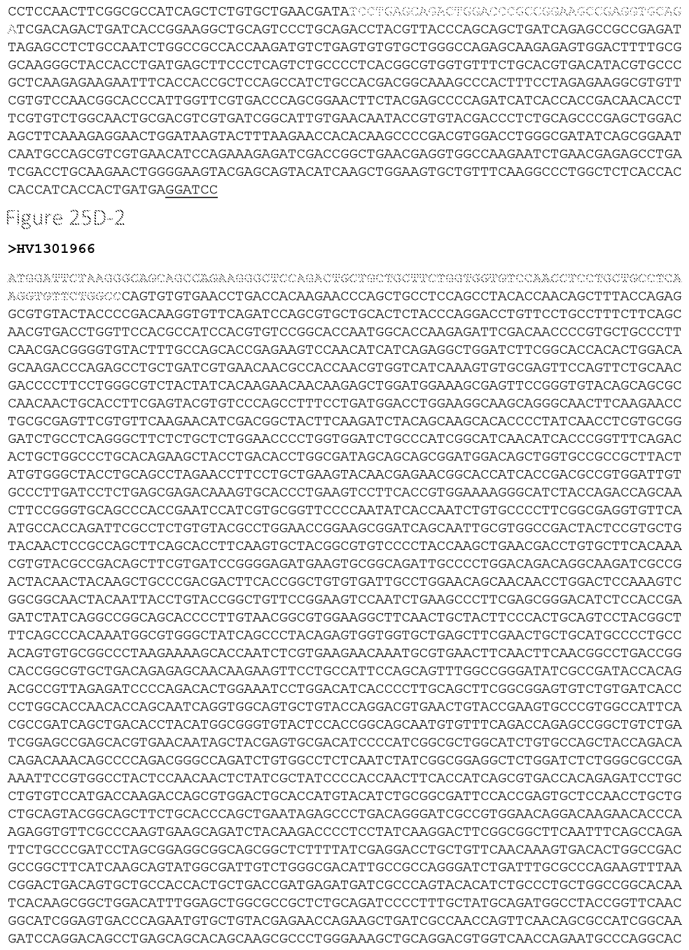

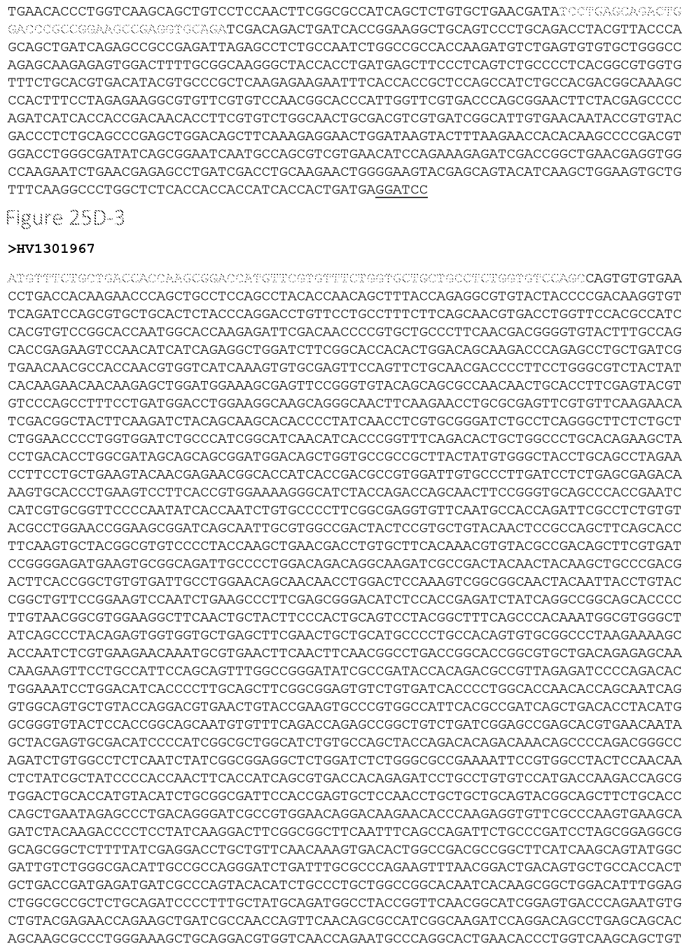

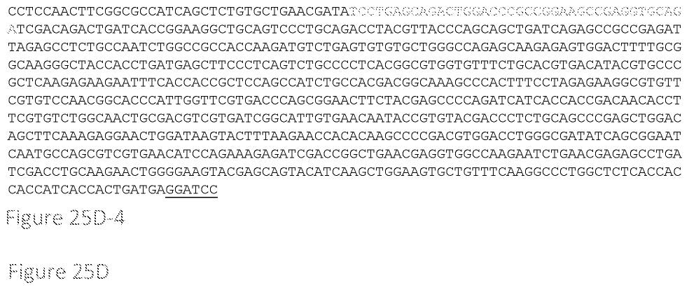

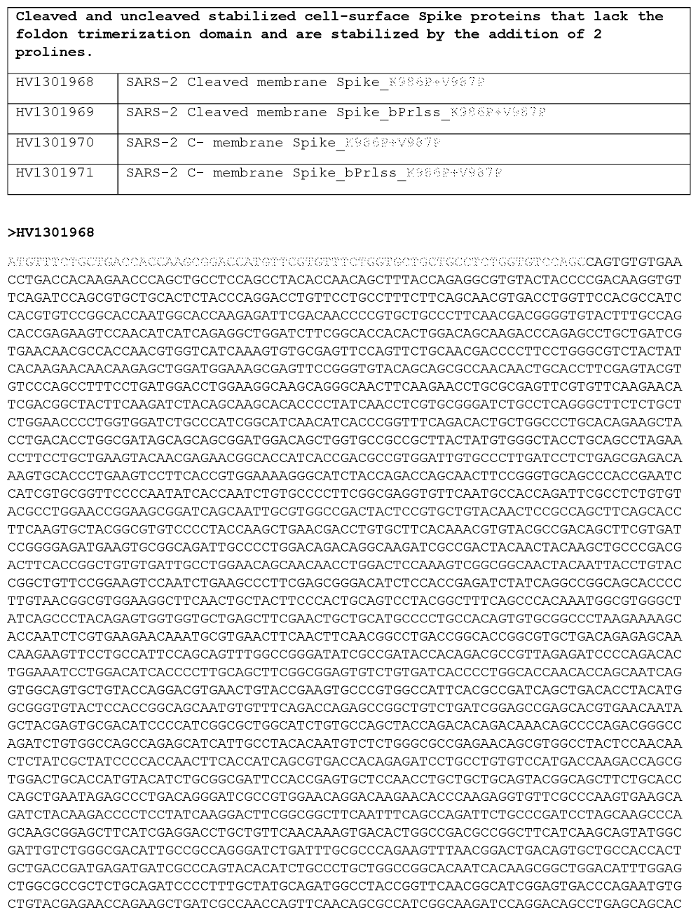

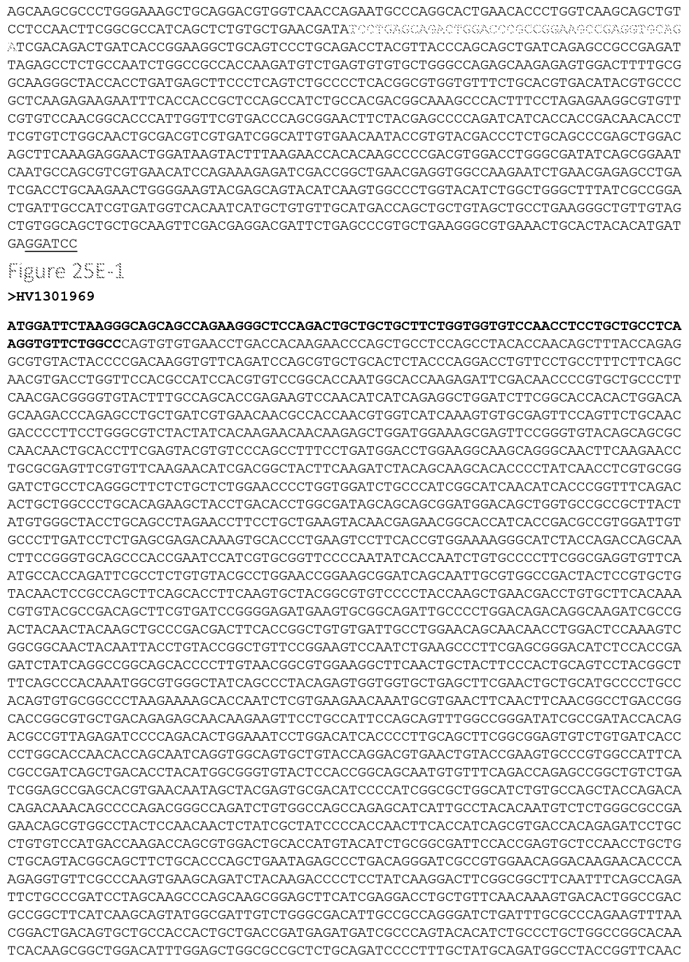

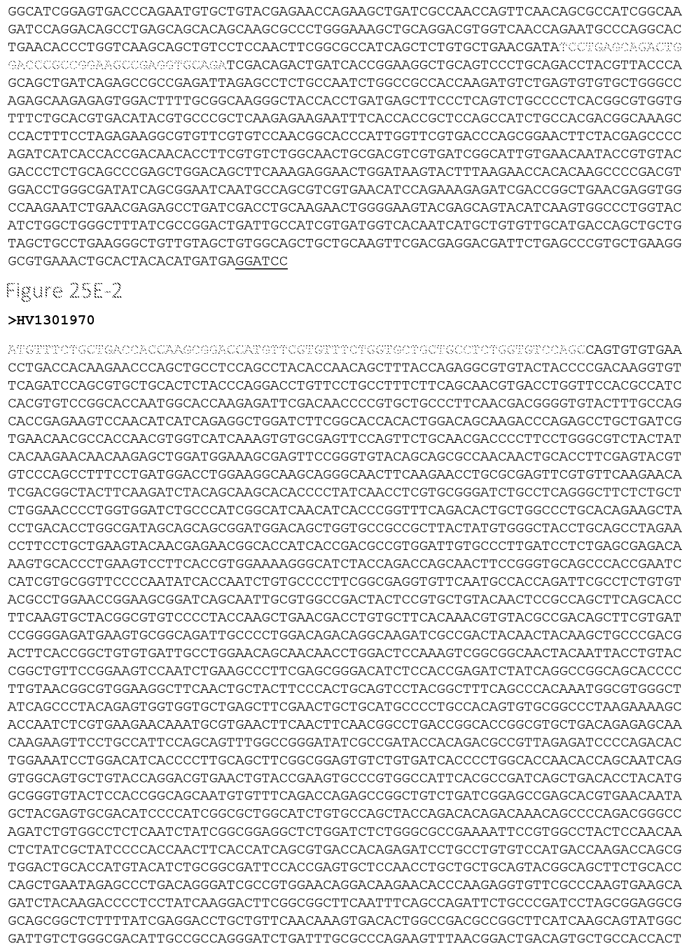

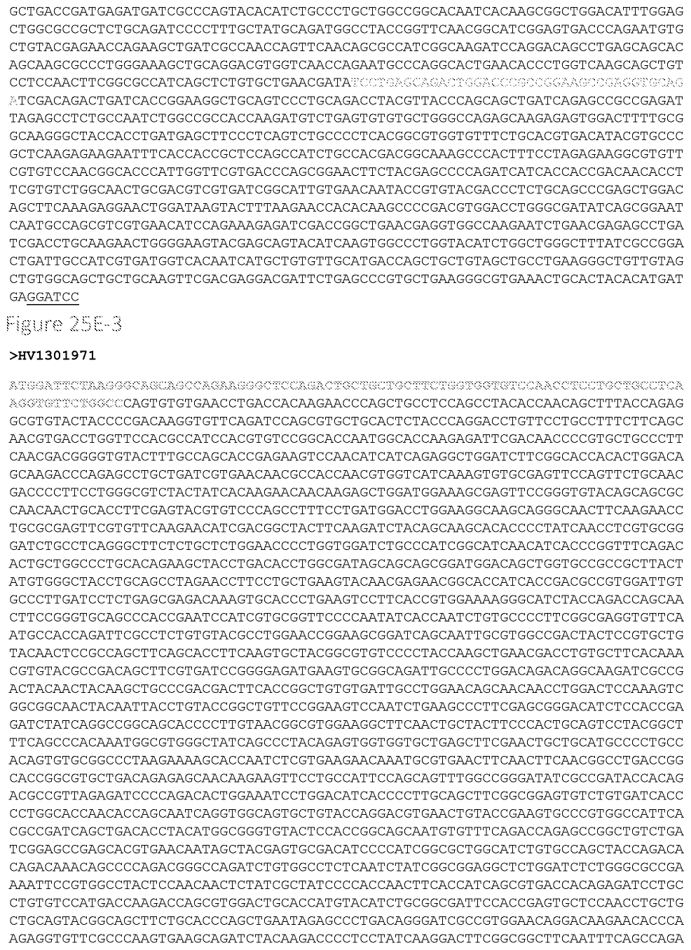

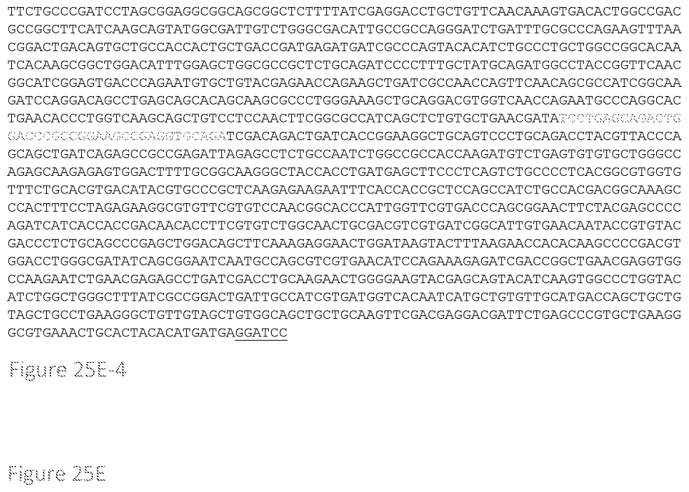

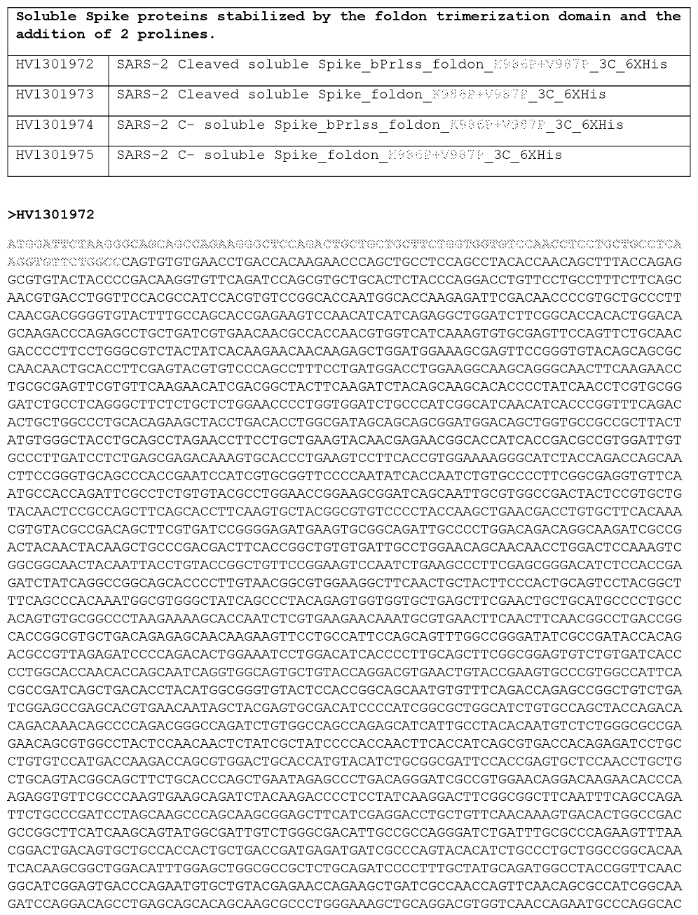

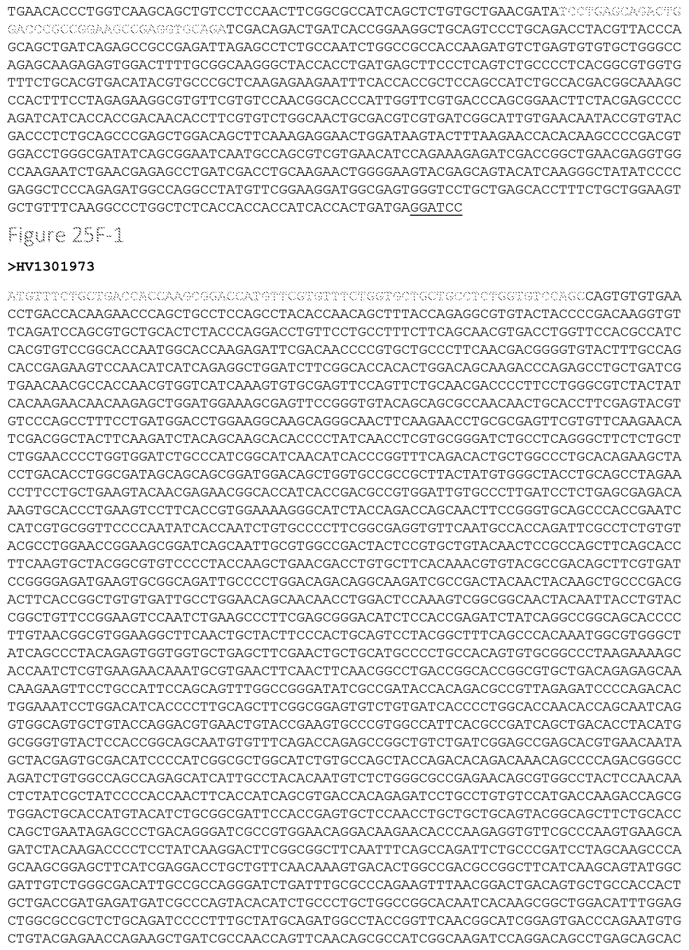

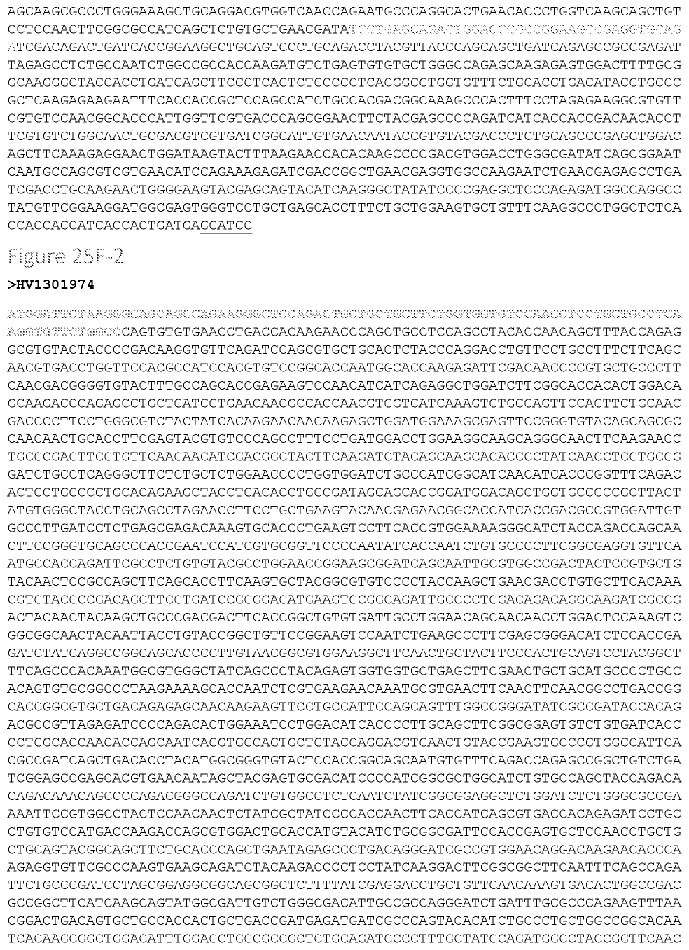

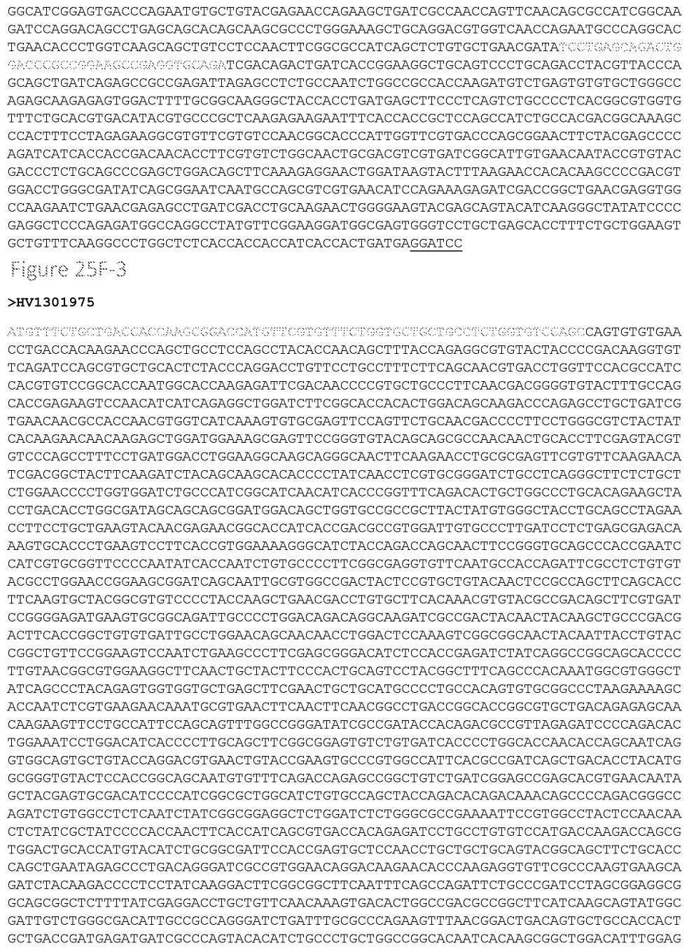

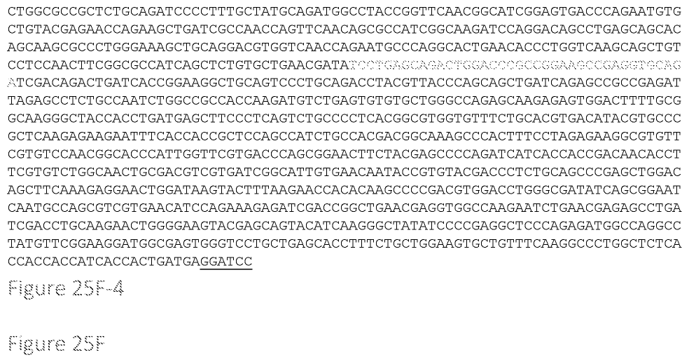

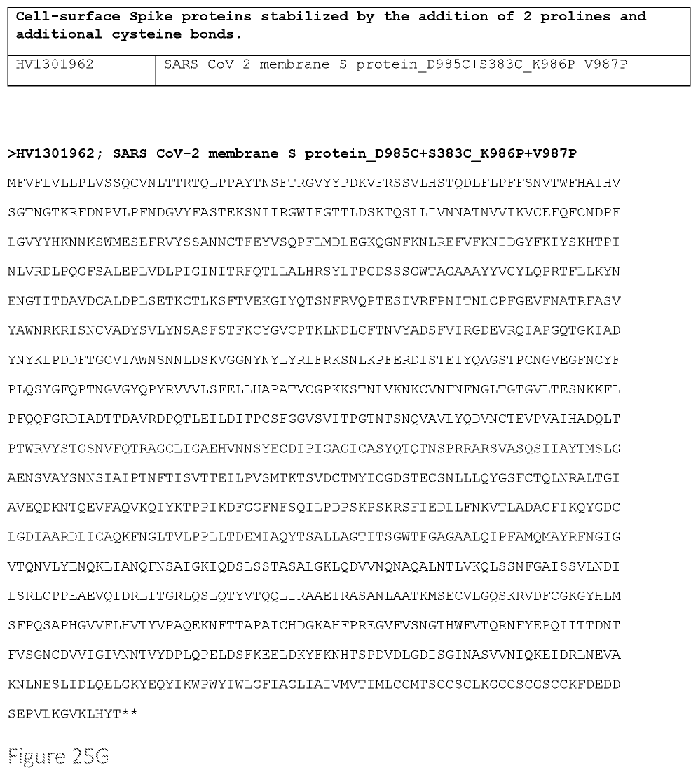

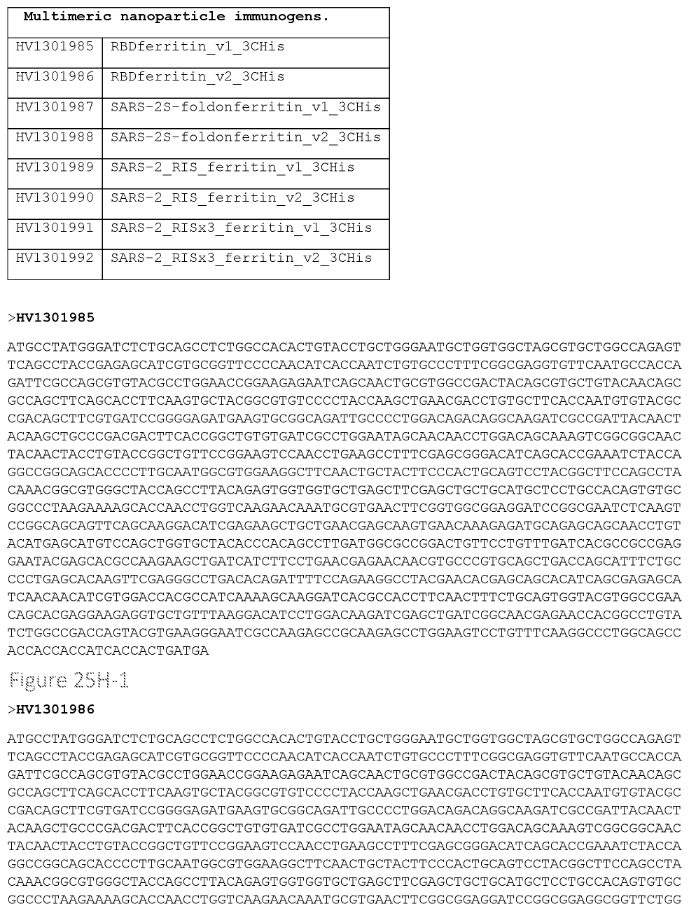

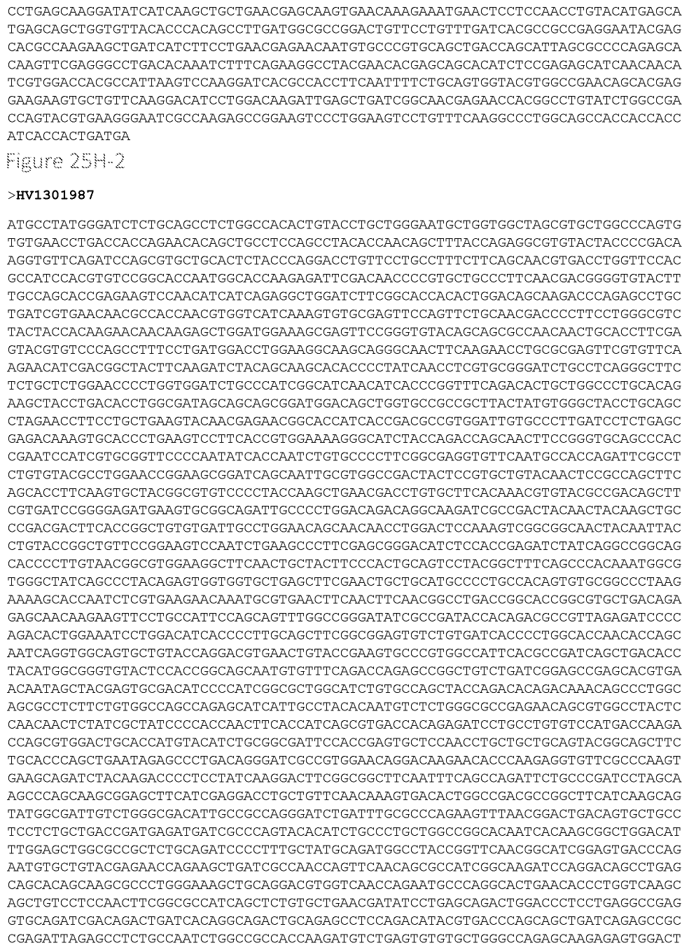

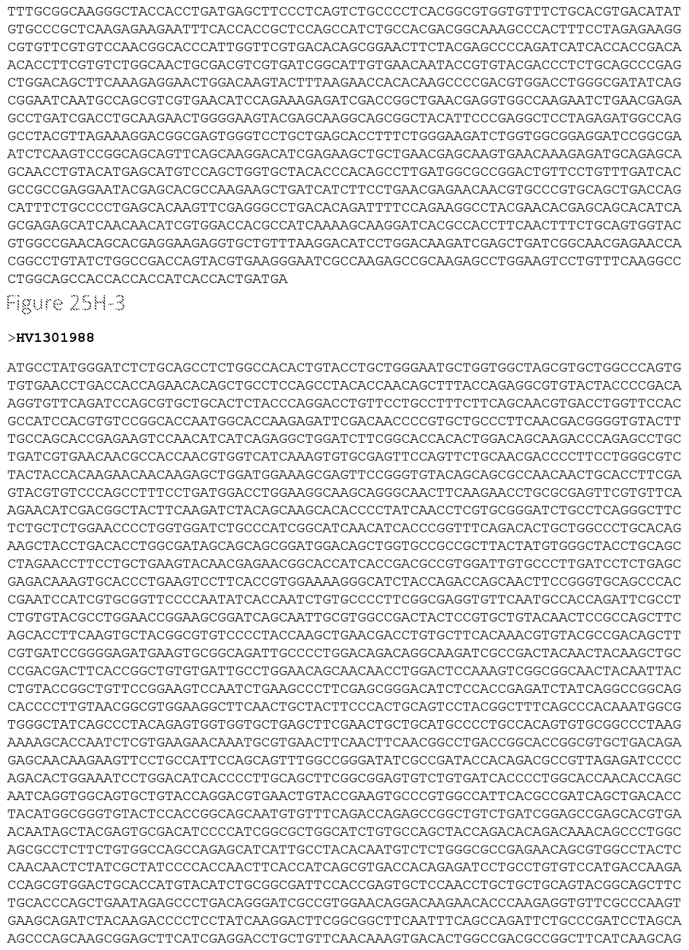

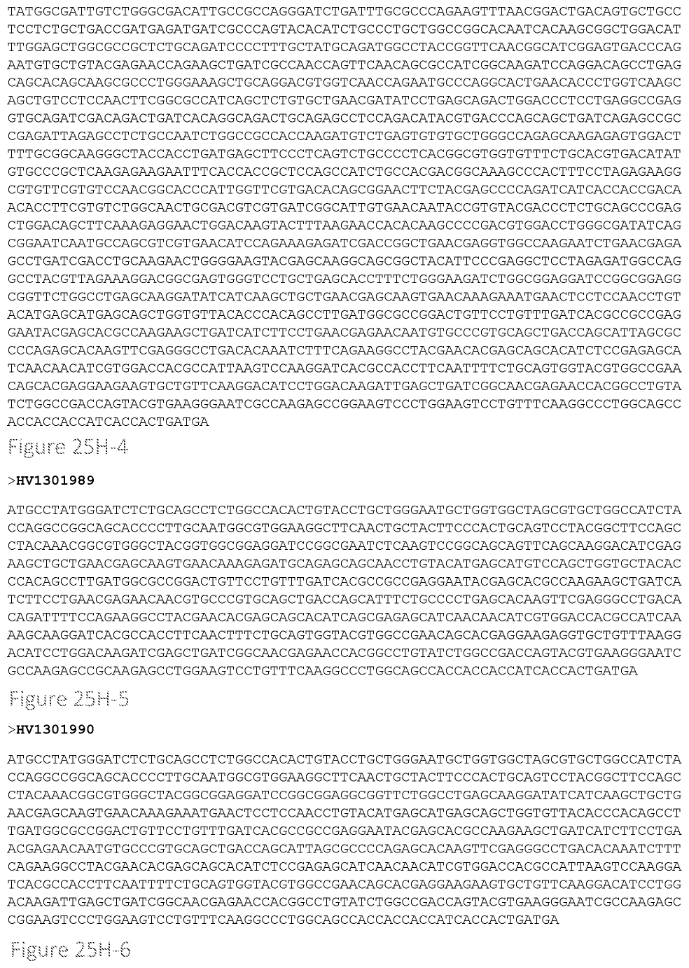

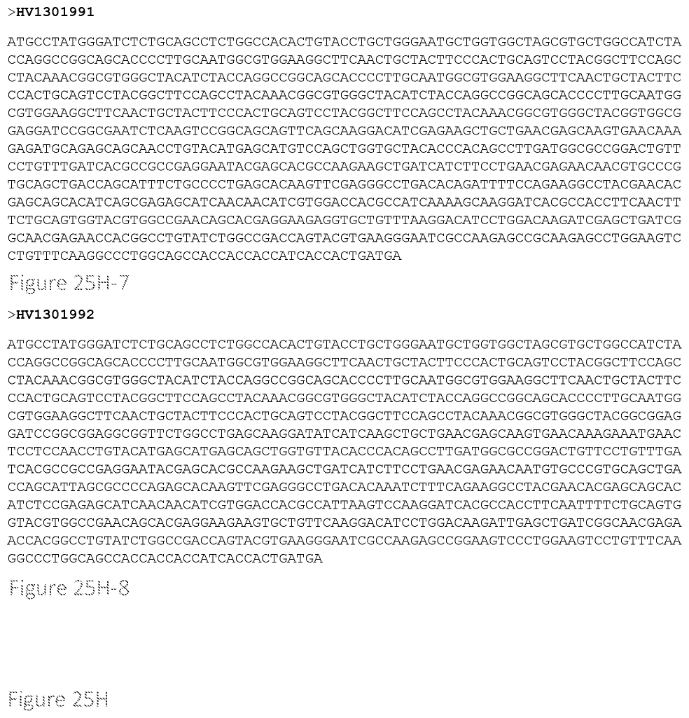

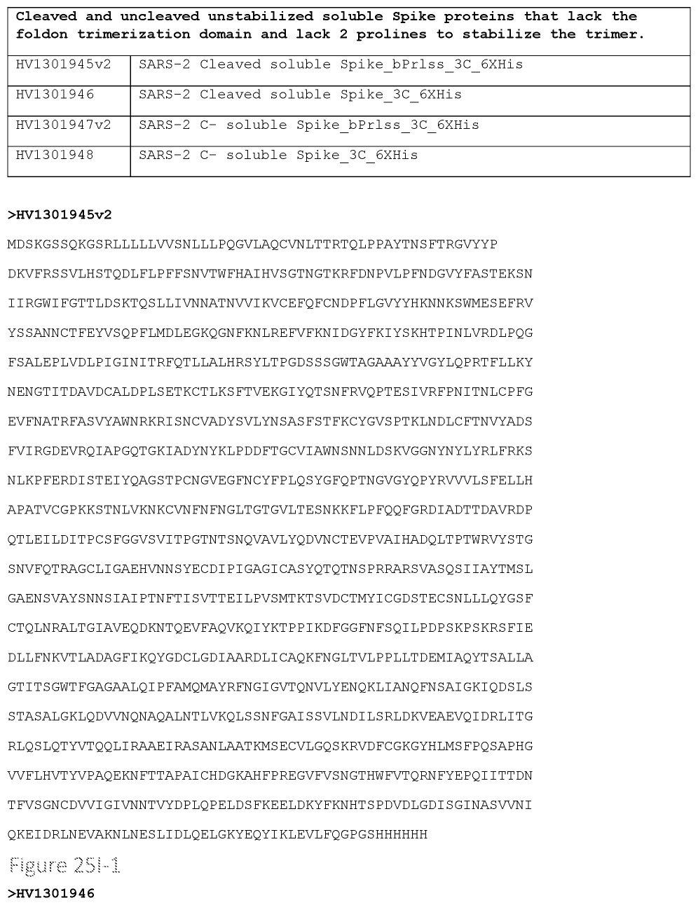

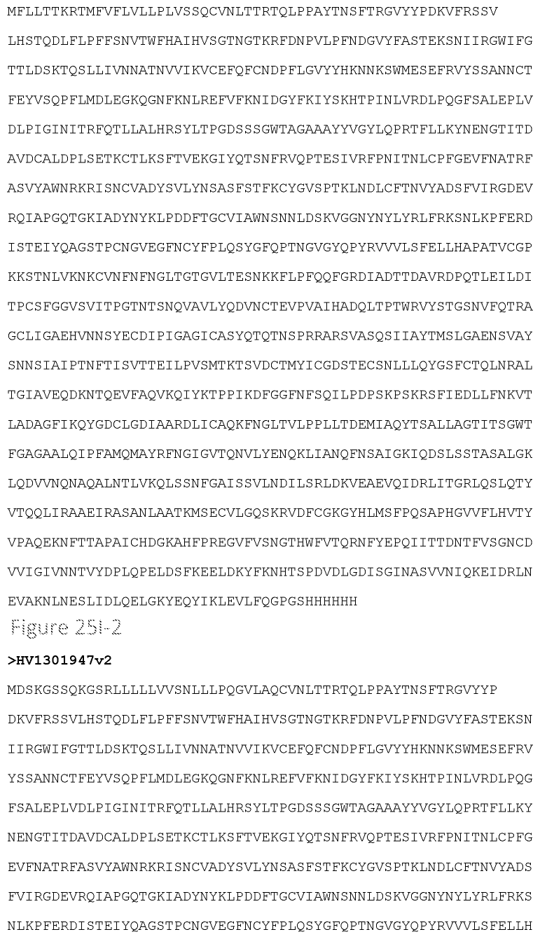

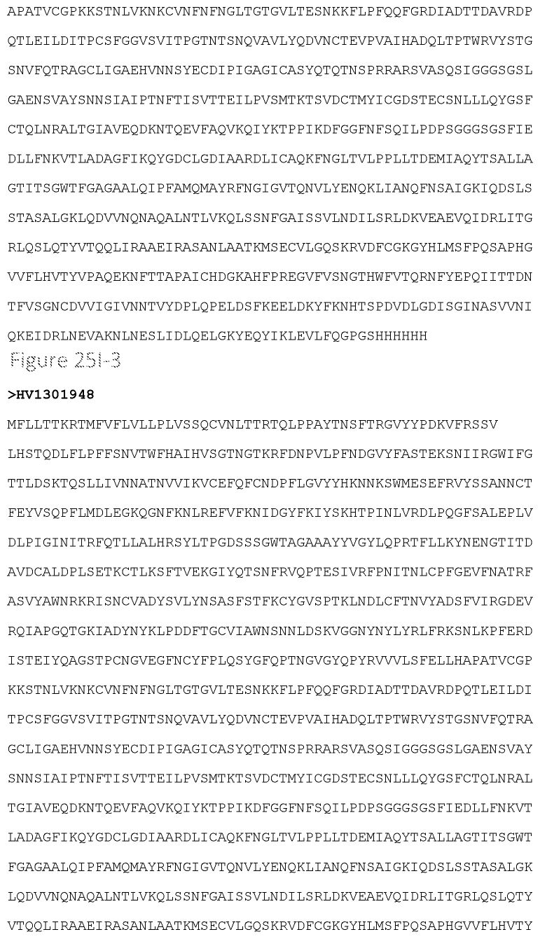

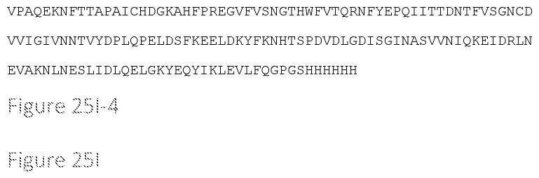

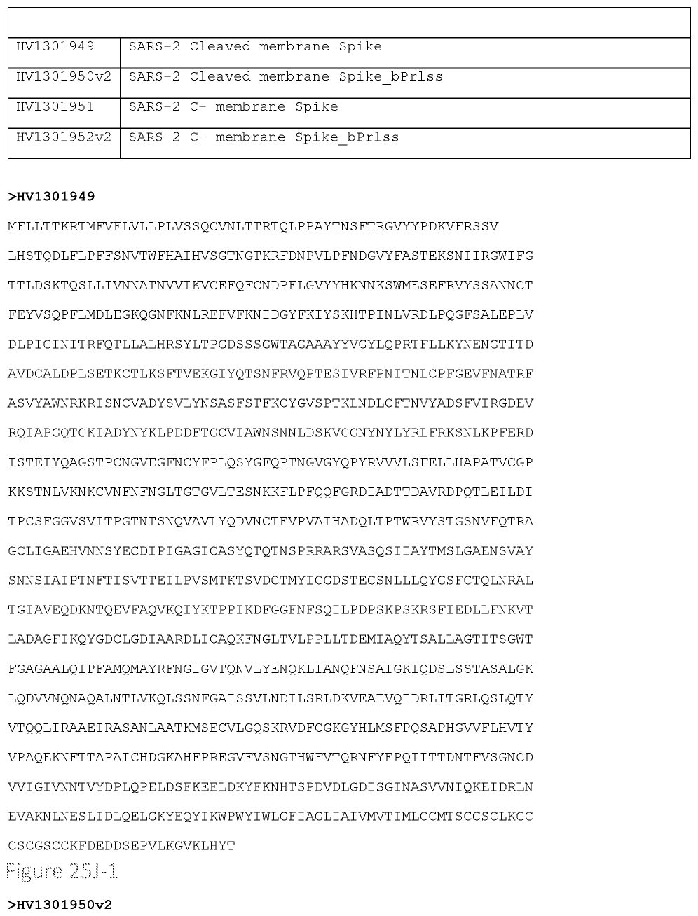

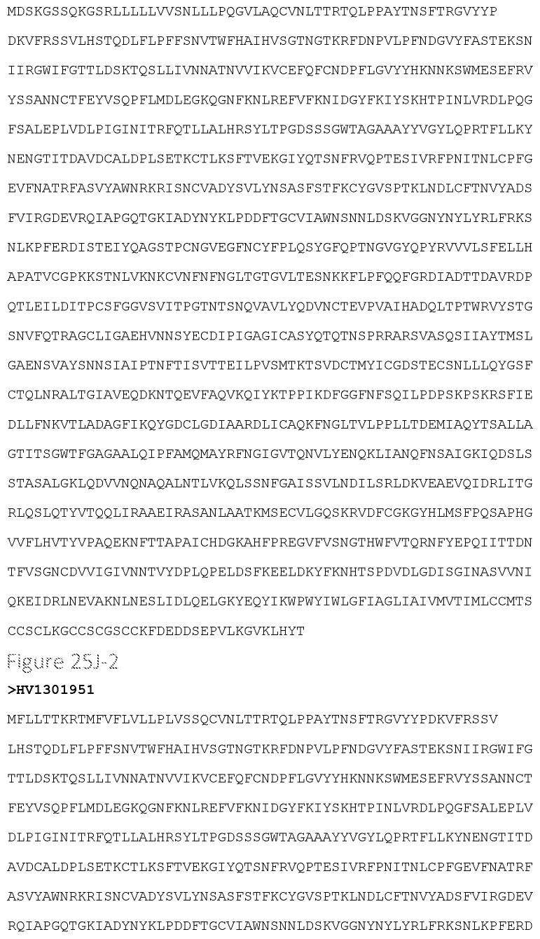

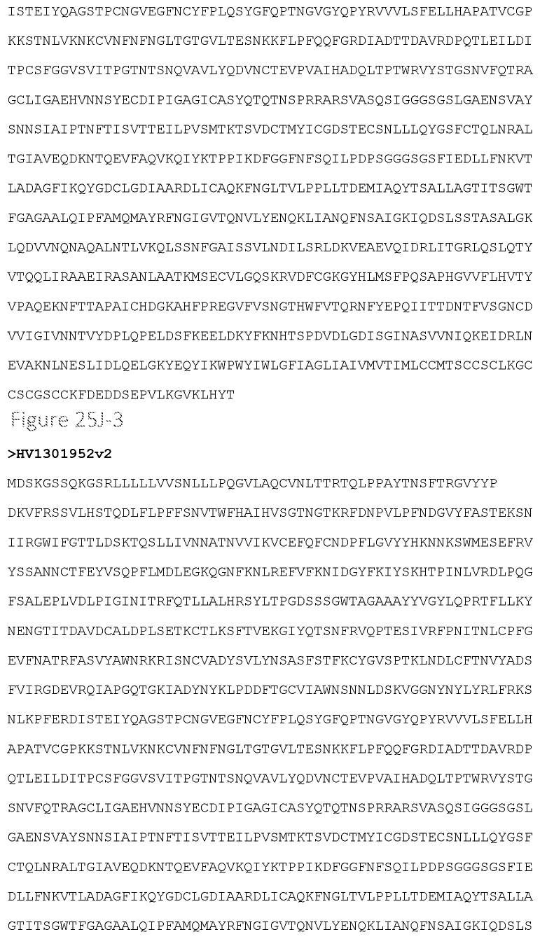

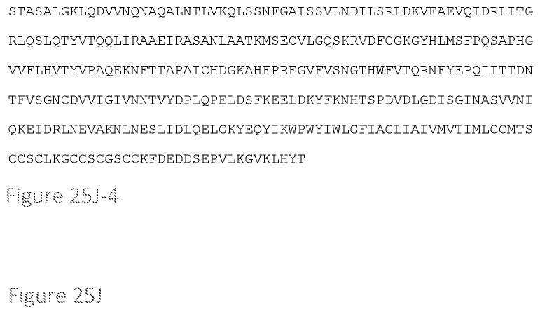

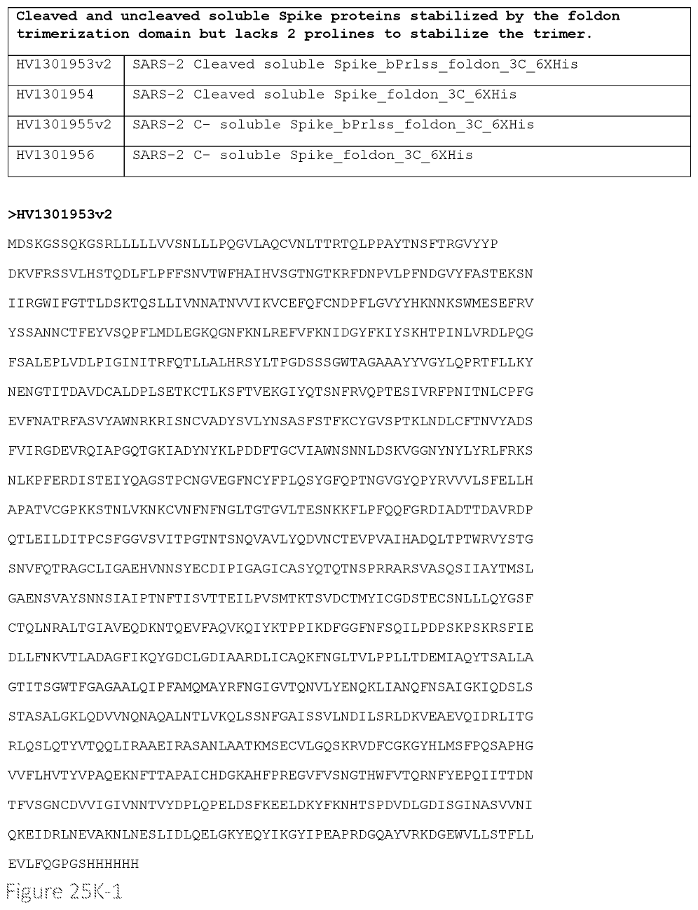

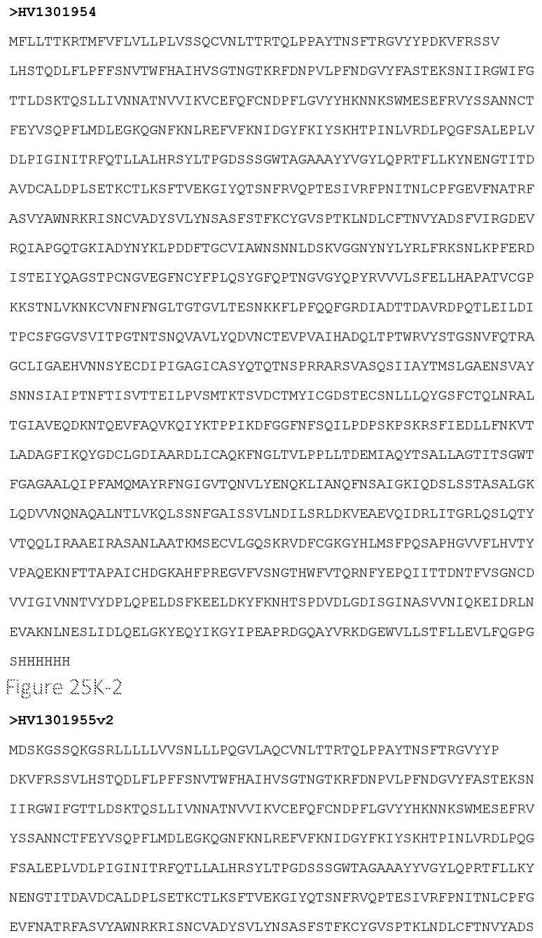

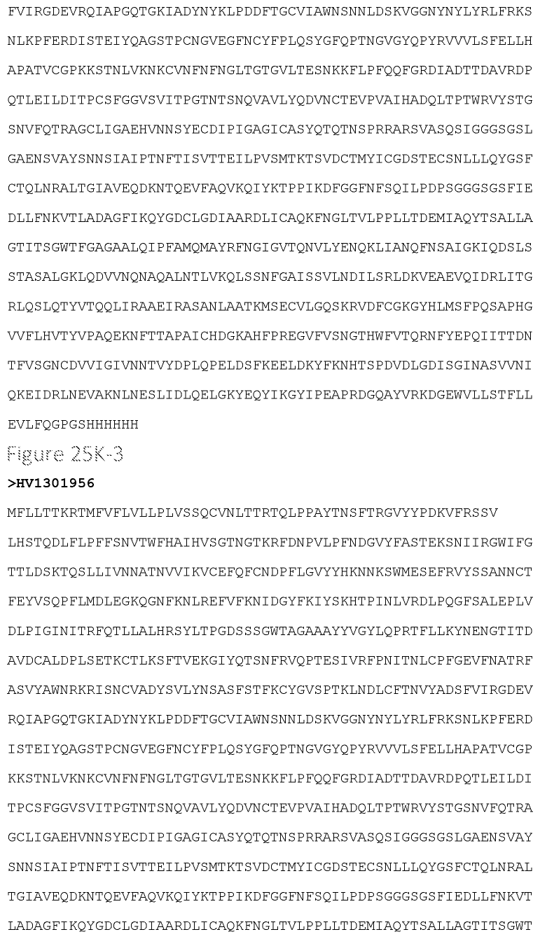

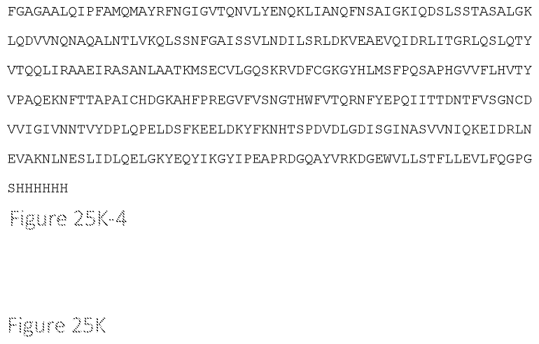

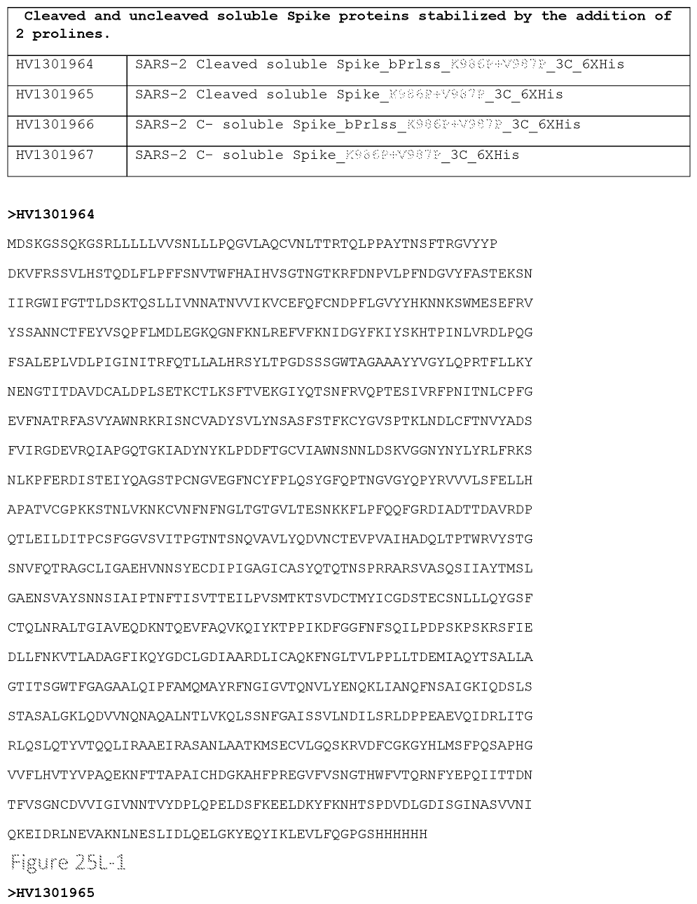

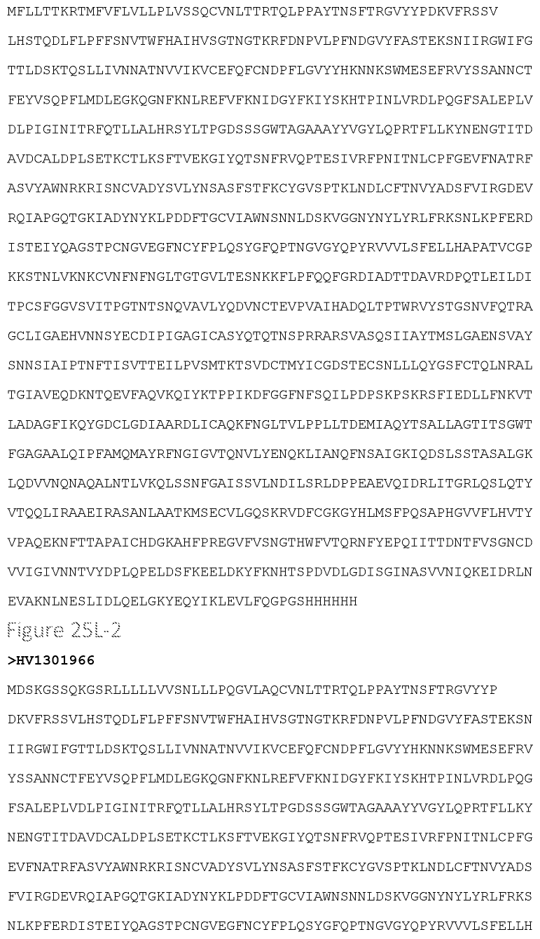

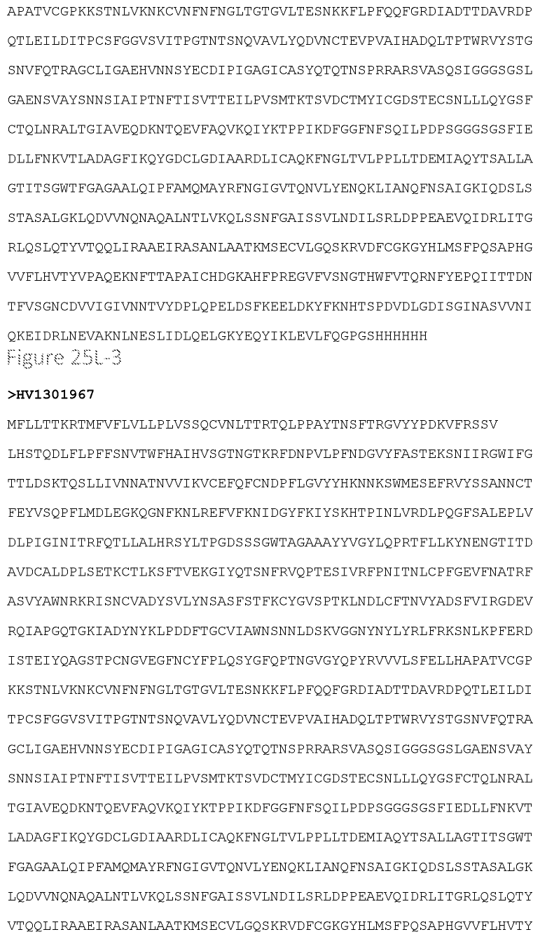

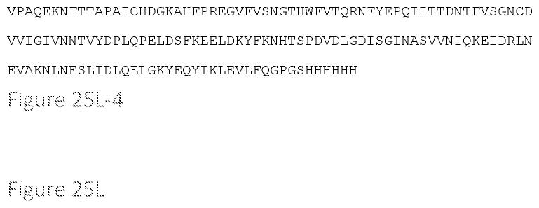

TABLE 1
Cleaved and uncleaved unstabilized soluble Spike proteins that lack the
foldon trimerization domain and lack 2 prolines to stabilize
the trimer. FIG. 25A shows non-limiting embodiments of nucleic
acids and FIG. 25I shows non-limiting embodiments
of amino acid sequences.

| | |
|---|---|
| HV1301945v2 | SARS-2 Cleaved soluble Spike_bPrlss_3C_6XHis |
| HV1301946 | SARS-2 Cleaved soluble Spike_3C_6XHis |
| HV1301947v2 | SARS-2 C-soluble Spike_bPrlss_3C_6XHis |
| HV1301948 | SARS-2 C-soluble Spike_3C_6XHis |

TABLE 2
Cleaved and uncleaved unstabilized cell-surface Spike proteins that
lack the foldon trimerization domain and lack 2 prolines to stabilize
the trimer. FIG. 25B shows non-limiting embodiments of nucleic
acids and FIG. 25J shows non-limiting embodiments
of amino acid sequences.

| | |
|---|---|
| HV1301949 | SARS-2 Cleaved membrane Spike |
| HV1301950v2 | SARS-2 Cleaved membrane Spike_bPrlss |
| HV1301951 | SARS-2 C-membrane Spike |
| HV1301952v2 | SARS-2 C-membrane Spike_bPrlss |

TABLE 3
Cleaved and uncleaved soluble Spike proteins stabilized by the foldon
trimerization domain but lacks 2 prolines to stabilize
the trimer. FIG. 25C shows non-limiting embodiments of nucleic acids
and FIG. 25K shows non-limiting embodiments of
amino acid sequences.

| | |
|---|---|
| HV1301953v2 | SARS-2 Cleaved soluble Spike_bPrlss_foldon_3C_6XHis |
| HV1301954 | SARS-2 Cleaved soluble Spike_foldon_3C_6XHis |
| HV1301955v2 | SARS-2 C-soluble Spike_bPrlss_foldon_3C_6XHis |
| HV1301956 | SARS-2 C-soluble Spike_foldon_3C_6XHis |

TABLE 4
Cleaved and uncleaved soluble Spike proteins stabilized by the
addition of 2 prolines. FIG. 25D shows non-limiting
embodiments of nucleic acids and FIG. 25L
shows non-limiting embodiments of amino acid sequences.

| | |
|---|---|
| HV1301964 | SARS-2 Cleaved soluble |
| | Spike_bPrlss_K986P + V987P_3C_6XHis |
| HV1301965 | SARS-2 Cleaved soluble Spike_K986P + V987P_3C_6XHis |
| HV1301966 | SARS-2 C-soluble |
| | Spike_bPrlss_K986P + V987P_3C_6XHis |
| HV1301967 | SARS-2 C-soluble Spike_K986P + V987P_3C_6XHis |

TABLE 5
Cleaved and uncleaved stabilized cell-surface Spike proteins that lack the
foldon trimerization domain and are stabilized by the
addition of 2 prolines. FIG. 25E shows non-limiting embodiments
of nucleic acids and FIG. 25M shows non-limiting
embodiments of amino acid sequences.

| | |
|---|---|
| HV1301968 | SARS-2 Cleaved membrane Spike_K986P + V987P |
| HV1301969 | SARS-2 Cleaved membrane Spike_bPrlss_K986P + V987P |
| HV1301970 | SARS-2 C-membrane Spike_K986P + V987P |
| HV1301971 | SARS-2 C-membrane Spike_bPrlss_K986P + V987P |

TABLE 6
Soluble Spike proteins stabilized by the foldon trimerization
domain and the addition of 2 prolines. FIG. 25F shows
non-limiting embodiments of nucleic acids and
FIG. 25N shows non-limiting embodiments of amino acid sequences.

| HV130197 | SARS-2 Cleaved soluble |
|---|---|
| 2 | Spike_bPrlss_foldon_K986P + V987P_3C_6XHis |
| HV130197 | SARS-2 Cleaved soluble |
| 3 | Spike_foldon_K986P + V987P_3C_6XHis |
| HV130197 | SARS-2 C-soluble |
| 4 | Spike_bPrlss_foldon_X986P + V987P_3C_6XHis |
| HV130197 | SARS-2 C-soluble Spike_foldon_K986P + V987P_3C 6XHis |
| 5 | |

TABLE 7
Soluble Spike proteins stabilized by the foldon trimerization domain, the
addition of 2 prolines, and additional cysteine bonds. Non-limiting
embodiments of sequences are shown in FIG. 8 and FIG. 25O.

| | |
|---|---|
| HV1301963_HV1301976 | nCoV-1 nCoV-2P_S383C_D985C |
| HV1301977 | nCoV-1 nCoV-2P_S383C_A570C_ |
| | G669C_T866C_L966C_D985C |
| HV1301978 | nCoV-1 nCoV-2P_K41C_A520C |
| HV1301979 | nCoV-1 nCoV-2P_F43C_S383C_ |
| | G566C_G669C_T866C_D985C |
| HV1301980 | nCoV-1 nCoV-2P_K41C_A520C_ |
| | A570C_G669C_T866C_L966C |

TABLE 8
Cell-surface Spike proteins stabilized by the addition of 2 prolines and
additional cysteine bonds. FIG. 25G and 25P shows
non-limiting embodiments of amino acid sequences.

| | |
|---|---|
| HV1301962 | SARS CoV-2 membrane S |
| | protein_D985C + S383C_K986P + V987P |

TABLE 9A
Multimeric nanoparticle immunogens. FIG. 25H shows non-limiting
embodiments of nucleic acids and FIG. 25Q shows
non-limiting embodiments of amino acid sequences.

| | |
|---|---|
| HV1301985 | RBDferritin_v1_3CHis |
| HV1301986 | RBDferritin_v2_3CHis |
| HV1301987 | SARS-2S-foldonferritin_v1_3CHis |
| HV1301988 | SARS-2S-foldonferritin_v2_3CHis |
| HV1301989 | SARS-2_RIS_ferritin_v1_3CHis |
| HV1301990 | SARS-2_RIS_ferritin_v2_3CHis |
| HV1301991 | SARS-2_RISx3_ferritin_v1_3CHis |
| HV1301992 | SARS-2_RISx3_ferritin_v2_3CHis |

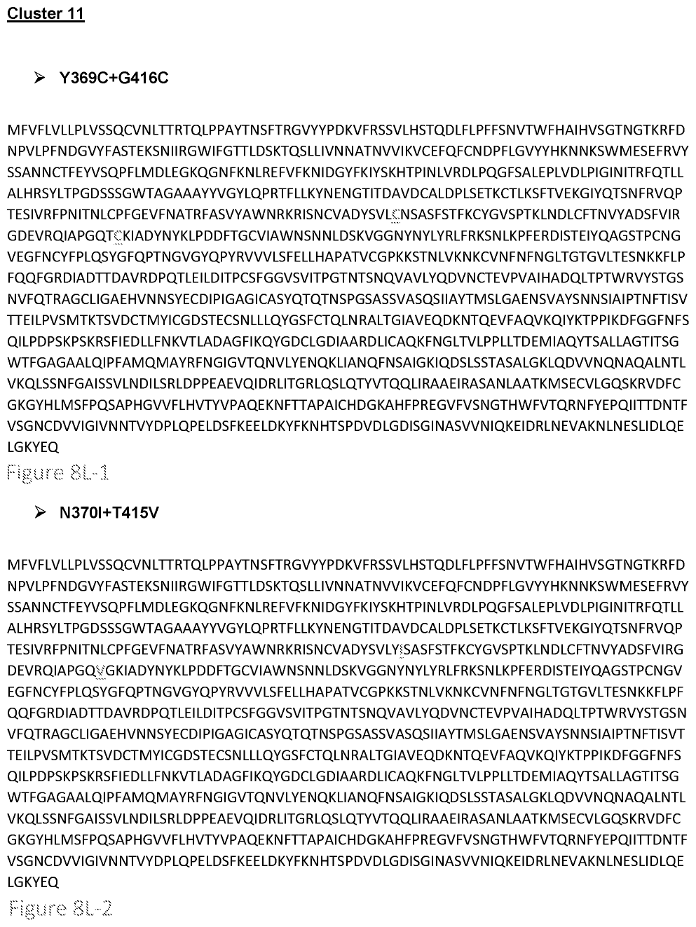

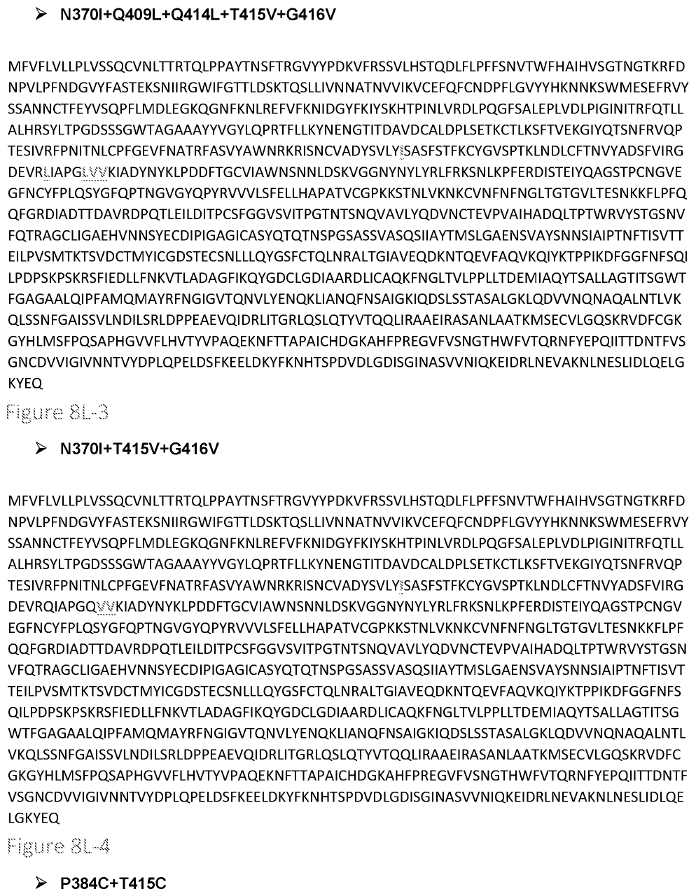

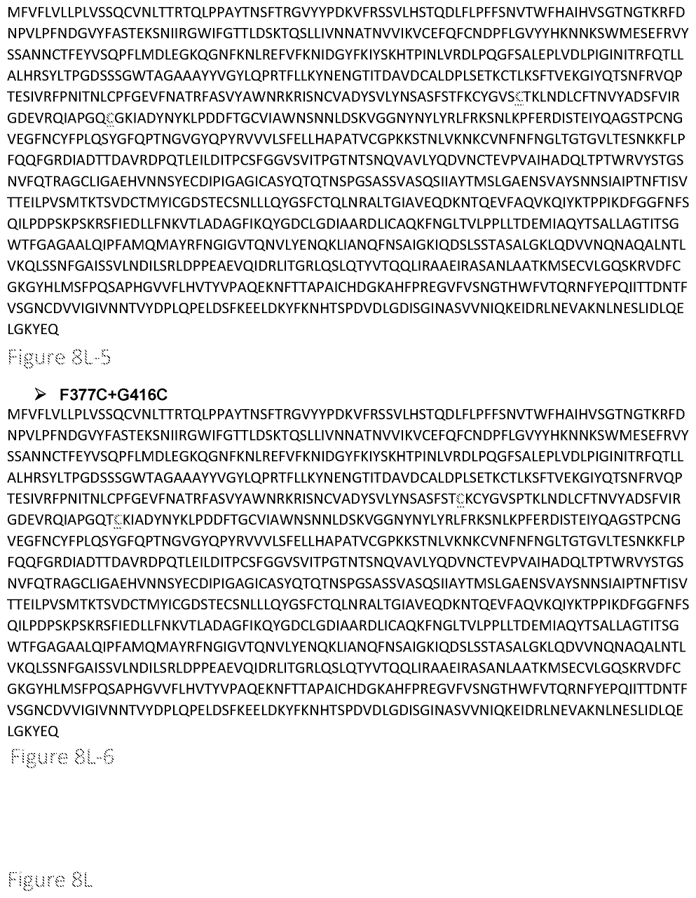

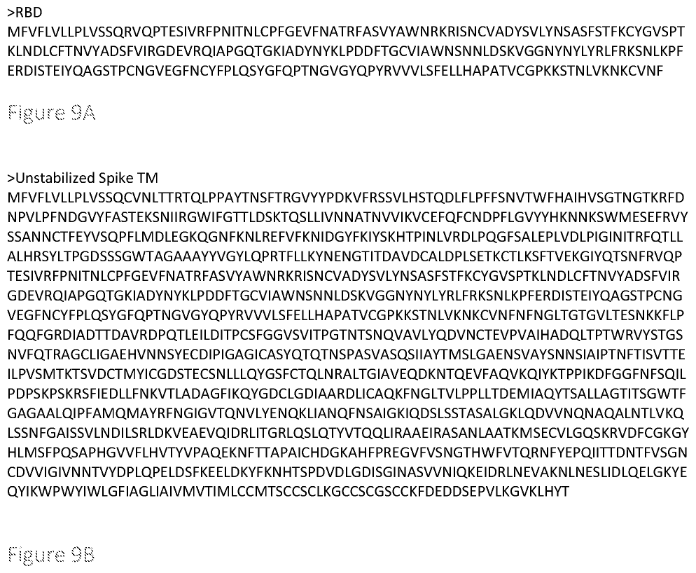

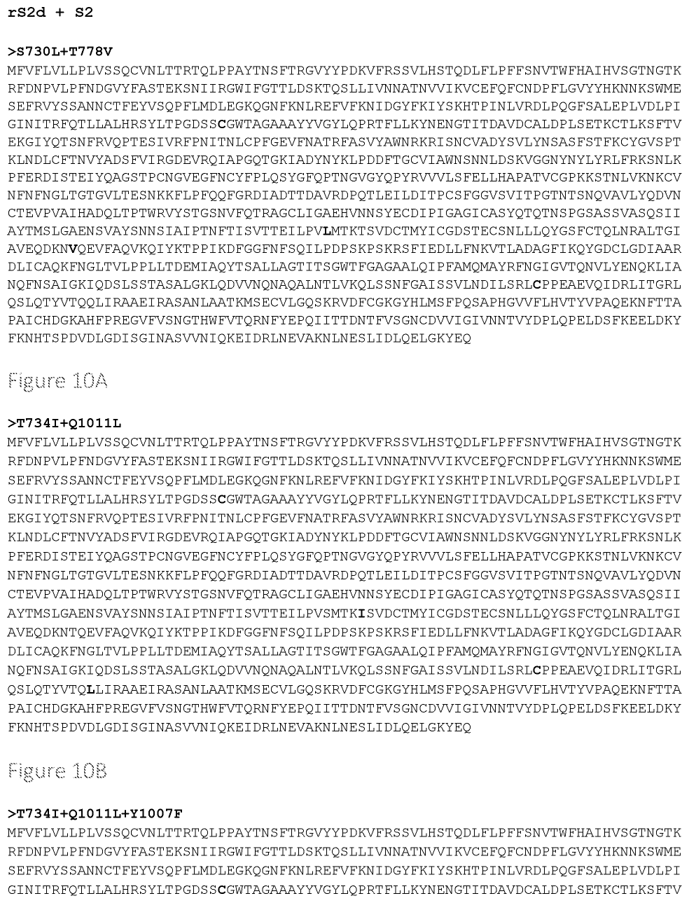

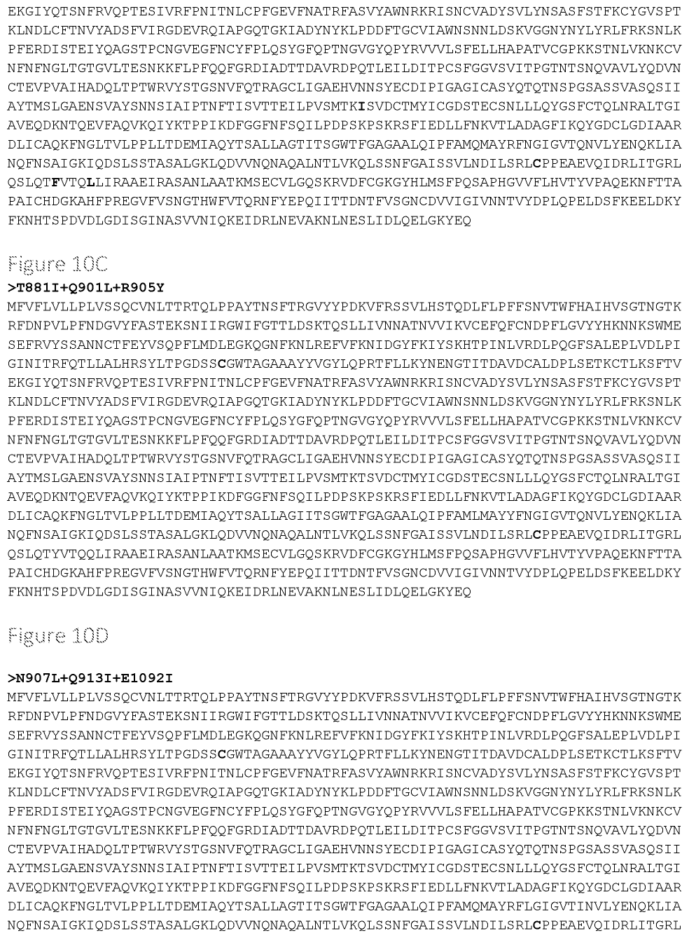

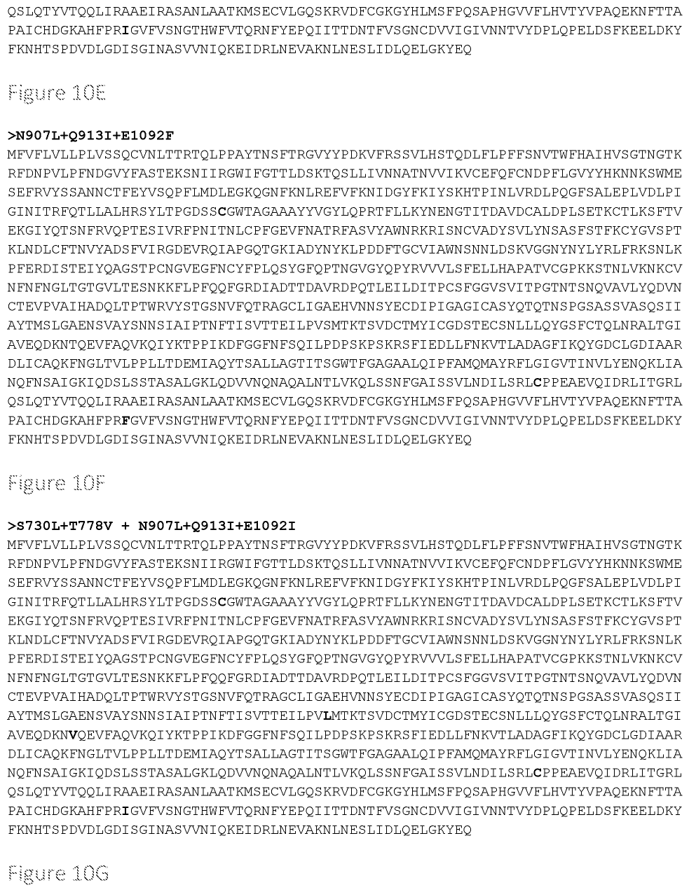

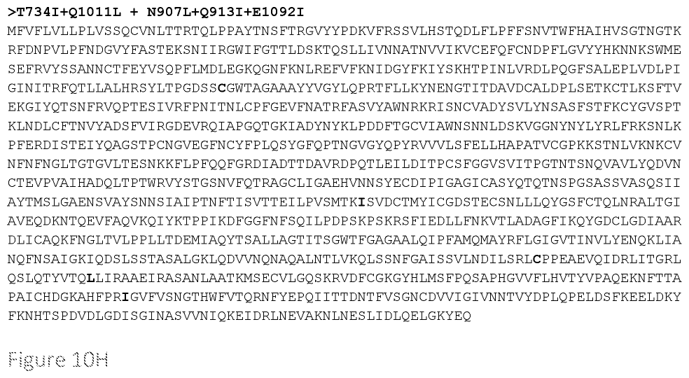

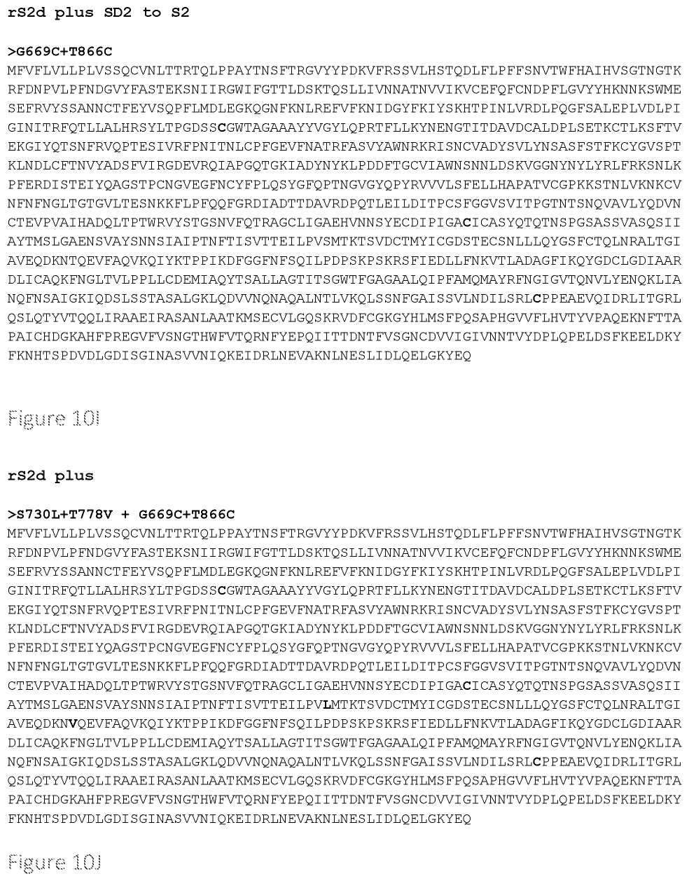

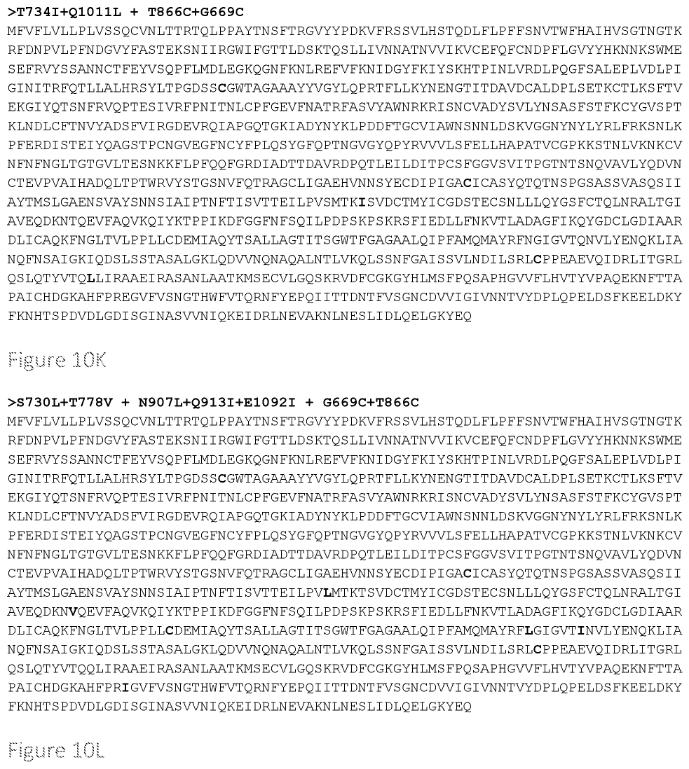

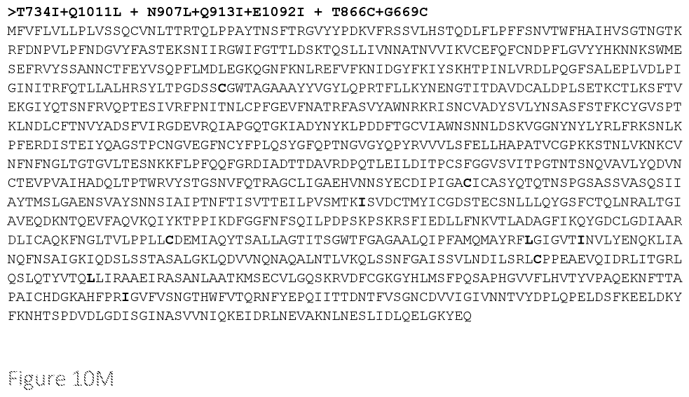

TABLE 9B
Summary of sequences from FIG.s 10A-M

| | A non-limiting |
|---|---|
| | embodiment of a |
| | sequence is shown in |
| Name | Figure. |
| rS2d plus S2 stabilization: | A non-limiting |
| S730L + T778V | embodiment of a |
| T734I + Q1011L | sequence is shown in |
| T734I + Q1011L + Y1007F | FIG. 10A-10H |
| T8811 + Q901L + R905Y | |
| N907L + Q913I + E1092I | |
| N907L + Q913I + E1092F | |
| S730L + T778V + N907L + Q913I + E1092I | |
| T734I + Q1011L + N907L + Q913I + E1092I | |
| rS2d plus SD2 to S2: | A non-limiting |
| G669C + T866C | embodiment of a |
| T866C + G669C | sequence is shown in |
| | FIG. 10I |
| rS2d plus S2 stabilization and SD2 to S2: | A non-limiting |
| S730L + T778V + G669C + T866C | embodiment of a |
| T734I + Q1011L + T866C + G669C | sequence is shown in |
| S730L + T778V + N907L + Q913I + | FIG. 10J-M |
| E1092I + G669C + T866C | |
| T734I + Q1011L + N907L + Q913I + | |
| E1092I + T866C + G669C | |

TABLE 9C
Summary of sequences of cluster mutations from FIG. 8.

| | A non-limiting |
|---|---|
| | embodiment of a |
| | sequence is shown in |
| Group | Figure. |
| Cluster 1 | FIG. 8B- |
| Cluster 2 | FIG. 8C |
| Cluster 3 | FIG. 8D |
| Cluster 4 | FIG. 8E |
| Cluster 5 | FIG. 8F |
| Cluster 6 | FIG. 8G |
| Cluster 7 | FIG. 8H |
| Cluster 8 | FIG. 8I |
| Cluster 9 | FIG. 8J |
| Cluster 10 | FIG. 8K |
| Cluster 11 | FIG. 8L |

### EXAMPLES

### Example 1A

Any of the SARS-2 designs, including without limitation as listed in FIG. 7, 8, 9, 10, 25 will be expressed, characterized and tested for antigenicity and immunogenicity. Immuonogenicity studies include animal challenge studies. A non-limiting embodiment of an animal study is outlined in Example 2.

### Example 1B

SARS-2 designs expressed as nucleic acids or proteins will be expressed, characterized and tested for antigenicity and immunogenicity. Immuonogenicity studies include animal challenge studies. A non-limiting embodiment of an animal study is outlined in Example 2.

### Example 2

Animal study NHP #174: non-human primates (NHPs) are immunized with SARS-2 immunogen designs of the invention. Immune response was evaluated and animals were challenge with SARS-2 stock. The animal study design and immunogen are summarized in FIG. 11A-B.

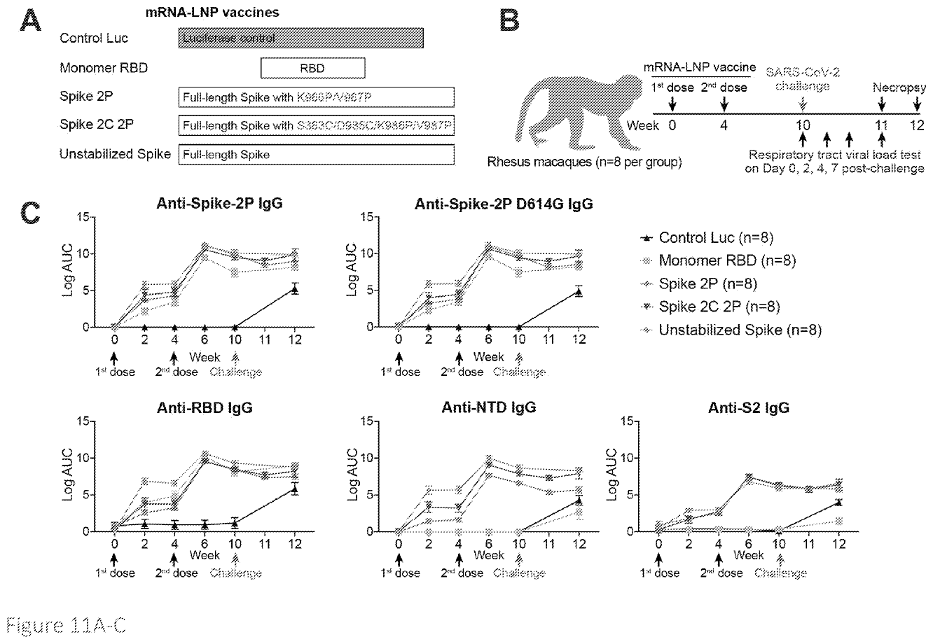

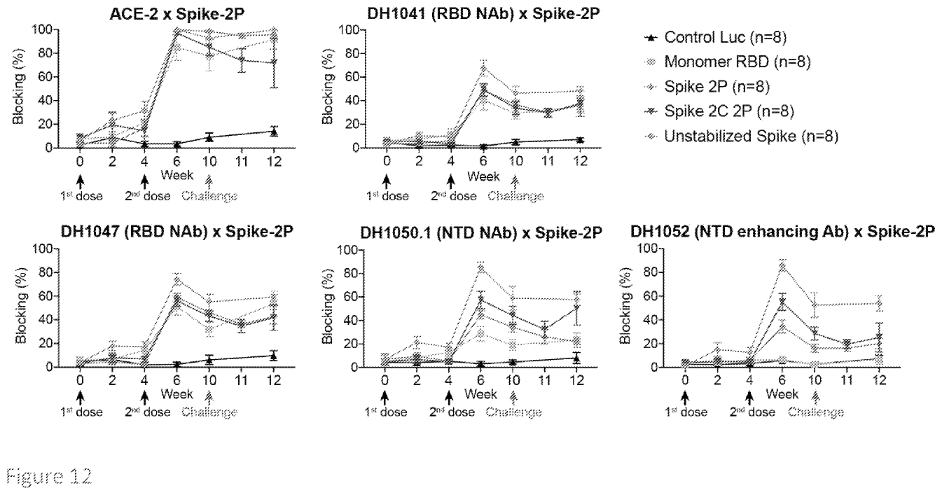

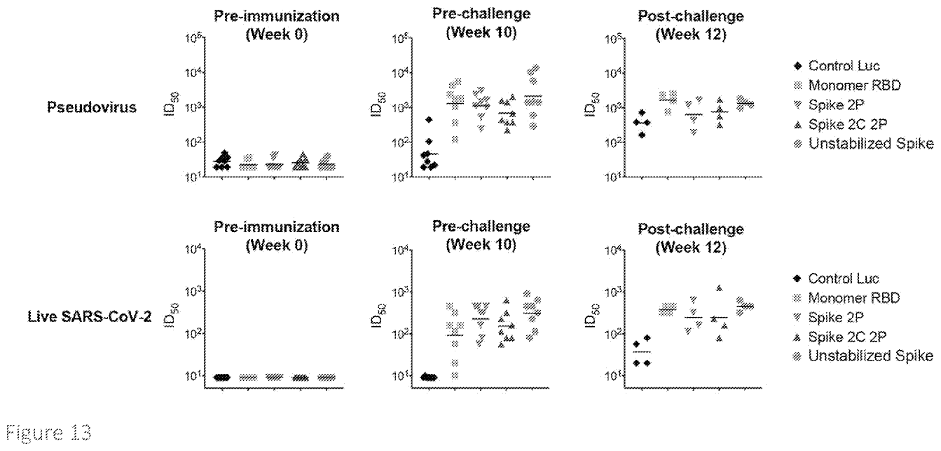

Data from the animal study are summarized in FIGS. 11-24.

These results show that immunization with the disulfide-stabilized spike ectodomain mRNA-LNP in rhesus macaques elicited IgG antibodies against the receptor binding, N-terminal, and S2 domains of SARS-CoV-2 spike protein. The serum from disulfide-stabilized spike ectodomain mRNA-LNP-immunized macaques blocked ACE2 binding to the receptor domain of SARS-CoV-2 spike protein. Consistent with blocking the ACE2 receptor binding to SARS-CoV-2 spike, the serum neutralized both pseudotyped virus and replication-competent SARS-CoV-2. The vaccine-induced immunity suppressed SARS-CoV-2 replication in the lower respiratory tract and to a lesser extent in the upper respiratory tract. Additionally, inflammatory cytokine production in the lung was decreased in disulfide-stabilized spike ectodomain mRNA-LNP-immunized compared to macaques that received mRNA-LNP encoding an irrelevant protein. Thus, immunization with disulfide-stabilized spike ectodomain mRNA-LNP-immunized generated immunity that protected against SARS-CoV-2 infection.

Further analyses of the animal study include immunogenicity, levels of antibodies, types of antibodies—neutralizing or not, serum neutralization of pseudo-virus, diversity of epitopes targeted by the induced antibodies, protection after challenge with virus, and any other suitable assay.

### Example 3A

Controlling the SARS-CoV-2 Spike Glycoprotein Conformation

Abstract

The coronavirus (CoV) viral host cell fusion spike (S) protein is the primary immunogenic target for virus neutralization and the current focus of many vaccine design efforts. The highly flexible S-protein, with its mobile domains, presents a moving target to the immune system. Here, to better understand S-protein mobility, we implemented a structure-based vector analysis of available β-CoV S-protein structures. We found that despite overall similarity in domain organization, different β-CoV strains display distinct S-protein configurations. Based on this analysis, we developed two soluble ectodomain constructs in which the highly immunogenic and mobile receptor binding domain (RBD) is locked in the all-RBDs ‘down’ position or is induced to display a previously unobserved in SARS-CoV-2 2-RBDs ‘up’ configuration. These results demonstrate that the conformation of the S-protein can be controlled via rational design and provide a framework for the development of engineered coronavirus spike proteins for vaccine applications.

### INTRODUCTION

The ongoing global pandemic of the new SARS-CoV-2 (SARS-2) coronavirus presents an urgent need for the development of effective preventative and treatment therapies. The viral S-protein is a prime target for such therapies owing to its critical role in the virus lifecycle. The S-protein is divided into two regions: an N-terminal S1 domain that caps the C-terminal S2 fusion domain. Binding to host receptor via the Receptor Binding Domain (RBD) in S1 is followed by proteolytic cleavage of the spike by host proteases¹. Large conformational changes in the S-protein result in S1 shedding and exposure of the fusion machinery in S2. Class I fusion proteins, such as the CoV-2 S-protein, undergo large conformational changes during the fusion process and must, by necessity, be highly flexible and dynamic. Indeed, cryo-electron microscopy (cryo-EM) structures of SARS-2 spike reveal considerable flexibility and dynamics in the S1 domain^{1,2}, especially around the RBD that exhibits two discrete conformational states—a ‘down’ state that is shielded from receptor binding, and an ‘up’ state that is receptor-accessible.

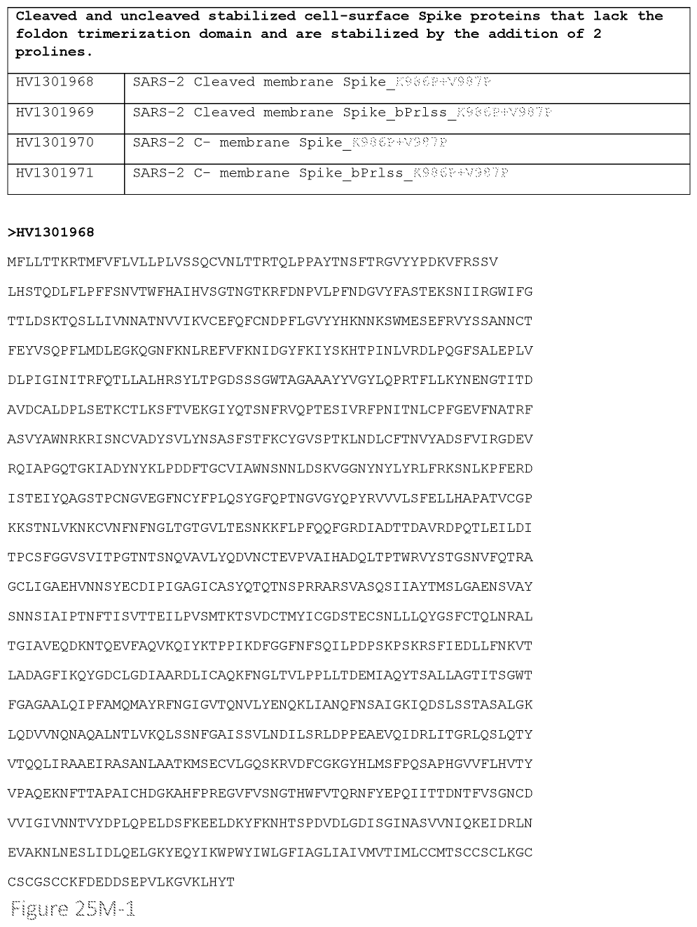

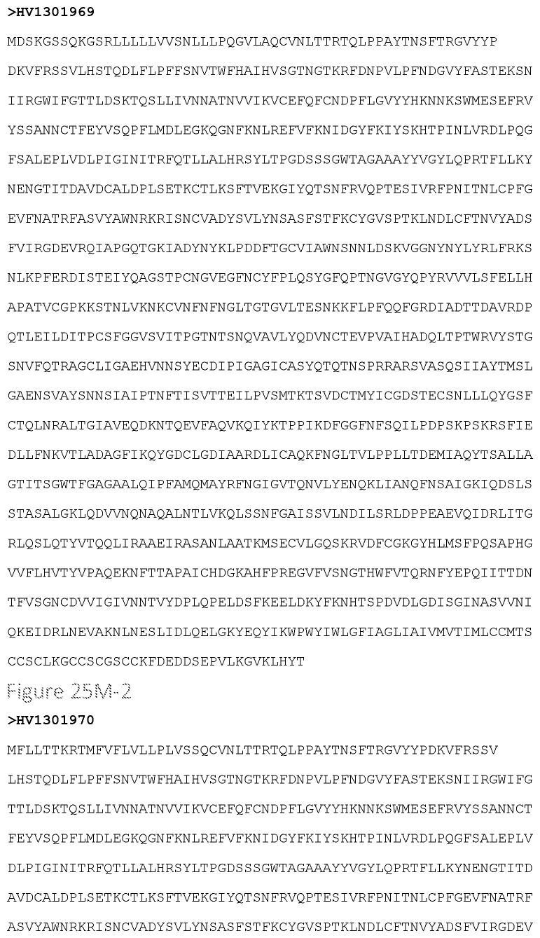

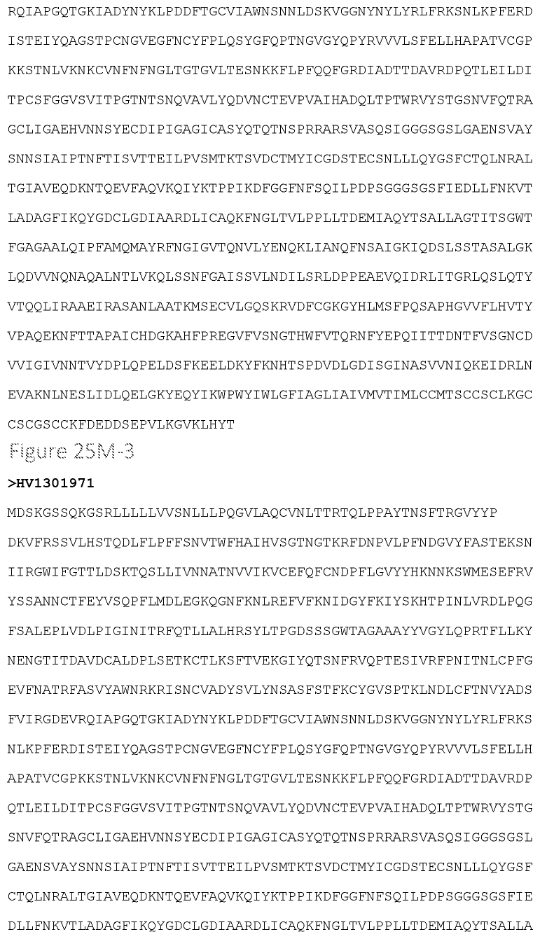

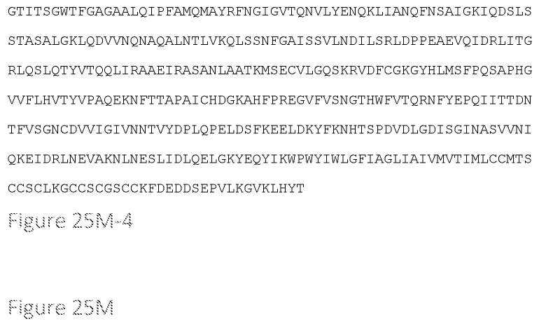

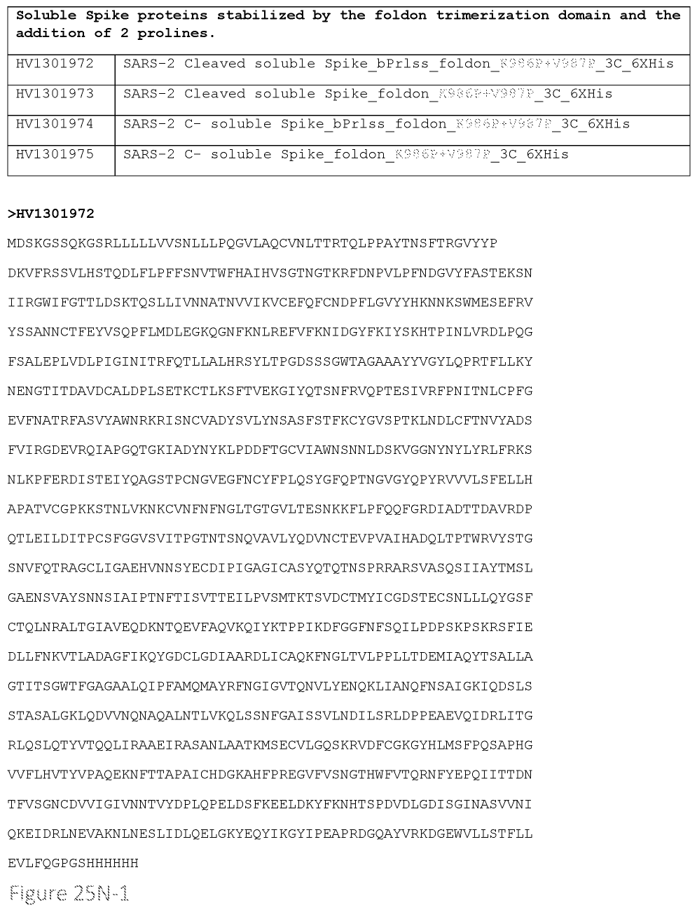

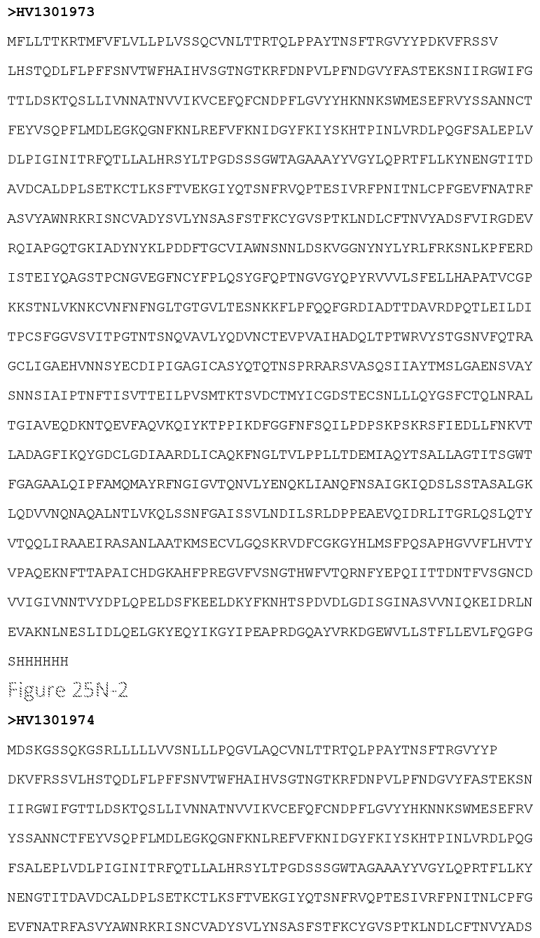

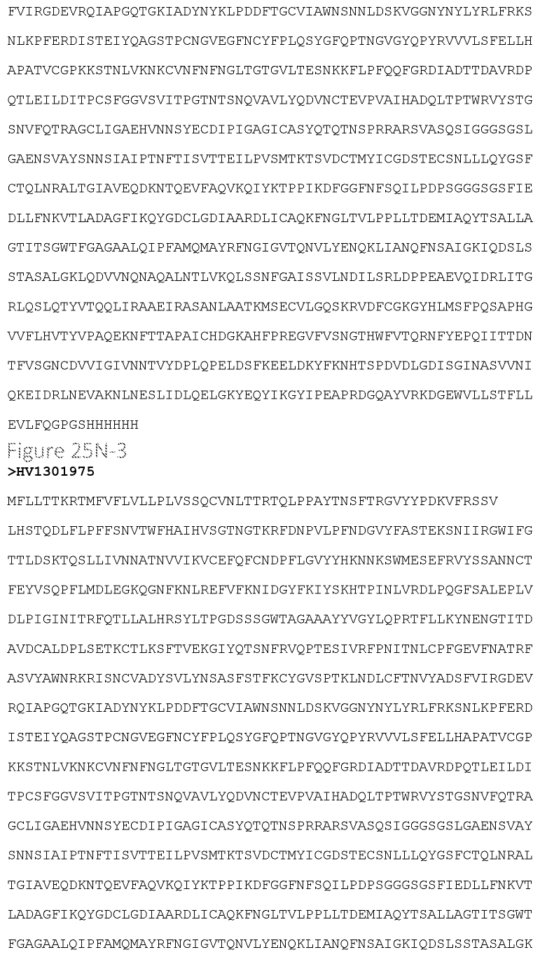

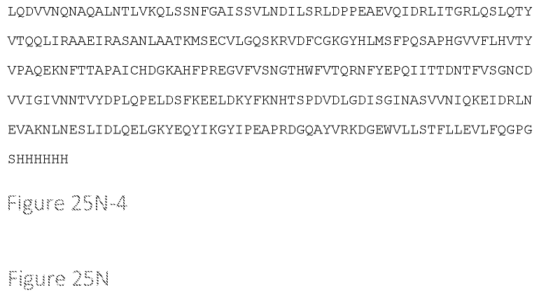

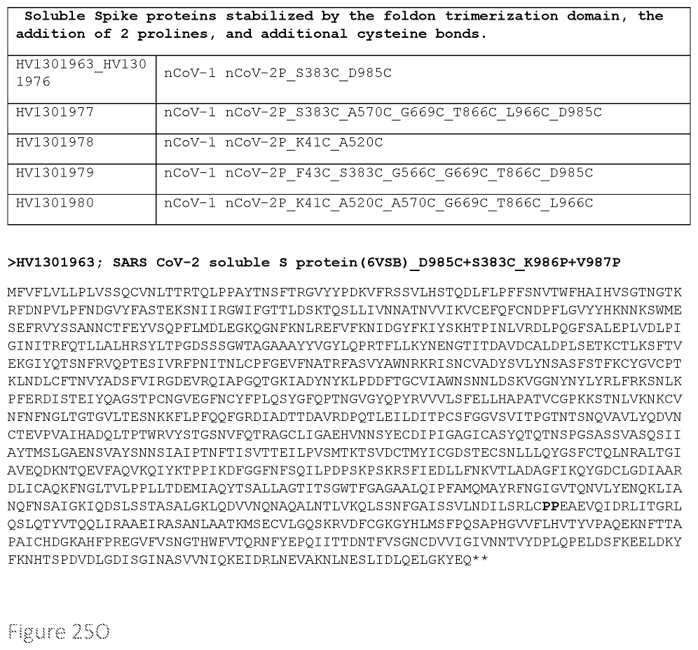

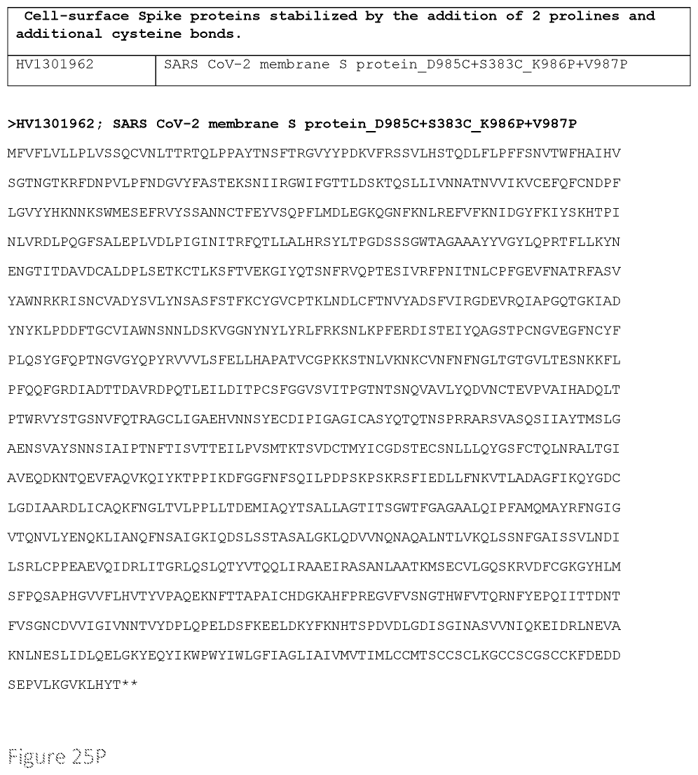

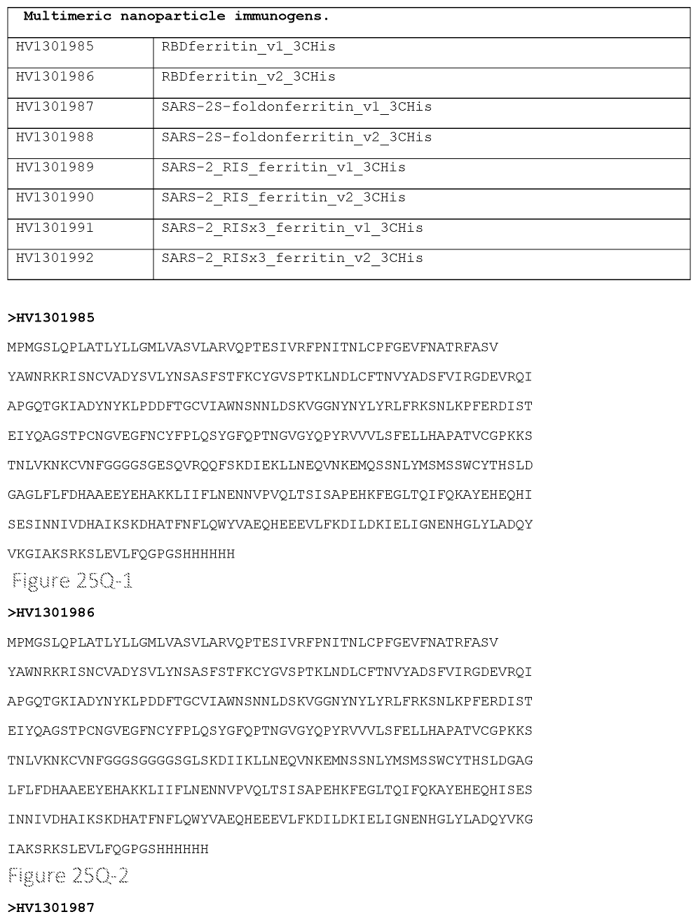

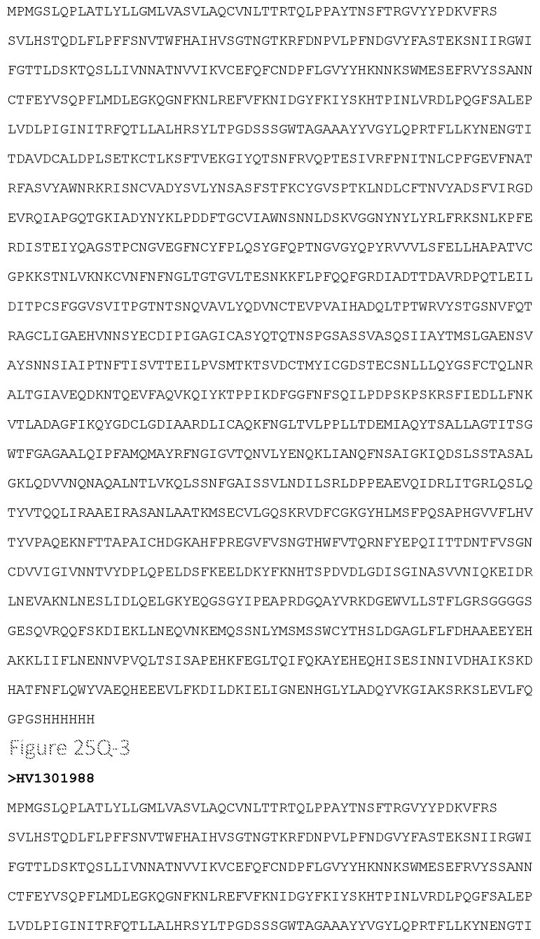

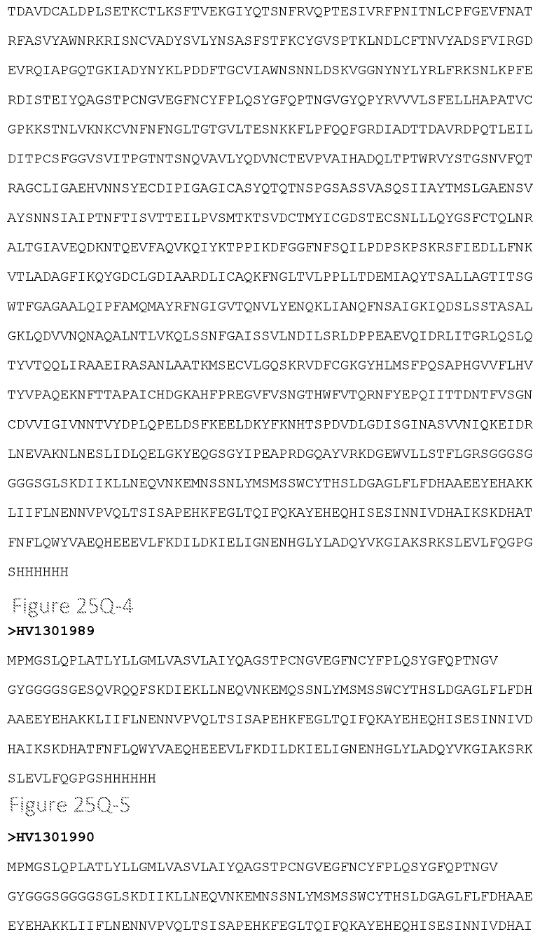

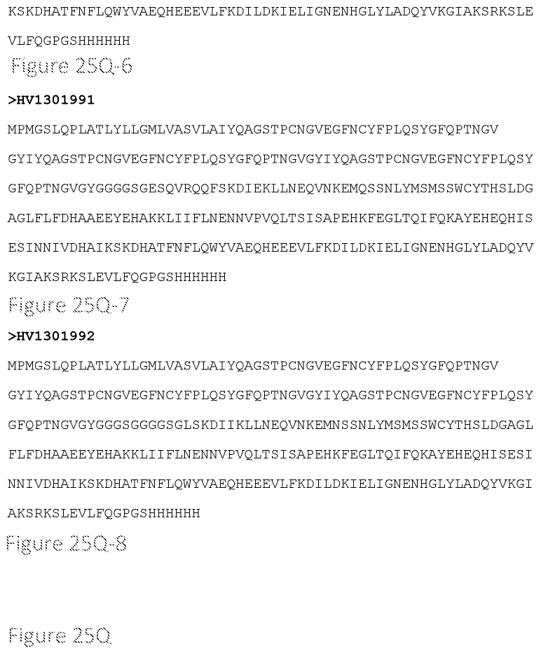

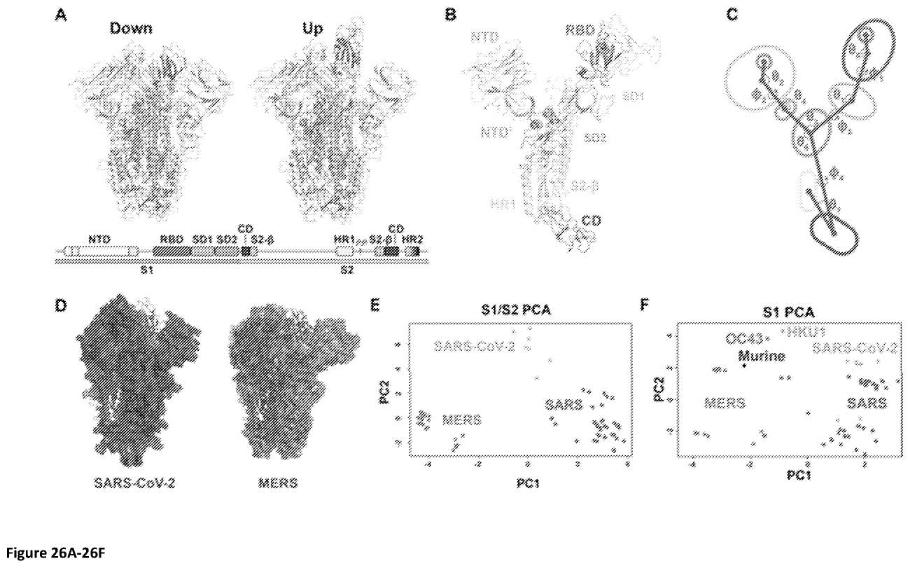

The wealth of structural information for β-CoV spike proteins, including the recently determined cryo-EM structures of the SARS-2 spike¹⁻¹¹, has provided a rich source of detailed geometric information from which to begin precise examination of the macromolecular transitions underlying triggering of this fusion machine. The transmembrane CoV S-protein spike trimer is composed of interwoven protomers that include an N-terminal receptor binding S1 domain and a C-terminal S2 domain that contains the fusion elements (FIGS. 26A and B).2 The S1 domain is subdivided into the N-terminal domain (NTD) followed by the receptor binding domain (RBD) and two structurally conserved subdomains (SD1 and SD2). Together these domains cap the S2 domain, protecting the conserved fusion machinery. Several structures of soluble ectodomain constructs that retain the complete S1 domain and the surface exposed S2 domain have been determined. These include SARS-2^{1,3}, SARS⁴⁻⁸, MERS^{4,9}, and other human^{2,10} and murine¹¹ β-CoV spike proteins. These structures revealed remarkable conformational heterogeneity in the S-protein spikes, especially in the RBD region. Within a single protomer, the RBD can adopt a closed ‘down’ state (FIG. 26A), in which the RBD covers the apical region of the S2 protein near the C-terminus of the first heptad repeat (HR1), or an open ‘up’ state in which the RBD is dissociated from the apical central axis of S2 and the NTD. Further, cryo-EM structures indicates a large degree of domain flexibility in both the ‘down’ and ‘up’ states in the NTD and RBD. While these structures have provided essential information to identify the relative arrangement of these domains, the degree to which conformational heterogeneity can be altered via mutation during the natural evolution of the virus and in a vaccine immunogen design context remains to be determined.

In this study we have quantified the variability in the S1 and S2 geometric arrangements to reveal important regions of flexibility to consider and to target for structure-based immunogen design. Based on these analyses, we have designed mutations that alter the conformational distribution of the domains in the S-protein. We visualized the effect of our designs using a structural determination pipeline relying first on single particle analysis by negative stain electron microscopy (NSEM) for rapid and low-cost assessment of the spike ectodomains at low resolution, followed by cryo-EM for high-resolution information on the changes introduced by these mutations. Our results reveal a heterogeneous conformational landscape of the SARS-CoV-2 spike that is highly susceptible to modification by the introduction of mutations at sites of contact between the S1 and S2 domains. We also present data on modified SARS-2 ectodomain constructs stabilized in conformations that have not yet been seen in the current available structures, with great interest and direct application in vaccine design.

Results

Detailed Structural Schema Defining the Geometry and Internal Rearrangements of Movable Domains of the SARS-2 Spike.

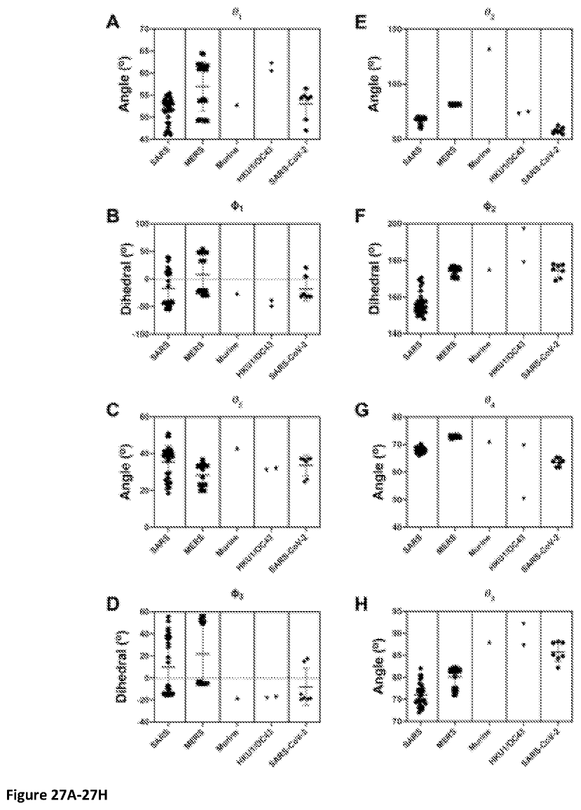

To characterize the unique arrangement of distinct domains in the CoV spike, we first aimed to develop a precise quantitative definition of their relative positions. Examination of available SARS and MERS S-protein structures revealed: 1) the NTD and RBD subdomains and internal S2 domain move as rigid bodies, and 2) these domains display a remarkable array of relative shifts between the domains in the S1 region and the S2 region's β-sheet motif and connector domain (CD) (FIG. 26B-F). In order to quantify these movements, we have analyzed the relevant regions of motion and their structural disposition in all available β-CoV ectodomain spike structures including 15 SARS^{4,5,7,8}, 10 MERS^{4,12}, a HKU1^{2,10}, an OC43^{2,10}, a murine β-CoV, and three SARS-2^{13,14} structures (FIGS. 26E-F and 27). Each protomer in those structures displaying asymmetric ‘up’/‘down’ RBD states was examined independently yielding a dataset of 76 structural states. The NTD was split into a primary N-terminal section and a secondary C-terminal section based upon visual inspection of this region in the various β-CoV structures (FIGS. 26B-C and 27). We next analyzed S-protein geometry using a vector-based approach. Specifically, vectors connecting each region's C_{α} centroids were generated and used to define the relative dispositions of the domains (FIGS. 26C and 27). The vector magnitudes and select angles and dihedrals were used to identify the breadth of differences in domain positioning and compare between strains. The results indicated that β-CoV spike proteins in various strains differ markedly from one another and that considerable variability in the domain arrangements within strains exists, especially in the SARS ectodomains (FIGS. 26E-F and 27A-H). For example, both θ₁ and ϕ₁ (FIG. 27A-B), describing the angle between the SD2 to SD1 and SD1 to RBD vectors as well as the SD1 to RBD dihedral, respectively, effectively report on the ‘up’ and ‘down’ configurations while indicating substantial differences between SARS and MERS in both the ‘up’ and ‘down’ states. The angular disposition of the NTD elements further indicated differences in SARS and MERS with a marked shift from the examined β-CoV spikes in the murine structure (FIG. 26E). Additional S1 differences are observed between vectors involving SD2. The disposition of the S2 domain relative to S1 defined by the dihedral about the vector connecting SD2 to the S2 CD differs markedly between MERS/SARS-2 and SARS as well with the angle between the vectors connecting the NTD′ to SD2 and SD2 to the CD demonstrating a shift in SARS-2. Finally, the disposition of the CD to the inner portion of S2 measured as an angle between a vector connected to an interior S2 β-sheet motif and the vector connecting the CD to SD2 indicates SARS differs from both MERS and SARS-2. Interestingly, the MERS disposition appears to respond to RBD triggering, displaying a bimodal distribution. These results demonstrate that, while the individual domain architectures and overall arrangements are conserved (FIG. 26D), important differences between these domains exists between strains, indicating that subtle differences in inter-domain contacts can play a major role in determining these distributions and thereby alter surface antigenicity and the propensity of the domains to access ‘up’ and ‘down’ RBD states.

Identification of Sites for Differential Stabilization of the SARS-2 Ectodomain Spike RBD Orientation.

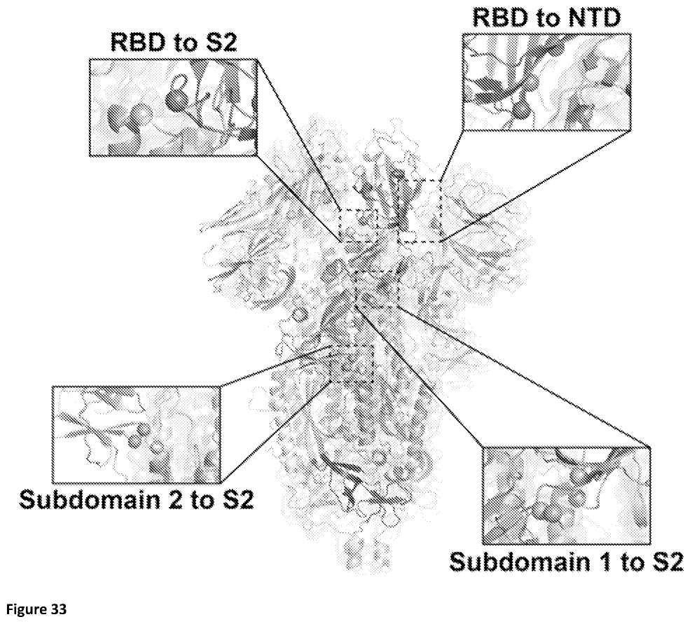

Based on the observed variability in the geometric analysis of β-CoV spikes, we asked whether the propensity for the RBD to display the ‘down’ and ‘up’ states can be modified via mutations without altering exposed antigenic surfaces. To this end, we identified protomer to protomer interactive sites amenable to modification and down selected mutations at these sites using the Schrödinger Biologics suite. In an effort to eliminate exposure of the receptor binding site of the RBD, we examined the potential for disulfide linkages between the RBD and its contact with S2 near the C-terminus of HR1 to prevent RBD exposure. We identified a double cysteine mutant, S383C and D985C (RBD to S2 double mutant; rS2d; FIG. 33), as a candidate for achieving this goal. The transition from the ‘down’ state to the ‘up’ state involves shifts in the RBD to NTD contacts. Therefore, in an effort to prevent these shifts, we identified a site in an RBD groove adjacent to the NTD for which we prepared a triple mutant, D398L, S514L, and E516L (RBD to NTD triple mutant; rNt, FIG. 33). As SD1 acts as a hinge point for the RBD ‘up’/‘down’ transitions (FIGS. 26A-C, 27I-J), without wishing to be bound by theory, enhanced hydrophobicity at the SD1 to S2 interface can shift the position of SD1, thus influencing the hinge and potentially the propensity for RBD triggering. A double mutant, N866I and A570L (Subdomain 1 to S2 double mutant; u1S2d, FIG. 33), as well as quadruple mutant, A570L, T572I, F855Y, and N8561 (Subdomain 1 to S2 quadruple mutant; u1S2q), were identified for this purpose. Finally, we asked whether linking SD2 to S2 can alter the conformational distribution of the RBDs. The double cysteine mutant, G669C and T866C (Subdomain 1 to S2 double mutant; u2S2d, FIG. 33), was identified for this purpose. These mutants were prepared in the context of a previously published SARS-2 ectodomain construct³.

NSEM Analysis of the SARS-2 Spike Ectodomain Proteins.

To assess the quality of the purified spike proteins and to obtain low resolution visualization of the structures, we performed NSEM analysis. The micrographs showed a reasonably uniform distribution of particles consistent with the size and shape of the SARS-2 spike ectodomain (FIG. 28). 2D class averages showed spike populations with well resolved domain features. The data were subjected to 3D classification followed by homogeneous refinement. The unmutated construct was resolved into two classes of roughly equal proportions. The two classes differed in the position of their RBD domains. One classy displayed all three RBDs in their ‘down’ positions, whereas, the other class, —displayed one RBD in the ‘up’ position. This was consistent with published cryo-EM results¹⁵ that described a 1:1 ratio between the ‘down’ and ‘1-up’ states of the SARS-2 spike ectodomain. The mutant spikes were analyzed using a similar workflow as the unmutated spike. All of the mutants displayed well-formed spikes in the micrographs, as well as in the 2D class averages. Following 3D classification, for the rS2d construct, we observed only the ‘down’ conformation; the 1-RBD up state that was seen for the unmutated spike was not found in this dataset. The u1S2q mutant presented another striking finding, where we observed a new conformational state with 2 RBDs in the ‘up’ position. The 2-RBD ‘up’ state has been reported before for the MERS CoV spike ectodomain¹² but has not been observed thus far for the SARS or the SARS-2 spikes. Based on the NSEM analysis we selected the rS2d and u1S2q constructs for high resolution analysis by cryo-EM.

Cryo-EM Analysis of the SARS-2 Spike Ectodomain Proteins.

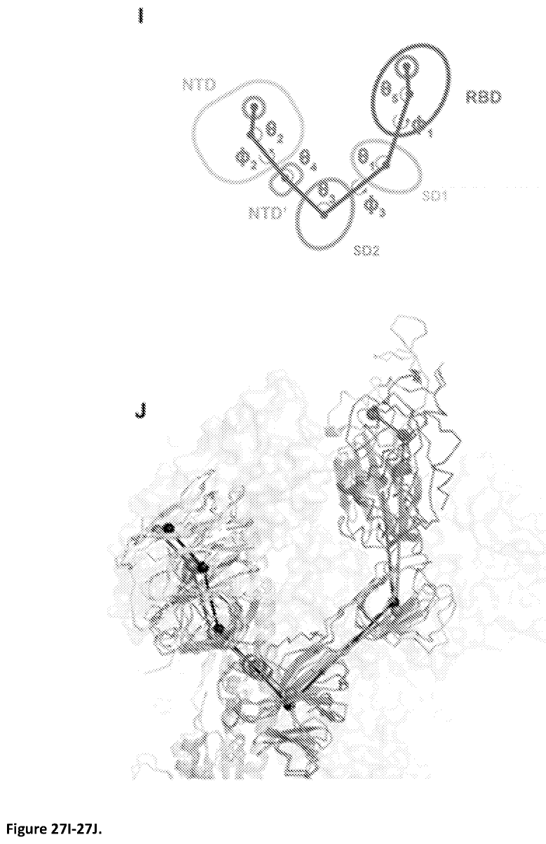

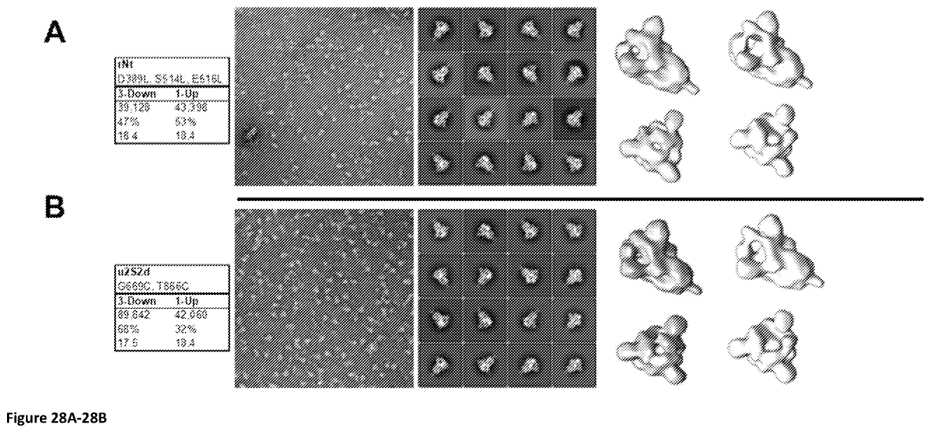

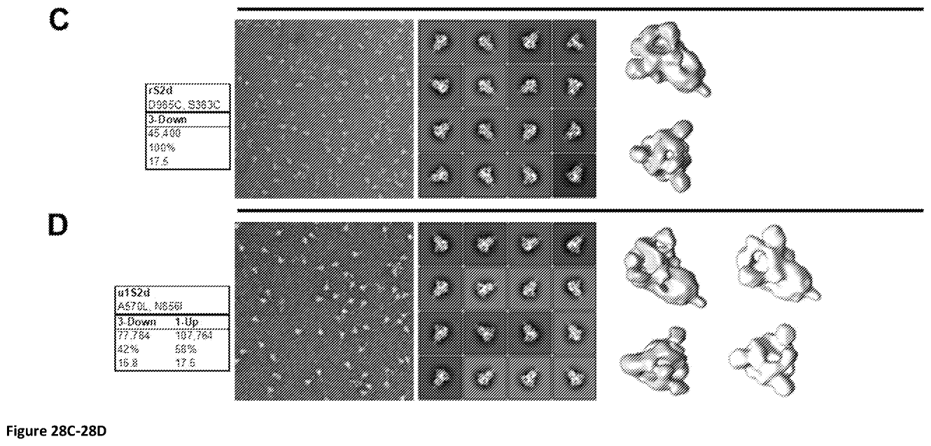

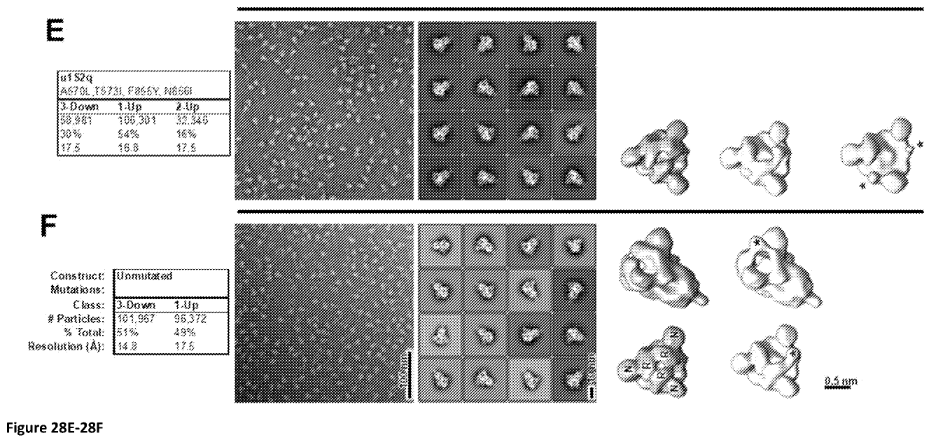

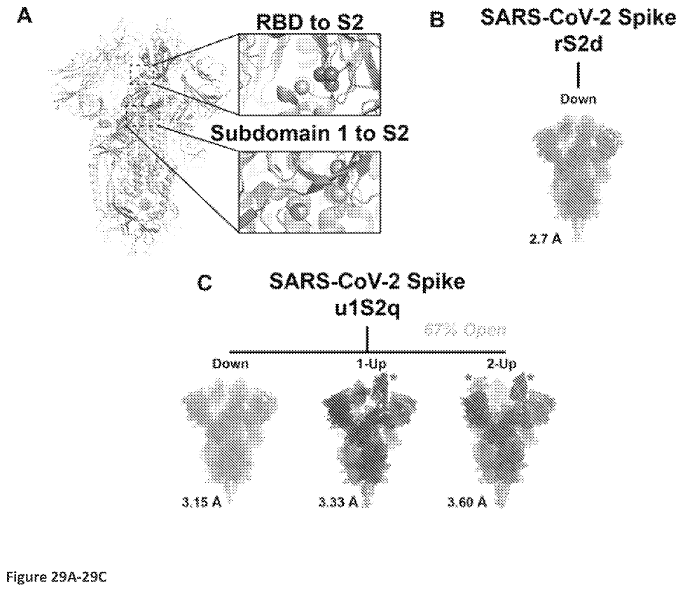

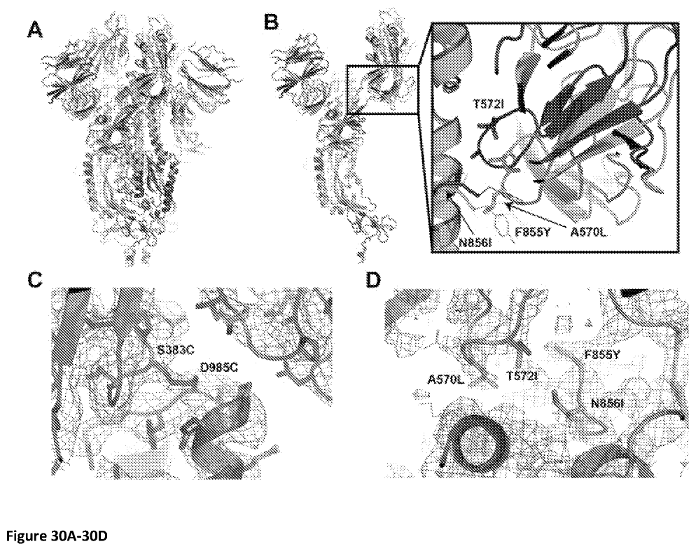

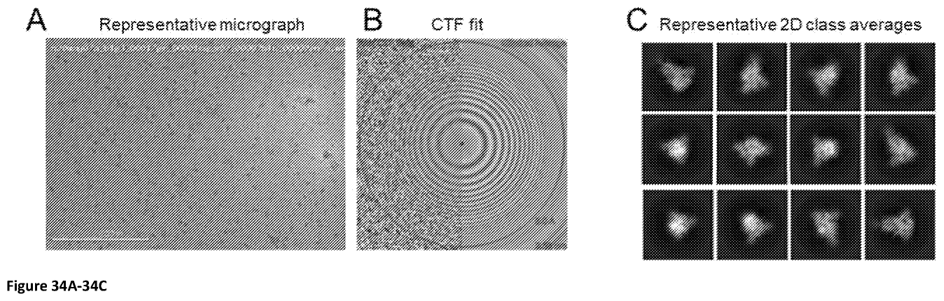

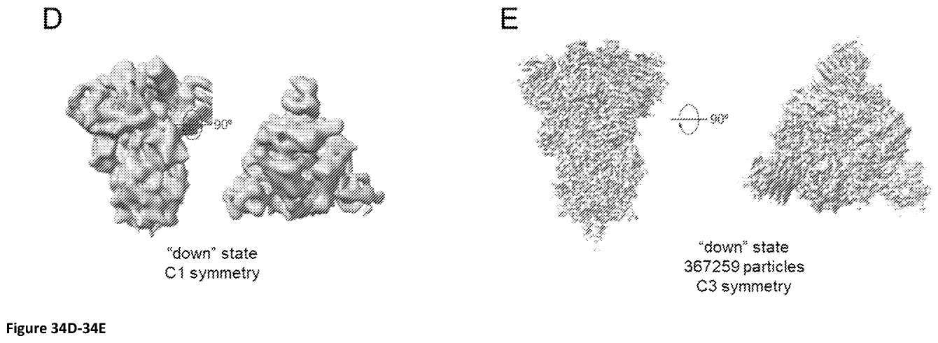

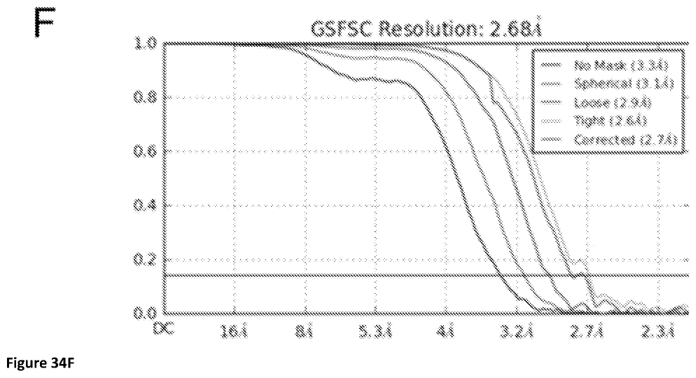

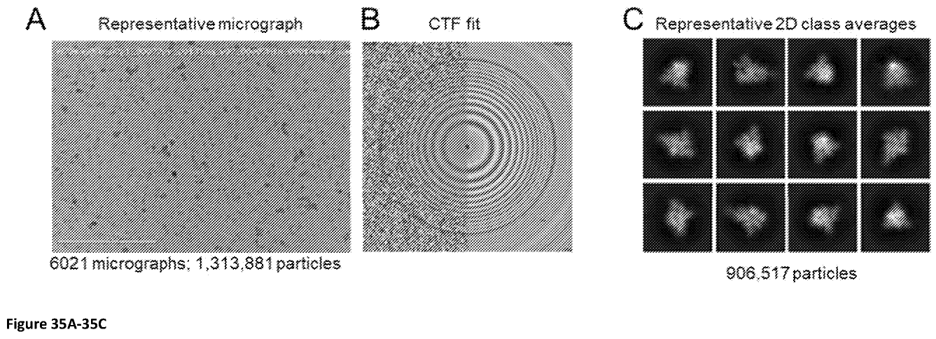

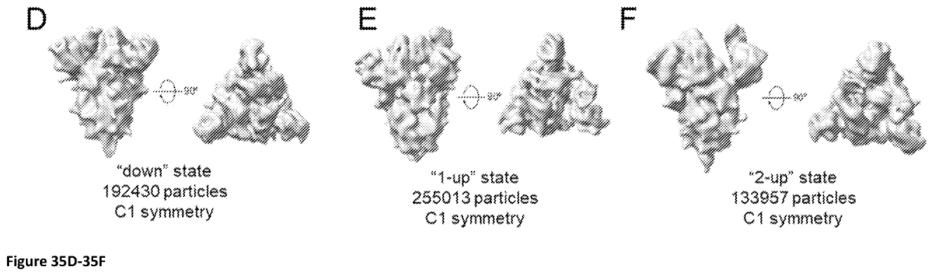

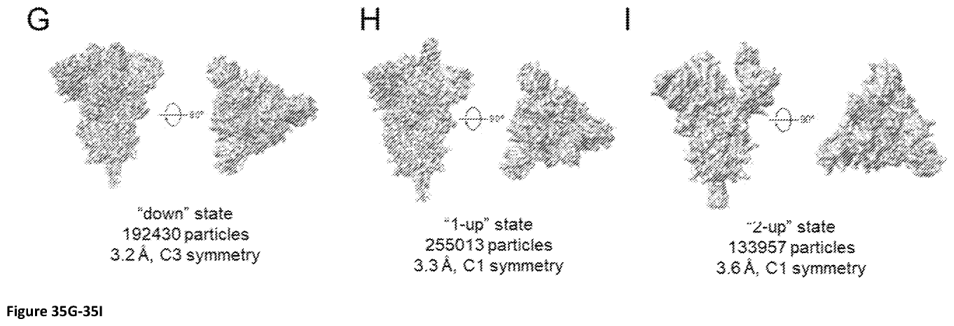

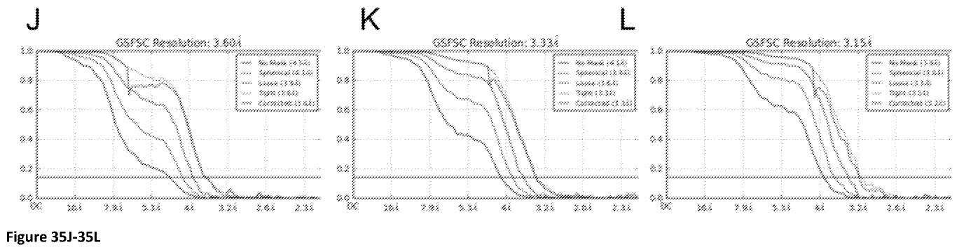

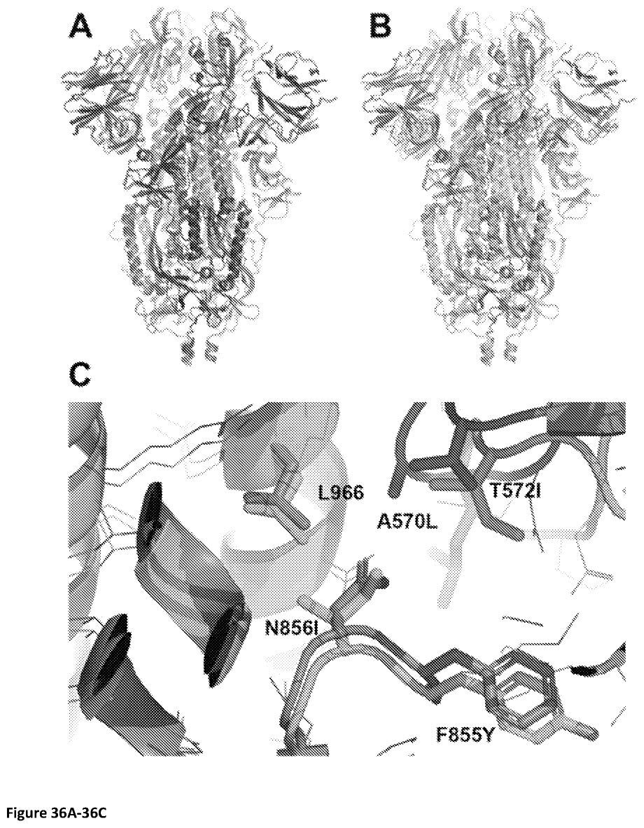

To visualize the mutations and their effect on the structure of the spike, we collected cryo-EM datasets for the rS2d and u1S2q constructs (FIG. 29-32, Table 10, FIGS. 34 and 35). Consistent with what was observed in the NSEM analysis, after multiple rounds of 2D and 3D-classification to remove junk particles and broken and/or misfolded spikes, we found a population of ‘down’ state spike in the rS2d dataset through ab initio classification in cryoSparc. We then implemented additional exhaustive ab initio classifications, as well as heterogeneous classifications using low-pass filtered maps of known open conformations of CoV spikes to search for open state spikes in the dataset. We were unable to find any such states, confirming that the SARS-2 spike was locked in its ‘down’ conformation in the rS2d mutant. The rS2d disulfide linked density at the mutation site confirmed disulfide formation in the double mutant (rS2d) (FIG. 30). Comparison of the domain arrangements of this construct with that of the unmutated ‘down’ closed state structure indicated the protein structure was otherwise unperturbed (Supplemental FIG. 36A).

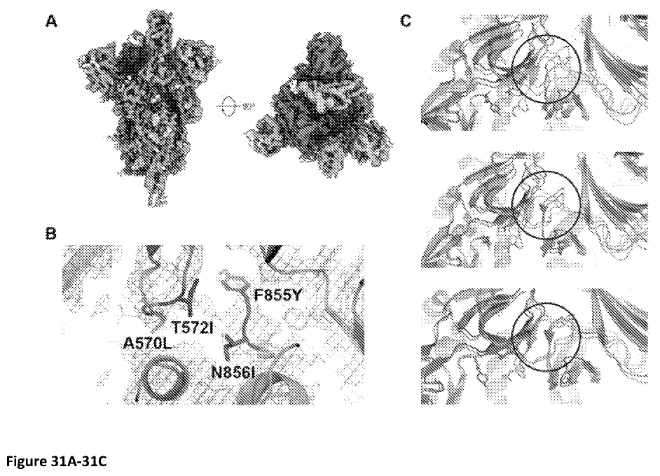

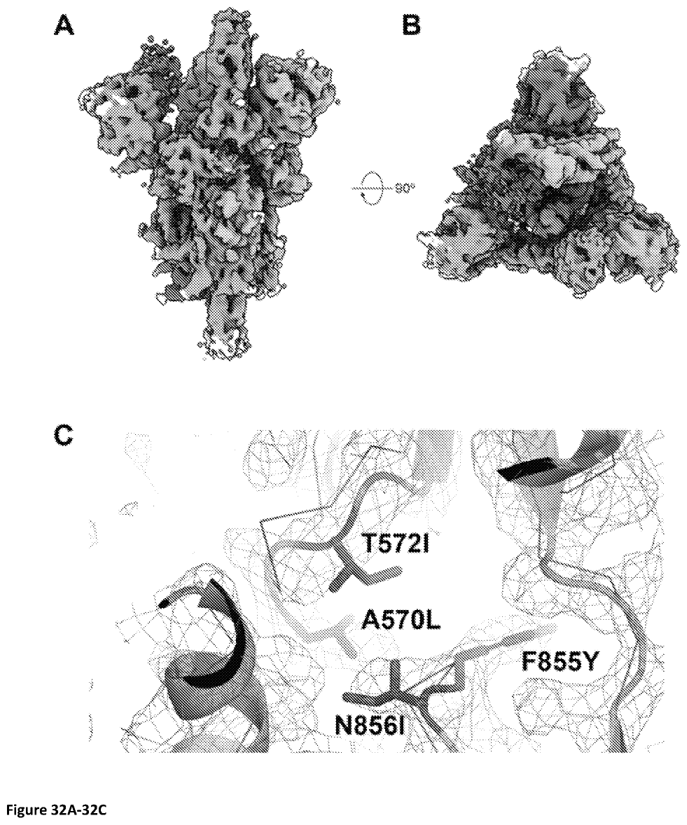

In contrast to the rS2d design, the u1S2q design displayed widespread rearrangement of the S1 domains (FIG. 33B). In the ‘down’ state structure, density in the mutated S2 position remained in the configuration observed in the unmutated construct with the N8551 and F856Y residue loop in close proximity the S2 residue L966 and S1 residue P589. This indicated that these mutations had little impact on the observed shifts. However, the S2 interactive SD1 displayed a rigid body movement relative to both the rS2d and unmutated constructs with θ₁ and ϕ₃ displacements of 3.4° and 1.8°, respectively (FIGS. 30A and B). This resulted in displacement of the A570L+T572I containing loop from the unmutated position which resides near the S2 L966 residue (FIG. 30B and FIG. 33C). The S2 contact disruption is accompanied by an angular shift of the NTD away from the primary trimer axis owing to subdomain-1 to NTD′ contacts, yielding θ₃ and ϕ₃ shifts of 5.4° and 7.7°, respectively (FIG. 31C). The subdomain rearrangement impacts the positioning of the RBD with only a minor shift in the ϕ1 dihedral of 0.1° indicating the RBD moved with SD1 indicated in the θ1/ϕ3 shifts. The newly acquired arrangement in both the RBD and NTD was further accompanied by an apparent increase in their flexibility indicating conformational heterogeneity. These down state shifts were observed in both the single RBD ‘up’ structure and the two RBD ‘up’ structures (FIG. 31). Interestingly, the extent to which the SD1 shift differed from that observed in the unmutated construct was context dependent in the 1 RBD up state. While the down state RBD in contact with the up state RBD displayed the large shift in position observed in the all down state, the down state RBD with its terminal position free displayed an intermediate SD1 configuration. The up state RBD in the u1S2q construct resided largely in the position occupied in the unmutated construct. This indicated the effect of the mutations was primarily isolated to the down state and indicated these mutations act to destabilize the down state rather than to stabilize the up state. These features were largely recapitulated in the u1S2q 2 RBD up state conformation with subdomain 1 retaining the shift in the down state RBD (FIG. 32). The structural details presented here indicate that, while locking the ‘down’ state RBD into its unmutated position had little impact on the overall configuration of S1, altering the disposition of SD1 had wide ranging impacts, consistent with the observed strain-to-strain differences in the geometric analysis described in FIGS. 26 and 27.

TABLE 10
Cryo-EM Data Collection and Refinement Statistics

| SARS-2 spike | | u1S2q construct | | |
|---|---|---|---|---|
| construct | r2S2d | | 1-RBD | 2-RBD |
| Conformation | ‘down’ | ‘down’ | ‘up’ | ‘up’ |
| Data Collection | FEI Titan | | FEI Titan | |
| Microscope | Krios | | Krios | |
| Voltage (kV) | 300 | | 300 | |
| Electron dose (e⁻/Å²) | 65.18 | | 66.82 | |
| Detector | Gatan K3 | | Gatan K3 | |
| Pixel Size (Å) | 1.06 | | 1.058 | |
| Defocus Range (μm) | 0.63-2.368 | | 0.55-2.94 | |
| Magnification | 81000 | | 81000 | |
| Micrographs | 6021 | | 7232 | |
| Collected | | | | |
| Reconstruction | | | | |
| Software | cryoSPARC | | cryoSPARC | |
| Particles | 367,259 | 192,430 | 255,013 | 133,957 |
| Symmetry | C3 | C3 | C1 | C1 |
| Box size (pix) | 300 | 300 | 300 | 300 |
| Resolution (Å)^{$} | 2.7 | 3.2 | 3.3 | 3.6 |
| Corrected | | | | |
| Refinement (Phenix) | | | | |
| ^{#} | | | | |
| Protein residues | 2916 | 2913 | 2875 | 2862 |
| Resolution (FSC_{0.5}) | 2.9 | 3.3 | 3.7 | 3.8 |
| EMRinger Score | 3.11 | 3.02 | 1.33 | 2.69 |
| R.m.s. deviations | | | | |
| Bond lengths (Å) | 0.009 | 0.005 | 0.013 | 0.011 |
| Bond angles (º) | 1.2 | 0.859 | 1.276 | 1.272 |
| Validation | | | | |
| Molprobity score | 1.58 | 1.52 | 0.75 | 1.84 |
| Clash score | 3.93 | 4.57 | 0.41 | 6.6 |
| Favored rotamers (%) | 99.41 | 98.75 | 99.34 | 97.46 |
| Ramachandran | | | | |
| Favored regions (%) | 94.23 | 95.88 | 97.5 | 92.37 |
| Disallowed regions | 0 | 0.04 | 0.07 | 0.11 |
| (%) | | | | |

^{$}Resolutions are reported according to the FSC 0.143 gold-standard criterion

### DISCUSSION

Conformational plasticity is a hallmark of enveloped-virus fusion-protein structure, owing to the necessity of protecting the conserved viral fusion elements from host immune responses while retaining a sufficiently steep free-energy gradient to enable host cell fusion¹⁶. Exposed elements can be well conditioned to be permissive and responsive to mutations through genetic drift and host immune adaptation. Conformational plasticity, however, presents an important difficulty in the context of vaccine and drug design. Indeed, lessons learned in the continued effort to produce a broadly protective HIV-1 vaccine have demonstrated the importance of a detailed understanding and control of fusion protein dynamics¹⁷⁻²⁸. The new SARS-CoV-2 is no exception in this regard and indeed the conformational plasticity of the SARS-2 S-protein appeared greater than that of the HIV-1 Env. We aimed to develop a quantitative understanding of β-CoV structural states between strains and within each RBD down and up state configuration. The wide breadth of domain arrangements along with the relatively small contact area between the S1 and S2 subunits observed here indicated that, despite a relatively low mutation rate, dramatic changes in S-protein structure can occur from few mutations. Indeed, recent evidence for a mutation in the SD2 to S2 contact region indicates a fitness gain for acquisition of such interfacial residues²⁹. Based upon our results, this mutant, D614G, can indeed alter the conformational landscape of the SARS-CoV-2 S-protein.

From the perspective of immunogen development, the constructs developed here present an opportunity to examine the ability of differentially stabilized S-protein particles to induce two different, yet important antibody responses. First, without wishing to be bound by theory, the disulfide linked ‘down’ state locked double mutant (rS2d) can eliminate receptor binding site targeting antibodies which make up the majority of observed responses^{30,31}. Indeed, a study of MERS responses indicate non-RBD responses (such as NTD and S2 epitopes) will play an important role in vaccine induced protection³². From a theoretical perspective, the wide control over the RBD ‘up’/‘down’ distribution available to the virus indicates that, by analogy to known difficult to neutralize HIV-1 strains, conformational blocking of antibody responses is not be unusual. Although this can result in a fitness cost to the virus, it does not necessarily make the virion non-infectious. Using the double mutant rS2d as an immunogen provides a platform from which to induce such non-RBD responses that can be needed to protect against such an evasion. The second area of interest comprises cryptic pocket targeting antibodies which have proven effective in the neutralization of SARS. These antibodies target an epitope presented only in the ‘up’ state RBDs and appear to require a two RBD ‘up’ configuration³³. The current stabilized ectodomain construct in wide use in SARS-CoV-2 clinical trials was demonstrated previously, and recapitulated here by NSEM, to display only the ‘down’ and one RBD ‘up’ states. However, the u1S2q, SD1/S2 targeting design developed here display a prominent two RBD ‘up’ state distribution compatible with these cryptic-epitope targeting MAbs. This indicates it can induce such antibodies. While complicating factors, such as vaccine enhancement, can favor the use of truncated, single domain constructs which can display fewer weakly or non-neutralizing epitopes, these, along with the designs presented here will allow for a detailed characterization of not only vaccine immunogenicity but also antigenicity, paving the way for next generation vaccines for the new SARS-CoV-2 and the development of a broadly neutralizing β-CoV vaccine. Thus, while the previous generation of stabilizing mutations ensure well folded trimer, the rational design approach developed here provides a means by which precisely controlling the RBD orientation distribution, thus allowing exploratory efforts to understand the role of conformational dynamics from the perspective of vaccine and drug development.

Methods

Vector Based Analysis

Vector analysis was performed using available cryo-EM structures for SARS-2^{13,14}, SARS^{4,5,7,8}, MERS^{4,12}, and other human^{2,10} and murine¹¹ β-CoV spike proteins. Domains for the vector analysis were selected based upon visual inspection of alignments between SARS, MERS, and SARS-CoV-2 structures. Specifically, C_{α} centroids for the S1 NTD, RBD, SD1, SD2 (SARS-CoV-2 residues, 27-43 and 54-271, 330-443 and 503-528, 323-329 and 529-590, 294-322 and 591-696, respectively; equivalent SARS/MERS/Murine/HKU1/OC43 residues selected based upon structural alignment with SARS-CoV-2) as well as a β-sheet motif in the NTD (residues 116-129 and 169-172) and a helix motif in the RBD (residues 403-410) were determined. The NTD was split into two regions with the SD1 contacting, SD2 adjacent portion referred to here as the NTD′ (residues 44-53 and 272-293). C_{α} centroids in the S2 domain were obtained for a β-sheet motif (residues 717-727 and 1047-1071) and the CD domain (711-716 and 1072-1122). Vector magnitudes, angles, and dihedrals between these centroids were determined and used in the subsequent analysis. Vector analysis was performed using the VMD³⁴ Tcl interface. Principal component analysis performed in R with the vector data centered and scaled³⁵.

Rational, Structure-Based Design

Structures for SARS (PDB ID 5X58⁴), MERS (PDB ID 6Q04³⁶), and SARS-CoV-2 (PDB ID 6VXX¹⁵) were prepared in Maestro³⁷ using the protein preparation wizard³⁸ followed by in silico mutagenesis using Schrödinger's cysteine mutation³⁹ and residue scanning⁴⁰ tools. Residue scanning was first performed for individual selected sites allowing mutations to Leu, Ile, Trp, Tyr, and Val followed by scanning of combinations for those which yielded a negative overall score. Scores and visual inspection were used in the selection of the prepared constructs.

Protein Expression and Purification

The SARS-CoV-2 ectodomain constructs were produced and purified as described previously. Briefly, a gene encoding residues 1-1208 of the SARS-CoV-2 S (GenBank: MN908947) with proline substitutions at residues 986 and 987, a “GSAS” (SEQ ID NO: 3) substitution at the furin cleavage site (residues 682-685), a C-terminal T4 fibritin trimerization motif, an HRV3C protease cleavage site, a TwinStrepTag and an 8XHisTag (SEQ ID NO: 4) was synthesized and cloned into the mammalian expression vector pαH. All mutants were introduced in this background. expression plasmids encoding the ectodomain sequence were used to transiently transfect FreeStyle293F cells using Turbo293 (SpeedBiosystems). Protein was purified on the sixth day post transfection from the filtered supernatant using StrepTactin resin (IBA).

Cryo-EM Sample Preparation and Data Collection

Purified SARS-CoV-2 spike preparations were diluted to a concentration of ˜1 mg/mL in 2 mM Tris pH 8.0, 200 mM NaCl and 0.02% NaN3. 2.5 uL of protein was deposited on a CF-1.2/1.3 grid that had been glow discharged for 30 seconds in a PELCO easiGlow™ Glow Discharge Cleaning System. After a 30 s incubation in >95% humidity, excess protein was blotted away for 2.5 seconds before being plunge frozen into liquid ethane using a Leica EM GP2 plunge freezer (Leica Microsystems). Frozen grids were imaged in a Titan Krios (Thermo Fisher) equipped with a K3 detector (Gatan). Data were acquired using the Leginon system⁴¹. The dose was fractionated over 50 raw frames and collected at 50 ms framerate. This dataset was energy-filtered with a slit width of 30 eV. Individual frames were aligned and dose-weighted. CTF estimation, particle picking, 2D classifications, ab initio model generation, heterogeneous refinements, and homogeneous 3D refinements were carried out in cryoSPARC⁴².

Cryo-EM Structure Fitting

Structures of the all ‘down’ state (PDB ID 6VXX) and single RBD ‘up’ state (PDB ID 6VYB) from the previously published SARS-CoV-2 ectodomain were used to fit the cryo-EM maps in Chimera⁴³. The 2 RBD ‘up’ state was generated in PyMol using the single RBD ‘up’ state structure. Mutations were made in PyMol⁴⁴. Coordinates were then fit manually in Coot⁴⁵ following iterative refinement using Phenix⁴⁶ real space refinement and subsequent manual coordinate fitting in Coot. Structure and map analysis was performed using PyMol and Chimera.

### REFERENCES

1 Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
2 Kirchdoerfer, R. N. et al. Pre-fusion structure of a human coronavirus spike protein. Nature 531, 118-121, doi:10.1038/nature17200 (2016).
3 Wrapp, D. et al. Cryo-EM structure of the 2019-nCoV spike in the prefusion conformation. Science 367, 1260-1263, doi:10.1126/science.abb2507 (2020).
4 Yuan, Y. et al. Cryo-EM structures of MERS-CoV and SARS-CoV spike glycoproteins reveal the dynamic receptor binding domains. Nature Communications 8, 15092, doi:10.1038/ncomms15092 (2017).
5 Gui, M. et al. Cryo-electron microscopy structures of the SARS-CoV spike glycoprotein reveal a prerequisite conformational state for receptor binding. Cell Research 27, 119-129, doi:10.1038/cr.2016.152 (2017).
6 Song, W., Gui, M., Wang, X. & Xiang, Y. Cryo-EM structure of the SARS coronavirus spike glycoprotein in complex with its host cell receptor ACE2. PLOS Pathogens 14, e1007236, doi:10.1371/journal.ppat.1007236 (2018).
7 Kirchdoerfer, R. N. et al. Stabilized coronavirus spikes are resistant to conformational changes induced by receptor recognition or proteolysis. Scientific Reports 8, 15701, doi:10.1038/s41598-018-34171-7 (2018).
8 Walls, A. C. et al. Unexpected Receptor Functional Mimicry Elucidates Activation of Coronavirus Fusion. Cell 176, 1026-1039.e1015, doi:https://doi.org/10.1016/j.cell.2018.12.028 (2019).
9 Pallesen, J. et al. Immunogenicity and structures of a rationally designed prefusion MERS-CoV spike antigen. Proceedings of the National Academy of Sciences 114, E7348, doi:10.1073/pnas.1707304114 (2017).
10 Tortorici, M. A. et al. Structural basis for human coronavirus attachment to sialic acid receptors. Nature Structural & Molecular Biology 26, 481-489, doi:10.1038/s41594-019-0233-y (2019).
11 Walls, A. C. et al. Cryo-electron microscopy structure of a coronavirus spike glycoprotein trimer. Nature 531, 114-117, doi:10.1038/nature16988 (2016).
12 Pallesen, J. et al. Immunogenicity and structures of a rationally designed prefusion MERS-CoV spike antigen. Proc Natl Acad Sci USA 114, E7348-E7357, doi:10.1073/pnas.1707304114 (2017).
13 Walls, A. C. et al. Structure, Function, and Antigenicity of the SARS-CoV-2 Spike Glycoprotein. Cell, doi:https://doi.org/10.1016/j.cell.2020.02.058 (2020).
14 Wrapp, D. et al. Cryo-EM structure of the 2019-nCoV spike in the prefusion conformation. Science 367, 1260, doi:10.1126/science.abb2507 (2020).
15 Walls, A. C. et al. Structure, Function, and Antigenicity of the SARS-CoV-2 Spike Glycoprotein. Cell 181, 281-292 e286, doi:10.1016/j.cell.2020.02.058 (2020).
16 Rey, F. A. & Lok, S.-M. Common Features of Enveloped Viruses and Implications for Immunogen Design for Next-Generation Vaccines. Cell 172, 1319-1334, doi:10.1016/j.cell.2018.02.054 (2018).
17 de Taeye, Steven W. et al. Immunogenicity of Stabilized HIV-1 Envelope Trimers with Reduced Exposure of Non-neutralizing Epitopes. Cell 163, 1702-1715, doi:https://doi.org/10.1016/j.cell.2015.11.056 (2015).
18 He, L. et al. HIV-1 vaccine design through minimizing envelope metastability. Science advances 4, eaau6769-eaau6769, doi:10.1126/sciadv.aau6769 (2018).
19 Zhang, P. et al. Interdomain Stabilization Impairs CD4 Binding and Improves Immunogenicity of the HIV-1 Envelope Trimer. Cell Host & Microbe 23, 832-844.e836, doi:https://doi.org/10.1016/j.chom.2018.05.002 (2018).
20 Chuang, G.-Y. et al. Structure-Based Design of a Soluble Prefusion-Closed HIV-1 Env Trimer with Reduced CD4 Affinity and Improved Immunogenicity. Journal of Virology 91, doi:10.1128/JVI.02268-16 (2017).
21 Torrents de la Pella, A. et al. Improving the Immunogenicity of Native-like HIV-1 Envelope Trimers by Hyperstabilization. Cell reports 20, 1805-1817, doi:10.1016/j.celrep.2017.07.077 (2017).
22 Medina-Ramirez, M. et al. Design and crystal structure of a native-like HIV-1 envelope trimer that engages multiple broadly neutralizing antibody precursors in vivo. The Journal of Experimental Medicine 214, 2573, doi:10.1084/jem.20161160 (2017).
23 Steichen, J. M. et al. HIV Vaccine Design to Target Germline Precursors of Glycan-Dependent Broadly Neutralizing Antibodies. Immunity 45, 483-496, doi:10.1016/j.immuni.2016.08.016 (2016).
24 Kulp, D. W. et al. Structure-based design of native-like HIV-1 envelope trimers to silence non-neutralizing epitopes and eliminate CD4 binding. Nature Communications 8, 1655, doi:10.1038/s41467-017-01549-6 (2017).
25 Yang, L. et al. Structure-Guided Redesign Improves NFL HIV Env Trimer Integrity and Identifies an Inter-Protomer Disulfide Permitting Post-Expression Cleavage. Frontiers in Immunology 9, 1631 (2018).
26 Sharma, S. K. et al. Cleavage-independent HIV-1 Env trimers engineered as soluble native spike mimetics for vaccine design. Cell reports 11, 539-550, doi:10.1016/j.celrep.2015.03.047 (2015).
27 Guenaga, J. et al. Structure-Guided Redesign Increases the Propensity of HIV Env To Generate Highly Stable Soluble Trimers. Journal of Virology 90, 2806, doi:10.1128/JVI.02652-15 (2016).
28 Sliepen, K. et al. Structure and immunogenicity of a stabilized HIV-1 envelope trimer based on a group-M consensus sequence. Nature communications 10, 2355-2355, doi:10.1038/s41467-019-10262-5 (2019).
29 Korber, B. et al. Spike mutation pipeline reveals the emergence of a more transmissible form of SARS-CoV-2. bioRxiv, 2020.2004.2029.069054, doi:10.1101/2020.04.29.069054 (2020).
30 Zost, S. J. et al. Rapid isolation and profiling of a diverse panel of human monoclonal antibodies targeting the SARS-CoV-2 spike protein. bioRxiv, 2020.2005.2012.091462, doi:10.1101/2020.05.12.091462 (2020).
31 Brouwer, P. J. M. et al. Potent neutralizing antibodies from COVID-19 patients define multiple targets of vulnerability. bioRxiv, 2020.2005.2012.088716, doi:10.1101/2020.05.12.088716 (2020).
32 Wang, L. et al. Importance of Neutralizing Monoclonal Antibodies Targeting Multiple Antigenic Sites on the Middle East Respiratory Syndrome Coronavirus Spike Glycoprotein To Avoid Neutralization Escape. Journal of virology 92, e02002-02017, doi:10.1128/JVI.02002-17 (2018).
33 Yuan, M. et al. A highly conserved cryptic epitope in the receptor binding domains of SARS-CoV-2 and SARS-CoV. Science 368, 630, doi:10.1126/science.abb7269 (2020).
34 Humphrey, W., Dalke, A. & Schulten, K. VMD: Visual molecular dynamics. Journal of Molecular Graphics 14, 33-38, doi:https://doi.org/10.1016/0263-7855(96)00018-5 (1996).
35 Team, R. C. R: A Language and Environment for Statistical Computing. (2017).
36 Park, Y.-J. et al. Structures of MERS-CoV spike glycoprotein in complex with sialoside attachment receptors. Nature Structural & Molecular Biology 26, 1151-1157, doi:10.1038/s41594-019-0334-7 (2019).
37 Schrödinger Release 2020-1: Maestro (Schrödinger, LLC, New York, NY, 2020).
38 Madhavi Sastry, G., Adzhigirey, M., Day, T., Annabhimoju, R. & Sherman, W. Protein and ligand preparation: parameters, protocols, and influence on virtual screening enrichments. Journal of Computer-Aided Molecular Design 27, 221-234, doi:10.1007/s10822-013-9644-8 (2013).
39 Salam, N. K., Adzhigirey, M., Sherman, W. & Pearlman, D. A. Structure-based approach to the prediction of disulfide bonds in proteins. Protein Engineering, Design and Selection 27, 365-374, doi:10.1093/protein/gzu017 (2014).
40 Beard, H., Cholleti, A., Pearlman, D., Sherman, W. & Loving, K. A. Applying Physics-Based Scoring to Calculate Free Energies of Binding for Single Amino Acid Mutations in Protein-Protein Complexes. PLOS ONE 8, e82849, doi:10.1371/journal.pone.0082849 (2013).
41 Suloway, C. et al. Automated molecular microscopy: the new Leginon system. J Struct Biol 151, 41-60, doi:10.1016/j.jsb.2005.03.010 (2005).
42 Punjani, A., Rubinstein, J. L., Fleet, D. J. & Brubaker, M. A. cryoSPARC: algorithms for rapid unsupervised cryo-EM structure determination. Nat Methods 14, 290-296, doi:10.1038/nmeth.4169 (2017).
43 Pettersen, E. F. et al. UCSF Chimera—A visualization system for exploratory research and analysis. Journal of Computational Chemistry 25, 1605-1612, doi:10.1002/jcc.20084 (2004).
44 Schrodinger, L. The PyMOL Molecular Graphics System. (2015).
45 —Features and development of Coot. —Acta crystallographica. Section D, Biological crystallography -66, -486-501, doi:-(2010).
46 Afonine, P. V. et al. Real-space refinement in PHENIX for cryo-EM and crystallography. Acta Crystallographica Section D 74, 531-544, doi:10.1107/S2059798318006551 (2018).

### Example 3B

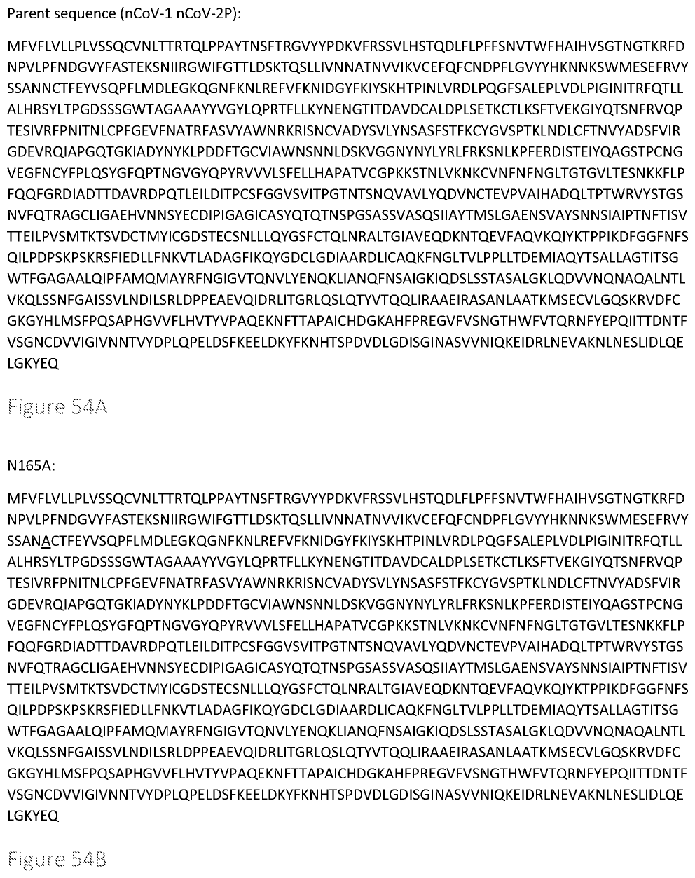

Without being bound by theory, the results in Example 2 indicate that the rs2d can require further modifications. FIG. 53 shows 383C D985C (RBD to S2 double mutant (rS2d) design comprising additional mutations referenced as hexapro mutations. Hexapro mutations are discussed in See Hsieh et al. Science 18 Sep. 2020: Vol. 369, Issue 6510, pp. 1501-1505, DOI: 10.1126/science.abd0826.

Rs2d designs comprising hexapro mutations are evaluated and discussed in Edwards et al. Nature Structural & Molecular Biology volume 28, pages128-131(2021).

FIG. 10 and Table 9B, show SARS-2 designs comprising additional modifications selected from the Cluster designs described in FIG. 8.

Any of the SARS-2 designs will be expressed as nucleic acids or proteins will be expressed, characterized and tested for antigenicity and immunogenicity. Immuonogenicity studies include animal challenge studies.

### Example 4

Glycans on the SARS-CoV-2 Spike Control the Receptor Binding Domain

Conformation

Abstract

The glycan shield of the beta-coronavirus (β-CoV) Spike (S) glycoprotein provides protection from host immune responses, acting as a steric block to potentially neutralizing antibody responses. The conformationally dynamic S-protein is the primary immunogenic target of vaccine design owing to its role in host-cell fusion, displaying multiple receptor binding domain (RBD) ‘up’ and ‘down’ state configurations. Here, we investigated the potential for RBD adjacent, N-terminal domain (NTD) glycans to influence the conformational equilibrium of these RBD states. Using a combination of antigenic screens and high-resolution cryo-EM structure determination, we show that an N-glycan deletion at position 234 results in a dramatically reduced population of the ‘up’ state RBD position. Conversely, glycan deletion at position N165 results in a discernable increase in ‘up’ state RBDs. This indicates the glycan shield acts not only as a passive hinderance to antibody meditated immunity but also as a conformational control element. Together, our results demonstrate this highly dynamic conformational machine is responsive to glycan modification with implications in viral escape and vaccine design.

Introduction

The ongoing SARS-CoV-2 (SARS-2) pandemic presents an urgent need for the development of a protective vaccine. The primary immunogenic target for the vaccines in development is the viral transmembrane S-protein trimer. Each protomer of the trimer is split into an N-terminal receptor binding S1 subunit and a C-terminal fusion element containing S2 subunit, demarcated by the presence of a host protease cleavage site. The S1 subunit is further split into an N-terminal domain (NTD), two subdomains (SD1 and SD2) as well as the receptor binding domain (RBD) that together cap the conserved elements of the S2 subunit. The fusion event is marked by the shedding of the S1 subunit and large conformational transitions in the S2 subunit. The necessity to maintain a large free energy gradient between the prefusion, immune protective state of the molecule and the post-fusion state results in a highly dynamic macromolecular structure. The S1 subunit is dynamic, presenting the RBD in two distinct states: a receptor binding site occluded ‘down’ state in which the RBDs rest against their adjacent protomer's NTD, and a receptor binding site exposed ‘up’ state. It is this RBD ‘up’ state to which the majority of neutralizing responses are observed in convalescent SARS-2 infected individuals′. As conformational evasion is a well-known virus escape mechanism, it is critical to understand the mechanism by which the dynamics are controlled.

Structural studies of the β-CoV S-protein have focused primarily on a soluble, ectodomain construct with and without stabilizing proline mutations (2P). This includes structures for SARS-2^{1,2}, SARS³⁻⁷, MERS^{3,8}, and other human^{9,10} and murine¹¹ β-CoV ectodomains. Structures for the SARS and MERS ectodomains revealed the presence of one and two RBD ‘up’ states with a three RBD ‘up’ state observed in the MERS ectodomain demonstrating the breadth of RBD configurations available to the spike. Interestingly, these states were not observed in the human β-CoVs HKU1 and OC43 nor in a Murine β-CoV, indicating mutations in the spike protein can confer dramatic differences in the propensity of the RBD to sample its available conformational space.

Our quantitative examination of the available β-CoV S-protein structures recently revealed the S1 and S2 subunit domains of different β-CoV viruses occupy a diverse array of configurations¹². Based upon this analysis we predicted the S-protein conformation was sensitive to mutations at the interfaces between domains and subunits. Indeed, mutations at these sites had major impacts on the configuration of the protein, especially on the RBD ‘up’/‘down’ distribution¹². While these and other studies^{13,14,15} have demonstrated the role of protein-protein contacts in determining the conformation of the S-protein, the influence on RBD configuration of glycosylation at or near interfacial domain regions is poorly understood.

Like other class I viral fusion proteins, the β-CoV S-proteins are heavily glycosylated, obscuring the spike surface and limiting the targetable area for immune responses. A recent site-specific analysis of the glycosylation patterns of the SARS-2 S-protein revealed variation in the glycan type, indicating marked differences in processing enzyme accessibility at each site¹⁶. Together, the wide variation in spike conformation coupled with the presence of glycans adjacent to the RBD indicates among the many factors affecting the RBD position, glycosylation patterns can provide a means by which to control its conformational equilibrium.

In this study we have investigated the potential for two SARS-2 NTD glycans in close proximity to the RBD to influence the conformational distribution of the RBD ‘up’ and ‘down’ states. Analysis of the available SARS-2 ‘up’ state structures indicated N165 and N234 glycans can interact with the ‘up’ state RBD acting as both direct stabilizers of the ‘up’ state and as steric blocks to transitions to the ‘down’ state. We combined binding studies by surface plasmon resonance, with structural studies using negative stain electron microscopy (NSEM) and single-particle cryo-electron microscopy (cryo-EM) to define shifts in the ‘up’/‘down’ state equilibrium in glycan-deleted mutants of the SARS-2 spike ectodomain. Together, our results demonstrate that RBD proximal glycans can influence the propensity of the S-protein adopt multiple configurations indicating a means for viral escape and therefore the need to consider non-RBD neutralizing responses in vaccine design.

Results

Structure Analysis Identifies Glycans with the Potential to Modify the S-Protein Conformation

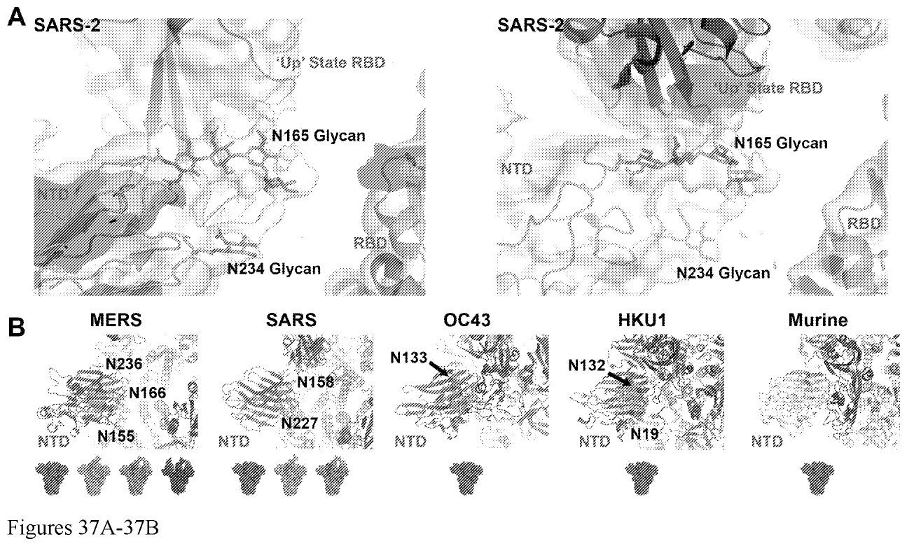

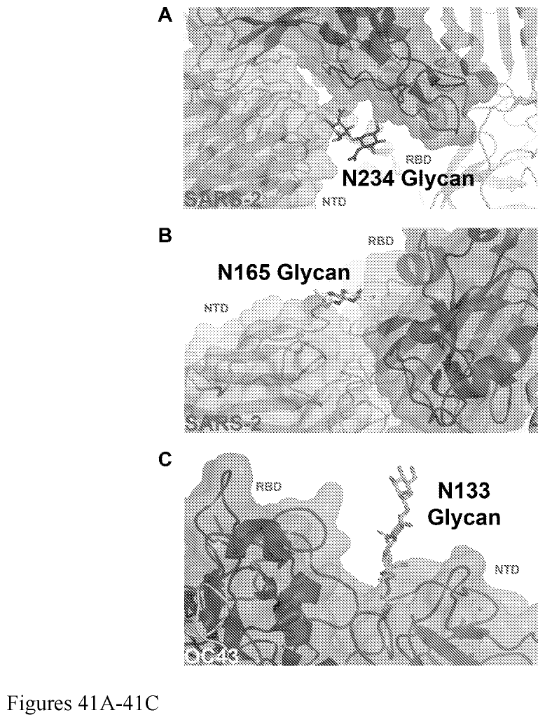

In order to establish whether glycans can indeed alter the RBD orientation, we first examined the SARS-2 glycan density at positions 165 and 234 in the cryo-EM maps from three previously published SARS-2 structures. In the ‘down’ state, the N234 glycan resides in a cleft formed by the NTD and RBD (FIG. 41A) while in the ‘up’ state, it occupies the region of the RBD ‘down’ state (FIG. 37A). This indicates that the solvated ‘up’ state configuration is preferred and must be shifted in order to accommodate the ‘down’ state. The presence of this glycan can act as a hinderance to ‘up’-to-‘down’ state transitions while sterically hindering the ‘down’ state by limiting RBD to NTD packing. An additional glycan at N165 residing toward the apical position of the NTD is in close proximity to the RBD and therefore can also influence the RBD position. Unlike the N234 glycan, the position of the N165 glycan presents no apparent restriction to the RBD positioning in the ‘down’ state (FIG. 41B). However, clear density for this glycan is observed occupying the region the RBD rest in the closed state, potentially forming interactions with the ‘up’ state RBD (FIG. 37A). This indicates this glycan can act to stabilize the RBD ‘up’ state. Alternatively, its presence near the RBD in the ‘down’ state can confer a degree of stability to the fully closed state. Together, these observations combined with our recent results indicating remarkable conformational sensitivity to mutations indicate these glycans can act to stabilize the observed RBD ‘up’/‘down’ equilibrium. We next asked whether RBD proximal NTD glycans occur in other β-CoVs for which high-resolution structural data is available. For this, we examined structures for MERS, SARS, OC43, HKU1, and a Murine β-CoV S-protein ectodomains, identifying three MERS (N155, N166, and N236), two SARS-2 synonymous SARS (N158 and N227), and one OC43 (N133), and two HKU1 (N132 and N19) glycosylation sites proximal to their respective RBDs (FIG. 37B). No RBD adjacent glycosylation sites were observed in the Murine S-protein. While the MERS and SARS glycans display similar extensions into the RBD space in the one ‘up’ state, the OC43 and HKU1 glycans do not. For example, while the HKU1 N132 glycan was poorly resolved, the OC43 N133 glycan occupying the same relative position is observed to extend upward, away from the RBD indicating this glycan does not influence the RBD conformation (FIG. 41C). Interestingly, while cryo-EM reconstructions for SARS-2, MERS, and SARS yield ‘up’ state RBDs, these states were not reported for any of the OC43, HKU1, or Murine datasets. Together, these observations indicate RBD proximal NTD glycans can indeed affect the conformational distribution of ‘up’/‘down’ RBD states.

RBD Conformation and Antigenicity of the N-Glycan Deleted S-Proteins Reveals Differential Stabilization of RBD ‘Up’ and ‘Down’ States

In order to examine the extent to which the N234 and N165 glycans influence the conformational distribution of the S-protein, we produced di-proline (2P) stabilized⁸ S-protein ectodomain² N234A and N165A mutants.

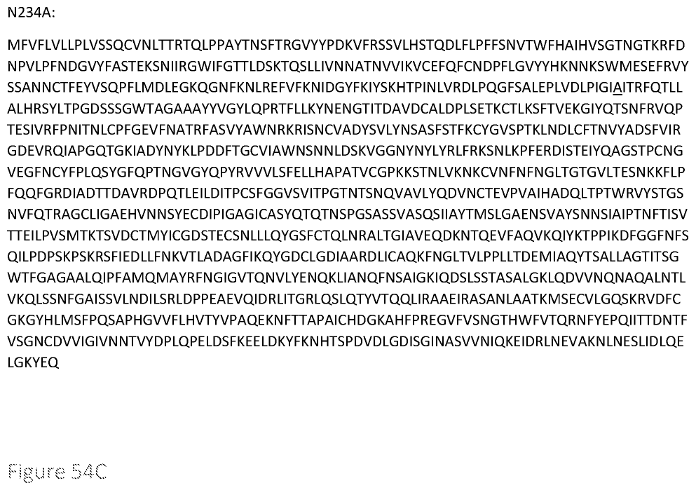

The parent nCoV sequence (“nCoV-1 nCoV-2P”) is shown in FIG. 54A.

The N165A mutant sequence is shown in FIG. 54B.

The N234A mutant sequence is shown in FIG. 54C.

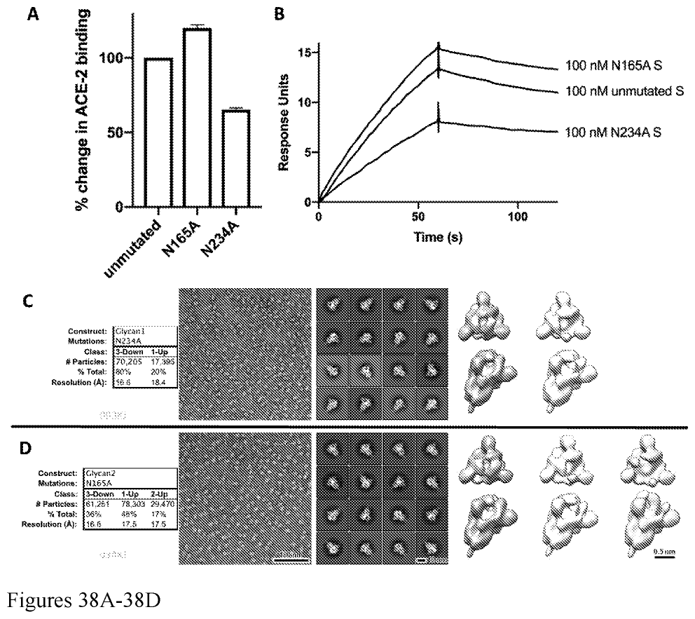

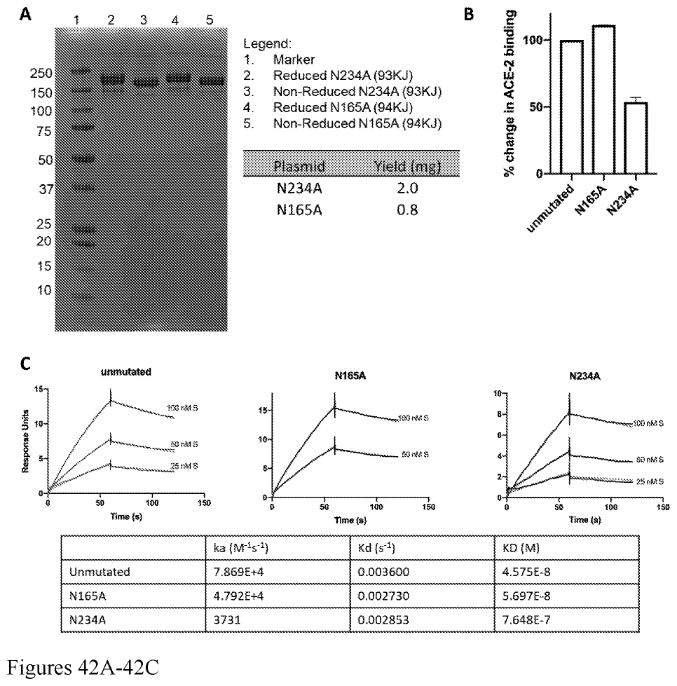

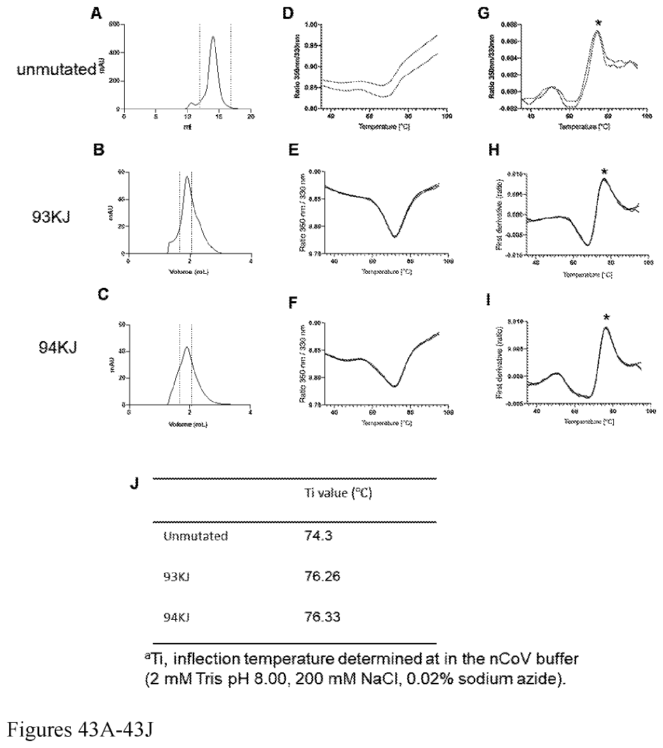

The protein yields after StrepTactin purification were 2.0 mg and 0.8 mg per 1 L culture supernatant, respectively for the N234A and the N165A mutant. (FIGS. 42 and 43). To assess the reactivity of the glycan-deleted spike ectodomain mutants to the ACE-2 receptor, we tested binding of the spike to an ACE-2 ectodomain construct bearing a C-terminal mouse Fc tag immobilized on an anti-Fc surface. SPR binding assays showed that while the N165A mutant displayed ˜10-20% increased binding levels to the unmutated constructs while the N234A mutant showed a decrease of ˜50-60% relative to unmutated construct levels (FIG. 38A and FIG. 42B). Because ACE-2 binding requires the RBD be in the up position, the SPR data indicates that the N165A mutant is more up (or open), whereas the N2345A mutant is more down (or closed).

We next examined the ‘up’/‘down’ state distribution of both mutants via negative stain electron microscopy (NSEM). Heterogenous classification of the N234A mutant particles revealed a dramatic shift from a ˜1:1 ‘up’ v. ‘down’ state distribution in the unmutated 2P^{2,12,17} to a ratio of ˜1:4 in the down state (FIG. 38A). Remarkably, the N165A mutant shifted the distribution in the opposite direction, displaying a higher propensity to adopt RBD “up” states yielding a ˜2:1 -‘up’ state to ‘down’ state ratio, with ˜17% of the “up” population being a 2-RBD “up” class (FIG. 38A). Together, the ACE-2 binding and the NSEM results demonstrated that both NTD N-glycan deletions have distinct impacts on the RBD distribution.

High-Resolution Cryo-EM Structures of the N-Glycan Deleted Constructs Indicates Modest Perturbation to S-Protein Configuration

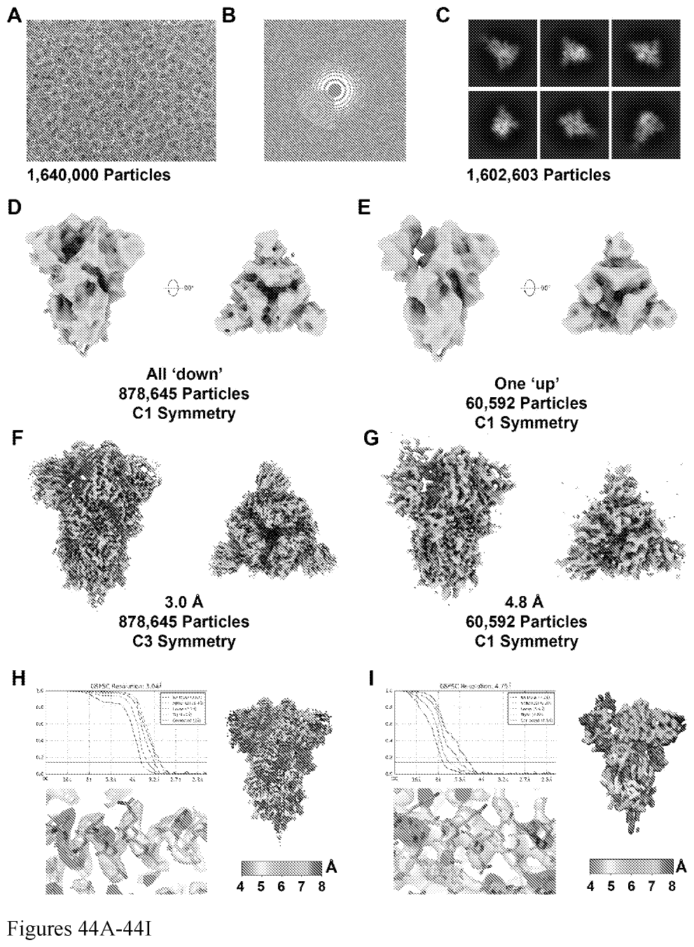

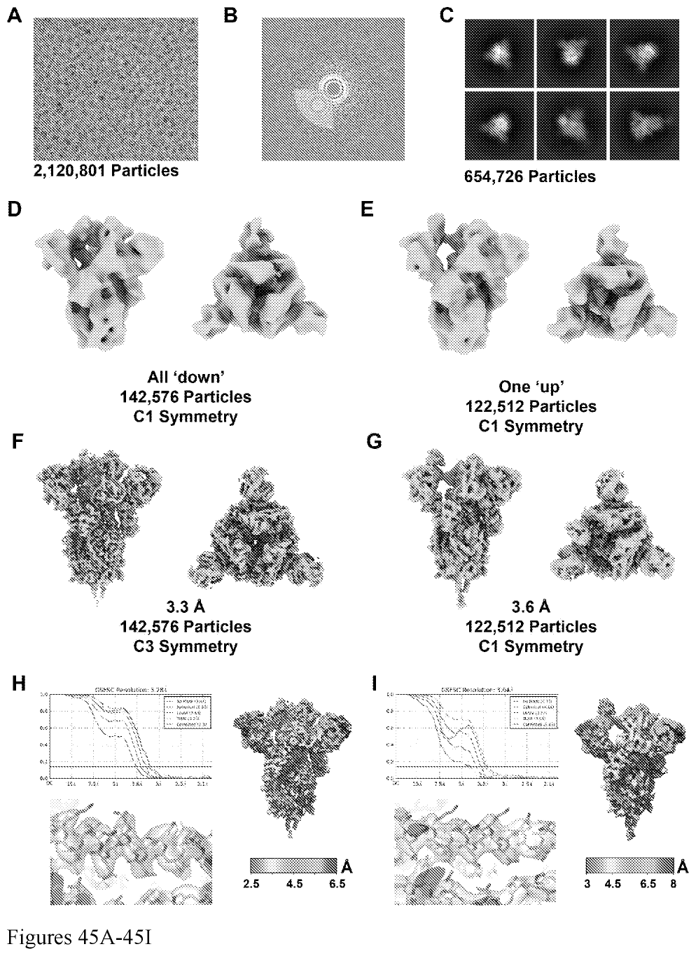

We next turned to cryo-EM for high resolution structure determination to visualize the impact of the glycan deletions on the local and global configuration of the S-protein domains. We collected and processed 7,269 and 8,068 images for the N165A and N234A mutant, respectively, to yield particle stacks cleaned up by 2D classification, that were then subjected to multiple rounds of ab initio classification and heterogenous refinement in cryoSPARC¹⁸ using 20 Å low pass filtered ‘up’ state and ‘down’ state maps generated from available SARS-2 structures. Initial maps for high resolution refinement were generated from sorted particles via ab initio reconstruction (FIGS. 44 and 45). The resulting particle distribution for the N234A mutant was predominantly ‘down’ with a minor, ˜6%, ‘up’ state population while that of the N165A mutant was ˜50% ‘down’ and 50% one ‘up’ as was observed for the unmutated spike previously^{2,12,17}. We were unable to identify a particle subset corresponding to a two ‘up’ state in the cryo-EM dataset. The ‘up’/‘down’ state populations obtained via NSEM for unmutated¹², glutaraldehyde fixed SARS-2 S-protein ectodomain match the previously observed cryo-EM distribution¹⁷. Here, using the same approach, we find that these distributions are dramatically and differentially shifted with mutation of the N165 or N234 to alanine with the SPR, NSEM, and cryo-EM distribution tracking in the same direction with the exception of the N165A cryo-EM particles for which a two RBD ‘up’ state was not observed. Considering the concordance between the SPR and NSEM results, this can be due to particle processing and the potential for a relatively disordered ‘up’ state RBDs in the two ‘up’ state with the glycan deletion.

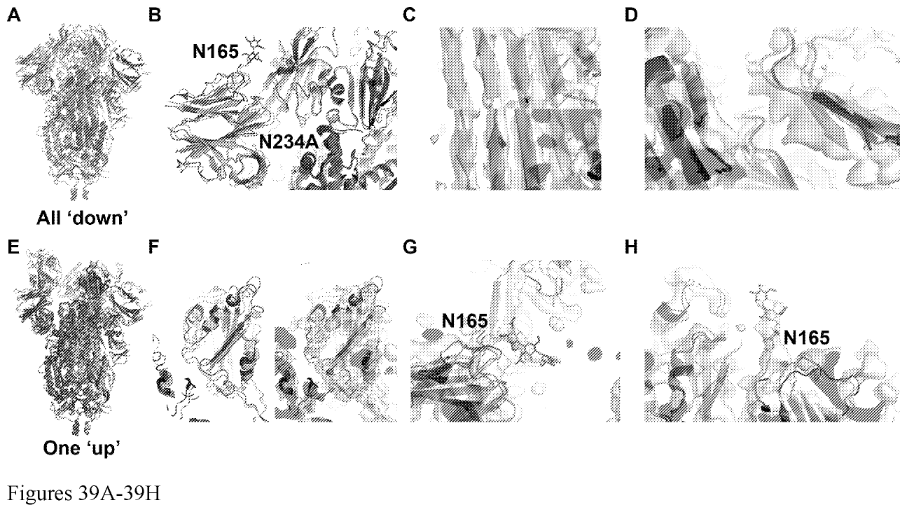

We next examined the high-resolution details of the cryo-EM maps. Refinement of the N234A mutant ‘down’ state using C3 symmetry resulted in a 3.0 Å map with coordinates fit to this map yielding a structure aligning to the unmutated 2P structure (PDB ID 6VXX) with a ˜0.6 Å RMSD. Alignment of the S2 subunit revealed the structures to be nearly identical in these regions (RMSD ˜0.4 Å). Examination of the NTD to RBD interface using this alignment revealed a shift of the NTD toward the RBD (FIG. 39A-D). Weak density for the N165 glycan was observed indicative of an overall similar position relative to that observed previously (FIGS. 39B and C) The one RBD ‘up’ state map was refined to 4.8 Å resolution using C1 symmetry. Comparison of the one RBD ‘up’ state structure fit to this map to its unmutated counterpart (PDB ID 6VYB) indicates a slight shift of the RBD with the N234A mutation (FIGS. 39E and F). However, the limited resolution of this structure limits close examination of this movement. Nevertheless, density for the N165 glycan was observed for the NTD adjacent to the vacant RBD site (‘up’ adjacent) and for the NTD glycan adjacent to the ‘down’ state RBD proximal to the vacant site (‘down’ free). Each occupies a configuration consistent with previous observations in the unmutated form. Interestingly, clear density for the N165 glycan is not observed for the NTD adjacent to the ‘down’ state RBD contacting the ‘up’ state RBD (‘down’ adjacent). Together, the structures show that the while clear differences between the unmutated and N234A mutant are observed, the overall configuration of the structures are similar to their respective unmutated counterparts. These differences do not appear to have significant impacts on the N165 glycan configuration.

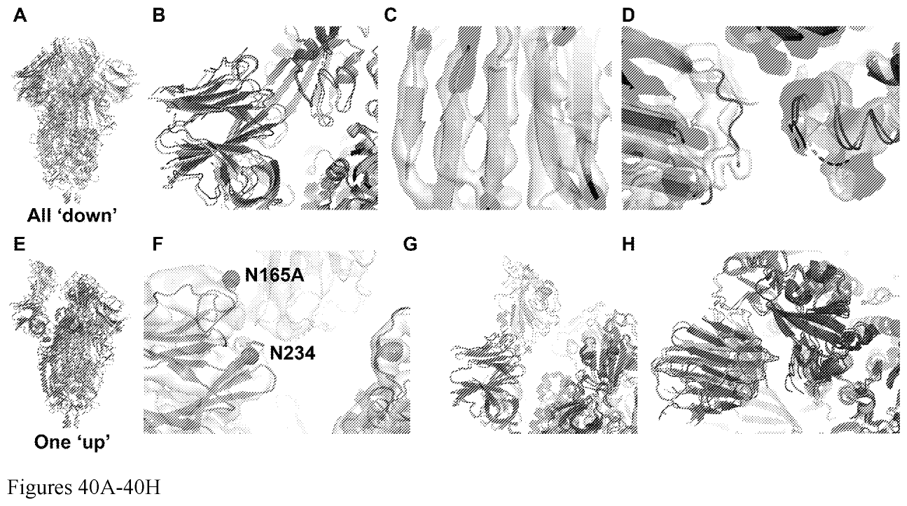

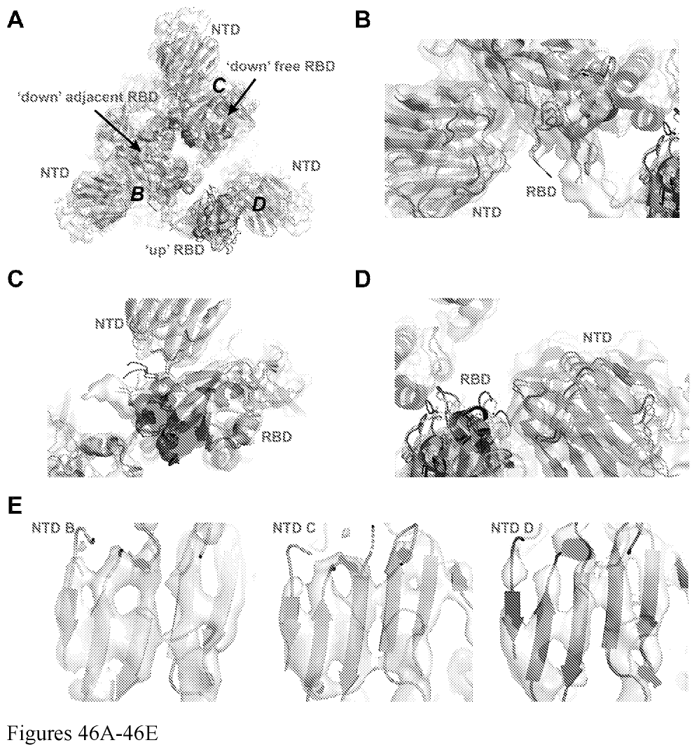

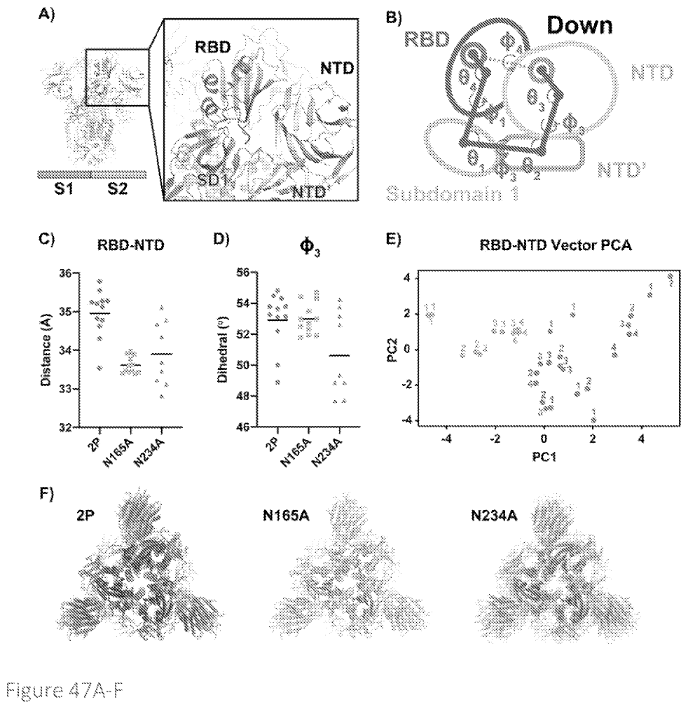

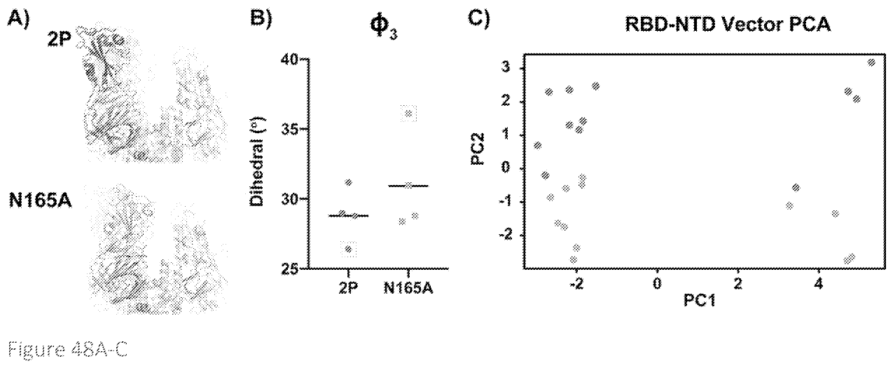

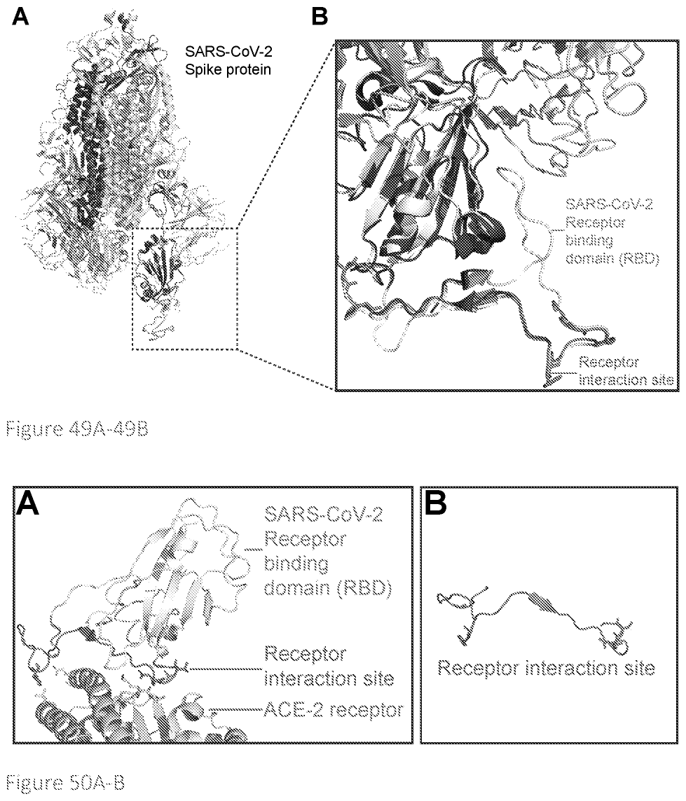

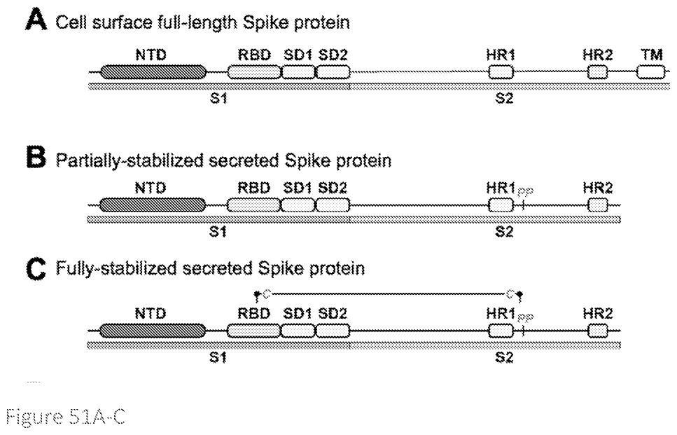

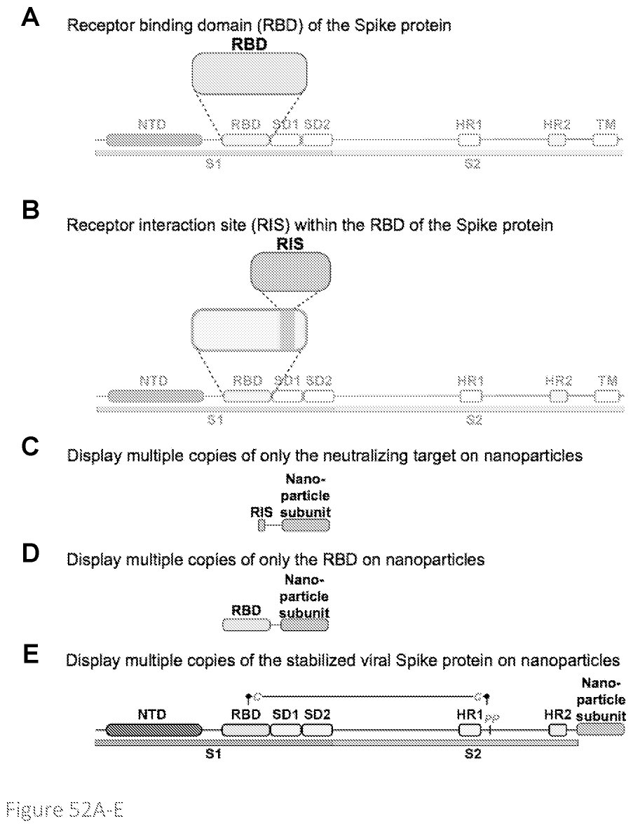

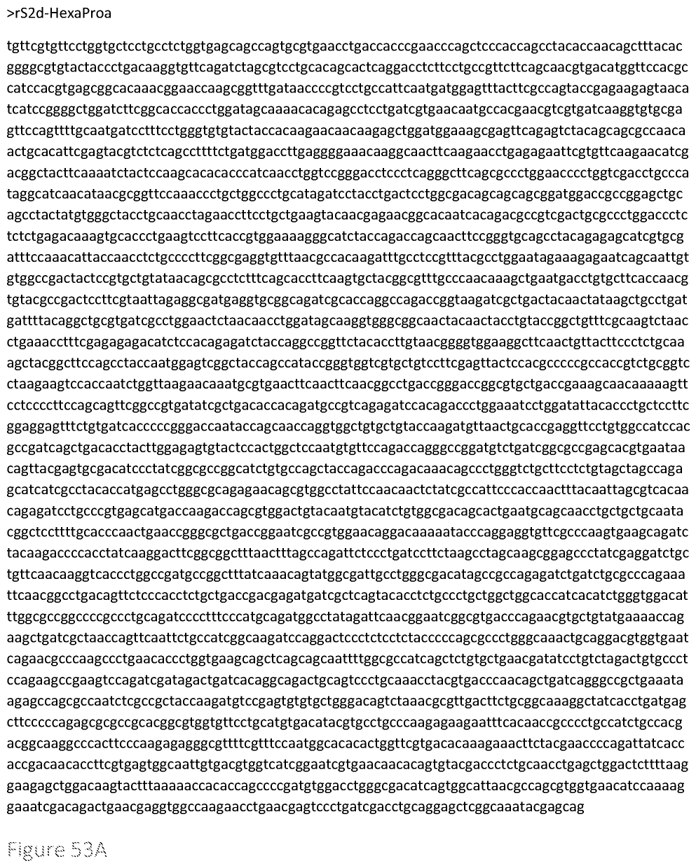

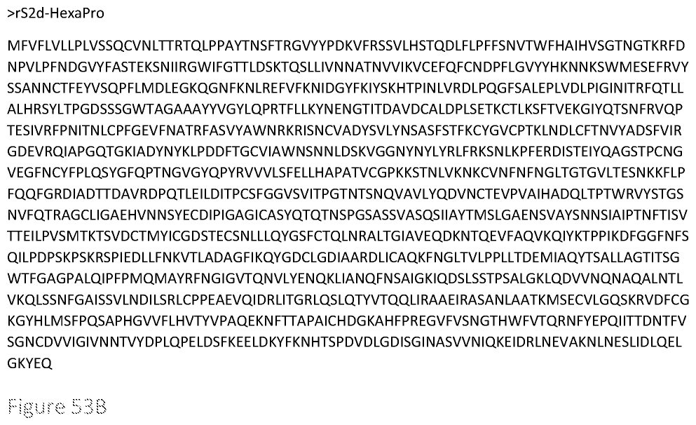

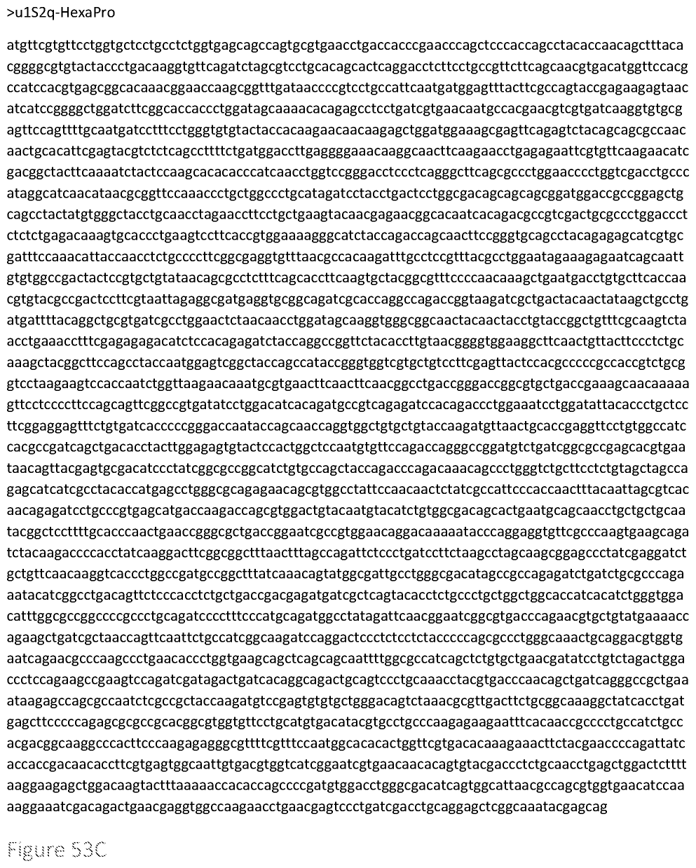

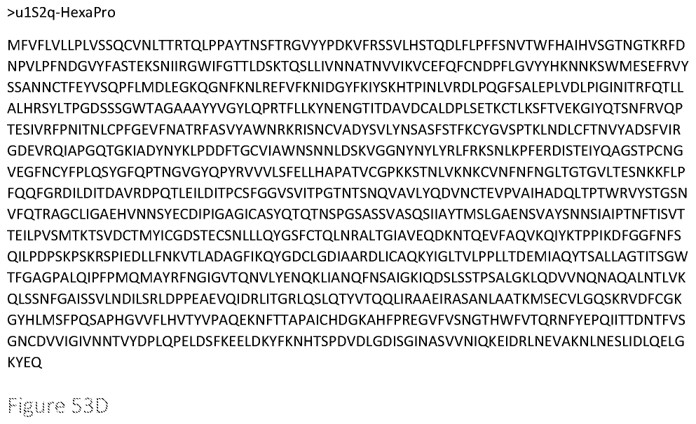

Refinement of the N165A ‘up’ and ‘down’ states resulted in maps with resolutions of 3.6 Å using C1 symmetry and 3.3 Å using C3 symmetry, respectively. Similar to the N234A mutant, the N165A mutant structures showed an overall similar arrangement of the various domains. Alignment of the ‘down’ state structure of the N165A mutant with that of the unmutated spike yielded an RMSD of 0.81 Å with an S2 subunit alignment RMSD of 0.36 Å. Unlike the N234A mutant, the N165A mutant NTD is shifted away from the adjacent RBD (FIG. 40A-D). Interestingly, clear density for the N234 glycan was not observed. The one ‘up’ state structure of the N165A mutant displayed a similar (FIG. 46), albeit slightly less shifted, arrangement of the NTD in the ‘down’ adjacent protomer (FIG. 46B). This shift is not observed in the other two NTDs indicating the NTD shift is sensitive to S1 and S2 subunit arrangements (FIGS. 46C and D). The 1-‘up’ RBD resides in largely the same position as that of the unmutated spike with only minor differences due potentially to the lower relative resolution of this region (FIG. 40E-H). Density for the N234 glycan was not observed for any of the protomers, consistent with the ‘down’ state map. Together, the results of the N165A and N234A structural analysis results indicates that these two glycans play a differential role in influencing the SARS-CoV-2 RBD arrangement, shifting the NTD toward or away from the adjacent RBDs.

Discussion

Viral fusion proteins are often heavily glycosylated with the SARS-2 S-protein being no exception. Though decorated with fewer glycans than the HIV-1 Envelope protein, with 22 glycans per protomer¹⁶, the SARS-2 spike is well shielded from immune surveillance. The SARS-2 spike protein has proven remarkably sensitive to domain-domain interfacial mutations^{12-15,19} which led us to ask whether glycans near the NTD-RBD interface can also impact the configuration of the spike. Here we have investigated the role of two NTD glycans at positions 234 and 165 in modulating S protein conformational dynamics by tracking the shift of RBD disposition in glycan-deleted mutants using binding to ACE-2 receptor, NSEM and cryo-EM analysis. While the specific magnitudes of differences vary between the different analysis methods, all the results track in the same direction to show that deletion of glycan 234 shifts the RBD dynamics more toward the “down” state, whereas deletion of glycan 165, retains or slightly enhances the distribution toward more “up” states. The 2-RBD “up” state observed in the NSEM analysis was not found in the cryo-EM data, indicating that the RBD up/down configuration in this construct can be sensitive to its environment. The shift in the position of the NTD toward the RBD in the ‘down’ state N234A mutant indicates the N234 glycan plays a direct role in destabilizing the ‘down’ state RBD position such that removal allows tighter packing of the RBD to the NTD. Additionally, the observed shift in the position of the ‘up’ state RBD indicates a role for the N234 glycan in modulating RBD stability. This is consistent with a recently released theoretical study investigating ‘up’ state RBD sensitivity to the presence of N165/N234 glycans via molecular simulation²⁰. This investigation found that the absence of these glycans resulted in a comparatively unstable ‘up’ state RBD. The results here confirm the prediction from these simulations that loss of the N234 glycan results in an increased prevalence of the ‘down’ state. Deletion of the glycan at position 165 here indicates an opposite effect on the conformation of the spike relative to the N234A mutant, with the NTD shifting away from the adjacent RBD. Though this appears to relieve strain caused by the restriction imposed by the N234 glycan, the resultant lack of packing between the RBD and NTD can be sufficient to favor transitions to the ‘up’ state. Further, this shift indicates the N165 glycan interacts directly with the RBD. Though direct interactions are not observed in the cryo-EM densities here or in previously published SARS-2 structures, the presence of ‘down’ state conformational heterogeneity evinced by the poor resolution of the RBD and NTD elements of the spike is consistent with the possibility of such an interaction. A more detailed examination of this heterogeneity and the influence of these glycans on the various states of the spike will require large datasets with improved orientational sampling to better resolve these apical regions. Nevertheless, the results here demonstrate that the conformational ensemble of the SARS-2 spike and β-CoV spikes are sensitive to glycosylation patterns, especially near the NTD-RBD interface.

Our results from this study lend insights into two key questions—what role do the glycans at positions 165 and 234 play in modulating RBD dynamics and the biology of the native SARS-2 spike and how do these findings impact vaccine design? Toward the first question, we recognize that the results we describe are in the context of a stabilized, ectodomain construct and differences between these and what occurs on the spike in its native context can be determined. Indeed, a recent report for a detergent solubilized, full-length SARS-2 spike indicated greater stability in the ‘down’ state RBD²¹. Yet our experimental results revealing the role of the N165 and N234 glycans in modulating the conformational landscape of the S protein, taken together with the findings from the computational analysis performed in the context of the full-length spike²⁰, and our analysis of the RBD-proximal NTD glycans of diverse β-CoVs (FIG. 37), provides strong support for a role for these glycans in controlling S protein conformation and dynamics. The differences in glycosylation in this region in different CoV spikes can be a contributor to determining their receptor specificity and thus their transmission. Toward the second question related to the utility for vaccine design, building upon our previous study where we demonstrated conformational control of RBD dynamics in the S protein ectodomain by modulating inter-domain protein-protein contacts, here we expand the tools for achieving such control to glycan-protein interactions, and demonstrate that RBD dynamics can be modulated by targeting key glycans at interdomain contacts. In so doing, we create two new ectodomain constructs with differential exposure of the immunodominant RBD for use as immunogens in vaccination regimens. Taken together, these investigations further demonstrate the remarkable plasticity of this conformational machine and indicate the S-protein has a diverse landscape of conformational escape mutations from which to select as genetic drift and host immune pressures direct its evolution.

Studies have shown that the NTD and RBD are quite mobile. We therefore asked whether the observed shifts in the NTD of the N165A and N234A mutants in the ‘down’ state are related to changes in the propensity of the domain to occupy positions or due to access to new states. We first classified the ‘down’ state 2P, N165A, and N234A particles using C1 symmetry yielding 4, 4, and 3 states, respectively. In order to quantify differences in the positions of the S1 domains, we generated a set of vectors between protomer RBD's and SD1's centroids and their adjacent NTD's centroids (FIGS. 47A and B).

Vector magnitudes and relevant angles and dihedrals were determined for each of the three RBD-NTD pairings. Examination of the distance between adjacent RBDs and NTDs revealed markedly shifted positions between the three constructs (FIG. 47C). The geometric mean distance of the 2P construct positions was 35.0 Å compared to 33.6 Å for the N165A construct and 33.9 Å for the N234A construct. The N234A results appeared roughly bimodal with a population average near that of the 2P construct of 34.7 Å and another nearer the N165A average with an average of 33.3 Å. A single 2P RBD-NTD pair reached this N165A like state. The N165A mutant displayed a tight distribution with a standard deviation of 0.2 Å compared to 0.6 Å and 0.8 Å for the 2P and N234A constructs, respectively. We next examined the disposition of the NTD relative to the RBD via a dihedral about the SD1 and NTD′ vector.

The results indicate the 2P and N165A constructs display similar angles with geometric means of 52.9 And 53.0 Å, respectively (FIG. 47D). As in the RBD to NTD distance metric, the N234A construct displays a bimodal distribution, one close to that of the 2P and N165A constructs with a geometric mean of 53.4 Å and another with a geometric mean of 48.5 Å. Two of the 2P RBD-NTD pairings display values near this lower angle state. As observed for the RBD-NTD distance metric, the N165A construct displays a tight distribution (1.0 Å SD) while those of the 2P and N234A are wider (1.8 and 2.7 Å, respectively). We next projected the vector dataset using the principal components analysis method (PCA) to examine aggregate differences between the pairing arrangements. The N165A pairings separated from the 2P and N234A along principal component one while principal component two provided limited separation between the 2P and N234A constructs. Examination of the pairings within each structure revealed marked similarity between N165A pairs while those of the 2P and N234A constructs largely dissimilar. This indicated that the N165A states were more symmetric than those of 2P and N234A constructs. Visualization of the alignments of the S2 regions of each construct's coordinates is consistent with this observation (FIG. 47F). These results indicate the N165 glycan plays a role in stabilizing asymmetric S1 arrangements while the N234 glycan appears to affect the relative stabilities of these states.

A previous molecular dynamics-based study of the one ‘up’ state RBD indicated the N165 glycan “props up” the RBD. We therefore classified the 2P and N165A construct ‘up’ states in order to determine the extent to which the RBD positions. Each classified into four states with some overlap in the relative position of the RBDs. However, the 2P construct displayed an RBD more distant from the primary trimer axis as compared to those of the N165A construct while the N165A construct displayed a state much closer to the primary axis (FIG. 48A). This is exemplified in the ϕ3 dihedral which shows that, while each contains three states that are quite similar, the 2P trimer axis distant state and the N165A close state differ (FIG. 48B). This is consistent with the previous observations, indicated the N165 glycan indeed limits access of the RBD to S2 region of the trimer. We next performed a PCA analysis of the ‘up’ state vectors. Principal component one separates the ‘up’ and ‘down’ state pairing while principal component two separates the two constructs (FIG. 48C). This indicates that the N165 glycan plays a role in not only propping up the RBD but also in determining the arrangement of the S1 domains.

Methods

Vector Based Analysis

Vector analysis was performed as previously described. Specifically, Cα centroids for the S1 NTD, RBD, S131, SD2 (SARS-CoV-2 residues, 27-43 and 54-271, 330-443 and 503-528, 323-329 and 529-590, 294-322 and 591-696, respectively) as well as a β-sheet motif in the NTD (residues 116-129 and 169-172) and a helix motif in the RBD (residues 403-410) were determined. The NTD was split into two regions with the SD1 contacting, SD2 adjacent portion referred to here as the NTD′ (residues 44-53 and 272-293). Cα centroids in the S2 subunit were obtained for a β-sheet motif (residues 717-727 and 1047-1071) and the CD domain (711-716 and 1072-1122). Vector magnitudes, angles, and dihedrals between these centroids were determined and used in the subsequent analysis. Vector analysis was performed using the VMD²² Tcl interface.

Protein Expression and Purification

The SARS-CoV-2 ectodomain constructs were produced and purified as described previously². Briefly, a gene encoding residues 1-1208 of the SARS-CoV-2 S (GenBank: MN908947) with proline substitutions at residues 986 and 987, a “GSAS” substitution at the furin cleavage site (residues 682-685), a C-terminal T4 fibritin trimerization motif, an HRV3C protease cleavage site, a TwinStrepTag and an 8XHisTag (SEQ ID NO: 4) was synthesized and cloned into the mammalian expression vector pall. All mutants were introduced in this background. Expression plasmids encoding the ectodomain sequence were used to transiently transfect FreeStyle293F cells using Turbo293 (SpeedBiosystems). Protein was purified on the sixth day post-transfection from the filtered supernatant using StrepTactin resin (IBA).

The ACE-2 gene was cloned as a fusion protein with a mouse Fc region attached to its C-terminal end. A 6× His-tag (SEQ ID NO: 5) was added to the C-terminal end of the Fc domain. ACE-2 with mouse FC tag was purified by Ni-NTA chromatography.

Thermal Shift Assay

The thermal shift assay was performed using Tycho NT. 6 (NanoTemper Technologies). Spike variants were diluted (0.15 mg/ml) in nCoV buffer (2 mM Tris, pH 8.0, 200 mM NaCl, 0.02% sodium azide) and run in duplicates in capillary tubes. Intrinsic fluorescence was recorded at 330 nm and 350 nm while heating the sample from 35-95° C. at a rate of 3° C./min. The ratio of fluorescence (350/330 nm) and the Ti were calculated by Tycho NT. 6.

Cryo-EM Sample Preparation, Data Collection and Processing

Purified SARS-CoV-2 spike preparations were diluted to a concentration of ˜1 mg/mL in 2 mM Tris pH 8.0, 200 mM NaCl and 0.02% NaN3. 2.5 μL of protein was deposited on a CF-1.2/1.3 grid that had been glow discharged for 30 seconds in a PELCO easiGlow™ Glow Discharge Cleaning System. After a 30 s incubation in >95% humidity, excess protein was blotted away for 2.5 seconds before being plunge frozen into liquid ethane using a Leica EM GP2 plunge freezer (Leica Microsystems). Frozen grids were imaged in a Titan Krios (Thermo Fisher) equipped with a K3 detector (Gatan). Data were acquired using the Leginon system²³. The dose was fractionated over 50 raw frames and collected at 50 ms framerate. This dataset was energy-filtered with a slit width of 30 eV. Individual frames were aligned and dose-weighted′. CTF estimation, particle picking, 2D classifications, ab initio model generation, heterogeneous refinements, homogeneous 3D refinements and local resolution calculations were carried out in cryoSPARC²⁵.

Cryo-EM Structure Fitting and Analysis

Structures of the all ‘down’ state (PDB ID 6VXX) and single RBD ‘up’ state (PDB ID 6VYB) from the previously published SARS-CoV-2 ectodomain were used to fit the cryo-EM maps in Chimera²⁶. Mutations were made in PyMol²⁷. Coordinates were fit to the maps first using ISOLDE²⁸ followed by iterative refinement using Phenix²⁹ real space refinement and subsequent manual coordinate fitting in Coot as needed. Structure and map analysis were performed using PyMol, Chimera²⁶ and ChimeraX³⁰.

Surface Plasmon Resonance

The binding of ACE-2 to the SARS-2 spike constructs was assessed by surface plasmon resonance on Biacore T-200 (GE-Healthcare) at 25° C. with HBS-EP+ (10 mM HEPES, pH 7.4, 150 mM NaCl, 3 mM EDTA, and 0.05% surfactant P-20) as the running buffer. ACE-2 tagged at its C-terminal end to a mouse Fc region was captured on an anti-Fc surface. Binding was assessed by flowing over different concentrations of the spike constructs over the ACE-2 surface. The surface was regenerated between injections by flowing over 3M MgCl2 solution for 10s with flow rate of 100 μl/min. Blank sensorgrams were obtained by injection of the same volume of HBS-EP+ buffer in place of IgGs and Fab solutions. Sensorgrams were corrected with corresponding blank curves. Sensorgram data were analyzed using the BiaEvaluation software (GE Healthcare).

### REFERENCES

1 Barnes, C. O. et al. Structures of human antibodies bound to SARS-CoV-2 spike reveal common epitopes and recurrent features of antibodies. Cell, doi:https://doi.org/10.1016/j.cell.2020.06.025 (2020).
2 Wrapp, D. et al. Cryo-EM structure of the 2019-nCoV spike in the prefusion conformation. Science 367, 1260-1263, doi:10.1126/science.abb2507 (2020).
3 Yuan, Y. et al. Cryo-EM structures of MERS-CoV and SARS-CoV spike glycoproteins reveal the dynamic receptor binding domains. Nature Communications 8, 15092, doi:10.1038/ncomms15092 (2017).
4 Gui, M. et al. Cryo-electron microscopy structures of the SARS-CoV spike glycoprotein reveal a prerequisite conformational state for receptor binding. Cell Research 27, 119-129, doi:10.1038/cr.2016.152 (2017).
5 Song, W., Gui, M., Wang, X. & Xiang, Y. Cryo-EM structure of the SARS coronavirus spike glycoprotein in complex with its host cell receptor ACE2. PLOS Pathogens 14, e1007236, doi:10.1371/journal.ppat.1007236 (2018).
6 Kirchdoerfer, R. N. et al. Stabilized coronavirus spikes are resistant to conformational changes induced by receptor recognition or proteolysis. Scientific Reports 8, 15701, doi:10.1038/s41598-018-34171-7 (2018).
7 Walls, A. C. et al. Unexpected Receptor Functional Mimicry Elucidates Activation of Coronavirus Fusion. Cell 176, 1026-1039.e1015, doi:https://doi.org/10.1016/j.cell.2018.12.028 (2019).
8 Pallesen, J. et al. Immunogenicity and structures of a rationally designed prefusion MERS-CoV spike antigen. Proceedings of the National Academy of Sciences 114, E7348, doi:10.1073/pnas.1707304114 (2017).
9 Kirchdoerfer, R. N. et al. Pre-fusion structure of a human coronavirus spike protein. Nature 531, 118-121, doi:10.1038/nature17200 (2016).
10 Tortorici, M. A. et al. Structural basis for human coronavirus attachment to sialic acid receptors. Nature Structural & Molecular Biology 26, 481-489, doi:10.1038/s41594-019-0233-y (2019).
11 Walls, A. C. et al. Cryo-electron microscopy structure of a coronavirus spike glycoprotein trimer. Nature 531, 114-117, doi:10.1038/nature16988 (2016).
12 Henderson, R. et al. Controlling the SARS-CoV-2 Spike Glycoprotein Conformation. bioRxiv, 2020.2005.2018.102087, doi:10.1101/2020.05.18.102087 (2020).
13 Hsieh, C.-L. et al. Structure-based Design of Prefusion-stabilized SARS-CoV-2 Spikes. bioRxiv, 2020.2005.2030.125484, doi:10.1101/2020.05.30.125484 (2020).
14 McCallum, M., Walls, A. C., Corti, D. & Veesler, D. Closing coronavirus spike glycoproteins by structure-guided design. bioRxiv, 2020.2006.2003.129817, doi:10.1101/2020.06.03.129817 (2020).
15 Xiong, X. et al. A thermostable, closed, SARS-CoV-2 spike protein trimer. bioRxiv, 2020.2006.2015.152835, doi:10.1101/2020.06.15.152835 (2020).
16 Watanabe, Y., Allen, J. D., Wrapp, D., McLellan, J. S. & Crispin, M. Site-specific glycan analysis of the SARS-CoV-2 spike. Science, eabb9983, doi:10.1126/science.abb9983 (2020).
17 Walls, A. C. et al. Structure, Function, and Antigenicity of the SARS-CoV-2 Spike Glycoprotein. Cell, doi:https://doi.org/10.1016/j.cell.2020.02.058 (2020).
18 Punjani, A., Rubinstein, J. L., Fleet, D. J. & Brubaker, M. A. cryoSPARC: algorithms for rapid unsupervised cryo-EM structure determination. Nature Methods 14, 290, doi:10.1038/nmeth.4169 https://www.nature.com/articles/nmeth.4169 #supplementary-information (2017).
19 Zhang, L. et al. The D614G mutation in the SARS-CoV-2 spike protein reduces S1 shedding and increases infectivity. bioRxiv, 2020.2006.2012.148726, doi:10.1101/2020.06.12.148726 (2020).
20 Casalino, L. et al. Shielding and Beyond: The Roles of Glycans in SARS-CoV-2 Spike Protein. bioRxiv, 2020.2006.2011.146522, doi:10.1101/2020.06.11.146522 (2020).
21 Cai, Y. et al. Distinct conformational states of SARS-CoV-2 spike protein. bioRxiv, 2020.2005.2016.099317, doi:10.1101/2020.05.16.099317 (2020).
22 Humphrey, W., Dalke, A. & Schulten, K. VMD: Visual molecular dynamics. Journal of Molecular Graphics 14, 33-38, doi:https://doi.org/10.1016/0263-7855(96)00018-5 (1996).
23 Suloway, C. et al. Automated molecular microscopy: the new Leginon system. J Struct Biol 151, 41-60, doi:10.1016/j.jsb.2005.03.010 (2005).
24 Zheng, S. Q. et al. MotionCor2: anisotropic correction of beam-induced motion for improved cryo-electron microscopy. Nat Methods 14, 331-332, doi:10.1038/nmeth.4193 (2017).
25 Punjani, A., Rubinstein, J. L., Fleet, D. J. & Brubaker, M. A. cryoSPARC: algorithms for rapid unsupervised cryo-EM structure determination. Nat Methods 14, 290-296, doi:10.1038/nmeth.4169 (2017).
26 Pettersen, E. F. et al. UCSF Chimera—A visualization system for exploratory research and analysis. Journal of Computational Chemistry 25, 1605-1612, doi:10.1002/jcc.20084 (2004).
27 Schrodinger, L. The PyMOL Molecular Graphics System. (2015).
28 Croll, T. ISOLDE: a physically realistic environment for model building into low-resolution electron-density maps. Acta Crystallographica Section D 74, 519-530, doi:10.1107/S2059798318002425 (2018).
29 Afonine, P. V. et al. Real-space refinement in PHENIX for cryo-EM and crystallography. Acta Crystallographica Section D 74, 531-544, doi:10.1107/S2059798318006551 (2018).
30 Goddard, T. D. et al. UCSF ChimeraX: Meeting modern challenges in visualization and analysis. Protein Sci 27, 14-25, doi:10.1002/pro.3235 (2018).

1. A modified SARS-2 spike protein comprising amino acid changes as described in FIG. 8, 10 or 25.

2. The modified SARS-2 spike protein of claim 1, wherein the protein comprises a recombinant protein comprising all the consecutive amino acids after the signal peptide of polypeptide sequences in FIG. 8, 10 or 25.

3. The modified SARS-2 spike protein of claim 1 comprising S383C D985C (RBD to S2 double mutant; rS2d) mutations.

4. A nucleic acid encoding the modified SARS-2 spike protein of claim 1.

5. The nucleic acid of claim 4, wherein the nucleic acid is a modified mRNA.

6. The nucleic acid of claim 5, wherein the mRNA is in a composition comprising lipid nanoparticles.

7. The nucleic acid of claim 4, wherein the nucleic acid is comprised in a vector and is operably linked to a promoter.

8. A composition comprising the modified SARS-2 spike protein of claim 2 or a nucleic acid encoding the modified SARS-2 spike protein, and a carrier.

9. (canceled)

10. A protein nanoparticle or virus-like particle (VLP), comprising the modified SARS-2 spike protein of claim 2.

11. (canceled)

12. A host cell comprising a nucleic acid molecule encoding the modified SARS-2 spike protein of claim 2.

13. An immunogenic composition comprising the modified SARS-2 spike protein of claim 1, a nucleic acid encoding the modified SARS-2 spike protein, a nanoparticle or VLP including the modified SARS-2 spike protein, and a pharmaceutically acceptable carrier.

14. A method for inducing an immune response to a SARS- 2 spike protein in a subject, comprising administering to the subject an effective amount of the modified SARS-2 spike protein of claim 1, a nucleic acid encoding the modified SARS spike protein, or a composition thereof.

15. A modified SARS-2 spike protein, comprising an amino acid sequence of an N165A variant or an N234A variant.

16. The modified SARS-2 spike protein of claim 15, comprising all the consecutive amino acids after the signal peptide of a modified SARS-2 spike protein comprising the amino acid sequence of the N165A variant or the N234A variant.

17-28. (canceled)
